SEQUENCE LISTING (58 sequences)

SEQ ID NO: 1 - protein, 7 aa, Artificial sequence
  Synthetic polypeptide
SSISSNY

SEQ ID NO: 2 - protein, 3 aa, Artificial sequence
  Synthetic polypeptide
RTS

SEQ ID NO: 3 - protein, 18 aa, Artificial sequence
  Synthetic polypeptide
QQGSSIPRTRSEGAPSWK

SEQ ID NO: 4 - protein, 8 aa, Artificial sequence
  Synthetic polypeptide
GYTFTSYW

SEQ ID NO: 5 - protein, 8 aa, Artificial sequence
  Synthetic polypeptide
IGPNSGGT

SEQ ID NO: 6 - protein, 22 aa, Artificial sequence
  Synthetic polypeptide
ARRMVKGCYGLLGPRDHGHRLL

SEQ ID NO: 7 - protein, 11 aa, Artificial sequence
  Synthetic polypeptide
QSIVHSNGNTY

SEQ ID NO: 8 - protein, 3 aa, Artificial sequence
  Synthetic polypeptide
KVS

SEQ ID NO: 9 - protein, 9 aa, Artificial sequence
  Synthetic polypeptide
FQGSHVPYT

SEQ ID NO: 10 - protein, 8 aa, Artificial sequence
  Synthetic polypeptide
GYTFTDYY

SEQ ID NO: 11 - protein, 8 aa, Artificial sequence
  Synthetic polypeptide
INPNNGGT

SEQ ID NO: 12 - protein, 11 aa, Artificial sequence
  Synthetic polypeptide
ARYDYAWYFDV

SEQ ID NO: 13 - protein, 107 aa, Artificial sequence
  Synthetic polypeptide
DIELTQSPTTMAASPGEKITITCSASSSISSNYLHWYQQKPGFSPKLLIYRTSNLASGVPARFSGSGSGTSYSLTIGTMEAEDVATYYCQQGSSIPRTRSEGAPSWK

SEQ ID NO: 14 - protein, 112 aa, Artificial sequence
  Synthetic polypeptide
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPYTFGGGTKLEIK

SEQ ID NO: 15 - protein, 117 aa, Artificial sequence
  Synthetic polypeptide
VKLQESGAELVKPGASVKLSCKASGYTFTSYWMHWVKQRPGRGLEWIGRIGPNSGGTKYNEKFKSKATLTVDKPSSTAYMQLSSLTSEDSAVYYCARRMVKGCYGLLGPRDHGHRLL

SEQ ID NO: 16 - protein, 117 aa, Artificial sequence
  Synthetic polypeptide
VKLQESGPELVKPGASVKISCKASGYTFTDYYMNWVKQSHGKSLEWIGDINPNNGGTSYNQKFKGKATLTVDKSSSTAYMELRSLTSEDSAVYYCARYDYAWYFDVWGQGTTVTVSS

SEQ ID NO: 17 - protein, 272 aa, Artificial sequence
  Synthetic polypeptide
HHHHHHVKLQESGAELVKPGASVKLSCKASGYTFTSYWMHWVKQRPGRGLEWIGRIGPNSGGTKYNEKFKSKATLTVDKPSSTAYMQLSSLTSEDSAVYYCARRMVKGCYGLLGPRDHGHRLLKGRIPAHWRPLLVDPSSVPSLASGGGGGSGGGGSWISAEFALDIELTQSPTTMAASPGEKITITCSASSSISSNYLHWYQQKPGFSPKLLIYRTSNLASGVPARFSGSGSGTSYSLTIGTMEAEDVATYYCQQGSSIPRTRSEGAPSWK

SEQ ID NO: 18 - protein, 274 aa, Artificial sequence
  Synthetic polypeptide
MSVPTQVLGLLLLWLTDARCVKLQESGPELVKPGASVKISCKASGYTFTDYYMNWVKQSHGKSLEWIGDINPNNGGTSYNQKFKGKATLTVDKSSSTAYMELRSLTSEDSAVYYCARYDYAWYFDVWGQGTTVTVSSGGGGSGGGGSGGGGDVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPYTFGGGTKLEIKRIEGRHHHHHH

SEQ ID NO: 19 - DNA, 321 bp, Artificial sequence
  Synthetic polynucleotide
gacattgagctcacccagtctccaaccaccatggctgcatctcccggggagaagatcactatcacctgcagtgccagctcaagtataagttccaattacttgcattggtatcagcagaagccaggattctcccctaaactcttgatttataggacatccaatctggcttctggagtcccagctcgcttcagtggcagtgggtctgggacctcttactctctcacaattggcaccatggaggctgaagatgttgccacttactactgccagcagggtagtagtataccacgtacacgttcggagggggcaccaagctggaaa

SEQ ID NO: 20 - DNA, 338 bp, Artificial sequence
  Synthetic polynucleotide
gatgttttgatgacccaaactccactctccctgcctgtcagtcttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttccgtacacgttcggaggggggaccaagctggaaataaaacg

SEQ ID NO: 21 - DNA, 352 bp, Artificial sequence
  Synthetic polynucleotide
gtgaaactgcaggagtcaggggctgagcttgtgaagcctggggcttcagtgaagctgtcctgcaaggcttctggctacaccttcaccagctactggatgcactgggtgaagcagaggcctggacgaggccttgagtggattggaaggattggtcctaatagtggtggtactaagtacaatgagaagttcaagagcaaggccacactgactgtagacaaaccctccagcacagcctacatgcagctcagcagcctgacatctgaggactctgcggtctattattgtgcaagaagaatggtaaaggggtgctatggactactggggccaagggaccacggtcaccgtctcctca

SEQ ID NO: 22 - DNA, 351 bp, Artificial sequence
  Synthetic polynucleotide
gtgaagctgcaggagtctggacctgagctggtgaagcctggggcttcagtgaagatatcctgtaaggcttctggatacacgttcactgactactacatgaactgggtgaagcagagccatggaaagagccttgagtggattggagatattaatcctaacaatggtggtactagctacaaccagaagttcaagggcaaggccacattgactgtagacaagtcctccagcacagcctacatggagctccgcagcctgacatctgaggactctgcagtctattactgtgcaagatatgattacgcttggtacttcgatgtctggggccaagggaccacggtcaccgtctcctca

SEQ ID NO: 23 - DNA, 837 bp, Artificial sequence
  Synthetic polynucleotide
gccgccaccatgagtgtgcccactcaggtcctggggttgctgctgctgtggcttacagatgccagatgtgtgaagctgcaggagtctggacctgagctggtgaagcctggggcttcagtgaagatatcctgtaaggcttctggatacacgttcactgactactacatgaactgggtgaagcagagccatggaaagagccttgagtggattggagatattaatcctaacaatggtggtactagctacaaccagaagttcaagggcaaggccacattgactgtagacaagtcctccagcacagcctacatggagctccgcagcctgacatctgaggactctgcagtctattactgtgcaagatatgattacgcttggtacttcgatgtctggggccaagggaccacggtcaccgtctcctcaggcggaggtgggtcgggtggcggcggatctggcggaggtggggatgttttgatgacccaaactccactctccctgcctgtcagtcttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttccgtacacgttcggaggggggaccaagctggaaataaaacggatcgaaggccggcatcaccatcatcaccactgatag

SEQ ID NO: 24 - DNA, 825 bp, Artificial sequence
  Synthetic polynucleotide
atgtccgtgcctacccaggtgctcggactcctgctgctgtggctcaccgacgccaggtgtgtgaagctgcaggagagcggacccgagctggtgaagcctggagcctccgtgaagatcagctgcaaggcttccggatacaccttcaccgactactatatgaactgggtgaagcagagccacggcaagagcctggagtggatcggcgacatcaaccctaacaacggcggcacctcctacaaccagaagttcaagggcaaggctacactgaccgtggacaagtcctccagcaccgcctacatggagctcaggagcctgacctccgaggattccgccgtctattactgtgcccggtacgactacgcctggtatttcgacgtgtggggccagggcacaaccgtcacagtctccagcggaggaggaggaagcggcggcggaggatccggaggcggaggcgatgtcctgatgacacagacacctctgagcctccccgtgagcctgggagaccaagcctccatctcctgcaggtcctcccagtccatcgtgcacagcaatggcaacacctacctggagtggtatctgcagaagcctggccagtcccccaagctgctgatctacaaggtgtccaaccggttcagcggcgtccctgacaggttctccggatccggaagcggcacagatttcaccctgaagatcagcagggtcgaggccgaggacctgggagtgtactactgcttccagggctcccatgtcccttacaccttcggcggcggcaccaaactggagatcaagcggatcgagggcaggcatcaccaccatcaccactga

SEQ ID NO: 25 - protein, 10 aa, Artificial sequence
  Synthetic polypeptide
KSVSTSGYSY

SEQ ID NO: 26 - protein, 3 aa, Artificial sequence
  Synthetic polypeptide
LVS

SEQ ID NO: 27 - protein, 16 aa, Artificial sequence
  Synthetic polypeptide
QHIRELTRSEGGPSWK

SEQ ID NO: 28 - protein, 8 aa, Artificial sequence
  Synthetic polypeptide
GYTFTSYW

SEQ ID NO: 29 - protein, 8 aa, Artificial sequence
  Synthetic polypeptide
INPSNGGT

SEQ ID NO: 30 - protein, 12 aa, Artificial sequence
  Synthetic polypeptide
ARRGIRLRHFDY

SEQ ID NO: 31 - protein, 6 aa, Artificial sequence
  Synthetic polypeptide
QDVGTA

SEQ ID NO: 32 - protein, 3 aa, Artificial sequence
  Synthetic polypeptide
WAS

SEQ ID NO: 33 - protein, 9 aa, Artificial sequence
  Synthetic polypeptide
QQYSSYPLT

SEQ ID NO: 34 - protein, 108 aa, Artificial sequence
  Synthetic polypeptide
DIVMTQSPASLAVSLGQRATISYRASKSVSTSGYSYMHWNQQKPGQPPRLLIYLVSNLESGVPARFSGSGSGTDFTLNIHPVEEEDAATYYCQHIRELTRSEGGPSWK

SEQ ID NO: 35 - protein, 107 aa, Artificial sequence
  Synthetic polypeptide
DIQMTQSPKFMSTSVGDRVSITCKASQDVGTAVAWYQQKPGQSPKLLIYWASTRHTGVPDRFTGSGSGTDFTLTISNVQSEDLADYFCQQYSSYPLTFGAGTKLELK

SEQ ID NO: 36 - protein, 117 aa, Artificial sequence
  Synthetic polypeptide
VKLQESGTELVKPGASVKLSCKASGYTPTSYWMHWVKQRPGQGLEWIGNINPSNGGTNYNEKFKSKATLTVDKSSSTAYMQLSSLTSEDSAVYYCARRGIRLRHFDYWGQGTTVTVS

SEQ ID NO: 37 - protein, 275 aa, Artificial sequence
  Synthetic polypeptide
VKLQESGTELVKPGASVKLSCKASGYTFTSYWMHWVKQRPGQGLEWIGNINPSNGGTNYNEKFKSKATLTVDKSSSTAYMQLSSLTSEDSAVYYCARRGIRLRHFDYWGQGTTVTVSSRANSADIHHTGGRSSMHLEGPIRPIVSRISGGGGGSGGGGSWISAEFALDIVMTQSPASLAVSLGQRATISYRASKSVSTSGYSYMHWNQQKPGQPPRLLIYLVSNLESGVPARFSGSGSGTDFTLNIHPVEEEDAATYYCQHIRELTRSEGGPSWK

SEQ ID NO: 38 - protein, 271 aa, Artificial sequence
  Synthetic polypeptide
MSVPTQVLGLLLLWLTDARCVKLQESGTELVKPGASVKLSCKASGYTFTSYWMHWVKQRPGQGLEWIGNINPSNGGTNYNEKFKSKATLTVDKSSSTAYMQLSSLTSEDSAVYYCARRGIRLRHFDYWGQGTTVTVSSGGGGSGGGGSGGGGSDIQMTQSPKFMSTSVGDRVSITCKASQDVGTAVAWYQQKPGQSPKLLIYWASTRHTGVPDRFTGSGSGTDFTLTISNVQSEDLADYFCQQYSSYPLTFGAGTKLELKRIEGRHHHHHH

SEQ ID NO: 39 - DNA, 324 bp, Artificial sequence
  Synthetic polynucleotide
gacattgtgatgacacagtctcctgcttccttagctgtatctctggggcagagggccaccatctcatacagggccagcaaaagtgtcagtacatctggctatagttatatgcactggaaccaacagaaaccaggacagccacccagactcctcatctatcttgtatccaacctagaatctggggtccctgccaggttcagtggcagtgggtctgggacagacttcaccctcaacatccatcctgtggaggaggaggatgctgcaacctattactgtcagcacattagggagcttacacgttcggaggggggaccaagctggaaa

SEQ ID NO: 40 - DNA, 322 bp, Artificial sequence
  Synthetic polynucleotide
gacatccagatgacccagtctcccaaattcatgtccacatcagtaggagacagggtcagcatcacctgcaaggccagtcaggatgtgggtactgctgtagcctggtatcaacagaaaccagggcaatctcctaaactactgatttactgggcatccacccggcacactggagtccctgatcgcttcacaggcagtggatctgggacagatttcactctcaccattagcaatgtgcagtctgaagacttggcagattatttctgtcagcaatatagcagctatcctctcacgttcggtgctgggaccaagctggagctgaaac

SEQ ID NO: 41 - DNA, 351 bp, Artificial sequence
  Synthetic polynucleotide
gtgaaactgcaggagtctgggactgaactggtgaagcctggggcttcagtgaagctgtcctgcaaggcttctggctacaccttcaccagctactggatgcactgggtgaagcagaggcctggacaaggccttgagtggattggaaatattaatcctagcaatggtggtactaactacaatgagaagttcaagagcaaggccacactgactgtagacaaatcctccagcacagcctacatgcagctcagcagcctgacatctgaggactctgcggtctattattgtgcaagaagaggcatacggttacgacactttgactactggggccaagggaccacggtcaccgtctcc

SEQ ID NO: 42 - DNA, 828 bp, Artificial sequence
  Synthetic polynucleotide
gccgccaccatgagtgtgcccactcaggtcctggggttgctgctgctgtggcttacagatgccagatgtgtgaaactgcaggagtctgggactgaactggtgaagcctggggcttcagtgaagctgtcctgcaaggcttctggctacaccttcaccagctactggatgcactgggtgaagcagaggcctggacaaggccttgagtggattggaaatattaatcctagcaatggtggtactaactacaatgagaagttcaagagcaaggccacactgactgtagacaaatcctccagcacagcctacatgcagctcagcagcctgacatctgaggactctgcggtctattattgtgcaagaagaggcatacggttacgacactttgactactggggccaagggaccacggtcaccgtctcctctggcggaggtgggtcgggtggcggcggatctggcggaggtgggtcggacatccagatgacccagtctcccaaattcatgtccacatcagtaggagacagggtcagcatcacctgcaaggccagtcaggatgtgggtactgctgtagcctggtatcaacagaaaccagggcaatctcctaaactactgatttactgggcatccacccggcacactggagtccctgatcgcttcacaggcagtggatctgggacagatttcactctcaccattagcaatgtgcagtctgaagacttggcagattatttctgtcagcaatatagcagctatcctctcacgttcggtgctgggaccaagctggagctgaaacggatcgaaggccggcatcaccatcatcaccactgatag

SEQ ID NO: 43 - DNA, 819 bp, Artificial sequence
  Synthetic polynucleotide
atgagcgtgcctacacaggtgctcggcctgctgctcctctggctgacagacgcccggtgtgtgaagctgcaggagtccggaaccgagctggtgaaacctggcgccagcgtgaaactgagctgcaaagccagcggatacaccttcacctcctactggatgcactgggtgaaacagaggcctggccagggcctggaatggattggcaacatcaaccccagcaacggcggcaccaactacaatgagaagttcaagagcaaggccaccctgaccgtggataagtcctcctccaccgcctacatgcagctgtcctccctcacctccgaggacagcgccgtctattactgtgccaggcggggcatcaggctgaggcacttcgactactggggccaaggcacaaccgtcaccgtgagctccggaggaggaggcagcggaggcggaggctccggcggaggcggaagcgacattcagatgacccagagccccaagttcatgtccacctccgtcggcgacagggtgagcatcacctgtaaggccagccaggatgtcggcacagctgtggcctggtaccagcagaagcccggccagtcccccaagctgctgatctactgggcttccacaaggcataccggcgtccccgataggttcacaggctccggctccggcaccgacttcacactcaccatcagcaacgtccagtccgaggacctggccgactacttctgccagcagtactccagctaccccctcaccttcggcgctggcaccaagctggaactcaagcggatcgagggcaggcatcaccaccatcaccactgatag

SEQ ID NO: 44 - protein, 392 aa, Homo sapiens [sequence omitted]

SEQ ID NO: 45 - protein, 381 aa, Homo sapiens
MTVARPSVPAALPLLGELPRLLLLVLLCLPAVWGDCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQATRSTPVSRTTKHFHETTPNKGSGTTSGTTRLLSGHTCFTLTGLLGTLVTMGLLT

SEQ ID NO: 46 - protein, 390 aa, Mus musculus
MIRGRAPRTRPSPPPPLLPLLSLSLLLLSPTVRGDCGPPPDIPNARPILGRHSKFAEQSKVAYSCNNGFKQVPDKSNIVVCLENGQWSSHETFCEKSCVAPERLSFASLKKEYLNMNFFPVGTIVEYECRPGFRKQPPLPGKATCLEDLVWSPVAQFCKKKSCPNPKDLDNGHINIPTGILFGSEINFSCNPGYRLVGVSSTFCSVTGNTVDWDDEFPVCTEIHCPEPPKINNGIMRGESDSYTYSQVVTYSCDKGFILVGNASIYCTVSKSDVGQWSSPPPRCIEKSKVPTKKPTINVPSTGTPSTPQKPTTESVPNPGDQPTPQKPSTVKVSATQHVPVTKTTVRHPIRTSTDKGEPNTGGDRYIYGHTCLITLTVLHVMLSLIGYLT

SEQ ID NO: 47 - protein, 128 aa, Homo sapiens
MGIQGGSVLFGLLLVLAVFCHSGHSLQCYNCPNPTADCKTAVNCSSDFDACLITKAGLQVYNKCWKFEHCNFNDVTTRLRENELTYYCCKKDLCNFNEQLENGGTSLSEKTVLLLVTPFLAAAWSLHP

SEQ ID NO: 48 - protein, 123 aa, Mus musculus
MRAQRGLILLLLLLAVFCSTAVSLTCYHCFQPVVSSCNMNSTCSPDQDSCLYAVAGMQVYQRCWKQSDCHGEIIMDQLEETKLKFRCCQFNLCNKSDGSLGKTPLLGTSVLVAILNLCFLSHL

SEQ ID NO: 49 - protein, 129 aa, Mus musculus
MRAQRGLILLLLLLAVFCSTAVSLKCYNCFQFVSSCKINTTCSPNLDSCLYAVAGRQVYQQCWKLSDCNSNYIMSRLDVAGIQSKCCQWGLCNKNLDGLEEPNNAETSSLRKTALLGTSVLVAILKFCF

SEQ ID NO: 50 - protein, 483 aa, Mus musculus
MEVSSRSSEPLDPVWLLVAFGRGGVKLEVLLLFLLPFTLGELRGGLGKHGHTVHREPAVNRLCADSKRWSGLPVSAQRPFPMGHCPAPSQLPSAKPINLTDESMFPIGTYLLYECLPGYIKRQFSITCKQDSTWTSAEDKCIRKQCKTPSDPENGLVHVHTGIQFGSRINYTCNQGYRLIGSSSAVCVITDQSVDWDTEAPICEWIPCEIPPGIPNGDFFSSTREDFHYGMVVTYRCNTDARGKALFNLVGEPSLYCTSNDGEIGVWSGPPPQCIELNKCTPPPYVENAVMLSENRSLFSLRDIVEFRCHPGFIMKGASSVHCQSLNKWEPELPSCFKGVICRLPQEMSGFQKGLGMKKEYYYGENVTLECEDGYTLEGSSQSQCQSDGSWNPLLAKCVSRSISGLIVGIFIGIIVFILVIIVFIWMILKYKKRNTTDEKYKEVGIHLNYKEDSCVRLQSLLTSQENSSTTSPARNSLTQEVS

SEQ ID NO: 51 - protein, 2039 aa, Homo sapiens
MGASSPRSPEPVGPPAPGLPFCCGGSLLAVVVLLALPVAWGQCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPEILHGEHTPSHQDNFSPGQEVFYSCEPGYDLRGAASLHCTPQGDWSPEAPRCAVKSCDDFLGQLPHGRVLFPLNLQLGAKVSFVCDEGFRLKGSSVSHCVLVGMRSLWNNSVPVCEHIFCPNPPAILNGRHTGTPSGDIPYGKEISYTCDPHPDRGMTFNLIGESTIRCTSDPHGNGVWSSPAPRCELSVRAGHCKTPEQFPFASPTIPINDFEFPVGTSLNYECRPGYFGKMFSISCLENLVWSSVEDNCRRKSCGPPPEPFNGMVHINTDTQFGSTVNYSCNEGFRLIGSPSTTCLVSGNNVTWDKKAPICEIISCEPPPTISNGDFYSNNRTSFHNGTVVTYQCHTGPDGEQLFELVGERSIYCTSKDDQVGVWSSPPPRCISTNKCTAPEVENAIRVPGNRSFFSLTEIIRFRCQPGFVMVGSHTVQCQTNGRWGPKLPHCSRVCQPPPEILHGEHTLSHQDNFSPGQEVFYSCEPSYDLRGAASLHCTPQGDWSPEAPRCTVKSCDDFLGQLPHGRVLLPLNLQLGAKVSFVCDEGFRLKGRSASHCVLAGMKALWNSSVPVCEQIFCPNPPAILNGRHTGTPFGDIPYGKEISYACDTHPDRGMTFNLIGESSIRCTSDPQGNGVWSSPAPRCELSVPAACPHPPKIQNGHYIGGHVSLYLPGMTISYTCDPGYLLVGKGFIFCTDQGIWSQLDHYCKEVNCSFPLFMNGISKELEMKKVYHYGDYVTLKCEDGYTLEGSPWSQCQADDRWDPPLAKCTSRAHDALIVGTLSGTIFFILLIIFLSWIILKHRKGNNAHENPKEVAIHLHSQGGSSVHPRTLQTNEENSRVLP

SEQ ID NO: 52 - protein, 1231 aa, Homo sapiens
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFFIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 53 - protein, 1234 aa, Mus musculus
MRLSARIIWLILWTVCAAEDCKGPPPRENSEILSGSWSEQLYPEGTQATYKCRPGYRTLGTIVKVCKNGKWVASNPSRICRKKPCGHPGDTPFGSFRLAVGSQFEFGAKVVYTCDDGYQLLGEIDYRECGADGWINDIPLCEVVKCLPVTELENGRIVSGAAETDQEYYFGQVVRFECNSGFKIEGHKEIHCSENGLWSNEKPRCVEILCTPPRVENGDGINVKPVYKENERYHYKCKHGYVPKERGDAVCTGSGWSSQPFCEEKRCSPPYILNGIYTPHRIIHRSDDEIRYECNYGFYPVTGSTVSKCTPTGWIPVPRCTLKPCEFPQFKYGRLYYEESLRPNFPVSIGNKYSYKCDNGFSPPSGYSWDYLRCTAQGWEPEVPCVRKCVFHYVENGDSAYWEKVYVQGQSLKVQCYNGYSLQNGQDTMTCTENGWSPPPKCIRIKTCSASDIHIDNGFLSESSSIYALNRETSYRCKQGYVTNTGEISGSITCLQNGWSPQPSCIKSCDMPVFENSITKNTRTWFKLNDKLDYECLVGFENEYKHTKGSITCTYYGWSDTPSCYERECSVPTLDRKLVVSPRKEKYRVGDLLEFSCHSGHRVGPDSVQCYHFGWSPGFPTCKGQVASCAPPLEILNGEINGAKKVEYSHGEVVKYDCKPRFLLKGPNKIQCVDGNWTTLPVCIEEERTCGDIPELEHGSAKCSVPPYHHGDSVEFICEENFFMIGHGSVSCISGKWTQLPKCVATDQLEKCRVLKSTGIEAIKPKLTEFFHNSTMDYKCRDKQEYERSICINGKWDPEPNCTSKTSCPPPPQIPNTQVIETTVKYLDGEKLSVLCQDNYLTQDSEEMVCKDGRWQSLPRCIEKIPCSQPPTIEHGSINLPRSSEERRDSIESSSHEHGTTFSYVCDDGFRIPEENRITCYMGKWSTPPRCVGLPCGPPPSIPLGTVSLELESYQHGEEVTYHCSTGFGIDGPAFIICEGGKWSDPPKCIKTDCDVLPTVKNAIIRGKSKKSYRTGEQVTFRCQSPYQMNGSDTVTCVNSRWIGQPVCKDNSCVDPPHVPNATIVTRTKNKYLHGDRVRYECNKPLELFGQVEVMCENGIWTEKPKCRDSTGKCGPPPPIDNGDITSLSLPVYEPLSSVEYQCQKYYLLKGKKTITCTNGKWSEPPTCLHACVIPENIMESHNIILKWRHTEKIYSHSGEDIEFGCKYGYYKARDSPPFRTKCINGTINYPTCV

SEQ ID NO: 54 - protein, 21 aa, Homo sapiens
MPMGSLQPLATLYLLGMLVAS

SEQ ID NO: 55 - protein, 17 aa, Homo sapiens
MGAAGLLGVFLALVAPG

SEQ ID NO: 56 - protein, 20 aa, Homo sapiens
MGAAGLLGVFLALVAPGVLG

SEQ ID NO: 57 - DNA, 927 bp, Artificial sequence
  Synthetic polynucleotide
gacacgtgatcagccgccaccatgcccatggggtctctgcaaccgctggccaccttgtacctgctggggatgctggtcgcttccgtgctagcgcatcatcatcatcatcatgtgaaactgcaggagtcaggggctgagcttgtgaagcctggggcttcagtgaagctgtcctgcaaggcttctggctacaccttcaccagctactggatgcactgggtgaagcagaggcctggacgaggccttgagtggattggaaggattggtcctaatagtggtggtactaagtacaatgagaagttcaagagcaaggccacactgactgtagacaaaccctccagcacagcctacatgcagctcagcagcctgacatctgaggactctgcggtctattattgtgcaagaagaatggtaaaggggtgctatggactactggggccaagggaccacggtcaccgtctcctcaaagggcgaattccagcacactggcggccgttactagtggatccgagctcggtaccaagcttggcgtcaggaggcggtggcggctcgggtggcggcggctcttggatatctgcagaattcgcccttgacattgagctcacccagtctccaaccaccatggctgcatctcccggggagaagatcactatcacctgcagtgccagctcaagtataagttccaattacttgcattggtatcagcagaagccaggattctcccctaaactcttgatttataggacatccaatctggcttctggagtcccagctcgcttcagtggcagtgggtctgggacctcttactctctcacaattggcaccatggaggctgaagatgttgccacttactactgccagcagggtagtagtataccacgtacacgttcggagggggcaccaagctggaaataatagactagtcgtgcg

SEQ ID NO: 58 - DNA, 954 bp, Artificial sequence
  Synthetic polynucleotide
gacacgaagcttgccgccaccatgcccatggggtctctgcaaccgctggccaccttgtacctgctggggatgctggtcgcttccgtgctagcgcatcatcatcatcatcatgtcaagctgcaggagtctgggactgaactggtgaagcctggggcttcagtgaagctgtcctgcaaggcttctggctacaccttcaccagctactggatgcactgggtgaagcagaggcctggacaaggccttgagtggattggaaatattaatcctagcaatggtggtactaactacaatgagaagttcaagagcaaggccacactgactgtagacaaatcctccagcacagcctacatgcagctcagcagcctgacatctgaggactctgcggtctattattgtgcaagaagaggcatacggttacgacactttgactactggggccaagggaccacggtcaccgtctcctcaagggcgaattctgcagatatccatcacactggcggccgctcgagcatgcatctagagggcccaattcgccctatagtgagtcgtatatcaggaggcggtggcggctcgggtggcggcggctcttggatatctgcagaattcgcccttgacattgtgatgacacagtctcctgcttccttagctgtatctctggggcagagggccaccatctcatacagggccagcaaaagtgtcagtacatctggctatagttatatgcactggaaccaacagaaaccaggacagccacccagactcctcatctatcttgtatccaacctagaatctggggtccctgccaggttcagtggcagtgggtctgggacagacttcaccctcaacatccatcctgtggaggaggaggatgctgcaacctattactgtcagcacattagggagcttacacgttcggaggggggaccaagctggaaataatagcccgggcgtgcg

(19) United States
(12) Patent Application Publication
(10) Pub. No.: US 2016/0083469 A1
(43) Pub. Date: Mar. 24, 2016

(54) TARGETING CONSTRUCTS BASED ON NATURAL ANTIBODIES AND USES THEREOF

(71) Applicants: The Regents of the University of Colorado, A Body Corporate, Denver, CO (US); MUSC Foundation For Reaearch Development, Charleston, SC (US); Department of Veterans Affairs (US), Washington, DC (US)

(72) Inventors: Baerbel M. Rohrer, Charleston, SC (US); Michael Holers, Denver, CO (US); Stephen Tomlinson, Mount Pleasant, SC (US); Joshua M. Thurman, Greenwood Village, CO (US); Carl Atkinson, Mount Pleasant, SC (US); Liudmila Kulik, Aurora, CO (US)

(21) Appl. No.: 14/807,521

(22) Filed: Jul. 23, 2015

Related U.S. Application Data
(63) Continuation of application No. PCT/US2014/012831, filed on Jan. 23, 2014.
(60) Provisional application No. 61/771,560, filed on Mar. 1, 2013.
(60) Provisional application No. 61/771,565, filed on Mar. 1, 2013.
(60) Provisional application No. 61/755,960, filed on Jan. 23, 2013.
(60) Provisional application No. 61/755,968, filed on Jan. 23, 2013.

(51) Int. Cl. C07K 16/28; A61K 45/06; A61K 39/395

(52) U.S. Cl. CPC C07K 16/28; A61K 39/3955; A61K 45/06; A61K 2039/505

(57) ABSTRACT

The present invention provides targeted delivery methods and constructs for treating inflammatory diseases and/or detecting in vivo tissue injuries in an individual. The targeted delivery approach utilizes an antibody that recognises an epitope found to be present at sites of inflammation. The invention also provides methods of inhibiting complement-driven inflammation in the eye in an individual, comprising administering to the individual an antibody or a fragment thereof or compositions thereof, wherein the antibody or fragment thereof specifically binds to Annexin IV or phospholipid. Also provided are related methods of treating a complement-associated ocular disease or an ocular disease involving oxidative damage. Additionally, the invention provides methods of detecting complement-mediated injury in an eye tissue of an individual, comprising administering to the individual a construct or compositions thereof, wherein the construct comprises (a) an antibody or fragment thereof that specifically binds to Annexin IV or phospholipid; and (b) a detectable moiety.

### CROSS-REFERENCE TO RELATED APPLICATIONS

This patent application is a continuation of International Patent Application No. PCT/US2014/012831, filed Jan. 23, 2014, which claims priority benefit of U.S. Provisional Patent Application No. 61/755,960 filed Jan. 23, 2013, U.S. Provisional Patent Application No. 61/755,968, filed Jan. 23, 2013, U.S. Provisional Patent Application No. 61/771,560, filed Mar. 1, 2013, and of U.S. Provisional Patent Application No. 61/771,565 filed Mar. 1, 2013, the entire content of each of which is incorporated herein by reference.

### STATEMENT REGARDING FEDERALLY SPONSORED RESEARCH OR DEVELOPMENT

This invention was made with Government support under Grant (Contract) Nos.: U.S. Army Medical Research and Materiel Command (MRMC) Awards W81XWH-06-1-0520 and W81XWH-07-1-0286, and National Institutes of Health RO1 AI31105, C06 RR015455, and R01EY019320 and Department of Veterans Affairs 101 RX000444; Beckman Institute for Macular Research.

### TECHNICAL FIELD

This application pertains to targeting constructs based on natural antibodies and uses thereof. This application also pertains to compositions and methods of treating ocular disease, specifically ocular diseases associated with oxidative stress.

### BACKGROUND

Natural antibodies exist in an immune competent individual and can be found in the serum or plasma of an individual not known to have been stimulated by a specific antigen to which the antibody binds. Previous studies by the present inventors and colleagues have shown that certain types of natural antibodies recognize epitopes on ischemic tissue and catalyze the initiation and subsequent development of ischemia-reperfusion injury (Fleming et al., 2002, J. Immunol. 169:2126-2133; Rehrig et al., 2001, J. Immunol. 167:5921-5927). Ischemia-reperfusion injury, as well as hypovolemic shock and subsequent tissue damage, is known to be caused by complement and Fc receptor activation and the recruitment and activation of neutrophils and other inflammatory cells (Rehrig et al., 2001, supra). It had also been shown that single monoclonal antibodies that react broadly with phospholipids and other extracellular or intracellular antigens such as DNA can cause ischemia-reperfusion injury in mice that lack other antibodies (i.e., B cell-deficient mice).

Ischemia-reperfusion (IR) injury refers to damage to a tissue caused when the blood supply returns to the tissue after a period of ischemia (restriction in blood supply). The absence of oxygen and nutrients from the blood creates a condition in which the restoration of circulation results in inflammation and oxidative damage, rather than restoration of normal function. Ischemia-reperfusion injury can be associated with traumatic injury, including hemorrhagic shock, as well as many other medical conditions such as stroke or large vessel occlusion, and is a major medical problem. More particularly, ischemia-reperfusion injury is important in heart attacks, stroke, kidney failure following vascular surgery, post-transplantation injury and chronic rejection, as well as in various types of traumatic injury, where hemorrhage will lead to organ hypoperfusion, and then subsequent reperfusion injury during fluid resuscitation. Ischemia-reperfusion injury, or an injury due to reperfusion and ischemic events, is also observed in a variety of autoimmune and inflammatory diseases. Independently of other factors, ischemia-reperfusion injury leads to increased mortality.

There is also increasing evidence of reperfusion injury that can be found in autoimmune and inflammatory diseases that are not traditionally thought of as reperfusion injury-related. For example, the synovium in rheumatoid arthritis patients is a site that is subjected to constant reperfusion stress (e.g., low pH, lots of tissue pressure and poor perfusion). The higher quantity of synovial fluid found in hypermobile patients having this disease causes an increase in the intra-articular pressure, which is then exacerbated by joint motion. This may aggravate local inflammation through a hypoxic/reperfusion mechanism, which in turn causes oxidative injury due to intermittent ischemia (e.g., see Punzi et al., Rheumatology 2001; 40: 202-204; Pianon et al., Reumatismo 1996; 48(Suppl. 1):93; and Jawed et al., Ann Rheum Dis 1997; 56:686-9). A variety of inflammatory and autoimmune diseases can also be associated with similar changes in cell stress responses of local cells that are similar to, or mimic, some changes in reperfusion injury.

Kulik et al. showed that pathogenic natural antibodies recognizing Annexin IV are required to develop intestinal ischemia-reperfusion injury. J. Immunol. 2009; 182:5363-5373. U.S. Patent Application Publication No. 2011/0014270 discloses lipids, annexins, and lipid-annexin complexes for use in the prevention and/or treatment of ischemia-reperfusion injury and reperfusion injury associated with a variety of diseases and conditions.

Natural antibodies are also involved in the pathology of ocular diseases. Age-related macular degeneration (AMD), which is characterized by progressive loss of central vision resulting from damage to the photoreceptor cells in the central area of the retina, the macula, is the leading cause of vision loss in the elderly of industrialized nations (Council, N. (1999) Vision research—a national plan: 1999-2003, executive summary. (National Eye Institute, N. i. o. h. ed., Washington, D.C.). Although AMD occurs in two forms, neovascular (wet) and atrophic (dry), both are associated with pathological lesions at the retinal pigmented epithelium (RPE)/choroid interface in the macular region (Nowak, J. Z. (2006) Pharmacol Rep 58, 353-363). Early AMD is characterized by a thickening of Bruch's membrane, which includes basal linear deposits and drusen (Hageman, G. S., Luthert, P. J., Victor Chong, N. H., Johnson, L. V., Anderson, D. H., and Mullins, R. F. (2001) Prog Retin Eye Res 20, 705-732). Additionally, changes in RPE morphology, pigmentation and deterioration of its function as a blood-retina barrier have been reported (McLeod, D. S., Taomoto, M., Otsuji, T., Green, W. R., Sunness, J. S., and Lutty, G. A. (2002) Invest Ophthalmol Vis Sci 43, 1986-1993). Advanced AMD is characterized by additional subtype-specific morphological features exacerbating the early pathological damage (Bhutto, I., and Lutty, G. (2012) Mol Aspects Med 33, 295-317). Dry AMD, or geographic atrophy, results from the loss of RPE followed by the loss of photoreceptors; whereas wet AMD is associated with choroidal neovascularization and leakage of these new vessels.

AMD is a complex disease with both genetic and environmental risk factors. The main environmental risk factor is persistent oxidative stress (Snodderly, D. M. (1995) Am J Clin Nutr 62, 1448S-1461S), whether that might be caused by smoking, nutritional deficits or even light exposure. A main genetic risk factor for the disease is polymorphisms in genes for complement proteins, including complement factor H (CFH), complement factor B (CFB), complement component 2 (C2) and complement component 3 (C3) (reviewed in (Charbel Issa, P., Chong, N. V., and Scholl, H. P. (2011) Graefes Arch Clin Exp Ophthalmol 249, 163-174)). Discovering complement genes as risk factors was consistent with prior clinical studies, which demonstrated that the complement system activation products were found locally in the eye in all stages of AMD (Hageman, G. S., Anderson, D. H., Johnson, L. V., Hancox, L. S., Taiber, A. J., Hardisty, L. I., Hageman, J. L., Stockman, H. A., Borchardt, J. D., Gehrs, K. M., Smith, R. J., Silvestri, G., Russell, S. R., Klaver, C. C., Barbazetto, I., Chang, S., Yannuzzi, L. A., Bartle, G. R., Merriam, J. C., Smith, R. T., Olsh, A. K., Bergeron, J., Zernant, J., Merriam, J. E., Gold, B., Dean, M., and Allikmets, R. (2005) Proc Natl Acad Sci USA 102, 7227-7232). Follow-up experiments in animal models, in particular of wet AMD, further support the hypothesis that inadequate control of complement-driven inflammation may be a major factor in the disease pathogenesis of AMD (e.g., (Nozaki, M., Raisler, B. J., Sakurai, E., Sarma, J. V., Barnum, S. R., Lambris, J. D., Chen, Y., Zhang, K., Ambati, B. K., Baffi, J. Z., and Ambati, J. (2006) Proc Natl Acad Sci USA 103, 2328-2333; Bora, P. S., Sohn, J. H., Cruz, J. M., Jha, P., Nishihori, H., Wang, Y., Kaliappan, S., Kaplan, H. J., and Bora, N. S. (2005) J Immunol 174, 491-497; Rohrer, B., Coughlin, B., Kunchithapautham, K., Long, Q., Tomlinson, S., Takahashi, K., and Holers, V. M. (2011) Mol Immunol 48, e1-8)). Although the current understanding of AMD is that chronic oxidative damage over time leads to alterations in photoreceptors, RPE/Bruch's membrane and the choriocapillaris complex, in particular in the macula, resulting in chronic inflammation and complement activation (Zarbin, M. A., and Rosenfeld, P. J. (2010) Retina 30, 1350-1367), it is unclear which components of the complement cascade are involved in causing damage, and what ligands or age-related changes in the these tissues enable complement activation. The complement cascade, an evolutionarily ancient and highly conserved system, is part of the innate and adaptive immune system, consisting of >40 soluble and membrane-bound components (Muller-Eberhard, H. J. (1988) Annu Rev Biochem 57, 321-347). Its normal role is to complement the ability of antibodies and phagocytic cells to eliminate pathogens. To spot these microorganisms, pattern recognition molecules complexed to inactive serum proteases circulate in the blood. Upon ligand interaction, the protease becomes activated to initiate the complement cascade. This results in the production of anaphylatoxins to recruit phagocytic cells, opsonins to tag material for removal, and the generation of the membrane attack complex (MAC) to rupture membranes of cells and leading to pro-inflammatory signaling in the target cell. Self cells are protected by either membrane-bound or soluble complement inhibitors. However, under pathological conditions, complement inhibition might be compromised, resulting in complement activation on self surfaces.

The complement system can be activated by one of three pathways, the classical, lectin and alternative pathway, each with its unique pattern recognition molecules. The classical pathway (CP) is activated when C1q binds to its ligands that include C-reactive protein (CRP); serum amyloid protein or IgG and IgM molecules present as immune complexes; the lectin pathway (LP) when mannan-binding lectin (MBL) or ficolin (H-ficolin, L-ficolin or M-ficolin) binds to specific carbohydrates or acetylated molecules on foreign cells or IgM molecules bound to antigens; and finally, the alternative pathway (AP) is spontaneously and continuously activated at a low level in a process called tickover, as well as when C3b is generated on cell surfaces by the CP or LP and becomes a substrate for the AP. All three pathways lead to the generation of a pathway-specific C3 convertase that then triggers the common terminal pathway with its above-described biological effects.

The disclosures of all publications, patents, patent applications and published patent applications referred to herein are hereby incorporated herein by reference in their entirety.

### BRIEF SUMMARY OF THE INVENTION

In one aspect, the present disclosure provides a method of inhibiting complement-mediated inflammation in a tissue having non-ischemic injury in an individual, comprising administering to the individual an effective amount of a composition comprising a construct, wherein the construct comprises (a) an antibody or a fragment thereof, wherein the antibody or a fragment thereof specifically binds to Annexin IV or a phospholipid; and (b) a complement inhibitor.

In another aspect, the present disclosure provides a method of treating an inflammatory disease in an individual, comprising administering to the individual an effective amount of a composition comprising a construct, wherein the construct comprises (a) an antibody or a fragment thereof, wherein the antibody or a fragment thereof specifically binds to Annexin IV or a phospholipid; and (b) a complement inhibitor.

In some embodiments of the methods above, the complement inhibitor is selected from the group consisting of an anti-05 antibody, anti-MASP antibody, an Eculizumab, an pexelizumab, an anti-C3b antibody, an anti-C6 antibody, an anti-C7 antibody, an anti-factor B antibody, an anti-factor D antibody, and an anti-properdin antibody, a membrane cofactor protein (MCP), a decay accelerating factor (DAF), a CD59, a Crry, a CR1, a factor H, a Factor I, a linear peptide, a cyclic peptide, a compstatin, an N-acetylaspartylglutamic acid (NAAGA), and a biologically active fragment of any the preceding. In some embodiments, the complement inhibitor is a specific inhibitor of the alternative pathway. In some embodiments, the complement inhibitor is a specific inhibitor of the lectin pathway.

In another aspect, the present disclosure provides a method of detecting complement-mediated injury in a tissue of an individual, comprising administering to the individual an effective amount of a composition comprising a construct, wherein the construct comprises (a) an antibody or a fragment thereof, wherein the antibody or a fragment thereof specifically binds to Annexin IV or phospholipid; and (b) a detectable moiety, wherein the presence of the detectable moiety at the tissue is indicative of a complement-mediated tissue injury. In some embodiments, the method further comprises detecting the detectable moiety. In some embodiments, the detectable moiety is selected from the group consisting of radioisotopes, fluorescent dyes, electron-dense reagents, enzymes, biotins, paramagnetic agents, magnetic agents, and nanoparticles.

In some embodiments of the methods above, the tissue injury results from any of from inflammatory disorders, transplant rejection, pregnancy-related diseases, adverse drug reactions, autoimmune or immune complex disorders. In some embodiments, the tissue is any one of eye, joint, kidney, brain, heart, spinal cord, and liver. In some embodiments, the tissue is any one of eye, joint, and kidney. In some embodiments, the disease is any one of an ocular disease, arthritis, or renal injury.

In some embodiments of the methods above, the antibody or fragment thereof specifically binds to Annexin IV. In some embodiments, the antibody or fragment thereof competitively inhibits the binding of monoclonal antibody B4 to Annexin IV. In some embodiments, the antibody or antibody fragment thereof binds to the same epitope as monoclonal antibody B4 (such as B4/14/12, ATCC Deposit No. PTA-13522). In some embodiments, the Annexin IV is present on the surface of a cell in an individual that is in or adjacent to a tissue undergoing non-ischemic injury.

In some embodiments of the methods, the antibody or fragment thereof specifically binds to a phospholipid. In some embodiments, the antibody or fragment thereof competitively inhibits the binding of monoclonal antibody C2 to phospholipid. In some embodiments, the antibody or fragment thereof binds to the same epitope as that of monoclonal antibody C2 (such as C2/19/8, ATCC Deposit No. PTA-13523). In some embodiments, the phospholipid is present on the surface of a cell in an individual that is in or adjacent to a tissue undergoing tissue injury and/or oxidative damage. In some embodiments, the phospholipid is selected from the group consisting of phosphatidylethanolamine (PE), cardiolipin (CL), and phosphatidylcholine (PC). In some embodiments, the phospholipid is MDA.

In some embodiments of the methods above, construct is a fusion protein. In some embodiments, the antibody or fragment thereof and the complement inhibitor or detectable moiety are linked via a peptide linker. In some embodiments, the antibody or fragment thereof is a scFv. In some embodiments, the antibody or fragment thereof is Fab, Fab′, or F(ab′)2.

In another aspect, the present disclosure provides a construct comprising: (a) an antibody or a fragment thereof, wherein the antibody or a fragment thereof specifically binds to Annexin IV; and (b) a complement modulator or a detectable moiety, wherein the antibody or fragment there of comprises: (i) a light chain variable domain comprising a sequence of SEQ ID NO:1 or 7, a sequence of SEQ ID NO:2 or 8, or a sequence of SEQ ID NO:3 or 9; and/or (ii) heavy chain variable domain comprising a sequence of SEQ ID NO:4 or 10, a sequence of SEQ ID NO:5 or 11, or a sequence of SEQ ID NO:6 or 12. In some embodiments, the antibody or fragment there of comprises: (i) a light chain variable domain comprising a sequence of SEQ ID NO:1 or 7; (ii) a light chain variable domain comprising a sequence of SEQ ID NO:2 or 8; and (iii) a light chain variable domain comprising a sequence of SEQ ID NO:3 or 9. In some embodiments, the antibody or fragment there of comprises: (i) heavy chain variable domain comprising a sequence of SEQ ID NO:4 or 10; (ii) heavy chain variable domain comprising a sequence of SEQ ID NO:5 or 11; and (iii) heavy chain variable domain comprising a sequence of SEQ ID NO:6 or 12. In some embodiments, the antibody or fragment there of comprises a light chain variable domain of SEQ ID NO:13 or 14. In some embodiments, the antibody or fragment there of comprises a heavy chain variable domain of SEQ ID NO:15 or 16. In some embodiments, the antibody or fragment is an scFv having the sequence of SEQ ID NO:17 or 18. In some embodiments, the antibody or fragment thereof competitively inhibits the binding of monoclonal antibody B4 to Annexin IV. In some embodiments, the antibody or fragment thereof binds to the same epitope as monoclonal antibody B4. In some embodiments, the Annexin IV is present on the surface of a cell in an individual that is in or adjacent to a tissue undergoing or is at risk of undergoing tissue injury.

In another aspect, the present disclosure provides a construct comprising: (a) an antibody or a fragment thereof, wherein the antibody or a fragment thereof specifically binds to a phospholipid; and (b) a complement modulator or a detectable moiety, wherein the antibody or fragment there of comprises: (i) a light chain variable domain comprising a sequence of SEQ ID NO:25 or 31, a sequence of SEQ ID NO:26 or 32, or a sequence of SEQ ID NO:27 or 33; and/or (ii) heavy chain variable domain comprising a sequence of SEQ ID NO:28, a sequence of SEQ ID NO:29, or a sequence of SEQ ID NO:30. In some embodiments, the antibody or fragment there of comprises: (i) a light chain variable domain comprising a sequence of SEQ ID NO:25 or 31; (ii) a light chain variable domain comprising a sequence of SEQ ID NO:26 or 32; and (iii) a light chain variable domain comprising a sequence of SEQ ID NO:27 or 33. In some embodiments, the antibody or fragment there of comprises: (i) heavy chain variable domain comprising a sequence of SEQ ID NO:28; (ii) heavy chain variable domain comprising a sequence of SEQ ID NO:29; and (iii) heavy chain variable domain comprising a sequence of SEQ ID NO:30. In some embodiments, the antibody or fragment there of comprises a light chain variable domain of SEQ ID NO:34 or 35. In some embodiments, the antibody or fragment there of comprises a heavy chain variable domain of SEQ ID NO:36. In some embodiments, the antibody or fragment is an scFv having the sequence of SEQ ID NO:37 or 38. In some embodiments, the antibody or fragment thereof competitively inhibits the binding of monoclonal antibody C2 to phospholipid. In some embodiments, the antibody or fragment thereof binds to the same epitope as monoclonal antibody C2.

In some embodiments, the phospholipid is present on the surface of a cell in an individual that is in or adjacent to a tissue undergoing or is at risk of undergoing tissue injury. In some embodiments, the phospholipid is selected from the group consisting of phosphatidylethanolamine (PE), cardiolipin (CL), and phosphatidylcholine (PC). In some embodiments, the phospholipid is MDA.

In some embodiments, the construct comprises a complement modulator. In some embodiments, the complement modulator is a complement inhibitor. In some embodiments, the complement inhibitor is selected from the group consisting of an anti-05 antibody, anti-MASP antibody, an Eculizumab, an pexelizumab, an anti-C3b antibody, an anti-C6 antibody, an anti-C7 antibody, an anti-factor B antibody, an anti-factor D antibody, and an anti-properdin antibody, a membrane cofactor protein (MCP), a decay accelerating factor (DAF), a CD59, a Crry, a CR1, a factor H, a Factor I, a linear peptide, a cyclic peptide, a compstatin, an N-acetylaspartylglutamic acid (NAAGA), and a biologically active fragment of any the preceding. In some embodiments, the complement inhibitor is a specific inhibitor of the alternative pathway. In some embodiments, the complement inhibitor is a specific inhibitor of the lectin pathway.

In some embodiments, the construct comprises a detectable moiety. In some embodiments, the detectable moiety is selected from the group consisting of radioisotopes, fluorescent dyes, electron-dense reagents, enzymes, biotins, paramagnetic agents, magnetic agents, and nanoparticles.

In some embodiments, the construct is a fusion protein. In some embodiments, the antibody or fragment thereof and the complement modulator or detectable moiety are linked by a peptide linker.

In another aspect, the present disclosure provides a pharmaceutical composition comprising a construct described above. Also provided is a method of inhibiting complement-mediated inflammation in an individual, comprising administering to the individual an effective amount of a pharmaceutical composition described herein. Additionally, the present disclosure provides a method of treating an inflammatory disease in an individual, comprising administering to the individual an effective amount of a pharmaceutical composition herein. Also provided herein is a composition comprising a construct comprising a detectable moiety, e.g., a radioisotope, a fluorescent dye, an electron-dense reagent, an enzyme, a biotin, a paramagnetic agent, a magnetic agent, and a nanoparticle. Also provided is a method of detecting complement-mediated injury in a tissue of an individual, comprising administering to the individual an effective amount of a composition of a construct comprising a detectable moiety.

In one aspect, the present disclosure provides a method of inhibiting complement-driven inflammation in the eye in an individual, comprising administering to the individual an effective amount of (a) an antibody or fragment thereof, wherein the antibody or fragment thereof does not activate complement activation, and wherein the antibody or fragment thereof specifically binds to Annexin IV or a phospholipid; or (b) a composition comprising a construct, wherein the construct comprises: (i) an antibody or a fragment thereof, wherein the antibody or fragment thereof specifically binds to Annexin IV or phospholipid, and (ii) a therapeutic agent.

In another aspect, the present disclosure provides a method of treating a complement-associated ocular disease or an ocular disease involving oxidative damage, comprising administering to the individual an effective amount of (a) an antibody or fragment thereof, wherein the antibody or fragment thereof does not activate complement activation, and wherein the antibody or fragment thereof specifically binds to Annexin IV or a phospholipid; or (b) a composition comprising a construct, wherein the construct comprises: (i) an antibody or a fragment thereof, wherein the antibody or fragment thereof specifically binds to Annexin IV or phospholipid, and (ii) a therapeutic agent.

In some embodiments, the methods comprise administration of an antibody or fragment thereof, wherein the antibody or fragment thereof does not activate complement activation, and wherein the antibody or fragment thereof specifically binds to Annexin IV or a phospholipid.

In some embodiments, the methods comprise administration of a composition comprising a construct, wherein the construct comprises: (i) an antibody or a fragment thereof, wherein the antibody or fragment thereof specifically binds to Annexin IV or phospholipid, and (ii) a therapeutic agent. In some embodiments, the therapeutic agent is a complement inhibitor. In some embodiments, the complement inhibitor is selected from, but not limited to, the group consisting of an anti-05 antibody, an Eculizumab, an pexelizumab, an anti-C3b antibody, an anti-C6 antibody, an anti-C7 antibody, an anti-factor B antibody, an anti-MASP antibody, an anti-factor D antibody, and an anti-properdin antibody, an anti-MBL antibody, a membrane cofactor protein (MCP), a decay accelerating factor (DAF), a CD59, a Crry, a CR1, a factor H, a Factor I, a linear peptide, a cyclic peptide, a compstatin, an N-acetylaspartylglutamic acid (NAAGA), and a biologically active fragment of any the preceding. In some embodiments, the complement inhibitor is a human complement inhibitor (e.g., a human MCP, a human DAF, a human CD59, a human CR1, a human Factor H, or another complement inhibitor derived from humans). In some embodiments, the complement inhibitor is a human complement inhibitor (e.g., a mouse DAF, a mouse CD59 (also known as isoform A), a mouse CD59 isoform B, a mouse Crry, a mouse Factor H, or another complement inhibitor derived from mouse). Complement inhibitors from other species and variant complement inhibitors are also contemplated.

In some embodiments of the methods, the ocular disease is selected from, but not limited to, the group consisting of age-related macular degeneration (AMD) (including wet AMD and dry AMD), cytomegalovirus (CMV) retinitis, macular edema, uveitis (anterior and posterior), glaucoma, open/wide-angle glaucoma, close/narrow-angle glaucoma, retinitis pigmentosa (RP), proliferative vitreoretinopathy, retinal detachment, corneal wound healing, corneal transplants, and ocular melanoma. In some embodiments, the ocular disease is AMD.

In another aspect, the present disclosure provides a method of detecting complement-mediated injury in an eye tissue of an individual, comprising administering to the individual an effective amount of a composition comprising a construct, wherein the construct comprises (a) an antibody or a fragment thereof, wherein the antibody or a fragment thereof specifically binds to Annexin IV or a phospholipid; and (b) a detectable moiety, wherein the presence of the detectable moiety at the tissue is indicative of a complement-mediated eye tissue injury. In some embodiments, the method further comprises detecting the detectable moiety. In some embodiments, the detectable moiety is selected from, but not limited to, the group consisting of radioisotopes, fluorescent dyes, electron-dense reagents, enzymes, biotins, paramagnetic agents, magnetic agents, and nanoparticles.

In some embodiments of the methods above, the antibody or fragment thereof specifically binds to Annexin IV. In some embodiments, the antibody or fragment thereof competitively inhibits the binding of monoclonal antibody B4 to Annexin IV. In some embodiments, the antibody or fragment thereof binds to the same epitope as that of monoclonal antibody B4 (such as B4/14/12, ATCC Deposit No. PAT-13522). In some embodiments, the Annexin IV is present on the surface of a cell, a basement membrane, or in a pathological structure in an individual that is in or adjacent to a tissue undergoing tissue injury and/or oxidative damage.

In some embodiments of the methods above, the antibody or fragment thereof specifically binds to a phospholipid. In some embodiments, the antibody or fragment thereof competitively inhibits the binding of monoclonal antibody C2 to phospholipid. In some embodiments, the antibody or fragment thereof binds to the same epitope as that of monoclonal antibody C2 (such as C2/19/8, ATCC Deposit No. PAT-13523). In some embodiments, the phospholipid is present on the surface of a cell, a basement membrane, or in a pathological structure in an individual that is in or adjacent to a tissue undergoing tissue injury and/or oxidative damage. In some embodiments, the phospholipid is selected from the group consisting of phosphatidylethanolamine (PE), cardiolipin (CL), and phosphatidylcholine (PC). In some embodiments the phospholipid is MDA.

In some embodiments of any of the methods above, the antibody or fragment thereof is a scFv. In some embodiments, the antibody or fragment thereof is Fab, Fab′, or F(ab′)2. In some embodiments, the construct is a fusion protein. In some embodiments, the antibody or a fragment thereof and the therapeutic agent or detectable moiety are joined by a linker. In some embodiments, the antibody or a fragment thereof and the therapeutic agent or detectable moiety are joined directly.

In some embodiments of any of the methods above, the administration is by injection into the eye. In some embodiments, the individual is human.

Also provided are unit dosage forms, kits, and articles of manufacture that are useful for methods described herein.

It is to be understood that one, some, or all of the properties of the various embodiments described herein may be combined to form other embodiments of the present invention.

### BRIEF DESCRIPTION OF THE FIGURES

FIGS. 1A-1C are a series of graphs showing characterization of B4scFv and B4scFv-Crry proteins. FIG. 1A) B4scFv, but not control C2scFv, bound directly to recombinant annexin IV in vitro. FIG. 1B) B4scFv competitively inhibited binding of B4 mAb to annexin IV. FIG. 1C) B4scFv-Crry inhibited complement activation in vitro similarly to positive control CR2-Crry.

FIGS. 2A-2B are a series of graphs showing that administration of B4 mAb and C2 mAb overcame protection from spinal cord injury (SCI) due to ischemic injury in Rag1^{-/-} mice and cerebral ischemia reperfusion injury. FIG. 2A) Locomotor activity after experimentally induced SCI in Rag1^{-/-} mice administered different mAbs. Rag1^{-/-} mice (filled squares) were protected from SCI 21 days post-injury while wild-type mice (filled circles) showed a reduction of locomotor activity 21 days post-injury. Injury in Rag1^{-/-} mice was reconstituted to levels comparable to wild-type mice when administered B4 mAb (empty circles) or C2 mAb (filled triangles). In comparison, Rag1^{-/-} mice administered control F632 mAb (empty diamond) remained protected from SCI 21 days post-injury. P<0.05, n=6-9. FIG. 2B) Post-ischemic infarct volume (TTC staining) in a mouse model of ischemic stroke (cerebral ischemia-reperfusion injury) after administering a targeting construct. Rag1^{-/-} mice were protected from cerebral infarct compared to C57Bl/6 wild-type mouse controls (#p<0.001). Reconstitution with increasing amounts of C2 or B4 mAbs restored injury to Rag1^{-/-} mice (@p=0.01). Reconstitution with 100 μg control antibody did not restore injury in Rag1^{-/-} mice. n=8-12.

FIGS. 3A-3B are a series of graphs showing that B4scFv-Crry targeting construct protected mice from SCI. FIG. 3A) Locomotor activity after experimentally induced SCI in wild-type mice administered a targeting construct. Targeted complement inhibitor B4scFv-Crry (0.2 mg), shown as B4-Crry, protected wild-type mice against SCI as compared to wild-type mice administered PBS. All mice had a score of 0 immediately after impact. p<0.05 from day 3. n=6. FIG. 3B) Morphometric analysis of tissue sparing 3 days after traumatic injury. Cross sectional area of 120 μm increments (H&E). B4-Crry indicates wild-type treated mice, WT indicates wild-type mice treated with PBS, all others were Rag1^{-/-} mice reconstituted with the indicated IgM mAbs, C2 mAb, B4 mAb, and F632 mAb or administered PBS (shown as Rag1^{-/-}). p<0.01 up to 1.2 mm each side of injury site. Mean+/−SD, n=6 per group.

FIGS. 4A-4E are immunofluorescence confocal analysis of IgM and C3 deposition. FIGS. 4A-4C) Spinal cord sections from untreated wild-type mice stained for FIG. 4A) IgM or FIG. 4B) C3 at 24 hours after injury. FIG. 4C) Merged image of IgM and C3 staining. FIG. 4D and FIG. 4E) Sections from wild-type mice treated with B4scFv-Crry and stained for FIG. 4D) IgM or FIG. 4E) C3 at 24 hours after injury.

FIG. 5 is a graph showing animal survival in a cecal ligation and puncture model of acute septic peritonitis after administration of B4scFv-Crry. The absence of C3 in C3 deficient mice (C3^{-/-}) resulted in death within 48 hours. There was no significant difference in survival seen between B4scFv-Crry (treated with 0.2 mg dose immediately post procedure) and wild-type untreated controls. n=5-6.

FIGS. 6A-6B show localization of B4 mAb in transplanted hearts after administration of B4 mAb to Rag1^{-/-} recipients. FIG. 6A) B4 mAb bound to endothelial cells of the transplanted heart but not the native heart. FIG. 6B) Co-localization of C3d (left panel) and endogenous IgM (middle panel) in a graft of wild-type mouse. Overlay image (right panel) shows co-localization of C3d and endogenous IgM.

FIGS. 7A-7F. FIGS. 7A-7D show a protective effect of B4scFv and B4scFv-Crry against cardiac ischemia reperfusion injury in transplanted heart. FIG. 7A) Graph showing decreased serum levels of cardiac troponin I with a single dose of B4scFv or B4scFv-Crry administered to recipient after transplant. ns=not significant. FIG. 7B) Graph summarizing histological scoring of cardiac damage showed a significant reduction in injury histology score with a single dose of B4scFv or B4scFv-Crry. ns=not significant. FIG. 7C) Immunofluorescence imaging of B4scFv-HisTag in vivo showing binding to endothelial cells. FIG. 7D) Immunofluorescent staining for C3d in allograft heart transplants after treatment of recipient with either 0.2 mg B4scFv-Crry (lower panels) or PBS control (upper panels). Only weak C3d vascular staining was seen in B4scFV-Crry treated animals 48 hours post treatment. Images representative of n=3. FIG. 7E and FIG. 7F) Shows the effect of B4scFv and B4scFvCrry treatment on IgM and C3d deposition in transplanted allografts. Recipient mice were treated with PBS, B4scFv or B4scFvCrry and allografts were isolated at 6 hours or 48 hours post-transplantation for analysis. Representative images of IgM and C3d deposition in grafts are shown. Semi-quantitative histological scoring of FIG. 7E) IgM and FIG. 7F) C3d deposition at 6 and 48 hours post-transplantation. *P<0.01, **P<0.001, Mean±SEM, n=6-8.

FIGS. 8A-8E are a series of graphs showing reduction of specific cytokines in allograft recipients after treatment with B4scFv or B4scFv-Crry. Cytokine levels for FIG. 8A) MCP-1, FIG. 8B) IL-6, FIG. 8C) KC, FIG. 8D) CXCL9, and FIG. 8E) IFN-γ. n=4-10, #p<0.01.

FIG. 9 is graph showing biodistribution of ¹²⁵I-labeled B4scFv-Crry in recipient mice administered immediately after heart transplantation and analyzed 6 hours later.

FIGS. 10A-10D show anti-IgM immunofluorescence images of B4 mAb binding to FIGS. 10A-10B) a mouse brain endothelial cell line (bEnd.3) and FIGS. 10C-10D) human umbilical vein endothelial cells (HUVEC), both subjected to 3 hours hypoxia (left panels) and 1 hour re-oxygenation (right panels). B4 mAb did not bind to either cell type not exposed to hypoxic conditions.

FIGS. 11A-11B are a series of graphs showing that administration of B4 mAb and C2 mAb overcame protection from hepatic ischemic reperfusion injury in Rag1^{-/-} mice. FIG. 11A) Serum ALT levels 6 hours post I/R in sham (Rage), wild-type, Rag1^{-/-} mice, and Rag1^{-/-} mice injected with 25 μg C2 mAb or B4 mAb. Mean±SD, n=4-9. FIG. 11B) Necrotic index 6 hours post IR in sham (Rag1^{-/-}), wild-type, Rag1^{-/-} mice and Rag1^{-/-} mice injected with 25 μg C2 mAb or B4 mAb. Mean±SD, n=7. ##, P<0.001 vs. wild-type;**, P<0.001 vs. Rag1^{-/-}; @, P<0.01 vs. Rage; *, P<0.05 vs. Rag1^{-/-}.

FIGS. 12A-12C are a series of graphs showing that administration of B4 mAb and C2 mAb stimulated liver regeneration following 70% partial hepatectomy in Rag1^{-/-} mice. FIG. 12A) Serum ALT levels 48 hours after PHx in Rag1^{-/-} mice and Rag1^{-/-} mice treated with 10 μg C2 mAb or B4 mAb. **, @@ P<0.01 vs. rag IR injury. Mean±SD, n=6. FIG. 12B) Necrotic index score (H&E staining) 48 hours post PHx in Rag1^{-/-} mice and Rag1^{-/-} mice treated with 10 μg C2 mAb or B4 mAb. ##, P<0.01 vs. wild-type; * and @, P<0.05 vs. Rag1^{-/-}. Mean±SD, n=4. FIG. 12C) Mitotic index 48 hours post PHx in Rag1^{-/-} mice and Rag1^{-/-} mice treated with 10 μg C2 mAb or B4 mAb. ##, P<0.01 vs. wild-type;**, P<0.01 vs. Rag1^{-/-}; @@, P<0.01 vs. Rag1^{-/-}. Mean±SD, n=4.

FIGS. 13A-13B are a series of graphs showing that administration of B4 mAb and C2 mAb stimulated liver regeneration following 70% partial hepatectomy in Rag1^{-/-} mice. FIG. 13A) Liver weight restitution 48 hours after PHx in Rag1^{-/-} mice and Rag1^{-/-} mice treated with 10 μg C2 mAb or B4 mAb. FIG. 13B) Brdu-positive cells 48 hours after PHx in Rag1^{-/-} mice and Rag1^{-/-} mice treated with 10 μg C2 mAb or B4 mAb. ***, P<0.001; **, P<0.01; *, P<0.05.

FIGS. 14A-14D. FIG. 14A is a series of immunohistochemistry pictures of IgM staining in liver sections 6 hours post IR in wild-type mice (wt), Rag1^{-/-} mice, and Rag1^{-/-} mice treated with C2 mAb or B4 mAb; FIG. 14B) a series of immunofluorescence images showing localization of endogenous IgM (left panel) and C3d (middle panel) in Rag1^{-/-} mice reconstituted with B4 mAb following hepatic IR. Overlay image (right panel) shows co-localization of C3d and endogenous IgM; FIG. 14C) immunohistochemical staining of IgM and C3d showed IgM and C3d staining in a sinusoidal pattern in WT and Rag1−/− mice reconstituted with B4 mAb or C2 mAb 48 hours after 70% PHx. No staining was seen in Rag1−/− treated with PBS. Representative images from 3 animals per group, magnification ×400; and FIG. 14D) immunofluorescence images showing localization of IgM (left panel) and C3d (middle panel) in Rag1−/− mice reconstituted with B4 mAb following 70% PHx. Overlay image (right panel) shows co-localization of C3d and endogenous IgM. Representative image from 3 animals, magnification ×520.

FIGS. 15A-15F are a series of graphs showing in vivo kinetics of B4scFv-Crry, biodistribution of IgM antibodies following IR or 70% Phx in Rag1−/− mice, and in vivo binding of B4 scFv construct. FIG. 15A) B4scFc-Crry had an initial rapid phase of elimination from the circulation with a half-life (t_{1/2}) of 27 minutes; FIG. 15B) a second prolonged phase with a half-life of 6.5 hours; FIG. 15C and FIG. 15D) Rag1−/− animals were reconstituted with 1¹²⁵ radiolabeled B4, C2, or isotype control (F632) IgMs following IR, PHx or sham surgeries. Tissues were harvested and radioactivity was measured at 6 h post surgery. FIG. 15C) Biodistribution of IgMs following hepatic IR showed increased levels of B4 and C2 in the liver compared to sham controls. Isotype control antibody F632 did not accumulate in any tissue. FIG. 15D) Biodistribution of IgMs in Rag1−/− following 70% PHx. B4 and C2 radiolabeled mAbs accumulated in the liver specifically. There was no tissue accumulation of antibody in sham or F632 isotype control treated animals following 70% PHx. Data was representative of 2 independent experiments, n=2 for each group; and FIG. 15E and FIG. 15F) Biodistribution of 1¹²⁵ radiolabeled B4 scFv in Rag1−/− following IR, PHx, or sham operation. Rag1−/− mice were given radiolabeled B4 scFv intraperitoneally immediately following surgeries, tissues were harvested and measured for radioactivity at 6 h post surgery. FIG. 15E) Biodistribution of B4 scFv in Rag1−/− mice following IR or sham operation showed accumulation of B4 scFv mainly in the liver of mice that underwent IR with no appreciable B4 scFv accumulation in sham animals. n=3 for each group, mean±SD. FIG. 15F) Biodistribution of radiolabeled B4 scFv in Rag1−/− following 70% PHx or sham operation, showed accumulation of B4 scFv in the liver of mice given 70% PHx with no accumulation in sham operated mice n=2 for each group. Biodistribution studies were representative of 2 separate experiments.

FIGS. 16A-16E show the protective effect of B4scFv and B4scFv-Crry against hepatic ischemic reperfusion injury. FIG. 16A) Serum ALT levels 24 hours post IR in sham, wild-type and wild-type mice injected with 25 μg B4scFv or B4scFv-Crry. n=3. FIGS. 16B-16E) H&E staining in liver 24 hours post reperfusion in FIG. 16B) sham, FIG. 16C) wild-type mouse, FIG. 16D) B4scFv treated mouse, and FIG. 16E) B4scFv-Crry treated mouse. Images taken at 40×zoom.

FIGS. 17A-17B are imaging analysis of B4 IgM deposition in human liver. FIG. 17A) Human liver section stained for IgM post-reperfusion of transplanted liver. FIG. 17B) Immunofluorescence confocal images of CD31 (endothelial marker, left panel) or B4 IgM (middle panel) stained ischemic non-reperfused human liver section. Right panel shows merged image of CD31 and B4 IgM staining.

FIGS. 18A-18C are a series of graphs showing human serum antibody levels after liver transplantation. FIG. 18A) total IgM antibodies, FIG. 18B) anti-albumin 2 antibodies, and FIG. 18C) total IgG antibodies.

FIGS. 19A-19D are a series of graphs showing human serum antibody levels after liver transplantation. FIG. 19A) anti-PE antibodies, FIG. 19B) anti-Annexin IV antibodies, FIG. 19C) anti-PC-BSA antibodies, and FIG. 19D) anti-cardiolipin antibodies.

FIGS. 20A-20B show B cell depletion with an anti-CD20 antibody reduced glomerular IgM deposition in mice with adriamycin nephropathy. Mice were injected with anti-CD20 monoclonal antibody to deplete their B cells prior to the induction of adriamycin nephropathy. FIG. 20A) Four weeks after induction of adriamycin nephropathy immunofluorescence microscopy was performed on kidneys to assess the abundance of glomerular IgM. Kidneys from three to five mice per group were examined. Thirty glomeruli per section were visualized, and the average for each mouse was determined. Mice injected with adriamycin demonstrated a substantial increase in glomerular IgM deposition compared to control animals. Mice treated with anti-CD20 demonstrated less glomerular IgM. Mice with adriamycin nephropathy that had been injected with the anti-CD20 but were also reconstituted with purified IgM eluted from the kidneys of mice with adriamycin nephropathy (Adria/anti-CD20/IgM) demonstrated an apparent increase in glomerular IgM. Glomeruli are indicated with arrowheads. Original magnification ×200. Scale bar=100 μM. After converting the images to grayscale, the brightness and contrast of the shown images were adjusted. All images were adjusted equally. FIG. 20B) Quantitative analysis of the glomerular IgM in the different treatment groups confirmed that glomerular IgM deposition was increased after injection with adriamycin, but that this increase was attenuated in mice injected with anti-CD20 therapy.

FIGS. 21A-21B show treatment with anti-CD20 reduced glomerular complement activation in mice with adriamycin nephropathy. Mice were injected with anti-CD20 monoclonal antibody to deplete B cells prior to the induction of adriamycin nephropathy. Four weeks after injection with adriamycin, complement activation in the glomeruli was examined by immunofluorescence microscopy. FIG. 21A) Staining for C4 demonstrated that glomerular C4 deposition increased after injection with adriamycin, but that treatment of the mice with anti-CD20 prevented this increase. FIG. 21B) Staining for C3 demonstrated that glomerular C3 deposition increased after injection with adriamycin. Treatment of the mice with anti-CD20 prevented this increase in C3 deposition. Glomeruli are indicated with arrowheads. Kidneys from three to five mice per group were examined. Thirty glomeruli per section were visualized, and the average for each mouse was determined. Original magnification ×200. Scale bar=100 μM. After converting the images to grayscale, the brightness and contrast of the shown images were adjusted. All images were adjusted equally.

FIGS. 22A-22D show that treatment with anti-CD20 reduced albuminuria in mice with adriamycin nephropathy. Mice were injected with anti-CD20 mAb to deplete B cells prior to the induction of adriamycin nephropathy. FIG. 22A) Urine albumin/creatinine levels were measured. Treatment of mice with adriamycin caused high-level albuminuria, but this was significantly attenuated by treatment with anti-CD20. Mice treated with anti-CD20 that were re-injected with IgM purified from diseased kidneys had levels of albuminuria similar to those seen in adriamycin treated mice. FIG. 22B) Glomerulosclerosis in the different treatment groups was assessed. Kidneys from three to five mice per group were examined. Forty glomeruli per section were visualized. Glomeruli are indicated with arrowheads. FIG. 22C) There was less glomerulosclerosis in mice treated with adriamycin and anti-CD20 compared to adriamycin treated mice, but this reduction was not statistically significant. FIG. 22D) Staining of kidneys for collagen IV demonstrated that injection of mice with adriamycin caused an increase in glomerular collagen IV deposition, and this was not significantly affected by treatment with anti-CD20. Kidneys from three mice per group were examined. Thirty glomeruli per section were visualized, and the average for each mouse was determined. Representative glomeruli from mice in each group are shown. Original magnification ×400. Scale bar=100 μM.

FIGS. 23A-23C show depletion of peritoneal B cells reduced the glomerular deposition of IgM, C3, and C4 in mice with adriamycin nephropathy. Peritoneal cells were depleted with hypotonic shock, starting two weeks prior to inducing adriamycin nephropathy. FIG. 23A) Immunofluorescence microscopy demonstrated that depletion of the peritoneal cells attenuated the glomerular deposition of IgM. FIG. 23B) Immunofluorescence microscopy for C4 demonstrated that depletion of the peritoneal cells also reduced C4 deposition within the glomeruli, although this did not reach statistical significance. FIG. 23C) Immunofluorescence microscopy for C3 demonstrated that depletion of the peritoneal cells prevented complement C3 activation within the glomerulus. Kidneys from three to four mice per group were examined. Thirty glomeruli per section were visualized, and the average for each mouse was determined. Glomeruli are indicated with arrowheads. Original magnification ×200. Scale bar=100 μM.

FIGS. 24A-24D show depletion of peritoneal B cells reduced albuminuria in mice with adriamycin nephropathy. Peritoneal cells were depleted with hypotonic shock, starting two weeks prior to the induction of adriamycin nephropathy. Urine albumin/creatinine levels were measured. Peritoneal depletion of B cells significantly attenuated the level of albuminuria one week (FIG. 24A) and four weeks (FIG. 24B) after injection of the adriamycin. FIG. 24C) Peritoneal cell depletion significantly reduced the degree of glomerulosclerosis compared to control mice that also had adriamycin nephropathy. Kidneys from three to seven mice per group were examined. Forty glomeruli per section were visualized, and the average for each mouse was determined. Representative glomeruli from mice in each group are shown. Glomeruli are indicated with arrowheads. Original magnification ×400. Scale bar=100 μM. FIG. 24D) Staining of kidneys for collagen IV demonstrated that injection of mice with adriamycin caused an increase in glomerular collagen IV deposition, and this was not significantly affected by depletion of peritoneal B cells. Kidneys from four mice per group were examined. Thirty glomeruli per section were visualized, and the average for each mouse was determined. Representative glomeruli from mice in each group are shown. Original magnification ×400.

FIGS. 25A-25B show IgM and C3d co-localized in glomeruli of patients with FSGS. FIG. 25A) Available tissue from 19 patients with FSGS was dual-stained for IgM and C3d. In biopsies that contained both IgM and C3d, the two immune factors co-localized within the glomeruli. FIG. 25B) Tissue from 19 patients with FSGS was dual-stained for IgM and C4. In biopsies that contained both IgM and C4, the two immune factors appeared to co-localize within the glomeruli. Original magnification ×400. Scale bar=100 μM. The brightness and contrast of the shown images were adjusted to improve localization of the factors in the overlay.

FIGS. 26A-26F show IgM deposited in mouse glomeruli after renal ischemia/reperfusion (I/R). Immunofluorescence microscopy revealed that IgM was present in the mesangium of mice 24 hours after sham treatment (FIG. 25A, FIG. 25C) or renal IR (FIG. 25B, FIG. 25D). FIG. 25E) Quantitative analysis confirmed that mesangial IgM deposition was increased after ischemia. FIG. 25F) Western blot analysis under reducing conditions of lysates made from kidneys subjected to sham treatment or IR also demonstrated IgM increase after ischemia. Arrowheads indicate glomeruli. FIG. 25A and FIG. 25B, Original magnification 3400; FIG. 25C and FIG. 25D, original magnification 3100.

FIGS. 27A-27B show anti-IgM immunofluorescence images of FIG. 27A) B4 mAb and FIG. 27B) C2 mAb localization to glomeruli in a renal IR injury mouse model.

FIGS. 28A-28B show immunofluorescence images of C3 deposition in glomerulus of FIG. 28A) wild-type (wt) mice and FIG. 28B) factor H deficient (fH−/−) mice.

FIGS. 29A-28B show immunofluorescence images of IgM deposition in glomerulus of FIG. 29A) wild-type (wt) mice at 3 months (left panel), 6 months (middle panel), and 9 months (right panel) of age; and FIG. 29B) factor H deficient (fH−/−) mice at 3 months (left panel), 6 months (middle panel), and 9 months (right panel) of age.

FIGS. 30A-30C show immunofluorescence images of C3 and IgM deposition in glomerulus of factor H deficient (fH−/−) mice at FIG. 30A) 3 months and FIG. 30B) 9 months of age. FIG. 30C) shows immunofluorescence images of C3 and IgM deposition in glomerulus of factor H deficient (fH−/μMT) mice at 9 months of age.

FIGS. 31A-31B show immunofluorescence images of FIG. 31A) IgM deposition and co-localization with synaptopodin in glomerulus of factor H deficient (fH−/−) mice and FIG. 31B) IgM deposition and co-localization with BM marker in glomerulus of factor H deficient (fH−/−) mice.

FIGS. 32A-32B show immunofluorescence images of C3 and C4 deposition in glomerulus of factor H deficient (fH−/−) mice at FIG. 32A) 3 months and FIG. 32B) 9 months of age.

FIGS. 33A-33B are a series of graphs showing FIG. 33A) serum urea nitrogen (SUN) levels and FIG. 33B) urine albumin to creatinine (Cr) ratio in wild-type, fH^{-/-}, and fH/μMT mice at 9 months of age.

FIGS. 34A-34J. FIGS. 34A-34D show a series of flow cytometry histograms of in vitro IgM and complement protein binding experiments with mesangial cells. FIG. 34A) IgM bound to mesangial cells, FIG. 34B) IgG did not bind to mesangial cells, FIG. 34C) C3 bound to mesangial cells, and FIG. 34D) C4 bound to mesangial cells. FIGS. 34E-34J) Monoclonal IgM clones demonstrated selective binding to glomerular cells in vivo and in vitro. B cell deficient mice were given an intravenous injection of monoclonal IgM. Kidney sections were assessed for presence of IgM by immunofluorescence. FIG. 34G) IgM deposition occurred following injection of the monoclonal IgM clone C2 into a B cell deficient mouse. Representative glomeruli are shown and are marked with arrows. FIG. 34H) The corresponding hematoxylin stained section highlighting the location of glomeruli with arrows. FIG. 34I) Mice injected with the monoclonal IgM clone D5 did not demonstrate evidence of IgM deposition. FIG. 34J) Corresponding hematoxylin stained section. Original magnification×200. Cultured murine mesangial cells were incubated with polyclonal IgM or seven different monoclonal IgM clones. Following incubation, cells were analyzed by flow cytometry to determine the degree of IgM binding. FIG. 34E) IgM antibodies that exhibited positive binding to mesangial cells are shown. FIG. 34F) The remaining five monoclonal IgM clones all of which did not demonstrate binding to mesangial cells are shown. Isotype control is represented by the shaded histogram.

FIG. 35 is a graph showing that administration of B4 mAb significantly worsened arthritic symptoms in a model of rheumatoid arthritis.

FIGS. 36A-36B provide a complement pathway analysis in oxidatively-stressed ARPE-19 cell monolayers. FIG. 36A) Oxidative stress was induced by treating cells with 500 μM H₂O₂, which sensitizes monolayers to complement attack when treating cells with 10% normal human serum (NHS) (Thurman, J. M., Renner, B., Kunchithapautham, K., Ferreira, V. P., Pangburn, M. K., Ablonczy, Z., Tomlinson, S., Holers, V. M., and Rohrer, B. (2009) J Biol Chem 284, 16939-16947). Pathway analysis was performed using serum depleted of specific complement components. Results shown are percentage of starting value in the presence of factor B-, C1q-, MBL-, and C1q/MBL-depleted sera, revealing that complement activation on H₂O₂-treated cells is triggered by lectin and amplified by the alternative pathway. FIG. 36B) Lectin pathway activation was probed in the absence of complement factors C2 or C4 to examine a potential bypass of these components. Elimination of either C2 or C4 from NHS eliminated the effect of H₂O₂+serum on TER, indicating that both components were for activity. Specificity of the depleted sera was confirmed by reconstituting with purified C2 and C4 protein, respectively.

FIGS. 37A-37C provide an analysis of the pattern recognition receptors involved in lectin pathway activation. FIG. 37A) Serum passed over the mannan column was analyzed for components of the lectin pathway. Western blot analysis showed depletion of MBL, MASP-2, and M-, L-, and H-ficolin in two samples (lanes 2 and 3), whereas C3 levels were unaffected. Normal human serum (lane 1) was used as control. FIG. 37B) Ficolin binding to ARPE monolayer was examined using NHS as the source in oxidatively-stressed cells, followed by specific antibody binding. Specific, saturable binding could be documented for M-ficolin and H-ficolin, whereas saturable binding was not seen for L-ficolin. Concentration of ficolins was calculated based on known concentrations in human serum. FIG. 37C) Reconstitution assays were performed to examine whether ligands on ARPE-19 cells triggering complement activation were recognized by ficolin or MBL, using TER as the readout. TER is reduced by H₂O₂+serum, but eliminated when serum is passed over a mannan binding column (MBL depl). Reduction in TER is reconstituted by adding MASP-2 together with one of the pattern recognition receptors; adding all three was not found to be additive.

FIGS. 38A-38D show the results of experiments performed to determine whether natural antibodies activate the lectin pathway. FIG. 38A) TER measurements were performed using serum from which either all antibodies (NHS depleted of all Igs) or IgM and IgD (serum from rag1−/− mice) was used, indicating that antibodies are required for complement activation in this assay. FIG. 38B) IgM binding to ARPE monolayer was examined on ARPE-19 cells cultured as monolayers in 96-well plates, using serum as a source of IgM, followed by colorimetric detection of IgM binding with anti-IgM antibodies. No difference in overall binding of IgM to ARPE-19 cells could be detected when comparing control and oxidatively-stressed cells. FIG. 38C) When using an IgM antibody specific for oxidative stress epitopes (IgM-C2), specific binding to cells was observed, which was augmented in the presence of oxidative stress. FIG. 38D) Reconstitution assays were performed to determine whether an IgM antibody known to recognize neoepitopes generated by oxidative stress, can activate the complement cascade in Ig-depleted serum. Reduction in TER is obliterated in Ig-depleted serum. Ig-depleted human serum used in the presence of IgM natural antibody, C2, was found to activate the complement cascade in this assay, whereas the control antibody, F1102, was ineffective.

FIGS. 39A-39D provide an epitope analysis of IgM-C2 natural antibody. FIG. 39A) ELISA analysis was performed, coating plates with BSA coupled to phosphatidylcholine (PC). Specific binding could be observed for IgM-C2 to this ligand as reported previously (Elvington, A., Atkinson, C., Kulik, L., Zhu, H., Yu, J., Kindy, M. S., Holers, V. M., and Tomlinson, S. (2012) J Immunol 188, 1460-1468), whereas the control IgM specific for annexin IV (IgM-B4) showed no binding. FIG. 39B) Since malondialdehyde (MDA) is generated on lipids by oxidative stress, it was examined whether specific binding of IgM-C2 to MDA-BSA could be documented. ELISA assays revealed binding of IgM-C2 to MDA-BSA, albeit possibly at a lower apparent affinity when compared to PC-BSA, or the MDA-BSA wells might have less capacity. No binding could be documented for the control IgM (IgMB4). FIG. 39C and FIG. 39D) To test whether reconstitution of Ig-depleted serum using IgM-C2 antibody is mediated by MDA-binding FIG. 39C) or unmodified lipid binding FIG. 39D), IgM-C2 was preabsorbed with either BSA-MDA or PC-BSA. BSA-MDA or PC-BSA was added to Ig-depleted serum as control. Reduction in TER can be mediated through binding of the IgM-C2 antibody to either one of the two lipid ligands.

FIGS. 40A-40B show the results of experiments performed to determine whether Malondialdehyde (MDA)-neoepitopes are present on oxidatively-stressed ARPE-19 cells. FIG. 40A) Immunofluorescence staining of ARPE cells using a-MDA in the presence and absence of H₂O₂. Specific staining was revealed in oxidatively-stressed cells when compared to control cells. Incubation without primary antibody was performed as a negative control. FIG. 40B) Both the anti-MDA (red; a-MDA) and the IgM-C2 antibody (green; a-C2) recognized epitopes present in a punctate fashion on ARPE cells.

FIGS. 41A-41B show the results of experiments performed to determine whether MDA-neoepitopes are present on oxidatively-stressed primary fetal human cells. Primary fetal human RPE cells were grown in monolayers and TER-assessed in response to 500 μM H₂O₂ and 10% normal human serum (NHS). FIG. 41A) Elimination of immunoglobulin (Ig-depleted serum) significantly reduced the drop in TER. The Ig-depleted serum could be reconstituted using the IgM-C2 and the IgM-B4 antibody, and not with a control (IgM-F1102) antibody. FIG. 41B) Reconstitution of Ig-depleted serum using IgM-C2 antibody is in part mediated by MDA-binding, as preabsorbing with BSA-MDA eliminated the effect. Oxidative stress-mediated generation of phospholipid neoepitopes is a more general phenomenon for RPE cells.

FIG. 42 shows the results of experiments performed to determine whether neoepitopes generated by oxidative stress serve as ligands for complement factor H (CFH) on oxidatively-stressed ARPE-19 cells. Malondialdehyde (MDA) has been postulated to serve as a ligand on cell surfaces to recruit CFH and prevent complement-mediated damage (Weismann, D., Hartvigsen, K., Lauer, N., Bennett, K. L., Scholl, H. P., Charbel Issa, P., Cano, M., Brandstatter, H., Tsimikas, S., Skerka, C., Superti-Furga, G., Handa, J. T., Zipfel, P. F., Witztum, J. L., and Binder, C. J. (2011) Nature 478, 76-81). Transepithelial resistance (TER) measurements were performed upon addition of 500 μM H₂O₂, +25% normal human serum (NHS); H₂O₂, +25% NHS supplemented with 375 μg of purified CFH; cells treated with H₂O₂, which was removed prior to the addition of HNH+exogenous CFH; or H₂O₂, +25% NHS supplemented with 50 μg of CR2-FH (a targeted inhibitory protein of the alternative pathway that targets the inhibitory domain of CFH to sites of C3d deposition). Only CR2-FH was able to block TER reduction induced by H₂O₂, +NHS, suggesting that neoepitopes generated by oxidative stress do not recruit CFH to the cell surface for protection.

FIGS. 43A-43B show the results of experiments performed to determine whether C2-IgM neoepitopes are generated in choroidal neovascularization (CNV) lesions and whether such serve to augment CNV growth in antibody-deficient mice reconstituted with C2-IgM. FIG. 43A) Immunofluorescence staining of CNV lesions using the IgM-C2 antibody. Specific staining was revealed when compared to controls in which the primary antibody was omitted. FIG. 43B) Antibody-deficient rag1−/− mice were reconstituted with three injections of C2-IgM, B4-IgM, or control IgM (F1102 and F632) during the course of CNV development. Both C2-IgM and B4-IgM injections resulted in a significant increase in lesion size when compared to the control antibody. CNV lesion size in wild type mice was unaffected.

### DETAILED DESCRIPTION OF THE INVENTION

The present invention provides targeted delivery methods and constructs for treating inflammatory diseases and/or detecting in vivo tissue injuries in an individual. The targeted delivery approach utilizes an antibody that recognizes an epitope found to be present at sites of inflammation. Specifically, it was found that monoclonal antibodies B4 and C2, initially identified as pathogenic IgM natural antibodies, recognize epitopes widely distributed on organs undergoing ischemia-reperfusion injury as well as sites of inflammation that are undergoing non-ischemic injury. These observations demonstrate the involvement of IgM natural antibodies in inflammatory disorders that go beyond ischemia-reperfusion injury, and suggest a widespread role of these natural antibodies in innate immune recognition of such disorders. The present application thus provides targeted delivery methods and constructs for treating inflammatory diseases and/or detecting in vivo tissue injury based on the binding properties of such natural antibodies.

Thus, the present application in one aspect provides a method of treating an inflammatory disease in an individual comprising administering to the individual an effective amount of a composition comprising a construct, wherein the construct comprises (a) an antibody or a fragment thereof, wherein the antibody or a fragment thereof specifically binds to Annexin IV or a phospholipid; and (b) a complement modulator (such as a complement inhibitor).

In another aspect, there is provided a method of detecting injury or inflammation in a tissue of an individual, comprising administering to the individual an effective amount of a composition comprising a construct, wherein the construct comprises (a) an antibody or a fragment thereof, wherein the antibody or a fragment thereof specifically binds to Annexin IV or a phospholipid; and (b) a detectable moiety.

In another aspect, there is provided a composition comprising a construct, wherein the construct comprises (a) an antibody or a fragment thereof, wherein the antibody or a fragment thereof specifically binds to Annexin IV or a phospholipid; and (b) a complement modulator or a detectable moiety.

Also provided are methods of delivering a complement modulator (such as a complement inhibitor) or a detectable moiety to a site of tissue injury in an individual, comprising administering to the individual an effective amount of a composition comprising a construct, wherein the construct comprises (a) an antibody or a fragment thereof, wherein the antibody or a fragment thereof specifically binds to Annexin IV or a phospholipid; and (b) a complement modulator (such as a complement inhibitor) or a detectable moiety.

The present invention also provides methods and compositions for treating ocular diseases. Using a combination of in vitro and in vivo techniques, it was shown that certain neoepitopes in the eye, namely, Annexin IV and phospholipid-based neoeptitopes, are involved during the development of ocular diseases such as age-related macular degeneration (“AMD”). The neoepitopes are recognized by natural antibodies, such as antibodies recognizing the same epitopes as IgM monoclonal antibodies B4 and C2. The binding of the natural antibodies to their respective epitopes in turn lead to the activation of lectin complement pathway and the alternative complement pathway. The present application thus for the first time defines mechanisms of complement activation in oxidatively stressed eye tissue (such as retinal pigmented epithelial monolayers (“RPE”), which provides a basis for the development of therapeutics and diagnostic agents for ocular diseases.

Thus, the present application in one aspect provides a method of inhibiting inflammation in the eye or treating an ocular disease in an individual, comprising administering to the individual an effective amount of an antibody or fragment thereof, wherein the antibody or fragment thereof does not activate complement activation, and wherein the antibody or fragment thereof: (i) specifically binds to Annexin IV (e.g., an epitope of Annexin IV); or (ii) specifically binds to a phospholipid (e.g., an epitope on a phospholipid).

In another aspect, there is provided a method of inhibiting inflammation in the eye or treating an ocular disease in an individual, comprising administering to the individual a composition comprising a construct, wherein the construct comprises: (a) an antibody or a fragment thereof, wherein the antibody or fragment thereof: (i) specifically binds to Annexin IV (e.g., an epitope of Annexin IV); or (ii) specifically binds to a phospholipid (e.g., an epitope on a phospholipid); and (b) a therapeutic agent (such as a complement inhibitor).

In another aspect, there is provided a method of detecting injury or inflammation in the eye in an individual, comprising administering to the individual an effective amount of a composition comprising a construct, wherein the construct comprises: (a) an antibody or a fragment thereof, wherein the antibody or fragment thereof: (i) specifically binds to Annexin IV (e.g., an epitope of Annexin IV); or (ii) specifically binds to a phospholipid (e.g., an epitope on a phospholipid); and (b) detectable moiety, wherein the presence of the detectable moiety in the eye is indicative of injury or inflammation in the eye.

Also provided are methods of delivering a complement modulator (such as a complement inhibitor) or a detectable moiety to a site of tissue injury in an individual, comprising administering to the individual an effective amount of a composition comprising a construct, wherein the construct comprises (a) an antibody or a fragment thereof, wherein the antibody or a fragment thereof specifically binds to Annexin IV and competitively inhibits the binding of monoclonal antibody B4 to Annexin IV; and (b) a complement modulator (such as a complement inhibitor) or a detectable moiety.

Also provided are unit dosage forms, kits, and articles of manufacture that are useful for methods described herein.

### DEFINITIONS

The term “individual” refers to a mammal, including humans. An individual includes, but is not limited to, human, bovine, horse, feline, canine, rodent, or primate. In some embodiments, the individual is human. In some embodiments, the individual is a human.

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing or improving the quality of life, increasing weight gain, and/or prolonging survival. Also encompassed by “treatment” is a reduction of pathological consequence of the disease. The methods of the invention contemplate any one or more of these aspects of treatment.

The term “effective amount” used herein refers to an amount of a compound or composition sufficient to treat a specified disorder, condition or disease such as ameliorate, palliate, lessen, and/or delay one or more of its symptoms.

As used herein, by “combination therapy” is meant that a first agent be administered in conjunction with another agent. “In conjunction with” refers to administration of one treatment modality in addition to another treatment modality, such as administration of a nanoparticle composition described herein in addition to administration of the other agent to the same individual. As such, “in conjunction with” refers to administration of one treatment modality before, during, or after delivery of the other treatment modality to the individual.

As used herein, by “pharmaceutically acceptable” or “pharmacologically compatible” is meant a material that is not biologically or otherwise undesirable, e.g., the material may be incorporated into a pharmaceutical composition administered to an individual or patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

As used herein and in the appended claims, the singular forms “a,” “an,” and “the” include plural reference unless the context clearly indicates otherwise.

Reference to “about” a value or parameter herein includes (and describes) embodiments that are directed to that value or parameter per se. For example, description referring to “about X” includes description of “X.”

It is understood that aspects, variations, and embodiments of the invention described herein include “consisting” and/or “consisting essentially of” aspects, variations, and embodiments.

### Methods of Treating Diseases

The present application in some embodiments provides a method of inhibiting complement activation, inhibiting inflammation, or treating an inflammatory disease in an individual, comprising administering to the individual an effective amount of a composition comprising a construct, wherein the construct comprises (a) an antibody or a fragment thereof, wherein the antibody or a fragment thereof specifically binds to Annexin IV or a phospholipid; and (b) a complement inhibitor. In some embodiments, the composition is administered by injection, such as parenteral, intravenous, subcutaneous, intraocular, intra-articular, or intramuscular injections. In some embodiments, there is provided a method of delivering a complement modulator (such as a complement inhibitor) to a site of tissue injury (such as non-ischemic tissue injury) in an individual, comprising administering to the individual an effective amount of a composition comprising a construct, wherein the construct comprises (a) an antibody or a fragment thereof, wherein the antibody or a fragment thereof specifically binds to Annexin IV or a phospholipid; and (b) a complement inhibitor.

In some embodiments, there is provided a method of inhibiting complement activation (or inhibiting inflammation for example complement-mediated inflammation) in a tissue having a non-ischemic injury in an individual, comprising administering to the individual an effective amount of a composition comprising a construct, wherein the construct comprises (a) an antibody or a fragment thereof, wherein the antibody or a fragment thereof specifically binds to Annexin IV; and (b) a complement inhibitor. In some embodiments, the antibody or fragment thereof competitively inhibits the binding of a pathogenic antibody (such as monoclonal antibody B4) to Annexin IV. In some embodiments, the antibody or antibody fragment thereof binds to the same epitope as a pathogenic antibody (such as monoclonal antibody B4) to Annexin IV. In some embodiments, the Annexin IV is present on the surface of a cell (and/or in a pathological structure) in an individual that is in or adjacent to a tissue undergoing (or is at risk of undergoing) tissue injury (such as non-ischemic injury) and/oxidative damage. In some embodiments, the Annexin IV is produced by a nucleated cell (such as a mammalian cell). In some embodiments, the Annexin IV is recombinant protein. In some embodiments, the construct is a fusion protein. In some embodiments, the targeting moiety and the active moiety are linked via a linker (such as a peptide linker). In some embodiments, the tissue is any one of liver or portal tract, heart, muscle, brain, central or peripheral nervous system, gastrointestinal tract, lung, limb, arterial or venous vascular system, skin, bone marrow cells including red blood cells, platelets and nucleated cells, pancreas, eye, joint, and kidney. In some embodiments, the tissue is any one of eye, joint, and kidney. In some embodiments, the inflammation (such as complement mediated inflammation) is associated with tissue damage resulting from inflammatory disorders, transplant rejection (cellular or antibody mediated), pregnancy-related diseases, adverse drug reactions, autoimmune or immune complex disorders. In some embodiments, at least about 10% (including for example at least about any of 2-%, 3-%, 40%, 50%, 60%, 70%, 80%, 90%, 95%, or 100%) complement activation or inflammation is inhibited.

In some embodiments, there is provided a method of inhibiting complement activation (or inhibiting inflammation for example complement-mediated inflammation) in a tissue having a non-ischemic injury in an individual, comprising administering to the individual an effective amount of a composition comprising a construct, wherein the construct comprises (a) an antibody or a fragment thereof, wherein the antibody or a fragment thereof specifically binds to a phospholipid; and (b) a complement inhibitor. In some embodiments, the antibody or fragment thereof competitively inhibits the binding of a pathogenic antibody (such as monoclonal antibody C2) to phospholipid. In some embodiments, the antibody or antibody fragment thereof binds to the same epitope as a pathogenic antibody (such as monoclonal antibody C2) to phospholipid. In some embodiments, the phospholipid is present on the surface of a cell (or in a pathological structure (e.g., drusen)) in an individual that is in or adjacent to a tissue undergoing (or is at risk of undergoing) tissue injury (such as non-ischemic injury) and/or oxidative damage. In some embodiments, the phospholipid is selected from the group consisting of phosphatidylethanolamine (PE), cardiolipin (CL), and phosphatidylcholine (PC). In some embodiments, the phospholipid is malondialdehyde (MDA). In some embodiments, the phospholipid is neutral. In some embodiments, the phospholipid is positively charged. In some embodiments, the phospholipid is oxidized. In some embodiments, the construct is a fusion protein. In some embodiments, the targeting moiety and the active moiety are linked via a linker (such as a peptide linker). In some embodiments, the tissue is any one of liver or portal tract, heart, muscle, brain, central or peripheral nervous system, gastrointestinal tract, lung, limb, arterial or venous vascular system, skin, bone marrow cells including red blood cells, platelets and nucleated cells, pancreas, eye, joint, and kidney. In some embodiments, the tissue is any one of eye, joint, and kidney. In some embodiments, the inflammation (such as complement mediated inflammation) is associated with tissue damage resulting from inflammatory disorders, transplant rejection (cellular or antibody mediated), pregnancy-related diseases, adverse drug reactions, autoimmune or immune complex disorders. In some embodiments, at least about 10% (including for example at least about any of 2-%, 3-%, 40%, 50%, 60%, 70%, 80%, 90%, 95%, or 100%) complement activation or inflammation is inhibited.

In some embodiments, there is provided a method of inhibiting complement activation (or inhibiting inflammation, for example complement-mediated inflammation) in a tissue having an oxidative damage in an individual, comprising administering to the individual an effective amount of a composition comprising a construct, wherein the construct comprises (a) an antibody or a fragment thereof, wherein the antibody or a fragment thereof specifically binds to Annexin IV; and (b) a complement inhibitor. In some embodiments, the antibody or fragment thereof competitively inhibits the binding of a pathogenic antibody (such as monoclonal antibody B4) to Annexin IV. In some embodiments, the antibody or antibody fragment thereof binds to the same epitope as a pathogenic antibody (such as monoclonal antibody B4) to Annexin IV. In some embodiments, the Annexin IV is present on the surface of a cell (and/or in a pathological structure) in an individual that is in or adjacent to a tissue undergoing (or is at risk of undergoing) tissue injury (such as non-ischemic injury) and/oxidative damage. In some embodiments, the Annexin IV is produced by a nucleated cell (such as a mammalian cell). In some embodiments, the Annexin IV is recombinant protein. In some embodiments, the construct is a fusion protein. In some embodiments, the targeting moiety and the active moiety are linked via a linker (such as a peptide linker). In some embodiments, the tissue is any one of liver or portal tract, heart, muscle, brain, central or peripheral nervous system, gastrointestinal tract, lung, limb, arterial or venous vascular system, skin, bone marrow cells including red blood cells, platelets and nucleated cells, pancreas, eye, joint, and kidney. In some embodiments, the tissue is any one of eye, joint, and kidney. In some embodiments, the inflammation (such as complement mediated inflammation) is associated with tissue damage resulting from inflammatory disorders, transplant rejection (cellular or antibody mediated), pregnancy-related diseases, adverse drug reactions, autoimmune or immune complex disorders. In some embodiments, at least about 10% (including for example at least about any of 2-%, 3-%, 40%, 50%, 60%, 70%, 80%, 90%, 95%, or 100%) complement activation or inflammation is inhibited.

In some embodiments, there is provided a method of inhibiting complement activation (or inhibiting inflammation, for example complement-mediated inflammation) in a tissue having an oxidative damage in an individual, comprising administering to the individual an effective amount of a composition comprising a construct, wherein the construct comprises (a) an antibody or a fragment thereof, wherein the antibody or a fragment thereof specifically binds to a phospholipid; and (b) a complement inhibitor. In some embodiments, the antibody or fragment thereof competitively inhibits the binding of a pathogenic antibody (such as monoclonal antibody C2) to phospholipid. In some embodiments, the antibody or antibody fragment thereof binds to the same epitope as a pathogenic antibody (such as monoclonal antibody C2) to phospholipid. In some embodiments, the phospholipid is present on the surface of a cell (or in a pathological structure (e.g., drusen)) in an individual that is in or adjacent to a tissue undergoing (or is at risk of undergoing) tissue injury (such as non-ischemic injury) and/or oxidative damage. In some embodiments, the phospholipid is selected from the group consisting of phosphatidylethanolamine (PE), cardiolipin (CL), and phosphatidylcholine (PC). In some embodiments, the phospholipid is malondialdehyde (MDA). In some embodiments, the phospholipid is neutral. In some embodiments, the phospholipid is positively charged. In some embodiments, the phospholipid is oxidized. In some embodiments, the construct is a fusion protein. In some embodiments, the targeting moiety and the active moiety are linked via a linker (such as a peptide linker). In some embodiments, the tissue is any one of liver or portal tract, heart, muscle, brain, central or peripheral nervous system, gastrointestinal tract, lung, limb, arterial or venous vascular system, skin, bone marrow cells including red blood cells, platelets and nucleated cells, pancreas, eye, joint, and kidney. In some embodiments, the tissue is any one of eye, joint, and kidney. In some embodiments, the inflammation (such as complement mediated inflammation) is associated with tissue damage resulting from inflammatory disorders, transplant rejection (cellular or antibody mediated), pregnancy-related diseases, adverse drug reactions, autoimmune or immune complex disorders. In some embodiments, at least about 10% (including for example at least about any of 2-%, 3-%, 40%, 50%, 60%, 70%, 80%, 90%, 95%, or 100%) complement activation or inflammation is inhibited.

In some embodiments, there is provided a method of treating an inflammatory disease (or a disease involving oxidative damage) in an individual, comprising administering to the individual an effective amount of a composition comprising a construct, wherein the construct comprises (a) an antibody or a fragment thereof, wherein the antibody or a fragment thereof specifically binds to Annexin IV; and (b) a complement inhibitor. In some embodiments, the antibody or fragment thereof competitively inhibits the binding of a pathogenic antibody (such as monoclonal antibody B4) to Annexin IV. In some embodiments, the antibody or antibody fragment thereof binds to the same epitope as a pathogenic antibody (such as monoclonal antibody B4) to Annexin IV. In some embodiments, the Annexin IV is present on the surface of a cell (and/or in a pathological structure) in an individual that is in or adjacent to a tissue undergoing (or is at risk of undergoing) tissue injury (such as non-ischemic injury) and/oxidative damage. In some embodiments, the Annexin IV is produced by a nucleated cell (such as a mammalian cell). In some embodiments, the Annexin IV is recombinant protein. In some embodiments, the construct is a fusion protein. In some embodiments, the targeting moiety and the active moiety are linked via a linker (such as a peptide linker). In some embodiments, the inflammatory disease is any of inflammatory disorders, transplant rejection (cellular or antibody mediated, such as hyperacute xenograft injection), pregnancy-related diseases, adverse drug reactions (such as drug allergy and IL-2 induced vascular leakage syndrome), autoimmune or immune complex disorders.

In some embodiments, there is provided a method of treating an inflammatory disease (or a disease involving oxidative damage) in an individual, comprising administering to the individual an effective amount of a composition comprising a construct, wherein the construct comprises (a) an antibody or a fragment thereof, wherein the antibody or a fragment thereof specifically binds to a phospholipid; and (b) a complement inhibitor. In some embodiments, the antibody or fragment thereof competitively inhibits the binding of a pathogenic antibody (such as monoclonal antibody C2) to phospholipid. In some embodiments, the antibody or antibody fragment thereof binds to the same epitope as a pathogenic antibody (such as monoclonal antibody C2) to phospholipid. In some embodiments, the phospholipid is present on the surface of a cell (or in a pathological structure (e.g., drusen)) in an individual that is in or adjacent to a tissue undergoing (or is at risk of undergoing) tissue injury (such as non-ischemic injury) and/or oxidative damage. In some embodiments, the phospholipid is selected from the group consisting of phosphatidylethanolamine (PE), cardiolipin (CL), and phosphatidylcholine (PC). In some embodiments, the phospholipid is malondialdehyde (MDA). In some embodiments, the phospholipid is neutral. In some embodiments, the phospholipid is positively charged. In some embodiments, the phospholipid is oxidized. In some embodiments, the construct is a fusion protein. In some embodiments, the targeting moiety and the active moiety are linked via a linker (such as a peptide linker). In some embodiments, the inflammatory disease is any of inflammatory disorders, transplant rejection (cellular or antibody mediated, such as hyperacute xenograft injection), pregnancy-related diseases, adverse drug reactions (such as drug allergy and IL-2 induced vascular leakage syndrome), autoimmune or immune complex disorders.

In some embodiments, the disease to be treated is an ocular disease. In some embodiments, the disease is an ocular disease associated with complement activation. In some embodiments, the disease is age-related macular degeneration (“AMD”), including wet AMD and dry AMD. Other ocular diseases that can be treated by methods described herein include, but are not limited to, CMV retinitis, macular edema, uveitis, glaucoma, diabetic retinopathy, retinitis pigmentosa, retinal detachment, proliferative vitreoretinopathy and ocular melanoma.

In some embodiments, the disease to be treated is inflammatory arthritis.

In some embodiments, the disease to be treated is a kidney disease, including but is not limited to, acute kidney injury, glomerulonephritis, chronic kidney disease, and focal segmental glomerulosclerosis.

In some embodiments, the disease to be treated is an inflammatory disorder, which include, but is not limited to, burns, endotoxemia, septic shock, adult respiratory distress syndrome, cardiopulmonary bypass, hemodialysis, anaphylactic shock, asthma, angioedema, Crohn's disease, sickle cell anemia, poststreptococcal glomerulonephritis, membranous nephritis, and pancreatitis.

In some embodiments, the disease to be treated is a pregnancy-related disease, which includes, but is not limited to, HELLP (Hemolytic anemia, elevated liver enzymes, and low platelet count), recurrent fetal loss, and pre-eclampsia.

In some embodiments, the disease to be treated is an autoimmune or immune complex disorder, which include, but is not limited to, myasthenia gravis, Alzheimer's disease, multiple sclerosis, neuromyelitis optica, rheumatoid arthritis, osteoarthritis, systemic lupus erythematosus, lupus nephritis, IgG4 associated diseases, insulin-dependent diabetes mellitus, acute disseminated encephalomyelitis, Addison's disease, antiphospholipid antibody syndrome, thrombotic thrombycytopenic purpura, autoimmune hepatitis, Crohn's disease, Goodpasture's syndromes, Graves' disease, Guillain-Barre syndrome, Hashimoto's disease, idiopathic thrombocytopenic purpura, pemphigus, Sjogren's syndrome, Takayasu's arteritis, autoimmune glomerulonephritis, membranoproliferative glomerulonephritis type II, membranous disease, paroxysmal nocturnal hemoglobinuria, age-related macular degeneration, diabetic maculopathy, uveitis, retinal degeneration disorders, diabetic nephropathy, focal segmental glomerulosclerosis, ANCA associated vasculitis, hemolytic uremic syndrome, Shiga-toxin-associated hemolytic uremic syndrome, and atypical hemolytic uremic syndrome. In some embodiments, the disease to be treated is an autoimmune glomerulonephritis, which includes, but is not limited to, immunoglobulin A nephropathy or membranoproliferative glomerularnephritis type I.

### Methods of Treating Ocular Diseases

The present application in some embodiments provides a method of inhibiting complement activation in the eye, inhibiting inflammation in the eye, or treating an ocular disease (for example an ocular disease involving oxidative damage or a complement-associated ocular disease) in an individual, comprising administering to the individual an effective amount of an antibody or fragment thereof, wherein the antibody or fragment thereof does not activate complement activation, and wherein the antibody or fragment thereof: (i) specifically binds to Annexin IV; or (ii) specifically binds to a phospholipid.

In some embodiments, there is provided a method of inhibiting complement activation or inhibiting inflammation (such as complement-driven inflammation) in the eye in an individual, comprising administering to the individual an effective amount of an antibody or fragment thereof, wherein the antibody or fragment thereof does not activate complement activation, and wherein the antibody or fragment thereof specifically binds to Annexin IV. In some embodiments, the antibody or fragment thereof competitively inhibits the binding of a pathogenic antibody (such as monoclonal antibody B4) to Annexin IV. In some embodiments, the antibody or antibody fragment thereof binds to the same epitope as a pathogenic antibody (such as monoclonal antibody B4) to Annexin IV. In some embodiments, the Annexin IV is present on the surface of a cell, a basement membrane (e.g., Bruch's membrane), or in a pathological structure (e.g., drusen) in an individual that is in or adjacent to a tissue undergoing (or is at risk of undergoing) tissue injury (such as non-ischemic injury) and/or oxidative damage. In some embodiments, the Annexin IV is produced by a nucleated cell (such as a mammalian cell). In some embodiments, the Annexin IV is recombinant protein. In some embodiments, at least about 10% (including for example at least about any of 20%, 30%, 40%, 50%, 60%, 70%, 80%, 90%, 95%, or 100%) complement activation or inflammation is inhibited.

In some embodiments, there is provided a method of inhibiting oxidative damage to eye (including for example oxidative damage to the photoreceptors, RPE/Bruch's membrane, macula, and/or choriocapillary complex) in an individual, comprising administering to the individual an effective amount of an antibody or fragment thereof, wherein the antibody or fragment thereof does not activate complement activation, and wherein the antibody or fragment thereof specifically binds to Annexin IV. In some embodiments, the antibody or fragment thereof competitively inhibits the binding of a pathogenic antibody (such as monoclonal antibody B4) to Annexin IV. In some embodiments, the antibody or antibody fragment thereof binds to the same epitope as a pathogenic antibody (such as monoclonal antibody B4) to Annexin IV. In some embodiments, the Annexin IV is present on the surface of a cell, a basement membrane (e.g., Bruch's membrane), or in a pathological structure (e.g., drusen) in an individual that is in or adjacent to a tissue undergoing (or is at risk of undergoing) tissue injury (such as non-ischemic injury) and/or oxidative damage. In some embodiments, the Annexin IV is produced by a nucleated cell (such as a mammalian cell). In some embodiments, the Annexin IV is recombinant protein. In some embodiments, at least about 10% (including for example at least about any of 20%, 30%, 40%, 50%, 60%, 70%, 80%, 90%, 95%, or 100%) oxidative damage is inhibited.

In some embodiments, there is provided a method of treating an ocular disease (such as a complement-associated ocular disease or an ocular disease involving oxidative damage) in an individual, comprising administering to the individual an effective amount of an antibody or fragment thereof, wherein the antibody or fragment thereof does not activate complement activation, and wherein the antibody or fragment thereof specifically binds to Annexin IV. In some embodiments, the antibody or fragment thereof competitively inhibits the binding of a pathogenic antibody (such as monoclonal antibody B4) to Annexin IV. In some embodiments, the antibody or antibody fragment thereof binds to the same epitope as a pathogenic antibody (such as monoclonal antibody B4) to Annexin IV. In some embodiments, the Annexin IV is present on the surface of a cell, a basement membrane (e.g., Bruch's membrane), or in a pathological structure (e.g., drusen) in an individual that is in or adjacent to a tissue undergoing (or is at risk of undergoing) tissue injury (such as non-ischemic injury) and/or oxidative damage. In some embodiments, the Annexin IV is produced by a nucleated cell (such as a mammalian cell). In some embodiments, the Annexin IV is recombinant protein. In some embodiments, the ocular disease is selected from the group consisting of age-related macular degeneration (AMD) (including wet AMD and dry AMD), cytomegalovirus (CMV) retinitis, macular edema, uveitis (anterior and posterior), glaucoma, open/wide-angle glaucoma, close/narrow-angle glaucoma, retinitis pigmentosa (RP), proliferative vitreoretinopathy, retinal detachment, corneal wound healing, corneal transplants, and ocular melanoma. In some embodiments, the ocular disease is AMD.

In some embodiments, there is provided a method of inhibiting complement activation or inhibiting inflammation (such as complement-driven inflammation) in the eye in an individual, comprising administering to the individual an effective amount of an antibody or fragment thereof, wherein the antibody or fragment thereof does not activate complement activation, and wherein the antibody or fragment thereof specifically binds to a phospholipid. In some embodiments, the antibody or fragment thereof competitively inhibits the binding of a pathogenic antibody (such as monoclonal antibody C2) to phospholipid. In some embodiments, the antibody or antibody fragment thereof binds to the same epitope as a pathogenic antibody (such as monoclonal antibody C2) to phospholipid. In some embodiments, the phospholipid is present on the surface of a cell, a basement membrane (e.g., Bruch's membrane), or in a pathological structure (e.g., drusen) in an individual that is in or adjacent to a tissue undergoing (or is at risk of undergoing) tissue injury (such as non-ischemic injury) and/or oxidative damage. In some embodiments, the phospholipid is selected from the group consisting of phosphatidylethanolamine (PE), cardiolipin (CL), and phosphatidylcholine (PC). In some embodiments, the phospholipid is malondiaidehyde (MDA). In some embodiments, the phospholipid is neutral. In some embodiments, the phospholipid is positively charged. In some embodiments, the phospholipid is oxidized. In some embodiments, at least about 10% (including for example at least about any of 20%, 30%, 40%, 50%, 60%, 70%, 80%, 90%, 95%, or 100%) complement activation or inflammation is inhibited.

In some embodiments, there is provided a method of inhibiting oxidative damage to eye (including for example oxidative damage to the photoreceptors, RPE/Bruch's membrane, macula, and/or choriocapillary complex) in an individual, comprising administering to the individual an effective amount of an antibody or fragment thereof, wherein the antibody or fragment thereof does not activate complement activation, and wherein the antibody or fragment thereof specifically binds to a phospholipid. In some embodiments, the antibody or fragment thereof competitively inhibits the binding of a pathogenic antibody (such as monoclonal antibody C2) to phospholipid. In some embodiments, the antibody or antibody fragment thereof binds to the same epitope as a pathogenic antibody (such as monoclonal antibody C2) to phospholipid. In some embodiments, the phospholipid is present on the surface of a cell, a basement membrane (e.g., Bruch's membrane), or in a pathological structure (e.g., drusen) in an individual that is in or adjacent to a tissue undergoing (or is at risk of undergoing) tissue injury (such as non-ischemic injury) and/or oxidative damage. In some embodiments, the phospholipid is selected from the group consisting of phosphatidylethanolamine (PE), cardiolipin (CL), and phosphatidylcholine (PC). In some embodiments, the phospholipid is malondialdehyde (MDA). In some embodiments, the phospholipid is neutral. In some embodiments, the phospholipid is positively charged. In some embodiments, the phospholipid is oxidized. In some embodiments, at least about 10% (including for example at least about any of 20%, 30%, 40%, 50%, 60%, 70%, 80%, 90%, 95%, or 100%) oxidative damage is inhibited.

In some embodiments, there is provided a method of treating an ocular disease (such as a complement-associated ocular disease or an ocular disease involving oxidative damage) in an individual, comprising administering to the individual an effective amount of an antibody or fragment thereof, wherein the antibody or fragment thereof does not activate complement activation, and wherein the antibody or fragment thereof specifically binds to a phospholipid. In some embodiments, the antibody or fragment thereof competitively inhibits the binding of a pathogenic antibody (such as monoclonal antibody C2) to phospholipid. In some embodiments, the antibody or antibody fragment thereof binds to the same epitope as a pathogenic antibody (such as monoclonal antibody C2) to phospholipid. In some embodiments, the phospholipid is present on the surface of a cell, a basement membrane (e.g., Bruch's membrane), or in a pathological structure (e.g., drusen) in an individual that is in or adjacent to a tissue undergoing (or is at risk of undergoing) tissue injury (such as non-ischemic injury) and/or oxidative damage. In some embodiments, the phospholipid is selected from the group consisting of phosphatidylethanolamine (PE), cardiolipin (CL), and phosphatidylcholine (PC). In some embodiments, the phospholipid is malondialdehyde (MDA). In some embodiments, the phospholipid is neutral. In some embodiments, the phospholipid is positively charged. In some embodiments, the ocular disease is selected from the group consisting of age-related macular degeneration (AMD) (including wet AMD and dry AMD), cytomegalovirus (CMV) retinitis, macular edema, uveitis (anterior and posterior), glaucoma, open/wide-angle glaucoma, close/narrow-angle glaucoma, retinitis pigmentosa (RP), proliferative vitreoretinopathy, retinal detachment, corneal wound healing, corneal transplants, and ocular melanoma. In some embodiments, the ocular disease is AMD.

In another aspect, there is provided a method of inhibiting inflammation in the eye or treating an ocular disease in an individual, comprising administering to the individual a composition comprising a construct, wherein the construct comprises: (a) an antibody or a fragment thereof, wherein the antibody or fragment thereof: (i) specifically binds to Annexin IV (e.g., an epitope of Annexin IV); or (ii) specifically binds to a phospholipid (e.g., an epitope on a phospholipid); and (b) a therapeutic agent (such as a complement inhibitor).

In some embodiments, there is provided a method of inhibiting complement activation or inhibiting inflammation (such as complement-driven inflammation) in the eye in an individual, comprising administering to the individual an effective amount of a composition comprising a construct, wherein the construct comprises: (a) an antibody or a fragment thereof, wherein the antibody or fragment thereof specifically binds to Annexin IV, and (b) a therapeutic agent (such as a complement inhibitor). In some embodiments, the antibody or fragment thereof competitively inhibits the binding of a pathogenic antibody (such as monoclonal antibody B4) to Annexin IV. In some embodiments, the antibody or antibody fragment thereof binds to the same epitope as a pathogenic antibody (such as monoclonal antibody B4) to Annexin IV. In some embodiments, the Annexin IV is present on the surface of a cell, a basement membrane (e.g., Bruch's membrane), or in a pathological structure (e.g., drusen) in an individual that is in or adjacent to a tissue undergoing (or is at risk of undergoing) tissue injury (such as non-ischemic injury) and/or oxidative damage. In some embodiments, the Annexin IV is produced by a nucleated cell (such as a mammalian cell). In some embodiments, the Annexin IV is recombinant protein. In some embodiments, at least about 10% (including for example at least about any of 20%, 30%, 40%, 50%, 60%, 70%, 80%, 90%, 95%, or 100%) complement activation or inflammation is inhibited.

In some embodiments, there is provided a method of inhibiting oxidative damage to eye (including for example oxidative damage to the photoreceptors, RPE/Bruch's membrane, macula, and/or choriocapillary complex) in an individual, comprising administering to the individual an effective amount of a composition comprising a construct, wherein the construct comprises: (a) an antibody or a fragment thereof, wherein the antibody or fragment thereof specifically binds to Annexin IV, and (b) a therapeutic agent (such as a complement inhibitor). In some embodiments, the antibody or fragment thereof competitively inhibits the binding of a pathogenic antibody (such as monoclonal antibody B4) to Annexin IV. In some embodiments, the antibody or antibody fragment thereof binds to the same epitope as a pathogenic antibody (such as monoclonal antibody B4) to Annexin IV. In some embodiments, the Annexin IV is present on the surface of a cell, a basement membrane (e.g., Bruch's membrane), or in a pathological structure (e.g., drusen) in an individual that is in or adjacent to a tissue undergoing (or is at risk of undergoing) tissue injury (such as non-ischemic injury) and/or oxidative damage. In some embodiments, the Annexin IV is produced by a nucleated cell (such as a mammalian cell). In some embodiments, the Annexin IV is recombinant protein. In some embodiments, at least about 10% (including for example at least about any of 20%, 30%, 40%, 50%, 60%, 70%, 80%, 90%, 95%, or 100%) oxidative damage is inhibited.

In some embodiments, there is provided a method of treating an ocular disease (such as a complement-associated ocular disease or an ocular disease involving oxidative damage) in an individual, comprising administering to the individual a composition comprising a construct, wherein the construct comprises: (a) an antibody or a fragment thereof, wherein the antibody or fragment thereof specifically binds to Annexin IV, and (b) a therapeutic agent (such as a complement inhibitor). In some embodiments, the antibody or fragment thereof competitively inhibits the binding of a pathogenic antibody (such as monoclonal antibody B4) to Annexin IV. In some embodiments, the antibody or antibody fragment thereof binds to the same epitope as a pathogenic antibody (such as monoclonal antibody B4) to Annexin IV. In some embodiments, the Annexin IV is present on the surface of a cell, a basement membrane (e.g., Bruch's membrane), or in a pathological structure (e.g., drusen) in an individual that is in or adjacent to a tissue undergoing (or is at risk of undergoing) tissue injury (such as non-ischemic injury) and/or oxidative damage. In some embodiments, the Annexin IV is produced by a nucleated cell (such as a mammalian cell). In some embodiments, the Annexin IV is recombinant protein. In some embodiments, the ocular disease is selected from the group consisting of age-related macular degeneration (AMD) (including wet AMD and dry AMD), cytomegalovirus (CMV) retinitis, macular edema, uveitis (anterior and posterior), glaucoma, open/wide-angle glaucoma, close/narrow-angle glaucoma, retinitis pigmentosa (RP), proliferative vitreoretinopathy, retinal detachment, corneal wound healing, corneal transplants, and ocular melanoma. In some embodiments, the ocular disease is AMD.

In some embodiments, there is provided a method of inhibiting complement activation or inhibiting inflammation (such as complement-driven inflammation) in the eye in an individual, comprising administering to the individual a composition comprising a construct, wherein the construct comprises: (a) an antibody or a fragment thereof, wherein the antibody or fragment thereof specifically binds to a phospholipid, and (b) a therapeutic agent (such as a complement inhibitor). In some embodiments, the antibody or fragment thereof competitively inhibits the binding of a pathogenic antibody (such as monoclonal antibody C2) to phospholipid. In some embodiments, the antibody or antibody fragment thereof binds to the same epitope as a pathogenic antibody (such as monoclonal antibody C2) to phospholipid. In some embodiments, the phospholipid is present on the surface of a cell, a basement membrane (e.g., Bruch's membrane), or in a pathological structure (e.g., drusen) in an individual that is in or adjacent to a tissue undergoing (or is at risk of undergoing) tissue injury (such as non-ischemic injury) and/or oxidative damage. In some embodiments, the phospholipid is selected from the group consisting of phosphatidylethanolamine (PE), cardiolipin (CL), and phosphatidylcholine (PC). In some embodiments, the phospholipid is malondialdehyde (MDA). In some embodiments, the phospholipid is neutral. In some embodiments, the phospholipid is positively charged. In some embodiments, the phospholipid is oxidized. In some embodiments, at least about 10% (including for example at least about any of 20%, 30%, 40%, 50%, 60%, 70%, 80%, 90%, 95%, or 100%) complement activation or inflammation is inhibited.

In some embodiments, there is provided a method of inhibiting oxidative damage to eye (including for example oxidative damage to the photoreceptors, RPE/Bruch's membrane, macula, and/or choriocapillary complex) in an individual, comprising administering to the individual a composition comprising a construct, wherein the construct comprises: (a) an antibody or a fragment thereof, wherein the antibody or fragment thereof specifically binds to a phospholipid, and (b) a therapeutic agent (such as a complement inhibitor). In some embodiments, the antibody or fragment thereof competitively inhibits the binding of a pathogenic antibody (such as monoclonal antibody C2) to phospholipid. In some embodiments, the antibody or antibody fragment thereof binds to the same epitope as a pathogenic antibody (such as monoclonal antibody C2) to phospholipid. In some embodiments, the phospholipid is present on the surface of a cell, a basement membrane (e.g., Bruch's membrane), or in a pathological structure (e.g., drusen) in an individual that is in or adjacent to a tissue undergoing (or is at risk of undergoing) tissue injury (such as non-ischemic injury) and/or oxidative damage. In some embodiments, the phospholipid is selected from the group consisting of phosphatidylethanolamine (PE), cardiolipin (CL), and phosphatidylcholine (PC). In some embodiments, the phospholipid is malondialdehyde (MDA). In some embodiments, the phospholipid is neutral. In some embodiments, the phospholipid is positively charged. In some embodiments, the phospholipid is oxidized. In some embodiments, at least about 10% (including for example at least about any of 20%, 30%, 40%, 50%, 60%, 70%, 80%, 90%, 95%, or 100%) oxidative damage is inhibited.

In some embodiments, there is provided a method of treating an ocular disease (such as a complement-associated ocular disease or an ocular disease involving oxidative damage) in an individual, comprising administering to the individual a composition comprising a construct, wherein the construct comprises: (a) an antibody or a fragment thereof, wherein the antibody or fragment thereof specifically binds to a phospholipid, and (b) a therapeutic agent (such as a complement inhibitor). In some embodiments, the antibody or fragment thereof competitively inhibits the binding of a pathogenic antibody (such as monoclonal antibody C2) to phospholipid. In some embodiments, the antibody or antibody fragment thereof binds to the same epitope as a pathogenic antibody (such as monoclonal antibody C2) to phospholipid. In some embodiments, the phospholipid is present on the surface of a cell, a basement membrane (e.g., Bruch's membrane), or in a pathological structure (e.g., drusen) in an individual that is in or adjacent to a tissue undergoing (or is at risk of undergoing) tissue injury (such as non-ischemic injury) and/or oxidative damage. In some embodiments, the phospholipid is selected from the group consisting of phosphatidylethanolamine (PE), cardiolipin (CL), and phosphatidylcholine (PC). In some embodiments, the phospholipid is malondialdehyde (MDA). In some embodiments, the phospholipid is neutral. In some embodiments, the phospholipid is positively charged. In some embodiments, the ocular disease is selected from the group consisting of age-related macular degeneration (AMD) (including wet AMD and dry AMD), cytomegalovirus (CMV) retinitis, macular edema, uveitis (anterior and posterior), glaucoma, open/wide-angle glaucoma, close/narrow-angle glaucoma, retinitis pigmentosa (RP), proliferative vitreoretinopathy, retinal detachment, corneal wound healing, corneal transplants, and ocular melanoma. In some embodiments, the ocular disease is AMD.

The methods described herein are also useful for any one of more of the following: (1) inhibiting, preventing, or delaying the formation of drusen in the eye; (2) inhibiting, preventing, or delaying loss of photoreceptor cells; (3) inhibiting, preventing, or delaying neovascularization associated with an ocular disease (such as AMD); (3) inhibiting, preventing, or delaying retinal detachment; (4) inhibiting, preventing, or delaying oxidative stress-mediated injury; and (5) improving visual acuity or visual field in the eye of an individual.

### Methods of Detecting Tissue Injury

The present application in some embodiments provides a method of detecting a complement-mediated tissue injury or inflammation or diagnosing an inflammatory disease in an individual, comprising administering to the individual an effective amount of a composition comprising a construct, wherein the construct comprises (a) an antibody or a fragment thereof, wherein the antibody or a fragment thereof specifically binds to Annexin IV or a phospholipid; and (b) a detectable moiety. In some embodiments, there is provided a method of delivering a detectable moiety to a site of complement-mediated tissue injury or inflammation in an individual, comprising administering to the individual an effective amount of a composition comprising a construct, wherein the construct comprises (a) an antibody or a fragment thereof, wherein the antibody or a fragment thereof specifically binds to Annexin IV or a phospholipid; and (b) a detectable moiety. In some embodiments, the method further comprises detecting the detectable moiety. In some embodiments, the composition is administered by injection, such as parenteral, intravenous, subcutaneous, intraocular, intra-articular, or intramuscular injections.

In some embodiments, there is provided a method of detecting a complement-mediated tissue injury or inflammation or diagnosing an inflammatory disease in an individual, comprising contacting a tissue of the individual with a composition comprising a construct, wherein the construct comprises (a) an antibody or a fragment thereof, wherein the antibody or a fragment thereof specifically binds to Annexin IV or a phospholipid; and (b) a detectable moiety. In some embodiments, the method further comprises detecting the detectable moiety.

In some embodiments, there is provided a method of detecting complement-mediated injury (or detecting inflammation for example complement-mediated inflammation) in a tissue of an individual, comprising administering to the individual an effective amount of a composition comprising a construct, wherein the construct comprises (a) an antibody or a fragment thereof, wherein the antibody or a fragment thereof specifically binds to Annexin IV; and (b) a detectable moiety, wherein the presence of the detectable moiety at the tissue is indicative of a complement-mediated tissue injury (or complement-mediated inflammation). In some embodiments, the method further comprises detecting the detectable moiety. In some embodiments, the antibody or fragment thereof competitively inhibits the binding of a pathogenic antibody (such as monoclonal antibody B4) to Annexin IV. In some embodiments, the antibody or antibody fragment thereof binds to the same epitope as a pathogenic antibody (such as monoclonal antibody B4) to Annexin IV. In some embodiments, the Annexin IV is present on the surface of a cell (and/or in a pathological structure) in an individual that is in or adjacent to a tissue undergoing (or is at risk of undergoing) tissue injury (such as non-ischemic injury) and/oxidative damage. In some embodiments, the Annexin IV is produced by a nucleated cell (such as a mammalian cell). In some embodiments, the Annexin IV is recombinant protein. In some embodiments, the construct is a fusion protein. In some embodiments, the targeting moiety and the active moiety are linked via a linker (such as a peptide linker). In some embodiments, the tissue is any one of liver or portal tract, heart, muscle, brain, central or peripheral nervous system, gastrointestinal tract, lung, limb, arterial or venous vascular system, skin, bone marrow cells including red blood cells, platelets and nucleated cells, pancreas, eye, joint, and kidney. In some embodiments, the tissue is any one of eye, joint, and kidney. In some embodiments, the tissue is any one of eye, joint, and kidney. In some embodiments, the inflammation (such as complement mediated inflammation) is associated with tissue damage resulting from inflammatory disorders, transplant rejection (cellular or antibody mediated), pregnancy-related diseases, adverse drug reactions, autoimmune or immune complex disorders.

In some embodiments, there is provided a method of detecting complement-mediated injury (or detecting inflammation for example complement-mediated inflammation) in a tissue of an individual, comprising administering to the individual an effective amount of a composition comprising a construct, wherein the construct comprises (a) an antibody or a fragment thereof, wherein the antibody or a fragment thereof specifically binds to a phospholipid; and (b) a detectable moiety, wherein the presence of the detectable moiety at the tissue is indicative of a complement-mediated tissue injury (or complement-mediated inflammation). In some embodiments, the method further comprises detecting the detectable moiety. In some embodiments, the antibody or fragment thereof competitively inhibits the binding of a pathogenic antibody (such as monoclonal antibody C2) to phospholipid. In some embodiments, the antibody or antibody fragment thereof binds to the same epitope as a pathogenic antibody (such as monoclonal antibody C2) to phospholipid. In some embodiments, the phospholipid is present on the surface of a cell (or in a pathological structure (e.g., drusen)) in an individual that is in or adjacent to a tissue undergoing (or is at risk of undergoing) tissue injury (such as non-ischemic injury) and/or oxidative damage. In some embodiments, the phospholipid is selected from the group consisting of phosphatidylethanolamine (PE), cardiolipin (CL), and phosphatidylcholine (PC). In some embodiments, the phospholipid is malondialdehyde (MDA). In some embodiments, the phospholipid is neutral. In some embodiments, the phospholipid is positively charged. In some embodiments, the phospholipid is oxidized. In some embodiments, the construct is a fusion protein. In some embodiments, the targeting moiety and the active moiety are linked via a linker (such as a peptide linker). In some embodiments, the tissue is any one of liver or portal tract, heart, muscle, brain, central or peripheral nervous system, gastrointestinal tract, lung, limb, arterial or venous vascular system, skin, bone marrow cells including red blood cells, platelets and nucleated cells, pancreas, eye, joint, and kidney. In some embodiments, the tissue is any one of eye, joint, and kidney. In some embodiments, the tissue is any one of eye, joint, and kidney. In some embodiments, the inflammation (such as complement mediated inflammation) is associated with tissue damage resulting from inflammatory disorders, transplant rejection (cellular or antibody mediated), pregnancy-related diseases, adverse drug reactions, autoimmune or immune complex disorders.

In some embodiments, there is provided a method of detecting oxidative damage (or an inflammatory disease involving oxidative damage) in a tissue in an individual, comprising administering to the individual an effective amount of a composition comprising a construct, wherein the construct comprises (a) an antibody or a fragment thereof, wherein the antibody or a fragment thereof specifically binds to Annexin IV; and (b) a detectable moiety, wherein the presence of the detectable moiety at the tissue is indicative of a oxidative damage in the tissue. In some embodiments, the method further comprises detecting the detectable moiety. In some embodiments, the antibody or fragment thereof competitively inhibits the binding of a pathogenic antibody (such as monoclonal antibody B4) to Annexin IV. In some embodiments, the antibody or antibody fragment thereof binds to the same epitope as a pathogenic antibody (such as monoclonal antibody B4) to Annexin IV. In some embodiments, the Annexin IV is present on the surface of a cell (and/or in a pathological structure) in an individual that is in or adjacent to a tissue undergoing (or is at risk of undergoing) tissue injury (such as non-ischemic injury) and/oxidative damage. In some embodiments, the Annexin IV is produced by a nucleated cell (such as a mammalian cell). In some embodiments, the Annexin IV is recombinant protein. In some embodiments, the construct is a fusion protein. In some embodiments, the targeting moiety and the active moiety are linked via a linker (such as a peptide linker). In some embodiments, the tissue is any one of liver or portal tract, heart, muscle, brain, central or peripheral nervous system, gastrointestinal tract, lung, limb, arterial or venous vascular system, skin, bone marrow cells including red blood cells, platelets and nucleated cells, pancreas, eye, joint, and kidney. In some embodiments, the tissue is any one of eye, joint, and kidney. In some embodiments, the tissue is any one of eye, joint, and kidney. In some embodiments, the oxidative damage is associated with tissue damage resulting from inflammatory disorders, transplant rejection (cellular or antibody mediated), pregnancy-related diseases, adverse drug reactions, autoimmune or immune complex disorders.

In some embodiments, there is provided a method of detecting oxidative damage (or an inflammatory disease involving oxidative damage) in a tissue in an individual, comprising administering to the individual an effective amount of a composition comprising a construct, wherein the construct comprises (a) an antibody or a fragment thereof, wherein the antibody or a fragment thereof specifically binds to a phospholipid; and (b) a detectable moiety, wherein the presence of the detectable moiety at the tissue is indicative of a oxidative damage in the tissue. In some embodiments, the method further comprises detecting the detectable moiety. In some embodiments, the antibody or fragment thereof competitively inhibits the binding of a pathogenic antibody (such as monoclonal antibody C2) to phospholipid. In some embodiments, the antibody or antibody fragment thereof binds to the same epitope as a pathogenic antibody (such as monoclonal antibody C2) to phospholipid. In some embodiments, the phospholipid is present on the surface of a cell (or in a pathological structure (e.g., drusen)) in an individual that is in or adjacent to a tissue undergoing (or is at risk of undergoing) tissue injury (such as non-ischemic injury) and/or oxidative damage. In some embodiments, the phospholipid is selected from the group consisting of phosphatidylethanolamine (PE), cardiolipin (CL), and phosphatidylcholine (PC). In some embodiments, the phospholipid is malondialdehyde (MDA). In some embodiments, the phospholipid is neutral. In some embodiments, the phospholipid is positively charged. In some embodiments, the phospholipid is oxidized. In some embodiments, the construct is a fusion protein. In some embodiments, the targeting moiety and the active moiety are linked via a linker (such as a peptide linker). In some embodiments, the tissue is any one of liver or portal tract, heart, muscle, brain, central or peripheral nervous system, gastrointestinal tract, lung, limb, arterial or venous vascular system, skin, bone marrow cells including red blood cells, platelets and nucleated cells, pancreas, eye, joint, and kidney. In some embodiments, the tissue is any one of eye, joint, and kidney. In some embodiments, the tissue is any one of eye, joint, and kidney. In some embodiments, the oxidative damage is associated with tissue damage resulting from inflammatory disorders, transplant rejection (cellular or antibody mediated), pregnancy-related diseases, adverse drug reactions, autoimmune or immune complex disorders.

In some embodiments, there is provided a method of detecting non-ischemic tissue injury (or an inflammatory disease involving non-ischemic tissue inury) in a tissue in an individual, comprising administering to the individual an effective amount of a composition comprising a construct, wherein the construct comprises (a) an antibody or a fragment thereof, wherein the antibody or a fragment thereof specifically binds to Annexin IV; and (b) a detectable moiety, wherein the presence of the detectable moiety at the tissue is indicative of non-ischemic tissue injury. In some embodiments, the method further comprises detecting the detectable moiety. In some embodiments, the antibody or fragment thereof competitively inhibits the binding of a pathogenic antibody (such as monoclonal antibody B4) to Annexin IV. In some embodiments, the antibody or antibody fragment thereof binds to the same epitope as a pathogenic antibody (such as monoclonal antibody B4) to Annexin IV. In some embodiments, the Annexin IV is present on the surface of a cell (and/or in a pathological structure) in an individual that is in or adjacent to a tissue undergoing (or is at risk of undergoing) tissue injury (such as non-ischemic injury) and/oxidative damage. In some embodiments, the Annexin IV is produced by a nucleated cell (such as a mammalian cell). In some embodiments, the Annexin IV is recombinant protein. In some embodiments, the construct is a fusion protein. In some embodiments, the targeting moiety and the active moiety are linked via a linker (such as a peptide linker). In some embodiments, the tissue is any one of liver or portal tract, heart, muscle, brain, central or peripheral nervous system, gastrointestinal tract, lung, limb, arterial or venous vascular system, skin, bone marrow cells including red blood cells, platelets and nucleated cells, pancreas, eye, joint, and kidney. In some embodiments, the tissue is any one of eye, joint, and kidney. In some embodiments, the tissue is any one of eye, joint, and kidney. In some embodiments, the non-ischemic tissue injury is associated with tissue damage resulting from inflammatory disorders, transplant rejection (cellular or antibody mediated), pregnancy-related diseases, adverse drug reactions, autoimmune or immune complex disorders.

In some embodiments, there is provided a method of detecting non-ischemic tissue injury (or an inflammatory disease involving non-ischemic tissue inury) in a tissue in an individual, comprising administering to the individual an effective amount of a composition comprising a construct, wherein the construct comprises (a) an antibody or a fragment thereof, wherein the antibody or a fragment thereof specifically binds to a phospholipid; and (b) a detectable moiety, wherein the presence of the detectable moiety at the tissue is indicative of non-ischemic tissue injury. In some embodiments, the method further comprises detecting the detectable moiety. In some embodiments, the antibody or fragment thereof competitively inhibits the binding of a pathogenic antibody (such as monoclonal antibody C2) to phospholipid. In some embodiments, the antibody or antibody fragment thereof binds to the same epitope as a pathogenic antibody (such as monoclonal antibody C2) to phospholipid. In some embodiments, the phospholipid is present on the surface of a cell (or in a pathological structure (e.g., drusen)) in an individual that is in or adjacent to a tissue undergoing (or is at risk of undergoing) tissue injury (such as non-ischemic injury) and/or oxidative damage. In some embodiments, the phospholipid is selected from the group consisting of phosphatidylethanolamine (PE), cardiolipin (CL), and phosphatidylcholine (PC). In some embodiments, the phospholipid is malondialdehyde (MDA). In some embodiments, the phospholipid is neutral. In some embodiments, the phospholipid is positively charged. In some embodiments, the phospholipid is oxidized. In some embodiments, the construct is a fusion protein. In some embodiments, the targeting moiety and the active moiety are linked via a linker (such as a peptide linker). In some embodiments, the tissue is any one of liver or portal tract, heart, muscle, brain, central or peripheral nervous system, gastrointestinal tract, lung, limb, arterial or venous vascular system, skin, bone marrow cells including red blood cells, platelets and nucleated cells, pancreas, eye, joint, and kidney. In some embodiments, the tissue is any one of eye, joint, and kidney. In some embodiments, the tissue is any one of eye, joint, and kidney. In some embodiments, the non-ischemic tissue injury is associated with tissue damage resulting from inflammatory disorders, transplant rejection (cellular or antibody mediated), pregnancy-related diseases, adverse drug reactions, autoimmune or immune complex disorders.

In some embodiments, there is provided a method of diagnosing (or assisting in diagnosing) an tissue-specific inflammatory disease (or a disease involving oxidative damage) in an individual, comprising administering to the individual an effective amount of a composition comprising a construct, wherein the construct comprises (a) an antibody or a fragment thereof, wherein the antibody or a fragment thereof specifically binds to Annexin IV; and (b) a detectable moiety, wherein the presence of the detectable moiety at the tissue is indicative of the inflammatory disease in the tissue. In some embodiments, the method further comprises detecting the detectable moiety. In some embodiments, the antibody or fragment thereof competitively inhibits the binding of a pathogenic antibody (such as monoclonal antibody B4) to Annexin IV. In some embodiments, the antibody or antibody fragment thereof binds to the same epitope as a pathogenic antibody (such as monoclonal antibody B4) to Annexin IV. In some embodiments, the Annexin IV is present on the surface of a cell (and/or in a pathological structure) in an individual that is in or adjacent to a tissue undergoing (or is at risk of undergoing) tissue injury (such as non-ischemic injury) and/oxidative damage. In some embodiments, the Annexin IV is produced by a nucleated cell (such as a mammalian cell). In some embodiments, the Annexin IV is recombinant protein. In some embodiments, the construct is a fusion protein. In some embodiments, the targeting moiety and the active moiety are linked via a linker (such as a peptide linker). In some embodiments, the inflammatory disease to be diagnosed is any of inflammatory disorders, transplant rejection (cellular or antibody mediated, such as hyperacute xenograft injection), pregnancy-related diseases, adverse drug reactions (such as drug allergy and IL-2 induced vascular leakage syndrome), autoimmune or immune complex disorders.

In some embodiments, there is provided a method of diagnosing (or assisting in diagnosing) an tissue-specific inflammatory disease (or a disease involving oxidative damage) in an individual, comprising administering to the individual an effective amount of a composition comprising a construct, wherein the construct comprises (a) an antibody or a fragment thereof, wherein the antibody or a fragment thereof specifically binds to a phospholipid; and (b) a detectable moiety, wherein the presence of the detectable moiety at the tissue is indicative of the inflammatory disease in the tissue. In some embodiments, the method further comprises detecting the detectable moiety. In some embodiments, the antibody or fragment thereof competitively inhibits the binding of a pathogenic antibody (such as monoclonal antibody C2) to phospholipid. In some embodiments, the antibody or antibody fragment thereof binds to the same epitope as a pathogenic antibody (such as monoclonal antibody C2) to phospholipid. In some embodiments, the phospholipid is present on the surface of a cell (or in a pathological structure (e.g., drusen)) in an individual that is in or adjacent to a tissue undergoing (or is at risk of undergoing) tissue injury (such as non-ischemic injury) and/or oxidative damage. In some embodiments, the phospholipid is selected from the group consisting of phosphatidylethanolamine (PE), cardiolipin (CL), and phosphatidylcholine (PC). In some embodiments, the phospholipid is malondialdehyde (MDA). In some embodiments, the phospholipid is neutral. In some embodiments, the phospholipid is positively charged. In some embodiments, the phospholipid is oxidized. In some embodiments, the construct is a fusion protein. In some embodiments, the targeting moiety and the active moiety are linked via a linker (such as a peptide linker). In some embodiments, the inflammatory disease to be diagnosed is any of inflammatory disorders, transplant rejection (cellular or antibody mediated, such as hyperacute xenograft injection), pregnancy-related diseases, adverse drug reactions (such as drug allergy and IL-2 induced vascular leakage syndrome), autoimmune or immune complex disorders.

In some embodiments, there is provided a method of diagnosing (or assisting in diagnosing) an tissue-specific inflammatory disease (or a disease involving oxidative damage) in an individual, comprising contacting a tissue sample from an individual with an effective amount of a composition comprising a construct, wherein the construct comprises (a) an antibody or a fragment thereof, wherein the antibody or a fragment thereof specifically binds to Annexin IV; and (b) a detectable moiety, wherein the presence of the detectable moiety at the tissue is indicative of the inflammatory disease in the tissue. In some embodiments, the method further comprises detecting the detectable moiety. In some embodiments, the antibody or fragment thereof competitively inhibits the binding of a pathogenic antibody (such as monoclonal antibody B4) to Annexin IV. In some embodiments, the antibody or antibody fragment thereof binds to the same epitope as a pathogenic antibody (such as monoclonal antibody B4) to Annexin IV. In some embodiments, the Annexin IV is present on the surface of a cell (and/or in a pathological structure) in an individual that is in or adjacent to a tissue undergoing (or is at risk of undergoing) tissue injury (such as non-ischemic injury) and/oxidative damage. In some embodiments, the Annexin IV is produced by a nucleated cell (such as a mammalian cell). In some embodiments, the Annexin IV is recombinant protein. In some embodiments, the construct is a fusion protein. In some embodiments, the targeting moiety and the active moiety are linked via a linker (such as a peptide linker). In some embodiments, the inflammatory disease to be diagnosed is any of inflammatory disorders, transplant rejection (cellular or antibody mediated, such as hyperacute xenograft injection), pregnancy-related diseases, adverse drug reactions (such as drug allergy and IL-2 induced vascular leakage syndrome), autoimmune or immune complex disorders.

In some embodiments, there is provided a method of diagnosing (or assisting in diagnosing) an tissue-specific inflammatory disease (or a disease involving oxidative damage) in an individual, comprising contacting a tissue sample from an individual with an effective amount of a composition comprising a construct, wherein the construct comprises (a) an antibody or a fragment thereof, wherein the antibody or a fragment thereof specifically binds to a phospholipid; and (b) a detectable moiety, wherein the presence of the detectable moiety at the tissue is indicative of the inflammatory disease in the tissue. In some embodiments, the method further comprises detecting the detectable moiety. In some embodiments, the antibody or fragment thereof competitively inhibits the binding of a pathogenic antibody (such as monoclonal antibody C2) to phospholipid. In some embodiments, the antibody or antibody fragment thereof binds to the same epitope as a pathogenic antibody (such as monoclonal antibody C2) to phospholipid. In some embodiments, the phospholipid is present on the surface of a cell (or in a pathological structure (e.g., drusen)) in an individual that is in or adjacent to a tissue undergoing (or is at risk of undergoing) tissue injury (such as non-ischemic injury) and/or oxidative damage. In some embodiments, the phospholipid is selected from the group consisting of phosphatidylethanolamine (PE), cardiolipin (CL), and phosphatidylcholine (PC). In some embodiments, the phospholipid is malondialdehyde (MDA). In some embodiments, the phospholipid is neutral. In some embodiments, the phospholipid is positively charged. In some embodiments, the phospholipid is oxidized. In some embodiments, the construct is a fusion protein. In some embodiments, the targeting moiety and the active moiety are linked via a linker (such as a peptide linker). In some embodiments, the inflammatory disease to be diagnosed is any of inflammatory disorders, transplant rejection (cellular or antibody mediated, such as hyperacute xenograft injection), pregnancy-related diseases, adverse drug reactions (such as drug allergy and IL-2 induced vascular leakage syndrome), autoimmune or immune complex disorders.

In some embodiments, the disease to be diagnosed is an ocular disease. In some embodiments, the disease is an ocular disease associated with complement activation. In some embodiments, the disease is age-related macular degeneration (“AMD”), including wet AMD and dry AMD. Other diseases that can be diagnosed by methods described herein include, but are not limited to, CMV retinitis, macular edema, uveitis, glaucoma, diabetic retinopathy, retinitis pigmentosa, retinal detachment, proliferative vitreoretinopathy and ocular melanoma.

In some embodiments, the disease to be diagnosed is inflammatory arthritis.

In some embodiments, the disease to be diagnosed is a kidney disease, including but is not limited to, acute kidney injury, glomerulonephritis, chronic kidney disease, and focal segmental glomerulosclerosis.

In some embodiments, the disease to be diagnosed is an inflammatory disorder, which include, but is not limited to, burns, endotoxemia, septic shock, adult respiratory distress syndrome, cardiopulmonary bypass, hemodialysis, anaphylactic shock, asthma, angioedema, Crohn's disease, sickle cell anemia, poststreptococcal glomerulonephritis, membranous nephritis, and pancreatitis.

In some embodiments, the disease to be diagnosed is a pregnancy-related disease, which includes, but is not limited to, HELLP (Hemolytic anemia, elevated liver enzymes, and low platelet count), recurrent fetal loss, and pre-eclampsia.

In some embodiments, the disease to be diagnosed is an autoimmune or immune complex disorder, which include, but is not limited to, myasthenia gravis, Alzheimer's disease, multiple sclerosis, neuromyelitis optica, rheumatoid arthritis, osteoarthritis, systemic lupus erythematosus, lupus nephritis, IgG4 associated diseases, insulin-dependent diabetes mellitus, acute disseminated encephalomyelitis, Addison's disease, antiphospholipid antibody syndrome, thrombotic thrombycytopenic purpura, autoimmune hepatitis, Crohn's disease, Goodpasture's syndromes, Graves' disease, Guillain-Barre syndrome, Hashimoto's disease, idiopathic thrombocytopenic purpura, pemphigus, Sjogren's syndrome, Takayasu's arteritis, autoimmune glomerulonephritis, membranoproliferative glomerulonephritis type II, membranous disease, paroxysmal nocturnal hemoglobinuria, age-related macular degeneration, diabetic maculopathy, uveitis, retinal degeneration disorders, diabetic nephropathy, focal segmental glomerulosclerosis, ANCA associated vasculitis, hemolytic uremic syndrome, Shiga-toxin-associated hemolytic uremic syndrome, and atypical hemolytic uremic syndrome. In some embodiments, the disease to be diagnosed is an autoimmune glomerulonephritis, which includes, but is not limited to, immunoglobulin A nephropathy or membranoproliferative glomerularnephritis type I.

Methods of Detecting Tissue Injury in the Eye

Also provided herein are methods of detecting injury or inflammation in the eye in an individual, comprising administering to the individual an effective amount of a composition comprising a construct, wherein the construct comprises: (a) an antibody or a fragment thereof, wherein the antibody or fragment thereof: (i) specifically binds to Annexin IV or (ii) specifically binds to phospholipid; and (b) detectable moiety, wherein the presence of the detectable moiety in the eye is indicative of injury or inflammation in the eye. In some embodiments, the method further comprises detecting the detectable moiety. In some embodiments, the composition is administered by injection, such as intraocular injections.

In some embodiments, the method is useful for detecting deterioration of the photoreceptor cells, retinal ganglion cells, RPE, Bruch's membrane, choriocapillary complex, macula, or cornea. In some embodiments, the method is useful for detecting inflammation in the of the photoreceptor cells, retinal ganglion cells, RPE, Bruch's membrane, choriocapillary complex, macula, or cornea. In some embodiments, the method is useful for detecting oxidative damage of the photoreceptor cells, retinal ganglion cells, RPE, Bruch's membrane, choriocapillary complex, macula, or cornea. In some embodiments, the method is useful for detecting pathological lesions at the RPE/choroid interface in the macula.

In some embodiments, there is provided a method of detecting complement-mediated injury (or detecting inflammation for example complement-mediated inflammation) in an eye tissue of an individual, comprising administering to the individual an effective amount of a composition comprising a construct, wherein the construct comprises (a) an antibody or a fragment thereof, wherein the antibody or a fragment thereof specifically binds to Annexin IV; and (b) a detectable moiety, wherein the presence of the detectable moiety at the tissue is indicative of a complement-mediated eye tissue injury (or complement-mediated inflammation). In some embodiments, the method further comprises detecting the detectable moiety. In some embodiments, the antibody or fragment thereof competitively inhibits the binding of a pathogenic antibody (such as monoclonal antibody B4) to Annexin IV. In some embodiments, the antibody or antibody fragment thereof binds to the same epitope as a pathogenic antibody (such as monoclonal antibody B4) to Annexin IV. In some embodiments, the Annexin IV is present on the surface of a cell, a basement membrane (e.g., Bruch's membrane), or in a pathological structure (e.g., drusen) in an individual that is in or adjacent to a tissue undergoing (or is at risk of undergoing) tissue injury (such as non-ischemic injury) and/oxidative damage. In some embodiments, the Annexin IV is produced by a nucleated cell (such as a mammalian cell). In some embodiments, the Annexin IV is recombinant protein. In some embodiments, the construct is a fusion protein. In some embodiments, the targeting moiety and the detectable moiety are linked via a linker (such as a peptide linker). In some embodiments, the eye tissue is the photoreceptor cell, retinal ganglion cell, RPE, Bruch's membrane, choriocapillary complex, macula, or cornea. In some embodiments, there is provided a method of detecting oxidative damage in an eye tissue in an individual, comprising administering to the individual an effective amount of a composition comprising a construct, wherein the construct comprises (a) an antibody or a fragment thereof, wherein the antibody or a fragment thereof specifically binds to Annexin IV; and (b) a detectable moiety, wherein the presence of the detectable moiety at the tissue is indicative of a oxidative damage in the eye tissue. In some embodiments, the method further comprises detecting the detectable moiety. In some embodiments, the antibody or fragment thereof competitively inhibits the binding of a pathogenic antibody (such as monoclonal antibody B4) to Annexin IV. In some embodiments, the antibody or antibody fragment thereof binds to the same epitope as a pathogenic antibody (such as monoclonal antibody B4) to Annexin IV. In some embodiments, the Annexin IV is present on the surface of a cell, a basement membrane (e.g., Bruch's membrane), or in a pathological structure (e.g., drusen) in an individual that is in or adjacent to a tissue undergoing (or is at risk of undergoing) tissue injury (such as non-ischemic injury) and/oxidative damage. In some embodiments, the Annexin IV is produced by a nucleated cell (such as a mammalian cell). In some embodiments, the Annexin IV is recombinant protein. In some embodiments, the construct is a fusion protein. In some embodiments, the targeting moiety and the detectable moiety are linked via a linker (such as a peptide linker). In some embodiments, the eye tissue is the photoreceptor cells, retinal ganglion cells, RPE, Bruch's membrane, choriocapillary complex, macula, or cornea.

In some embodiments, there is provided a method of diagnosing (or assisting in diagnosing) an inflammatory ocular disease (or an ocular disease involving oxidative damage) in an individual, comprising administering to the individual an effective amount of a composition comprising a construct, wherein the construct comprises (a) an antibody or a fragment thereof, wherein the antibody or a fragment thereof specifically binds to Annexin IV; and (b) a detectable moiety, wherein the presence of the detectable moiety at the eye tissue is indicative of the inflammatory ocular disease (or an ocular disease involving oxidative damage) in the tissue. In some embodiments, the method further comprises detecting the detectable moiety. In some embodiments, the antibody or fragment thereof competitively inhibits the binding of a pathogenic antibody (such as monoclonal antibody B4) to Annexin IV. In some embodiments, the antibody or antibody fragment thereof binds to the same epitope as a pathogenic antibody (such as monoclonal antibody B4) to Annexin IV. In some embodiments, the Annexin IV is present on the surface of a cell, a basement membrane (e.g., Bruch's membrane), or in a pathological structure (e.g., drusen) in an individual that is in or adjacent to a tissue undergoing (or is at risk of undergoing) tissue injury (such as non-ischemic injury) and/oxidative damage. In some embodiments, the Annexin IV is produced by a nucleated cell (such as a mammalian cell). In some embodiments, the Annexin IV is recombinant protein. In some embodiments, the construct is a fusion protein. In some embodiments, the targeting moiety and the detectable moiety are linked via a linker (such as a peptide linker). In some embodiments, the ocular disease is selected from the group consisting of age-related macular degeneration (AMD) (including wet AMD and dry AMD), cytomegalovirus (CMV) retinitis, macular edema, uveitis (anterior and posterior), glaucoma, open/wide-angle glaucoma, close/narrow-angle glaucoma, retinitis pigmentosa (RP), proliferative vitreoretinopathy, retinal detachment, corneal wound healing, corneal transplants, and ocular melanoma. In some embodiments, the ocular disease is AMD.

In some embodiments, there is provided a method of detecting complement-mediated injury (or detecting inflammation for example complement-mediated inflammation) in an eye tissue of an individual, comprising administering to the individual an effective amount of a composition comprising a construct, wherein the construct comprises (a) an antibody or a fragment thereof, wherein the antibody or a fragment thereof specifically binds to a phospholipid; and (b) a detectable moiety, wherein the presence of the detectable moiety at the tissue is indicative of a complement-mediated eye tissue injury (or complement-mediated inflammation). In some embodiments, the method further comprises detecting the detectable moiety. In some embodiments, the antibody or fragment thereof competitively inhibits the binding of a pathogenic antibody (such as monoclonal antibody C2) to phospholipid. In some embodiments, the antibody or antibody fragment thereof binds to the same epitope as a pathogenic antibody (such as monoclonal antibody C2) to phospholipid. In some embodiments, the phospholipid is present on the surface of a cell, a basement membrane (e.g., Bruch's membrane), or in a pathological structure (e.g., drusen) in an individual that is in or adjacent to a tissue undergoing (or is at risk of undergoing) tissue injury (such as non-ischemic injury) and/or oxidative damage. In some embodiments, the phospholipid is selected from the group consisting of phosphatidylethanolamine (PE), cardiolipin (CL), and phosphatidylcholine (PC). In some embodiments, the phospholipid is malondialdehyde (MDA). In some embodiments, the phospholipid is neutral. In some embodiments, the phospholipid is positively charged. In some embodiments, the eye tissue is the photoreceptor cells, retinal ganglion cells, RPE, Bruch's membrane, choriocapillary complex, macula, or cornea.

In some embodiments, there is provided a method of detecting oxidative damage in an eye tissue in an individual, comprising administering to the individual an effective amount of a composition comprising a construct, wherein the construct comprises (a) an antibody or a fragment thereof, wherein the antibody or a fragment thereof specifically binds to a phospholipid; and (b) a detectable moiety, wherein the presence of the detectable moiety at the tissue is indicative of a oxidative damage in the eye tissue. In some embodiments, the method further comprises detecting the detectable moiety. In some embodiments, the antibody or fragment thereof competitively inhibits the binding of a pathogenic antibody (such as monoclonal antibody C2) to phospholipid. In some embodiments, the antibody or antibody fragment thereof binds to the same epitope as a pathogenic antibody (such as monoclonal antibody C2) to phospholipid. In some embodiments, the phospholipid is present on the surface of a cell, a basement membrane (e.g., Bruch's membrane), or in a pathological structure (e.g., drusen) in an individual that is in or adjacent to a tissue undergoing (or is at risk of undergoing) tissue injury (such as non-ischemic injury) and/or oxidative damage. In some embodiments, the phospholipid is selected from the group consisting of phosphatidylethanolamine (PE), cardiolipin (CL), and phosphatidylcholine (PC). In some embodiments, the phospholipid is malondialdehyde (MDA). In some embodiments, the phospholipid is neutral. In some embodiments, the phospholipid is positively charged. In some embodiments, the eye tissue is the photoreceptor cells, retinal ganglion cells, RPE, Bruch's membrane, choriocapillary complex, macula, or cornea.

In some embodiments, there is provided a method of diagnosing (or assisting in diagnosing) an inflammatory ocular disease (or an ocular disease involving oxidative damage) in an individual, comprising administering to the individual an effective amount of a composition comprising a construct, wherein the construct comprises (a) an antibody or a fragment thereof, wherein the antibody or a fragment thereof specifically binds to a phospholipid; and (b) a detectable moiety, wherein the presence of the detectable moiety at the eye tissue is indicative of the inflammatory ocular disease (or an ocular disease involving oxidative damage) in the tissue. In some embodiments, the method further comprises detecting the detectable moiety. In some embodiments, the antibody or fragment thereof competitively inhibits the binding of a pathogenic antibody (such as monoclonal antibody C2) to phospholipid. In some embodiments, the antibody or antibody fragment thereof binds to the same epitope as a pathogenic antibody (such as monoclonal antibody C2) to phospholipid. In some embodiments, the phospholipid is present on the surface of a cell, a basement membrane (e.g., Bruch's membrane), or in a pathological structure (e.g., drusen) in an individual that is in or adjacent to a tissue undergoing (or is at risk of undergoing) tissue injury (such as non-ischemic injury) and/or oxidative damage. In some embodiments, the phospholipid is selected from the group consisting of phosphatidylethanolamine (PE), cardiolipin (CL), and phosphatidylcholine (PC). In some embodiments, the phospholipid is malondialdehyde (MDA). In some embodiments, the phospholipid is neutral. In some embodiments, the phospholipid is positively charged. In some embodiments, the ocular disease is selected from the group consisting of age-related macular degeneration (AMD) (including wet AMD and dry AMD), cytomegalovirus (CMV) retinitis, macular edema, uveitis (anterior and posterior), glaucoma, open/wide-angle glaucoma, close/narrow-angle glaucoma, retinitis pigmentosa (RP), proliferative vitreoretinopathy, retinal detachment, corneal wound healing, corneal transplants, and ocular melanoma. In some embodiments, the ocular disease is AMD.

Targeting Constructs

The present application in some embodiments provides targeted constructs which can be useful, but are not limited, any one or more of the methods described herein. It is to be understood that any of the constructs described in the section herein can be used for any of the methods described in the sections above. The present application further provides methods of delivering any of the complement modulator or detectable moiety disclosed herein to a site of complement activation, a site of tissue injury (such as non-ischemic tissue injury), or a site of complement-associated disease in an individual by administering to the individual any one of the target constructs described herein.

In some embodiments, there is provided a construct (or a composition comprising the construct such as a pharmaceutical composition), wherein the construct comprises (a) an antibody or a fragment thereof, wherein the antibody or a fragment thereof specifically binds to Annexin IV; and (b) a therapeutic agent or a detectable moiety. In some embodiments, the construct comprises a therapeutic agent (such as a complement modulator, for example a complement inhibitor). In some embodiments, the construct comprises a detectable moiety. In some embodiments, the construct is a fusion protein. In some embodiments, the antibody or fragment thereof (hereinafter also referred to as the “targeting moiety” and the detectable moiety (hereinafter also referred to as “the active moiety” are linked via a linker (such as a peptide linker). In some embodiments, the targeting moiety and the active moiety are directly linked.

In some embodiments, there is provided a construct (or a composition comprising the construct such as a pharmaceutical composition), wherein the construct comprises (a) an antibody or a fragment thereof, wherein the antibody or a fragment thereof specifically binds to Annexin IV; and (b) a complement modulator or a detectable moiety, wherein the antibody or fragment there of comprises: (i) a light chain variable domain comprising a sequence of SEQ ID NO:1, a sequence of SEQ ID NO:2, or a sequence of SEQ ID NO:3; and/or (ii) heavy chain variable domain comprising a sequence of SEQ ID NO:4, a sequence of SEQ ID NO:5, or a sequence of SEQ ID NO:6. In some embodiments, there is provided a construct (or a composition comprising the construct such as a pharmaceutical composition), wherein the construct comprises (a) an antibody or a fragment thereof, wherein the antibody or a fragment thereof specifically binds to Annexin IV; and (b) a complement modulator or a detectable moiety, wherein the antibody or fragment there of comprises: (i) a light chain variable domain comprising a sequence of SEQ ID NO:7, a sequence of SEQ ID NO:8, or a sequence of SEQ ID NO:9; and/or (ii) heavy chain variable domain comprising a sequence of SEQ ID NO:10, a sequence of SEQ ID NO:11, or a sequence of SEQ ID NO:12. In some embodiments, the antibody or fragment thereof competitively inhibits the binding of a pathogenic antibody (such as monoclonal antibody B4) to Annexin IV. In some embodiments, the antibody or fragment thereof binds to the same epitope as a pathogenic antibody (such as a monoclonal antibody B4) to Annexin IV. In some embodiments, the Annexin IV is present on the surface of a cell (and/or in a pathological structure) in an individual that is in or adjacent to a tissue undergoing (or is at risk of undergoing) tissue injury (such as non-ischemic injury) and/oxidative damage. In some embodiments, the Annexin IV is produced by a nucleated cell (such as a mammalian cell). In some embodiments, the Annexin IV is recombinant protein. In some embodiments, the construct comprises a complement modulator (such as a complement inhibitor). In some embodiments, the construct comprises a detectable moiety. In some embodiments, the construct is a fusion protein. In some embodiments, the targeting moiety and the active moiety are linked via a linker (such as a peptide linker). In some embodiments, the targeting moiety and the active moiety are directly linked.

In some embodiments, there is provided a construct (or a composition comprising the construct such as a pharmaceutical composition), wherein the construct comprises (a) an antibody or a fragment thereof, wherein the antibody or a fragment thereof specifically binds to Annexin IV; and (b) a complement modulator or a detectable moiety, wherein the antibody or fragment there of comprises: (i) a light chain variable domain comprising a sequence of SEQ ID NO:1; (ii) a light chain variable domain comprising a sequence of SEQ ID NO:2; and (iii) a light chain variable domain comprising a sequence of SEQ ID NO:3. In some embodiments, there is provided a construct (or a composition comprising the construct such as a pharmaceutical composition), wherein the construct comprises (a) an antibody or a fragment thereof, wherein the antibody or a fragment thereof specifically binds to Annexin IV; and (b) a complement modulator or a detectable moiety, wherein the antibody or fragment there of comprises: (i) a light chain variable domain comprising a sequence of SEQ ID NO:7; (ii) a light chain variable domain comprising a sequence of SEQ ID NO:8; and (iii) a light chain variable domain comprising a sequence of SEQ ID NO:9. In some embodiments, the antibody or fragment thereof competitively inhibits the binding of a pathogenic antibody (such as monoclonal antibody B4) to Annexin IV. In some embodiments, the antibody or fragment thereof binds to the same epitope as a pathogenic antibody (such as a monoclonal antibody B4) to Annexin IV. In some embodiments, the Annexin IV is present on the surface of a cell (and/or in a pathological structure) in an individual that is in or adjacent to a tissue undergoing (or is at risk of undergoing) tissue injury (such as non-ischemic injury) and/oxidative damage. In some embodiments, the Annexin IV is produced by a nucleated cell (such as a mammalian cell). In some embodiments, the Annexin IV is recombinant protein. In some embodiments, the construct comprises a complement modulator (such as a complement inhibitor). In some embodiments, the construct comprises a detectable moiety. In some embodiments, the construct is a fusion protein. In some embodiments, the targeting moiety and the active moiety are linked via a linker (such as a peptide linker). In some embodiments, the targeting moiety and the active moiety are directly linked.

In some embodiments, there is provided a construct (or a composition comprising the construct such as a pharmaceutical composition), wherein the construct comprises (a) an antibody or a fragment thereof, wherein the antibody or a fragment thereof specifically binds to Annexin IV; and (b) a complement modulator or a detectable moiety, wherein the antibody or fragment there of comprises: (i) heavy chain variable domain comprising a sequence of SEQ ID NO:4; (ii) heavy chain variable domain comprising a sequence of SEQ ID NO:5; and (iii) heavy chain variable domain comprising a sequence of SEQ ID NO:6. In some embodiments, there is provided a construct (or a composition comprising the construct such as a pharmaceutical composition), wherein the construct comprises (a) an antibody or a fragment thereof, wherein the antibody or a fragment thereof specifically binds to Annexin IV; and (b) a complement modulator or a detectable moiety, wherein the antibody or fragment there of comprises: (i) heavy chain variable domain comprising a sequence of SEQ ID NO:10; (ii) heavy chain variable domain comprising a sequence of SEQ ID NO:11; and (iii) heavy chain variable domain comprising a sequence of SEQ ID NO:12. In some embodiments, the antibody or fragment thereof competitively inhibits the binding of a pathogenic antibody (such as monoclonal antibody B4) to Annexin IV. In some embodiments, the antibody or fragment thereof binds to the same epitope as a pathogenic antibody (such as a monoclonal antibody B4) to Annexin IV. In some embodiments, the Annexin IV is present on the surface of a cell (and/or in a pathological structure) in an individual that is in or adjacent to a tissue undergoing (or is at risk of undergoing) tissue injury (such as non-ischemic injury) and/oxidative damage. In some embodiments, the Annexin IV is produced by a nucleated cell (such as a mammalian cell). In some embodiments, the Annexin IV is recombinant protein. In some embodiments, the construct comprises a complement modulator (such as a complement inhibitor). In some embodiments, the construct comprises a detectable moiety. In some embodiments, the construct is a fusion protein. In some embodiments, the targeting moiety and the active moiety are linked via a linker (such as a peptide linker). In some embodiments, the targeting moiety and the active moiety are directly linked.

In some embodiments, there is provided a construct (or a composition comprising the construct such as a pharmaceutical composition), wherein the construct comprises (a) an antibody or a fragment thereof, wherein the antibody or a fragment thereof specifically binds to Annexin IV; and (b) a complement modulator or a detectable moiety, wherein the antibody or fragment there of comprises: (i) a light chain variable domain comprising a sequence of SEQ ID NO:1; (ii) a light chain variable domain comprising a sequence of SEQ ID NO:2; (iii) a light chain variable domain comprising a sequence of SEQ ID NO:3; (iv) heavy chain variable domain comprising a sequence of SEQ ID NO:4; (v) heavy chain variable domain comprising a sequence of SEQ ID NO:5; and (vi) heavy chain variable domain comprising a sequence of SEQ ID NO:6. In some embodiments, there is provided a construct (or a composition comprising the construct such as a pharmaceutical composition), wherein the construct comprises (a) an antibody or a fragment thereof, wherein the antibody or a fragment thereof specifically binds to Annexin IV; and (b) a complement modulator or a detectable moiety, wherein the antibody or fragment there of comprises: (i) a light chain variable domain comprising a sequence of SEQ ID NO:7; (ii) a light chain variable domain comprising a sequence of SEQ ID NO:8; (iii) a light chain variable domain comprising a sequence of SEQ ID NO:9; (iv) heavy chain variable domain comprising a sequence of SEQ ID NO:10; (v) heavy chain variable domain comprising a sequence of SEQ ID NO:11; and (vi) heavy chain variable domain comprising a sequence of SEQ ID NO:12. In some embodiments, the antibody or fragment thereof competitively inhibits the binding of a pathogenic antibody (such as monoclonal antibody B4) to Annexin IV. In some embodiments, the antibody or fragment thereof binds to the same epitope as a pathogenic antibody (such as a monoclonal antibody B4) to Annexin IV. In some embodiments, the Annexin IV is present on the surface of a cell (and/or in a pathological structure) in an individual that is in or adjacent to a tissue undergoing (or is at risk of undergoing) tissue injury (such as non-ischemic injury) and/oxidative damage. In some embodiments, the Annexin IV is produced by a nucleated cell (such as a mammalian cell). In some embodiments, the Annexin IV is recombinant protein. In some embodiments, the construct comprises a complement modulator (such as a complement inhibitor). In some embodiments, the construct comprises a detectable moiety. In some embodiments, the construct is a fusion protein. In some embodiments, the targeting moiety and the active moiety are linked via a linker (such as a peptide linker). In some embodiments, the targeting moiety and the active moiety are directly linked.

In some embodiments, there is provided a construct (or a composition comprising the construct such as a pharmaceutical composition), wherein the construct comprises (a) an antibody or a fragment thereof, wherein the antibody or a fragment thereof specifically binds to Annexin IV; and (b) a complement modulator or a detectable moiety, wherein the antibody or fragment there of comprises: (i) a light chain CDR1 of SEQ ID NO:1; (ii) a light chain CDR2 of SEQ ID NO:2; (iii) a light chain CDR3 of SEQ ID NO:3; (iv) heavy chain CDR1 of SEQ ID NO:4; (v) heavy chain CDR2 of SEQ ID NO:5; and (vi) heavy chain CDR3 of SEQ ID NO:6. In some embodiments, there is provided a construct (or a composition comprising the construct such as a pharmaceutical composition), wherein the construct comprises (a) an antibody or a fragment thereof, wherein the antibody or a fragment thereof specifically binds to Annexin IV; and (b) a complement modulator or a detectable moiety, wherein the antibody or fragment there of comprises: (i) a light chain CDR1 of SEQ ID NO:7; (ii) a light chain CDR2 of SEQ ID NO:8; (iii) a light chain CDR3 of SEQ ID NO:9; (iv) heavy chain CDR1 of SEQ ID NO:10; (v) heavy chain CDR2 of SEQ ID NO:11; and (vi) heavy chain CDR3 of SEQ ID NO:12. In some embodiments, the antibody or fragment thereof competitively inhibits the binding of a pathogenic antibody (such as monoclonal antibody B4) to Annexin IV. In some embodiments, the antibody or fragment thereof binds to the same epitope as a pathogenic antibody (such as a monoclonal antibody B4) to Annexin IV. In some embodiments, the Annexin IV is present on the surface of a cell (and/or in a pathological structure) in an individual that is in or adjacent to a tissue undergoing (or is at risk of undergoing) tissue injury (such as non-ischemic injury) and/oxidative damage. In some embodiments, the Annexin IV is produced by a nucleated cell (such as a mammalian cell). In some embodiments, the Annexin IV is recombinant protein. In some embodiments, the construct comprises a complement modulator (such as a complement inhibitor). In some embodiments, the construct comprises a detectable moiety. In some embodiments, the construct is a fusion protein. In some embodiments, the targeting moiety and the active moiety are linked via a linker (such as a peptide linker). In some embodiments, the targeting moiety and the active moiety are directly linked.

In some embodiments, there is provided a construct (or a composition comprising the construct such as a pharmaceutical composition), wherein the construct comprises (a) an antibody or a fragment thereof, wherein the antibody or a fragment thereof specifically binds to Annexin IV; and (b) a complement modulator or a detectable moiety, wherein the antibody or fragment there of comprises a light chain variable domain of SEQ ID NO:13. In some embodiments, there is provided a construct (or a composition comprising the construct such as a pharmaceutical composition), wherein the construct comprises (a) an antibody or a fragment thereof, wherein the antibody or a fragment thereof specifically binds to Annexin IV; and (b) a complement modulator or a detectable moiety, wherein the antibody or fragment there of comprises a heavy chain variable domain of SEQ ID NO:15. In some embodiments, there is provided a construct (or a composition comprising the construct such as a pharmaceutical composition), wherein the construct comprises (a) an antibody or a fragment thereof, wherein the antibody or a fragment thereof specifically binds to Annexin IV; and (b) a complement modulator or a detectable moiety, wherein the antibody or fragment there of comprises a light chain variable domain of SEQ ID NO:14. In some embodiments, there is provided a construct (or a composition comprising the construct such as a pharmaceutical composition), wherein the construct comprises (a) an antibody or a fragment thereof, wherein the antibody or a fragment thereof specifically binds to Annexin IV; and (b) a complement modulator or a detectable moiety, wherein the antibody or fragment there of comprises a heavy chain variable domain of SEQ ID NO:16. In some embodiments, the antibody or fragment thereof competitively inhibits the binding of a pathogenic monoclonal antibody (such as monoclonal antibody B4) to Annexin IV. I In some embodiments, the antibody or fragment thereof competitively inhibits the binding of a pathogenic antibody (such as monoclonal antibody B4) to Annexin IV. In some embodiments, the antibody or fragment thereof binds to the same epitope as a pathogenic antibody (such as a monoclonal antibody B4) to Annexin IV. In some embodiments, the Annexin IV is present on the surface of a cell (and/or in a pathological structure) in an individual that is in or adjacent to a tissue undergoing (or is at risk of undergoing) tissue injury (such as non-ischemic injury) and/oxidative damage. In some embodiments, the Annexin IV is produced by a nucleated cell (such as a mammalian cell). In some embodiments, the Annexin IV is recombinant protein. In some embodiments, the construct comprises a complement modulator (such as a complement inhibitor). In some embodiments, the construct comprises a detectable moiety. In some embodiments, the construct is a fusion protein. In some embodiments, the targeting moiety and the active moiety are linked via a linker (such as a peptide linker). In some embodiments, the targeting moiety and the active moiety are directly linked.

In some embodiments, there is provided a construct (or a composition comprising the construct such as a pharmaceutical composition), wherein the construct comprises (a) an antibody or a fragment thereof, wherein the antibody or a fragment thereof specifically binds to Annexin IV; and (b) a complement modulator or a detectable moiety, wherein the antibody or fragment there of comprises: (i) a light chain variable domain of SEQ ID NO:13; and (ii) heavy chain variable domain of SEQ ID NO:15. In some embodiments, there is provided a construct (or a composition comprising the construct such as a pharmaceutical composition), wherein the construct comprises (a) an antibody or a fragment thereof, wherein the antibody or a fragment thereof specifically binds to Annexin IV; and (b) a complement modulator or a detectable moiety, wherein the antibody or fragment there of comprises: (i) a light chain variable domain of SEQ ID NO:14; and (ii) heavy chain variable domain of SEQ ID NO:16. In some embodiments, the antibody or fragment thereof competitively inhibits the binding of a pathogenic antibody (such as monoclonal antibody B4) to Annexin IV. In some embodiments, the antibody or fragment thereof binds to the same epitope as a pathogenic antibody (such as a monoclonal antibody B4) to Annexin IV. In some embodiments, the Annexin IV is present on the surface of a cell (and/or in a pathological structure) in an individual that is in or adjacent to a tissue undergoing (or is at risk of undergoing) tissue injury (such as non-ischemic injury) and/oxidative damage. In some embodiments, the Annexin IV is produced by a nucleated cell (such as a mammalian cell). In some embodiments, the Annexin IV is recombinant protein. In some embodiments, the construct comprises a complement modulator (such as a complement inhibitor). In some embodiments, the construct comprises a detectable moiety. In some embodiments, the construct is a fusion protein. In some embodiments, the targeting moiety and the active moiety are linked via a linker (such as a peptide linker). In some embodiments, the targeting moiety and the active moiety are directly linked.

In some embodiments, there is provided a construct (or a composition comprising the construct such as a pharmaceutical composition), wherein the construct comprises (a) an antibody or a fragment thereof, wherein the antibody or a fragment thereof specifically binds to Annexin IV; and (b) a complement modulator or a detectable moiety, wherein the antibody or fragment is a scFv having the sequence of SEQ ID NO:17. In some embodiments, there is provided a construct (or a composition comprising the construct such as a pharmaceutical composition), wherein the construct comprises (a) an antibody or a fragment thereof, wherein the antibody or a fragment thereof specifically binds to Annexin IV; and (b) a complement modulator or a detectable moiety, wherein the antibody or fragment is a scFv having the sequence of SEQ ID NO:18. In some embodiments, the antibody or fragment thereof competitively inhibits the binding of a pathogenic antibody (such as monoclonal antibody B4) to Annexin IV. In some embodiments, the antibody or fragment thereof binds to the same epitope as a pathogenic antibody (such as a monoclonal antibody B4) to Annexin IV. In some embodiments, the Annexin IV is present on the surface of a cell (and/or in a pathological structure) in an individual that is in or adjacent to a tissue undergoing (or is at risk of undergoing) tissue injury (such as non-ischemic injury) and/oxidative damage. In some embodiments, the Annexin IV is produced by a nucleated cell (such as a mammalian cell). In some embodiments, the Annexin IV is recombinant protein. In some embodiments, the construct comprises a complement modulator (such as a complement inhibitor). In some embodiments, the construct comprises a detectable moiety. In some embodiments, the construct is a fusion protein. In some embodiments, the targeting moiety and the active moiety are linked via a linker (such as a peptide linker). In some embodiments, the targeting moiety and the active moiety are directly linked.

In some embodiments, there is provided a targeting construct (or a composition comprising the construct such as a pharmaceutical composition), wherein the targeting construct comprises (a) an antibody or a fragment thereof, wherein the antibody or a fragment thereof specifically binds to a phospholipid (such as PE, CL, MDA, and/or PC); and (b) a an active moiety, wherein the antibody or fragment thereof comprises: (i) a light chain variable domain comprising a sequence of SEQ ID NO:25, a sequence of SEQ ID NO:26, or a sequence of SEQ ID NO:27; and/or (ii) heavy chain variable domain comprising a sequence of SEQ ID NO:28, a sequence of SEQ ID NO:29, or a sequence of SEQ ID NO:30. In some embodiments, there is provided a construct (or a composition comprising the construct such as a pharmaceutical composition), wherein the construct comprises (a) an antibody or a fragment thereof, wherein the antibody or a fragment thereof specifically binds to a phospholipid (such as PE, CL, MDA, and/or PC); and (b) a complement modulator or a detectable moiety, wherein the antibody or fragment thereof comprises: (i) a light chain variable domain comprising a sequence of SEQ ID NO:31, a sequence of SEQ ID NO:32, or a sequence of SEQ ID NO:33; and/or (ii) heavy chain variable domain comprising a sequence of SEQ ID NO:28, a sequence of SEQ ID NO:29, or a sequence of SEQ ID NO:30. In some embodiments, the antibody or fragment thereof competitively inhibits the binding of a pathogenic antibody (such as monoclonal antibody C2) to phospholipid. In some embodiments, the antibody or antibody fragment thereof binds to the same epitope as a pathogenic antibody (such as monoclonal antibody C2) to phospholipid. In some embodiments, the phospholipid is present on the surface of a cell, a basement membrane (e.g., Bruch's membrane), or in a pathological structure (e.g., drusen) in an individual that is in or adjacent to a tissue undergoing (or is at risk of undergoing) tissue injury (such as non-ischemic injury) and/or oxidative damage. In some embodiments, the phospholipid is selected from the group consisting of phosphatidylethanolamine (PE), cardiolipin (CL), and phosphatidylcholine (PC). In some embodiments, the phospholipid is malondialdehyde (MDA). In some embodiments, the phospholipid is neutral. In some embodiments, the phospholipid is positively charged. In some embodiments, the phospholipid is oxidized. In some embodiments, the construct comprises an active moiety that comprises a therapeutic moiety (such as a complement inhibitor). In some embodiments, the construct comprises an active moiety that is a detectable moiety. In some embodiments, the construct is a fusion protein. In some embodiments, the targeting moiety and the active moiety are linked via a linker (such as a peptide linker).

In some embodiments, there is provided a targeting construct (or a composition comprising the construct such as a pharmaceutical composition), wherein the targeting construct comprises (a) an antibody or a fragment thereof, wherein the antibody or a fragment thereof specifically binds to a phospholipid (such as PE, CL, MDA, and/or PC); and (b) an active moiety (e.g., a therapeutic moiety or a detectable moiety), wherein the antibody or fragment thereof comprises: (i) a light chain variable domain comprising a sequence of SEQ ID NO:25; (ii) a light chain variable domain comprising a sequence of SEQ ID NO:26; and (iii) a light chain variable domain comprising a sequence of SEQ ID NO:27. In some embodiments, there is provided a targeting construct (or a composition comprising the construct such as a pharmaceutical composition), wherein the targeting construct comprises (a) an antibody or a fragment thereof, wherein the antibody or a fragment thereof specifically binds to a phospholipid (such as PE, CL, MDA, and/or PC); and (b) an active moiety (e.g., a therapeutic moiety or a detectable moiety), wherein the antibody or fragment thereof comprises: (i) a light chain variable domain comprising a sequence of SEQ ID NO:31; (ii) a light chain variable domain comprising a sequence of SEQ ID NO:32; and (iii) a light chain variable domain comprising a sequence of SEQ ID NO:33. In some embodiments, the antibody or fragment thereof competitively inhibits the binding of a pathogenic antibody (such as monoclonal antibody C2) to phospholipid. In some embodiments, the antibody or antibody fragment thereof binds to the same epitope as a pathogenic antibody (such as monoclonal antibody C2) to phospholipid. In some embodiments, the phospholipid is present on the surface of a cell, a basement membrane (e.g., Bruch's membrane), or in a pathological structure (e.g., drusen) in an individual that is in or adjacent to a tissue undergoing (or is at risk of undergoing) tissue injury (such as non-ischemic injury) and/or oxidative damage. In some embodiments, the phospholipid is selected from the group consisting of phosphatidylethanolamine (PE), cardiolipin (CL), and phosphatidylcholine (PC). In some embodiments, the phospholipid is malondialdehyde (MDA). In some embodiments, the phospholipid is neutral. In some embodiments, the phospholipid is positively charged. In some embodiments, the phospholipid is oxidized. In some embodiments, the construct comprises an active moiety that comprises a therapeutic moiety (such as a complement inhibitor). In some embodiments, the construct comprises an active moiety that is a detectable moiety. In some embodiments, the construct is a fusion protein. In some embodiments, the targeting moiety and the active moiety are linked via a linker (such as a peptide linker).

In some embodiments, there is provided a targeting construct (or a composition comprising the construct such as a pharmaceutical composition), wherein the construct comprises (a) an antibody or a fragment thereof, wherein the antibody or a fragment thereof specifically binds to a phospholipid (such as PE, CL, MDA, and/or PC); and (b) an active moiety (e.g., a therapeutic moiety or a detectable moiety), wherein the antibody or fragment thereof comprises: (i) heavy chain variable domain comprising a sequence of SEQ ID NO:28; (ii) heavy chain variable domain comprising a sequence of SEQ ID NO:29; and (iii) heavy chain variable domain comprising a sequence of SEQ ID NO:30. In some embodiments, the antibody or fragment thereof competitively inhibits the binding of a pathogenic antibody (such as monoclonal antibody C2) to phospholipid. In some embodiments, the antibody or antibody fragment thereof binds to the same epitope as a pathogenic antibody (such as monoclonal antibody C2) to phospholipid. In some embodiments, the phospholipid is present on the surface of a cell, a basement membrane (e.g., Bruch's membrane), or in a pathological structure (e.g., drusen) in an individual that is in or adjacent to a tissue undergoing (or is at risk of undergoing) tissue injury (such as non-ischemic injury) and/or oxidative damage. In some embodiments, the phospholipid is selected from the group consisting of phosphatidylethanolamine (PE), cardiolipin (CL), and phosphatidylcholine (PC). In some embodiments, the phospholipid is malondialdehyde (MDA). In some embodiments, the phospholipid is neutral. In some embodiments, the phospholipid is positively charged. In some embodiments, the phospholipid is oxidized. In some embodiments, the construct comprises an active moiety that comprises a therapeutic moiety (such as a complement inhibitor). In some embodiments, the construct comprises an active moiety that is a detectable moiety. In some embodiments, the construct is a fusion protein. In some embodiments, the targeting moiety and the active moiety are linked via a linker (such as a peptide linker).

In some embodiments, there is provided a targeting construct (or a composition comprising the construct such as a pharmaceutical composition), wherein the targeting construct comprises (a) an antibody or a fragment thereof, wherein the antibody or a fragment thereof specifically binds to a phospholipid (such as PE, CL, MDA, and/or PC); and (b) an active moiety (e.g., a therapeutic moiety or a detectable moiety), wherein the antibody or fragment thereof comprises: (i) a light chain variable domain comprising a sequence of SEQ ID NO:25; (ii) a light chain variable domain comprising a sequence of SEQ ID NO:26; (iii) a light chain variable domain comprising a sequence of SEQ ID NO:27; (iv) heavy chain variable domain comprising a sequence of SEQ ID NO:28; (v) heavy chain variable domain comprising a sequence of SEQ ID NO:29; and (vi) heavy chain variable domain comprising a sequence of SEQ ID NO:30. In some embodiments, there is provided a targeting construct (or a composition comprising the construct such as a pharmaceutical composition), wherein the targeting construct comprises (a) an antibody or a fragment thereof, wherein the antibody or a fragment thereof specifically binds to a phospholipid (such as PE, CL, MDA, and/or PC); and (b) an active moiety (e.g., a therapeutic moiety or a detectable moiety), wherein the antibody or fragment thereof comprises: (i) a light chain variable domain comprising a sequence of SEQ ID NO:31; (ii) a light chain variable domain comprising a sequence of SEQ ID NO:32; (iii) a light chain variable domain comprising a sequence of SEQ ID NO:33; (iv) heavy chain variable domain comprising a sequence of SEQ ID NO:28; (v) heavy chain variable domain comprising a sequence of SEQ ID NO:29; and (vi) heavy chain variable domain comprising a sequence of SEQ ID NO:30. In some embodiments, the antibody or fragment thereof competitively inhibits the binding of a pathogenic antibody (such as monoclonal antibody C2) to phospholipid. In some embodiments, the antibody or antibody fragment thereof binds to the same epitope as a pathogenic antibody (such as monoclonal antibody C2) to phospholipid. In some embodiments, the phospholipid is present on the surface of a cell, a basement membrane (e.g., Bruch's membrane), or in a pathological structure (e.g., drusen) in an individual that is in or adjacent to a tissue undergoing (or is at risk of undergoing) tissue injury (such as non-ischemic injury) and/or oxidative damage. In some embodiments, the phospholipid is selected from the group consisting of phosphatidylethanolamine (PE), cardiolipin (CL), and phosphatidylcholine (PC). In some embodiments, the phospholipid is malondialdehyde (MDA). In some embodiments, the phospholipid is neutral. In some embodiments, the phospholipid is positively charged. In some embodiments, the phospholipid is oxidized. In some embodiments, the construct comprises an active moiety that comprises a therapeutic moiety (such as a complement inhibitor). In some embodiments, the construct comprises an active moiety that is a detectable moiety. In some embodiments, the construct is a fusion protein. In some embodiments, the targeting moiety and the active moiety are linked via a linker (such as a peptide linker).

In some embodiments, there is provided a targeting construct (or a composition comprising the construct such as a pharmaceutical composition), wherein the targeting construct comprises (a) an antibody or a fragment thereof, wherein the antibody or a fragment thereof specifically binds to a phospholipid (such as PE, CL, MDA, and/or PC); and (b) an active moiety (e.g., a therapeutic moiety or a detectable moiety), wherein the antibody or fragment thereof comprises: (i) a light chain CDR1 of SEQ ID NO:25; (ii) a light chain CDR2 of SEQ ID NO:26; (iii) a light chain CDR3 of SEQ ID NO:27; (iv) heavy chain CDR1 of SEQ ID NO:28; (v) heavy chain CDR2 of SEQ ID NO:29; and (vi) heavy chain CDR3 of SEQ ID NO:30. In some embodiments, there is provided a targeting construct (or a composition comprising the construct such as a pharmaceutical composition), wherein the targeting construct comprises (a) an antibody or a fragment thereof, wherein the antibody or a fragment thereof specifically binds to a phospholipid (such as PE, CL, MDA, and/or PC); and (b) an active moiety (e.g., a therapeutic moiety or a detectable moiety), wherein the antibody or fragment thereof comprises: (i) a light chain CDR1 of SEQ ID NO:31; (ii) a light chain CDR2 of SEQ ID NO:32; (iii) a light chain CDR3 of SEQ ID NO:33; (iv) heavy chain CDR1 of SEQ ID NO:28; (v) heavy chain CDR2 of SEQ ID NO:29; and (vi) heavy chain CDR3 of SEQ ID NO:30. In some embodiments, the antibody or fragment thereof competitively inhibits the binding of a pathogenic antibody (such as monoclonal antibody C2) to phospholipid. In some embodiments, the antibody or antibody fragment thereof binds to the same epitope as a pathogenic antibody (such as monoclonal antibody C2) to phospholipid. In some embodiments, the phospholipid is present on the surface of a cell, a basement membrane (e.g., Bruch's membrane), or in a pathological structure (e.g., drusen) in an individual that is in or adjacent to a tissue undergoing (or is at risk of undergoing) tissue injury (such as non-ischemic injury) and/or oxidative damage. In some embodiments, the phospholipid is selected from the group consisting of phosphatidylethanolamine (PE), cardiolipin (CL), and phosphatidylcholine (PC). In some embodiments, the phospholipid is malondialdehyde (MDA). In some embodiments, the phospholipid is neutral. In some embodiments, the phospholipid is positively charged. In some embodiments, the phospholipid is oxidized. In some embodiments, the construct comprises an active moiety that comprises a therapeutic moiety (such as a complement inhibitor). In some embodiments, the construct comprises an active moiety that is a detectable moiety. In some embodiments, the construct is a fusion protein. In some embodiments, the targeting moiety and the active moiety are linked via a linker (such as a peptide linker).

In some embodiments, there is provided a targeting construct (or a composition comprising the construct such as a pharmaceutical composition), wherein the targeting construct comprises (a) an antibody or a fragment thereof, wherein the antibody or a fragment thereof specifically binds to a phospholipid (such as PE, CL, MDA, and/or PC); and (b) an active moiety (e.g., a therapeutic moiety or a detectable moiety), wherein the antibody or fragment thereof comprises a light chain variable domain of SEQ ID NO:34. In some embodiments, there is provided a targeting construct (or a composition comprising the construct such as a pharmaceutical composition), wherein the targeting construct comprises (a) an antibody or a fragment thereof, wherein the antibody or a fragment thereof specifically binds to a phospholipid (such as PE, CL, MDA, and/or PC); and (b) an active moiety (e.g., a therapeutic moiety a detectable moiety), wherein the antibody or fragment thereof comprises a heavy chain variable domain of SEQ ID NO:36. In some embodiments, there is provided a targeting construct (or a composition comprising the construct such as a pharmaceutical composition), wherein the targeting construct comprises (a) an antibody or a fragment thereof, wherein the antibody or a fragment thereof specifically binds to a phospholipid (such as PE, CL, MDA, and/or PC); and (b) an active moiety (e.g., a therapeutic moiety or a detectable moiety), wherein the antibody or fragment thereof comprises a light chain variable domain of SEQ ID NO:35. In some embodiments, the antibody or fragment thereof competitively inhibits the binding of a pathogenic antibody (such as monoclonal antibody C2) to phospholipid. In some embodiments, the antibody or antibody fragment thereof binds to the same epitope as a pathogenic antibody (such as monoclonal antibody C2) to phospholipid. In some embodiments, the phospholipid is present on the surface of a cell, a basement membrane (e.g., Bruch's membrane), or in a pathological structure (e.g., drusen) in an individual that is in or adjacent to a tissue undergoing (or is at risk of undergoing) tissue injury (such as non-ischemic injury) and/or oxidative damage. In some embodiments, the phospholipid is selected from the group consisting of phosphatidylethanolamine (PE), cardiolipin (CL), and phosphatidylcholine (PC). In some embodiments, the phospholipid is malondialdehyde (MDA). In some embodiments, the phospholipid is neutral. In some embodiments, the phospholipid is positively charged. In some embodiments, the phospholipid is oxidized. In some embodiments, the construct comprises an active moiety that comprises a therapeutic moiety (such as a complement inhibitor). In some embodiments, the construct comprises an active moiety that is a detectable moiety. In some embodiments, the construct is a fusion protein. In some embodiments, the targeting moiety and the active moiety are linked via a linker (such as a peptide linker).

In some embodiments, there is provided a targeting construct (or a composition comprising the construct such as a pharmaceutical composition), wherein the targeting construct comprises (a) an antibody or a fragment thereof, wherein the antibody or a fragment thereof specifically binds to a phospholipid (such as PE, CL, MDA, and/or PC); and (b) an active moiety (e.g., a therapeutic moiety or a detectable moiety), wherein the antibody or fragment thereof comprises: (i) a light chain variable domain of SEQ ID NO:34; and (ii) heavy chain variable domain of SEQ ID NO:36. In some embodiments, there is provided a targeting construct (or a composition comprising the construct such as a pharmaceutical composition), wherein the targeting construct comprises (a) an antibody or a fragment thereof, wherein the antibody or a fragment thereof specifically binds to a phospholipid (such as PE, CL, MDA, and/or PC); and (b) an active moiety (e.g., a therapeutic moiety or a detectable moiety), wherein the antibody or fragment thereof comprises: (i) a light chain variable domain of SEQ ID NO:35; and (ii) heavy chain variable domain of SEQ ID NO:36. In some embodiments, the antibody or fragment thereof competitively inhibits the binding of a pathogenic antibody (such as monoclonal antibody C2) to phospholipid. In some embodiments, the antibody or antibody fragment thereof binds to the same epitope as a pathogenic antibody (such as monoclonal antibody C2) to phospholipid. In some embodiments, the phospholipid is present on the surface of a cell, a basement membrane (e.g., Bruch's membrane), or in a pathological structure (e.g., drusen) in an individual that is in or adjacent to a tissue undergoing (or is at risk of undergoing) tissue injury (such as non-ischemic injury) and/or oxidative damage. In some embodiments, the phospholipid is selected from the group consisting of phosphatidylethanolamine (PE), cardiolipin (CL), and phosphatidylcholine (PC). In some embodiments, the phospholipid is malondialdehyde (MDA). In some embodiments, the phospholipid is neutral. In some embodiments, the phospholipid is positively charged. In some embodiments, the phospholipid is oxidized. In some embodiments, the construct comprises an active moiety that comprises a therapeutic moiety (such as a complement inhibitor). In some embodiments, the construct comprises an active moiety that is a detectable moiety. In some embodiments, the construct is a fusion protein. In some embodiments, the targeting moiety and the active moiety are linked via a linker (such as a peptide linker).

In some embodiments, there is provided a targeting construct (or a composition comprising the construct such as a pharmaceutical composition), wherein the construct comprises (a) an antibody or a fragment thereof, wherein the antibody or a fragment thereof specifically binds to a phospholipid (such as PE, CL, MDA, and/or PC); and (b) an active moiety (e.g., a therapeutic moiety or a detectable moiety), wherein the antibody or fragment is a scFv having the sequence of SEQ ID NO:37. In some embodiments, there is provided a targeting construct (or a composition comprising the construct such as a pharmaceutical composition), wherein the targeting construct comprises (a) an antibody or a fragment thereof, wherein the antibody or a fragment thereof specifically binds to a phospholipid (such as PE, CL, MDA, and/or PC); and (b) an active moiety (e.g., a therapeutic moiety or detectable moiety), wherein the antibody or fragment is a scFv having the sequence of SEQ ID NO:38. In some embodiments, the antibody or fragment thereof competitively inhibits the binding of a pathogenic antibody (such as monoclonal antibody C2) to phospholipid. In some embodiments, the antibody or antibody fragment thereof binds to the same epitope as a pathogenic antibody (such as monoclonal antibody C2) to phospholipid. In some embodiments, the phospholipid is present on the surface of a cell, a basement membrane (e.g., Bruch's membrane), or in a pathological structure (e.g., drusen) in an individual that is in or adjacent to a tissue undergoing (or is at risk of undergoing) tissue injury (such as non-ischemic injury) and/or oxidative damage. In some embodiments, the phospholipid is selected from the group consisting of phosphatidylethanolamine (PE), cardiolipin (CL), and phosphatidylcholine (PC). In some embodiments, the phospholipid is malondialdehyde (MDA). In some embodiments, the phospholipid is neutral. In some embodiments, the phospholipid is positively charged. In some embodiments, the phospholipid is oxidized. In some embodiments, the construct comprises an active moiety that comprises a therapeutic moiety (such as a complement inhibitor). In some embodiments, the construct comprises an active moiety that is a detectable moiety. In some embodiments, the construct is a fusion protein. In some embodiments, the targeting moiety and the active moiety are linked via a linker (such as a peptide linker).

In some embodiments, the targeting moiety and the active moiety are directly bonded, covalently bonded, or, reversibly bonded.

A “construct” or “targeting construct” used herein refers to a non-naturally occurring molecule comprising a “targeting moiety” and an “active moiety”. The targeting moiety is capable of specifically binding to Annexin IV. The targeting moiety of the targeting construct is responsible for targeted delivery of the molecule to the sites of, e.g., complement activation. The active moiety is responsible for therapeutic activity, e.g., specifically inhibiting complement activation, or detection, e.g., permitting the detection and or localization of the targeting moiety. The targeting moiety and the active moiety of a targeting construct molecule can be linked together by any methods known in the art, as long as the desired functionalities of the two portions are maintained.

The targeting construct described herein thus generally has the dual functions of binding to an epitope recognized by an antibody described herein and exerting therapeutic activity or allowing detection. A “epitope of monoclonal antibody B4 antibody” refers to any molecule that binds to a naturally occurring B4 or C2 antibody, which include, epitopes that bind to a B4 or C2 antibody with a binding affinity that is about any of 10%, 20%, 30%, 40%, 50%, 60%, 70%, 80%, 90%, or 100% of the epitope that naturally binds a B4 antibody. Binding affinity can be determined by any method known in the art, including for example, surface plasmon resonance, calorimetry titration, ELISA, and flow cytometry.

In some embodiments, a targeting construct described herein is generally capable of inhibiting complement activation (for example inhibiting activation of the alternative pathway and/or lectin pathway). The targeting construct may be a more potent complement inhibitor than the naturally occurring antibody as described herein. For example, in some embodiments, the targeting construct has a complement inhibitory activity that is about any of 1.5, 2, 2.5, 3, 3.5, 4, 5, 6, 7, 8, 9, 10, 12, 14, 16, 18, 20, 25, 30, 40, or more fold of that of a B4 or C2 antibody. In some embodiments, the targeting construct has an EC50 of less than about any of 100 nM, 90 nM, 80 nM, 70 nM, 60 nM, 50 nM, 40 nM, 30 nM, 20 nM, or 10 nM, inclusive, including any values in between these numbers. In some embodiments, the targeting construct molecule has an EC50 of about 5 to 60 nM, including for example any of 8 to 50 nM, 8 to 20 nM, 10 to 40 nM, and 20 to 30 nM. In some embodiments, the targeting construct molecule has complement inhibitory activity that is about any of 50%, 60%, 70%, 80%, 90%, or 100% of that of a B4 or C2 antibody.

Complement inhibition can be evaluated based on any methods known in the art, including for example, in vitro zymosan assays, assays for lysis of erythrocytes, antibody or immune complex activation assays, alternative pathway activation assays, and mannan activation assays.

In some embodiments, the targeting construct is a fusion protein. “Fusion protein” used herein refers to two or more peptides, polypeptides, or proteins operably linked to each other. In some embodiments, the targeting moiety and the active moiety are directly fused to each other. In some embodiments, the targeting moiety and the active moiety are linked by an amino acid linker sequence. Examples of linker sequences are known in the art, and include, for example, (Gly4Ser), (Gly4Ser)2, (Gly4Ser)3, (Gly3Ser)4, (SerGly4), (SerGly4)2, (SerGly4)3, and (SerGly4)4. Linking sequences can also comprise “natural” linking sequences found between different domains of complement factors. The order of targeting moiety and active moiety in the fusion protein can vary. For example, in some embodiments, the C-terminus of the targeting moiety is fused (directly or indirectly) to the N-terminus of the active moiety of the targeting construct. In some embodiments, the N-terminus of the targeting moiety is fused (directly or indirectly) to the C-terminus of the active moiety of the targeting construct.

In some embodiments, the targeting moiety of a targeting construct is encoded by a polynucleotide comprising a nucleic acid sequence of any of SEQ ID NOs: 19-24 and 57. In some embodiments, the targeting construct molecule is encoded by a polynucleotide comprising a nucleic acid sequence that is at least about 50%, 60%, 70%, 80%, 90%, 91%, 92%, 93%, 94%, 95%, 96%, 97%, 98%, or 99% identical to that of any of SEQ ID NOs: 19-24 and 57.

In some embodiments, the targeting moiety of a targeting construct is encoded by a polynucleotide comprising a nucleic acid sequence of any of SEQ ID NOs: 37 or 38. In some embodiments, the targeting construct molecule is encoded by a polynucleotide comprising a nucleic acid sequence that is at least about 50%, 60%, 70%, 80%, 90%, 91%, 92%, 93%, 94%, 95%, 96%, 97%, 98%, or 99% identical to that of any of SEQ ID NOs: 37 or 38.

In some embodiments, the targeting construct comprises a targeting moiety and an active moiety linked via a chemical cross-linker. Linking of the two portions can occur on reactive groups located on the two moieties. Reactive groups that can be targeted using a crosslinker include primary amines, sulfhydryls, carbonyls, carbohydrates, and carboxylic acids, or active groups that can be added to proteins. Examples of chemical linkers are well known in the art and include, but are not limited to, bismaleimidohexane, maleimidobenzoyl-N-hydroxysuccinimide ester, NHS-Esters-Maleimide Crosslinkers such as SPDP, carbodiimide, glutaraldehyde, MBS, Sulfo-MBS, SMPB, sulfo-SMPB, GMBS, Sulfo-GMBS, EMCS, Sulfo-EMCS, imidoester crosslinkers such as DMA, DMP, DMS, DTBP, EDC and DTME.

In some embodiments, the targeting moiety and the active moiety are non-covalently linked. For example, the two portions may be brought together by two interacting bridging proteins (such as biotin and streptavidin), each linked to a targeting moiety or an active moiety.

In some embodiments, the targeting construct comprises two or more (same or different) targeting moieties described herein. In some embodiments, the targeting construct comprises two or more (same or different) active moieties described herein. These two or more targeting (or active) moieties may be tandemly linked (such as fused) to each other. In some embodiments, the targeting construct comprises a targeting moiety and two or more (such as three, four, five, or more) active moieties. In some embodiments, the targeting construct comprises an active moiety and two or more (such as three, four, five, or more) targeting moieties. In some embodiments, the targeting construct comprises two or more targeting moieties and two or more active moieties.

In some embodiments, there is provided an isolated targeting construct. In some embodiments, the targeting constructs form dimers or multimers.

The active moiety and the targeting moiety in the targeting construct can be from the same species (such as human or mouse), or from different species.

Also provided herein are targeting constructs and compositions (such as pharmaceutical compositions) comprising a targeting construct molecule. The present application further provides methods of delivering any of the complement modulator or detectable moiety disclosed herein to a site of complement activation, a site of tissue injury (such as non-ischemic tissue injury), or a site of complement-associated disease in an individual by administering to the individual any one of the target constructs described herein.

A “targeting construct” used herein refers to a non-naturally occurring molecule comprising a “targeting moiety” and an “active moiety”. In certain embodiments, the targeting moiety is capable of binding to Annexin IV. In certain embodiments, the targeting moiety is capable of binding a phospholipid, such as PC, PE, and/or CL. The targeting moiety of the targeting construct is thus responsible for targeted delivery of the molecule to the sites of, e.g., complement activation. The active moiety is responsible for therapeutic activity, e.g., specifically inhibiting complement activation, or detection, e.g., permitting the detection and or localization of the targeting moiety. The targeting moiety and the active moiety of a targeting construct molecule can be linked together by any methods known in the art, as long as the desired functionalities of the two portions are maintained.

The targeting construct described herein thus generally has the dual functions of binding to an epitope recognized by an antibody described herein and exerting therapeutic activity or allowing detection. A “epitope of a B4 or C2 antibody” refers to any molecule that binds to a naturally occurring B4 or C2 antibody, which include, epitopes that bind to a B4 or C2 antibody with a binding affinity that is about any of 10%, 20%, 30%, 40%, 50%, 60%, 70%, 80%, 90%, or 100% of the epitope that naturally binds a B4 or C2 antibody. Binding affinity can be determined by any method known in the art, including for example, surface plasmon resonance, calorimetry titration, ELISA, and flow cytometry.

In some embodiments, a targeting construct described herein is generally capable of inhibiting complement activation (for example inhibiting activation of the alternative pathway and/or lectin pathway). The targeting construct may be a more potent complement inhibitor than the naturally occurring antibody as described herein. For example, in some embodiments, the targeting construct has a complement inhibitory activity that is about any of 1.5, 2, 2.5, 3, 3.5, 4, 5, 6, 7, 8, 9, 10, 12, 14, 16, 18, 20, 25, 30, 40, or more fold of that of a B4 or C2 antibody. In some embodiments, the targeting construct has an EC50 of less than about any of 100 nM, 90 nM, 80 nM, 70 nM, 60 nM, 50 nM, 40 nM, 30 nM, 20 nM, or 10 nM. In some embodiments, the targeting construct molecule has an EC50 of about 5-60 nM, including for example any of 8-50 nM, 8-20 nM, 10-40 nM, and 20-30 nM. In some embodiments, the targeting construct molecule has complement inhibitory activity that is about any of 50%, 60%, 70%, 80%, 90%, or 100% of that of a B4 or C2 antibody.

Complement inhibition can be evaluated based on any methods known in the art, including for example, in vitro zymosan assays, assays for lysis of erythrocytes, immune complex activation assays, and mannan activation assays.

In some embodiments, the targeting construct is a fusion protein. “Fusion protein” used herein refers to two or more peptides, polypeptides, or proteins operably linked to each other. In some embodiments, the targeting moiety and the active moiety are directly fused to each other. In some embodiments, the targeting moiety and the active moiety are linked by an amino acid linker sequence. Examples of linker sequences are known in the art, and include, for example, (Gly4Ser), (Gly4Ser)2, (Gly4Ser)3, (Gly3Ser)4, (SerGly4), (SerGly4)2, (SerGly4)3, and (SerGly4)4. Linking sequences can also comprise “natural” linking sequences found between different domains of complement factors. The order of targeting moiety and active moiety in the fusion protein can vary. For example, in some embodiments, the C-terminus of the targeting moiety is fused (directly or indirectly) to the N-terminus of the active moiety of the targeting construct. In some embodiments, the N-terminus of the targeting moiety is fused (directly or indirectly) to the C-terminus of the active moiety of the targeting construct.

In some embodiments, the targeting moiety of a targeting construct is encoded by a polynucleotide comprising a nucleic acid sequence of any of SEQ ID NOs: 19-24, 39-43, 57 and 58. In some embodiments, the targeting construct molecule is encoded by a polynucleotide comprising a nucleic acid sequence that is at least about 50%, 60%, 70%, 80%, 90%, 91%, 92%, 93%, 94%, 95%, 96%, 97%, 98%, or 99% identical to that of any of SEQ ID NOs: 19-24 and 39-43.

In some embodiments, the targeting construct comprises a targeting moiety and an active moiety linked via a chemical cross-linker. Linking of the two portions can occur on reactive groups located on the two moieties. Reactive groups that can be targeted using a crosslinker include primary amines, sulfhydryls, carbonyls, carbohydrates, and carboxylic acids, or active groups that can be added to proteins. Examples of chemical linkers are well known in the art and include, but are not limited to, bismaleimidohexane, maleimidobenzoyl-N-hydroxysuccinimide ester, NHS-Esters-Maleimide Crosslinkers such as SPDP, carbodiimide, glutaraldehyde, MBS, Sulfo-MBS, SMPB, sulfo-SMPB, GMBS, Sulfo-GMBS, EMCS, Sulfo-EMCS, imidoester crosslinkers such as DMA, DMP, DMS, DTBP, EDC and DTME.

In some embodiments, the targeting moiety and the active moiety are non-covalently linked. For example, the two portions may be brought together by two interacting bridging proteins (such as biotin and streptavidin), each linked to a targeting moiety or an active moiety.

In some embodiments, the targeting construct comprises two or more (same or different) targeting moieties described herein. In some embodiments, the targeting construct comprises two or more (same or different) active moieties described herein. These two or more targeting (or active) moieties may be tandemly linked (such as fused) to each other. In some embodiments, the targeting construct comprises a targeting moiety and two or more (such as three, four, five, or more) active moieties. In some embodiments, the targeting construct comprises an active moiety and two or more (such as three, four, five, or more) targeting moieties. In some embodiments, the targeting construct comprises two or more targeting moieties and two or more active moieties.

In some embodiments, there is provided an isolated targeting construct. In some embodiments, the targeting constructs form dimers or multimers.

The active moiety and the targeting moiety in the targeting construct can be from the same species (such as human or mouse), or from different species.

In some embodiments, there is provided a construct (or a composition comprising the construct such as a pharmaceutical composition), wherein the construct comprises (a) an antibody or a fragment thereof, wherein the antibody or a fragment thereof specifically binds to Annexin IV; and (b) a therapeutic agent or a detectable moiety. In some embodiments, the construct comprises a therapeutic agent (such as a complement modulator, for example a complement inhibitor). In some embodiments, the construct comprises a detectable moiety. In some embodiments, the construct is a fusion protein. In some embodiments, the antibody or fragment thereof (hereinafter also referred to as the “targeting moiety” and the detectable moiety (hereinafter also referred to as “the active moiety” are linked via a linker (such as a peptide linker). In some embodiments, the targeting moiety and the active moiety are directly linked.

In some embodiments, there is provided a construct (or a composition comprising the construct such as a pharmaceutical composition), wherein the construct comprises (a) an antibody or a fragment thereof, wherein the antibody or a fragment thereof specifically binds to Annexin IV; and (b) a complement modulator or a detectable moiety, wherein the antibody or fragment there of comprises: (i) a light chain variable domain comprising a sequence of SEQ ID NO:1, a sequence of SEQ ID NO:2, or a sequence of SEQ ID NO:3; and/or (ii) heavy chain variable domain comprising a sequence of SEQ ID NO:4, a sequence of SEQ ID NO:5, or a sequence of SEQ ID NO:6. In some embodiments, there is provided a construct (or a composition comprising the construct such as a pharmaceutical composition), wherein the construct comprises (a) an antibody or a fragment thereof, wherein the antibody or a fragment thereof specifically binds to Annexin IV; and (b) a complement modulator or a detectable moiety, wherein the antibody or fragment there of comprises: (i) a light chain variable domain comprising a sequence of SEQ ID NO:7, a sequence of SEQ ID NO:8, or a sequence of SEQ ID NO:9; and/or (ii) heavy chain variable domain comprising a sequence of SEQ ID NO:10, a sequence of SEQ ID NO:11, or a sequence of SEQ ID NO:12. In some embodiments, the antibody or fragment thereof competitively inhibits the binding of a pathogenic antibody (such as monoclonal antibody B4) to Annexin IV. In some embodiments, the antibody or fragment thereof binds to the same epitope as a pathogenic antibody (such as a monoclonal antibody B4) to Annexin IV. In some embodiments, the Annexin IV is present on the surface of a cell (and/or in a pathological structure) in an individual that is in or adjacent to a tissue undergoing (or is at risk of undergoing) tissue injury (such as non-ischemic injury) and/oxidative damage. In some embodiments, the Annexin IV is produced by a nucleated cell (such as a mammalian cell). In some embodiments, the Annexin IV is recombinant protein. In some embodiments, the construct comprises a complement modulator (such as a complement inhibitor). In some embodiments, the construct comprises a detectable moiety. In some embodiments, the construct is a fusion protein. In some embodiments, the targeting moiety and the active moiety are linked via a linker (such as a peptide linker). In some embodiments, the targeting moiety and the active moiety are directly linked.

In some embodiments, there is provided a construct (or a composition comprising the construct such as a pharmaceutical composition), wherein the construct comprises (a) an antibody or a fragment thereof, wherein the antibody or a fragment thereof specifically binds to Annexin IV; and (b) a complement modulator or a detectable moiety, wherein the antibody or fragment there of comprises: (i) a light chain variable domain comprising a sequence of SEQ ID NO:1; (ii) a light chain variable domain comprising a sequence of SEQ ID NO:2; and (iii) a light chain variable domain comprising a sequence of SEQ ID NO:3. In some embodiments, there is provided a construct (or a composition comprising the construct such as a pharmaceutical composition), wherein the construct comprises (a) an antibody or a fragment thereof, wherein the antibody or a fragment thereof specifically binds to Annexin IV; and (b) a complement modulator or a detectable moiety, wherein the antibody or fragment there of comprises: (i) a light chain variable domain comprising a sequence of SEQ ID NO:7; (ii) a light chain variable domain comprising a sequence of SEQ ID NO:8; and (iii) a light chain variable domain comprising a sequence of SEQ ID NO:9. In some embodiments, the antibody or fragment thereof competitively inhibits the binding of a pathogenic antibody (such as monoclonal antibody B4) to Annexin IV. In some embodiments, the antibody or fragment thereof binds to the same epitope as a pathogenic antibody (such as a monoclonal antibody B4) to Annexin IV. In some embodiments, the Annexin IV is present on the surface of a cell (and/or in a pathological structure) in an individual that is in or adjacent to a tissue undergoing (or is at risk of undergoing) tissue injury (such as non-ischemic injury) and/oxidative damage. In some embodiments, the Annexin IV is produced by a nucleated cell (such as a mammalian cell). In some embodiments, the Annexin IV is recombinant protein. In some embodiments, the construct comprises a complement modulator (such as a complement inhibitor). In some embodiments, the construct comprises a detectable moiety. In some embodiments, the construct is a fusion protein. In some embodiments, the targeting moiety and the active moiety are linked via a linker (such as a peptide linker). In some embodiments, the targeting moiety and the active moiety are directly linked.

In some embodiments, there is provided a construct (or a composition comprising the construct such as a pharmaceutical composition), wherein the construct comprises (a) an antibody or a fragment thereof, wherein the antibody or a fragment thereof specifically binds to Annexin IV; and (b) a complement modulator or a detectable moiety, wherein the antibody or fragment there of comprises: (i) heavy chain variable domain comprising a sequence of SEQ ID NO:4; (ii) heavy chain variable domain comprising a sequence of SEQ ID NO:5; and (iii) heavy chain variable domain comprising a sequence of SEQ ID NO:6. In some embodiments, there is provided a construct (or a composition comprising the construct such as a pharmaceutical composition), wherein the construct comprises (a) an antibody or a fragment thereof, wherein the antibody or a fragment thereof specifically binds to Annexin IV; and (b) a complement modulator or a detectable moiety, wherein the antibody or fragment there of comprises: (i) heavy chain variable domain comprising a sequence of SEQ ID NO:10; (ii) heavy chain variable domain comprising a sequence of SEQ ID NO:11; and (iii) heavy chain variable domain comprising a sequence of SEQ ID NO:12. In some embodiments, the antibody or fragment thereof competitively inhibits the binding of a pathogenic antibody (such as monoclonal antibody B4) to Annexin IV. In some embodiments, the antibody or fragment thereof binds to the same epitope as a pathogenic antibody (such as a monoclonal antibody B4) to Annexin IV. In some embodiments, the Annexin IV is present on the surface of a cell (and/or in a pathological structure) in an individual that is in or adjacent to a tissue undergoing (or is at risk of undergoing) tissue injury (such as non-ischemic injury) and/oxidative damage. In some embodiments, the Annexin IV is produced by a nucleated cell (such as a mammalian cell). In some embodiments, the Annexin IV is recombinant protein. In some embodiments, the construct comprises a complement modulator (such as a complement inhibitor). In some embodiments, the construct comprises a detectable moiety. In some embodiments, the construct is a fusion protein. In some embodiments, the targeting moiety and the active moiety are linked via a linker (such as a peptide linker). In some embodiments, the targeting moiety and the active moiety are directly linked.

In some embodiments, there is provided a construct (or a composition comprising the construct such as a pharmaceutical composition), wherein the construct comprises (a) an antibody or a fragment thereof, wherein the antibody or a fragment thereof specifically binds to Annexin IV; and (b) a complement modulator or a detectable moiety, wherein the antibody or fragment there of comprises: (i) a light chain variable domain comprising a sequence of SEQ ID NO:1; (ii) a light chain variable domain comprising a sequence of SEQ ID NO:2; (iii) a light chain variable domain comprising a sequence of SEQ ID NO:3; (iv) heavy chain variable domain comprising a sequence of SEQ ID NO:4; (v) heavy chain variable domain comprising a sequence of SEQ ID NO:5; and (vi) heavy chain variable domain comprising a sequence of SEQ ID NO:6. In some embodiments, there is provided a construct (or a composition comprising the construct such as a pharmaceutical composition), wherein the construct comprises (a) an antibody or a fragment thereof, wherein the antibody or a fragment thereof specifically binds to Annexin IV; and (b) a complement modulator or a detectable moiety, wherein the antibody or fragment there of comprises: (i) a light chain variable domain comprising a sequence of SEQ ID NO:7; (ii) a light chain variable domain comprising a sequence of SEQ ID NO:8; (iii) a light chain variable domain comprising a sequence of SEQ ID NO:9; (iv) heavy chain variable domain comprising a sequence of SEQ ID NO:10; (v) heavy chain variable domain comprising a sequence of SEQ ID NO:11; and (vi) heavy chain variable domain comprising a sequence of SEQ ID NO:12. In some embodiments, the antibody or fragment thereof competitively inhibits the binding of a pathogenic antibody (such as monoclonal antibody B4) to Annexin IV. In some embodiments, the antibody or fragment thereof binds to the same epitope as a pathogenic antibody (such as a monoclonal antibody B4) to Annexin IV. In some embodiments, the Annexin IV is present on the surface of a cell (and/or in a pathological structure) in an individual that is in or adjacent to a tissue undergoing (or is at risk of undergoing) tissue injury (such as non-ischemic injury) and/oxidative damage. In some embodiments, the Annexin IV is produced by a nucleated cell (such as a mammalian cell). In some embodiments, the Annexin IV is recombinant protein. In some embodiments, the construct comprises a complement modulator (such as a complement inhibitor). In some embodiments, the construct comprises a detectable moiety. In some embodiments, the construct is a fusion protein. In some embodiments, the targeting moiety and the active moiety are linked via a linker (such as a peptide linker). In some embodiments, the targeting moiety and the active moiety are directly linked.

In some embodiments, there is provided a construct (or a composition comprising the construct such as a pharmaceutical composition), wherein the construct comprises (a) an antibody or a fragment thereof, wherein the antibody or a fragment thereof specifically binds to Annexin IV; and (b) a complement modulator or a detectable moiety, wherein the antibody or fragment there of comprises: (i) a light chain CDR1 of SEQ ID NO:1; (ii) a light chain CDR2 of SEQ ID NO:2; (iii) a light chain CDR3 of SEQ ID NO:3; (iv) heavy chain CDR1 of SEQ ID NO:4; (v) heavy chain CDR2 of SEQ ID NO:5; and (vi) heavy chain CDR3 of SEQ ID NO:6. In some embodiments, there is provided a construct (or a composition comprising the construct such as a pharmaceutical composition), wherein the construct comprises (a) an antibody or a fragment thereof, wherein the antibody or a fragment thereof specifically binds to Annexin IV; and (b) a complement modulator or a detectable moiety, wherein the antibody or fragment there of comprises: (i) a light chain CDR1 of SEQ ID NO:7; (ii) a light chain CDR2 of SEQ ID NO:8; (iii) a light chain CDR3 of SEQ ID NO:9; (iv) heavy chain CDR1 of SEQ ID NO:10; (v) heavy chain CDR2 of SEQ ID NO:11; and (vi) heavy chain CDR3 of SEQ ID NO:12. In some embodiments, the antibody or fragment thereof competitively inhibits the binding of a pathogenic antibody (such as monoclonal antibody B4) to Annexin IV. In some embodiments, the antibody or fragment thereof binds to the same epitope as a pathogenic antibody (such as a monoclonal antibody B4) to Annexin IV. In some embodiments, the Annexin IV is present on the surface of a cell (and/or in a pathological structure) in an individual that is in or adjacent to a tissue undergoing (or is at risk of undergoing) tissue injury (such as non-ischemic injury) and/oxidative damage. In some embodiments, the Annexin IV is produced by a nucleated cell (such as a mammalian cell). In some embodiments, the Annexin IV is recombinant protein. In some embodiments, the construct comprises a complement modulator (such as a complement inhibitor). In some embodiments, the construct comprises a detectable moiety. In some embodiments, the construct is a fusion protein. In some embodiments, the targeting moiety and the active moiety are linked via a linker (such as a peptide linker). In some embodiments, the targeting moiety and the active moiety are directly linked.

In some embodiments, there is provided a construct (or a composition comprising the construct such as a pharmaceutical composition), wherein the construct comprises (a) an antibody or a fragment thereof, wherein the antibody or a fragment thereof specifically binds to Annexin IV; and (b) a complement modulator or a detectable moiety, wherein the antibody or fragment there of comprises a light chain variable domain of SEQ ID NO:13. In some embodiments, there is provided a construct (or a composition comprising the construct such as a pharmaceutical composition), wherein the construct comprises (a) an antibody or a fragment thereof, wherein the antibody or a fragment thereof specifically binds to Annexin IV; and (b) a complement modulator or a detectable moiety, wherein the antibody or fragment there of comprises a heavy chain variable domain of SEQ ID NO:15. In some embodiments, there is provided a construct (or a composition comprising the construct such as a pharmaceutical composition), wherein the construct comprises (a) an antibody or a fragment thereof, wherein the antibody or a fragment thereof specifically binds to Annexin IV; and (b) a complement modulator or a detectable moiety, wherein the antibody or fragment there of comprises a light chain variable domain of SEQ ID NO:14. In some embodiments, there is provided a construct (or a composition comprising the construct such as a pharmaceutical composition), wherein the construct comprises (a) an antibody or a fragment thereof, wherein the antibody or a fragment thereof specifically binds to Annexin IV; and (b) a complement modulator or a detectable moiety, wherein the antibody or fragment there of comprises a heavy chain variable domain of SEQ ID NO:16. In some embodiments, the antibody or fragment thereof competitively inhibits the binding of a pathogenic monoclonal antibody (such as monoclonal antibody B4) to Annexin IV. I In some embodiments, the antibody or fragment thereof competitively inhibits the binding of a pathogenic antibody (such as monoclonal antibody B4) to Annexin IV. In some embodiments, the antibody or fragment thereof binds to the same epitope as a pathogenic antibody (such as a monoclonal antibody B4) to Annexin IV. In some embodiments, the Annexin IV is present on the surface of a cell (and/or in a pathological structure) in an individual that is in or adjacent to a tissue undergoing (or is at risk of undergoing) tissue injury (such as non-ischemic injury) and/oxidative damage. In some embodiments, the Annexin IV is produced by a nucleated cell (such as a mammalian cell). In some embodiments, the Annexin IV is recombinant protein. In some embodiments, the construct comprises a complement modulator (such as a complement inhibitor). In some embodiments, the construct comprises a detectable moiety. In some embodiments, the construct is a fusion protein. In some embodiments, the targeting moiety and the active moiety are linked via a linker (such as a peptide linker). In some embodiments, the targeting moiety and the active moiety are directly linked.

In some embodiments, there is provided a construct (or a composition comprising the construct such as a pharmaceutical composition), wherein the construct comprises (a) an antibody or a fragment thereof, wherein the antibody or a fragment thereof specifically binds to Annexin IV; and (b) a complement modulator or a detectable moiety, wherein the antibody or fragment there of comprises: (i) a light chain variable domain of SEQ ID NO:13; and (ii) heavy chain variable domain of SEQ ID NO:15. In some embodiments, there is provided a construct (or a composition comprising the construct such as a pharmaceutical composition), wherein the construct comprises (a) an antibody or a fragment thereof, wherein the antibody or a fragment thereof specifically binds to Annexin IV; and (b) a complement modulator or a detectable moiety, wherein the antibody or fragment there of comprises: (i) a light chain variable domain of SEQ ID NO:14; and (ii) heavy chain variable domain of SEQ ID NO:16. In some embodiments, the antibody or fragment thereof competitively inhibits the binding of a pathogenic antibody (such as monoclonal antibody B4) to Annexin IV. In some embodiments, the antibody or fragment thereof binds to the same epitope as a pathogenic antibody (such as a monoclonal antibody B4) to Annexin IV. In some embodiments, the Annexin IV is present on the surface of a cell (and/or in a pathological structure) in an individual that is in or adjacent to a tissue undergoing (or is at risk of undergoing) tissue injury (such as non-ischemic injury) and/oxidative damage. In some embodiments, the Annexin IV is produced by a nucleated cell (such as a mammalian cell). In some embodiments, the Annexin IV is recombinant protein. In some embodiments, the construct comprises a complement modulator (such as a complement inhibitor). In some embodiments, the construct comprises a detectable moiety. In some embodiments, the construct is a fusion protein. In some embodiments, the targeting moiety and the active moiety are linked via a linker (such as a peptide linker). In some embodiments, the targeting moiety and the active moiety are directly linked.

In some embodiments, there is provided a construct (or a composition comprising the construct such as a pharmaceutical composition), wherein the construct comprises (a) an antibody or a fragment thereof, wherein the antibody or a fragment thereof specifically binds to Annexin IV; and (b) a complement modulator or a detectable moiety, wherein the antibody or fragment is a scFv having the sequence of SEQ ID NO:17. In some embodiments, there is provided a construct (or a composition comprising the construct such as a pharmaceutical composition), wherein the construct comprises (a) an antibody or a fragment thereof, wherein the antibody or a fragment thereof specifically binds to Annexin IV; and (b) a complement modulator or a detectable moiety, wherein the antibody or fragment is a scFv having the sequence of SEQ ID NO:18. In some embodiments, the antibody or fragment thereof competitively inhibits the binding of a pathogenic antibody (such as monoclonal antibody B4) to Annexin IV. In some embodiments, the antibody or fragment thereof binds to the same epitope as a pathogenic antibody (such as a monoclonal antibody B4) to Annexin IV. In some embodiments, the Annexin IV is present on the surface of a cell (and/or in a pathological structure) in an individual that is in or adjacent to a tissue undergoing (or is at risk of undergoing) tissue injury (such as non-ischemic injury) and/oxidative damage. In some embodiments, the Annexin IV is produced by a nucleated cell (such as a mammalian cell). In some embodiments, the Annexin IV is recombinant protein. In some embodiments, the construct comprises a complement modulator (such as a complement inhibitor). In some embodiments, the construct comprises a detectable moiety. In some embodiments, the construct is a fusion protein. In some embodiments, the targeting moiety and the active moiety are linked via a linker (such as a peptide linker). In some embodiments, the targeting moiety and the active moiety are directly linked.

The present application in some embodiments provides various targeting constructs for targeted delivery. In some embodiments, there is provided a targeting construct (or a composition comprising the construct such as a pharmaceutical composition), wherein the targeting construct comprises (a) an antibody or a fragment thereof, wherein the antibody or a fragment thereof specifically binds to a phospholipid (such as PE, CL, MDA, and/or PC); and (b) a an active moiety, wherein the antibody or fragment thereof comprises: (i) a light chain variable domain comprising a sequence of SEQ ID NO:25, a sequence of SEQ ID NO:26, or a sequence of SEQ ID NO:27; and/or (ii) heavy chain variable domain comprising a sequence of SEQ ID NO:28, a sequence of SEQ ID NO:29, or a sequence of SEQ ID NO:30. In some embodiments, there is provided a construct (or a composition comprising the construct such as a pharmaceutical composition), wherein the construct comprises (a) an antibody or a fragment thereof, wherein the antibody or a fragment thereof specifically binds to a phospholipid (such as PE, CL, MDA, and/or PC); and (b) a complement modulator or a detectable moiety, wherein the antibody or fragment thereof comprises: (i) a light chain variable domain comprising a sequence of SEQ ID NO:31, a sequence of SEQ ID NO:32, or a sequence of SEQ ID NO:33; and/or (ii) heavy chain variable domain comprising a sequence of SEQ ID NO:28, a sequence of SEQ ID NO:29, or a sequence of SEQ ID NO:30. In some embodiments, the antibody or fragment thereof competitively inhibits the binding of a pathogenic antibody (such as monoclonal antibody C2) to phospholipid. In some embodiments, the antibody or antibody fragment thereof binds to the same epitope as a pathogenic antibody (such as monoclonal antibody C2) to phospholipid. In some embodiments, the phospholipid is present on the surface of a cell, a basement membrane (e.g., Bruch's membrane), or in a pathological structure (e.g., drusen) in an individual that is in or adjacent to a tissue undergoing (or is at risk of undergoing) tissue injury (such as non-ischemic injury) and/or oxidative damage. In some embodiments, the phospholipid is selected from the group consisting of phosphatidylethanolamine (PE), cardiolipin (CL), and phosphatidylcholine (PC). In some embodiments, the phospholipid is malondialdehyde (MDA). In some embodiments, the phospholipid is neutral. In some embodiments, the phospholipid is positively charged. In some embodiments, the phospholipid is oxidized. In some embodiments, the construct comprises an active moiety that comprises a therapeutic moiety (such as a complement inhibitor). In some embodiments, the construct comprises an active moiety that is a detectable moiety. In some embodiments, the construct is a fusion protein. In some embodiments, the targeting moiety and the active moiety are linked via a linker (such as a peptide linker).

In some embodiments, there is provided a targeting construct (or a composition comprising the construct such as a pharmaceutical composition), wherein the targeting construct comprises (a) an antibody or a fragment thereof, wherein the antibody or a fragment thereof specifically binds to a phospholipid (such as PE, CL, MDA, and/or PC); and (b) an active moiety (e.g., a therapeutic moiety or a detectable moiety), wherein the antibody or fragment thereof comprises: (i) a light chain variable domain comprising a sequence of SEQ ID NO:25; (ii) a light chain variable domain comprising a sequence of SEQ ID NO:26; and (iii) a light chain variable domain comprising a sequence of SEQ ID NO:27. In some embodiments, there is provided a targeting construct (or a composition comprising the construct such as a pharmaceutical composition), wherein the targeting construct comprises (a) an antibody or a fragment thereof, wherein the antibody or a fragment thereof specifically binds to a phospholipid (such as PE, CL, MDA, and/or PC); and (b) an active moiety (e.g., a therapeutic moiety or a detectable moiety), wherein the antibody or fragment thereof comprises: (i) a light chain variable domain comprising a sequence of SEQ ID NO:31; (ii) a light chain variable domain comprising a sequence of SEQ ID NO:32; and (iii) a light chain variable domain comprising a sequence of SEQ ID NO:33. In some embodiments, the antibody or fragment thereof competitively inhibits the binding of a pathogenic antibody (such as monoclonal antibody C2) to phospholipid. In some embodiments, the antibody or antibody fragment thereof binds to the same epitope as a pathogenic antibody (such as monoclonal antibody C2) to phospholipid. In some embodiments, the phospholipid is present on the surface of a cell, a basement membrane (e.g., Bruch's membrane), or in a pathological structure (e.g., drusen) in an individual that is in or adjacent to a tissue undergoing (or is at risk of undergoing) tissue injury (such as non-ischemic injury) and/or oxidative damage. In some embodiments, the phospholipid is selected from the group consisting of phosphatidylethanolamine (PE), cardiolipin (CL), and phosphatidylcholine (PC). In some embodiments, the phospholipid is malondialdehyde (MDA). In some embodiments, the phospholipid is neutral. In some embodiments, the phospholipid is positively charged. In some embodiments, the phospholipid is oxidized. In some embodiments, the construct comprises an active moiety that comprises a therapeutic moiety (such as a complement inhibitor). In some embodiments, the construct comprises an active moiety that is a detectable moiety. In some embodiments, the construct is a fusion protein. In some embodiments, the targeting moiety and the active moiety are linked via a linker (such as a peptide linker).

In some embodiments, there is provided a targeting construct (or a composition comprising the construct such as a pharmaceutical composition), wherein the construct comprises (a) an antibody or a fragment thereof, wherein the antibody or a fragment thereof specifically binds to a phospholipid (such as PE, CL, MDA, and/or PC); and (b) an active moiety (e.g., a therapeutic moiety or a detectable moiety), wherein the antibody or fragment thereof comprises: (i) heavy chain variable domain comprising a sequence of SEQ ID NO:28; (ii) heavy chain variable domain comprising a sequence of SEQ ID NO:29; and (iii) heavy chain variable domain comprising a sequence of SEQ ID NO:30. In some embodiments, the antibody or fragment thereof competitively inhibits the binding of a pathogenic antibody (such as monoclonal antibody C2) to phospholipid. In some embodiments, the antibody or antibody fragment thereof binds to the same epitope as a pathogenic antibody (such as monoclonal antibody C2) to phospholipid. In some embodiments, the phospholipid is present on the surface of a cell, a basement membrane (e.g., Bruch's membrane), or in a pathological structure (e.g., drusen) in an individual that is in or adjacent to a tissue undergoing (or is at risk of undergoing) tissue injury (such as non-ischemic injury) and/or oxidative damage. In some embodiments, the phospholipid is selected from the group consisting of phosphatidylethanolamine (PE), cardiolipin (CL), and phosphatidylcholine (PC). In some embodiments, the phospholipid is malondialdehyde (MDA). In some embodiments, the phospholipid is neutral. In some embodiments, the phospholipid is positively charged. In some embodiments, the phospholipid is oxidized. In some embodiments, the construct comprises an active moiety that comprises a therapeutic moiety (such as a complement inhibitor). In some embodiments, the construct comprises an active moiety that is a detectable moiety. In some embodiments, the construct is a fusion protein. In some embodiments, the targeting moiety and the active moiety are linked via a linker (such as a peptide linker).

In some embodiments, there is provided a targeting construct (or a composition comprising the construct such as a pharmaceutical composition), wherein the targeting construct comprises (a) an antibody or a fragment thereof, wherein the antibody or a fragment thereof specifically binds to a phospholipid (such as PE, CL, MDA, and/or PC); and (b) an active moiety (e.g., a therapeutic moiety or a detectable moiety), wherein the antibody or fragment thereof comprises: (i) a light chain variable domain comprising a sequence of SEQ ID NO:25; (ii) a light chain variable domain comprising a sequence of SEQ ID NO:26; (iii) a light chain variable domain comprising a sequence of SEQ ID NO:27; (iv) heavy chain variable domain comprising a sequence of SEQ ID NO:28; (v) heavy chain variable domain comprising a sequence of SEQ ID NO:29; and (vi) heavy chain variable domain comprising a sequence of SEQ ID NO:30. In some embodiments, there is provided a targeting construct (or a composition comprising the construct such as a pharmaceutical composition), wherein the targeting construct comprises (a) an antibody or a fragment thereof, wherein the antibody or a fragment thereof specifically binds to a phospholipid (such as PE, CL, MDA, and/or PC); and (b) an active moiety (e.g., a therapeutic moiety or a detectable moiety), wherein the antibody or fragment thereof comprises: (i) a light chain variable domain comprising a sequence of SEQ ID NO:31; (ii) a light chain variable domain comprising a sequence of SEQ ID NO:32; (iii) a light chain variable domain comprising a sequence of SEQ ID NO:33; (iv) heavy chain variable domain comprising a sequence of SEQ ID NO:28; (v) heavy chain variable domain comprising a sequence of SEQ ID NO:29; and (vi) heavy chain variable domain comprising a sequence of SEQ ID NO:30. In some embodiments, the antibody or fragment thereof competitively inhibits the binding of a pathogenic antibody (such as monoclonal antibody C2) to phospholipid. In some embodiments, the antibody or antibody fragment thereof binds to the same epitope as a pathogenic antibody (such as monoclonal antibody C2) to phospholipid. In some embodiments, the phospholipid is present on the surface of a cell, a basement membrane (e.g., Bruch's membrane), or in a pathological structure (e.g., drusen) in an individual that is in or adjacent to a tissue undergoing (or is at risk of undergoing) tissue injury (such as non-ischemic injury) and/or oxidative damage. In some embodiments, the phospholipid is selected from the group consisting of phosphatidylethanolamine (PE), cardiolipin (CL), and phosphatidylcholine (PC). In some embodiments, the phospholipid is malondialdehyde (MDA). In some embodiments, the phospholipid is neutral. In some embodiments, the phospholipid is positively charged. In some embodiments, the phospholipid is oxidized. In some embodiments, the construct comprises an active moiety that comprises a therapeutic moiety (such as a complement inhibitor). In some embodiments, the construct comprises an active moiety that is a detectable moiety. In some embodiments, the construct is a fusion protein. In some embodiments, the targeting moiety and the active moiety are linked via a linker (such as a peptide linker).

In some embodiments, there is provided a targeting construct (or a composition comprising the construct such as a pharmaceutical composition), wherein the targeting construct comprises (a) an antibody or a fragment thereof, wherein the antibody or a fragment thereof specifically binds to a phospholipid (such as PE, CL, MDA, and/or PC); and (b) an active moiety (e.g., a therapeutic moiety or a detectable moiety), wherein the antibody or fragment thereof comprises: (i) a light chain CDR1 of SEQ ID NO:25; (ii) a light chain CDR2 of SEQ ID NO:26; (iii) a light chain CDR3 of SEQ ID NO:27; (iv) heavy chain CDR1 of SEQ ID NO:28; (v) heavy chain CDR2 of SEQ ID NO:29; and (vi) heavy chain CDR3 of SEQ ID NO:30. In some embodiments, there is provided a targeting construct (or a composition comprising the construct such as a pharmaceutical composition), wherein the targeting construct comprises (a) an antibody or a fragment thereof, wherein the antibody or a fragment thereof specifically binds to a phospholipid (such as PE, CL, MDA, and/or PC); and (b) an active moiety (e.g., a therapeutic moiety or a detectable moiety), wherein the antibody or fragment thereof comprises: (i) a light chain CDR1 of SEQ ID NO:31; (ii) a light chain CDR2 of SEQ ID NO:32; (iii) a light chain CDR3 of SEQ ID NO:33; (iv) heavy chain CDR1 of SEQ ID NO:28; (v) heavy chain CDR2 of SEQ ID NO:29; and (vi) heavy chain CDR3 of SEQ ID NO:30. In some embodiments, the antibody or fragment thereof competitively inhibits the binding of a pathogenic antibody (such as monoclonal antibody C2) to phospholipid. In some embodiments, the antibody or antibody fragment thereof binds to the same epitope as a pathogenic antibody (such as monoclonal antibody C2) to phospholipid. In some embodiments, the phospholipid is present on the surface of a cell, a basement membrane (e.g., Bruch's membrane), or in a pathological structure (e.g., drusen) in an individual that is in or adjacent to a tissue undergoing (or is at risk of undergoing) tissue injury (such as non-ischemic injury) and/or oxidative damage. In some embodiments, the phospholipid is selected from the group consisting of phosphatidylethanolamine (PE), cardiolipin (CL), and phosphatidylcholine (PC). In some embodiments, the phospholipid is malondialdehyde (MDA). In some embodiments, the phospholipid is neutral. In some embodiments, the phospholipid is positively charged. In some embodiments, the phospholipid is oxidized. In some embodiments, the construct comprises an active moiety that comprises a therapeutic moiety (such as a complement inhibitor). In some embodiments, the construct comprises an active moiety that is a detectable moiety. In some embodiments, the construct is a fusion protein. In some embodiments, the targeting moiety and the active moiety are linked via a linker (such as a peptide linker).

In some embodiments, there is provided a targeting construct (or a composition comprising the construct such as a pharmaceutical composition), wherein the targeting construct comprises (a) an antibody or a fragment thereof, wherein the antibody or a fragment thereof specifically binds to a phospholipid (such as PE, CL, MDA, and/or PC); and (b) an active moiety (e.g., a therapeutic moiety or a detectable moiety), wherein the antibody or fragment thereof comprises a light chain variable domain of SEQ ID NO:34. In some embodiments, there is provided a targeting construct (or a composition comprising the construct such as a pharmaceutical composition), wherein the targeting construct comprises (a) an antibody or a fragment thereof, wherein the antibody or a fragment thereof specifically binds to a phospholipid (such as PE, CL, MDA, and/or PC); and (b) an active moiety (e.g., a therapeutic moiety a detectable moiety), wherein the antibody or fragment thereof comprises a heavy chain variable domain of SEQ ID NO:36. In some embodiments, there is provided a targeting construct (or a composition comprising the construct such as a pharmaceutical composition), wherein the targeting construct comprises (a) an antibody or a fragment thereof, wherein the antibody or a fragment thereof specifically binds to a phospholipid (such as PE, CL, MDA, and/or PC); and (b) an active moiety (e.g., a therapeutic moiety or a detectable moiety), wherein the antibody or fragment thereof comprises a light chain variable domain of SEQ ID NO:35. In some embodiments, the antibody or fragment thereof competitively inhibits the binding of a pathogenic antibody (such as monoclonal antibody C2) to phospholipid. In some embodiments, the antibody or antibody fragment thereof binds to the same epitope as a pathogenic antibody (such as monoclonal antibody C2) to phospholipid. In some embodiments, the phospholipid is present on the surface of a cell, a basement membrane (e.g., Bruch's membrane), or in a pathological structure (e.g., drusen) in an individual that is in or adjacent to a tissue undergoing (or is at risk of undergoing) tissue injury (such as non-ischemic injury) and/or oxidative damage. In some embodiments, the phospholipid is selected from the group consisting of phosphatidylethanolamine (PE), cardiolipin (CL), and phosphatidylcholine (PC). In some embodiments, the phospholipid is malondialdehyde (MDA). In some embodiments, the phospholipid is neutral. In some embodiments, the phospholipid is positively charged. In some embodiments, the phospholipid is oxidized. In some embodiments, the construct comprises an active moiety that comprises a therapeutic moiety (such as a complement inhibitor). In some embodiments, the construct comprises an active moiety that is a detectable moiety. In some embodiments, the construct is a fusion protein. In some embodiments, the targeting moiety and the active moiety are linked via a linker (such as a peptide linker).

In some embodiments, there is provided a targeting construct (or a composition comprising the construct such as a pharmaceutical composition), wherein the targeting construct comprises (a) an antibody or a fragment thereof, wherein the antibody or a fragment thereof specifically binds to a phospholipid (such as PE, CL, MDA, and/or PC); and (b) an active moiety (e.g., a therapeutic moiety or a detectable moiety), wherein the antibody or fragment thereof comprises: (i) a light chain variable domain of SEQ ID NO:34; and (ii) heavy chain variable domain of SEQ ID NO:36. In some embodiments, there is provided a targeting construct (or a composition comprising the construct such as a pharmaceutical composition), wherein the targeting construct comprises (a) an antibody or a fragment thereof, wherein the antibody or a fragment thereof specifically binds to a phospholipid (such as PE, CL, MDA, and/or PC); and (b) an active moiety (e.g., a therapeutic moiety or a detectable moiety), wherein the antibody or fragment thereof comprises: (i) a light chain variable domain of SEQ ID NO:35; and (ii) heavy chain variable domain of SEQ ID NO:36. In some embodiments, the antibody or fragment thereof competitively inhibits the binding of a pathogenic antibody (such as monoclonal antibody C2) to phospholipid. In some embodiments, the antibody or antibody fragment thereof binds to the same epitope as a pathogenic antibody (such as monoclonal antibody C2) to phospholipid. In some embodiments, the phospholipid is present on the surface of a cell, a basement membrane (e.g., Bruch's membrane), or in a pathological structure (e.g., drusen) in an individual that is in or adjacent to a tissue undergoing (or is at risk of undergoing) tissue injury (such as non-ischemic injury) and/or oxidative damage. In some embodiments, the phospholipid is selected from the group consisting of phosphatidylethanolamine (PE), cardiolipin (CL), and phosphatidylcholine (PC). In some embodiments, the phospholipid is malondialdehyde (MDA). In some embodiments, the phospholipid is neutral. In some embodiments, the phospholipid is positively charged. In some embodiments, the phospholipid is oxidized. In some embodiments, the construct comprises an active moiety that comprises a therapeutic moiety (such as a complement inhibitor). In some embodiments, the construct comprises an active moiety that is a detectable moiety. In some embodiments, the construct is a fusion protein. In some embodiments, the targeting moiety and the active moiety are linked via a linker (such as a peptide linker).

In some embodiments, there is provided a targeting construct (or a composition comprising the construct such as a pharmaceutical composition), wherein the construct comprises (a) an antibody or a fragment thereof, wherein the antibody or a fragment thereof specifically binds to a phospholipid (such as PE, CL, MDA, and/or PC); and (b) an active moiety (e.g., a therapeutic moiety or a detectable moiety), wherein the antibody or fragment is a scFv having the sequence of SEQ ID NO:37. In some embodiments, there is provided a targeting construct (or a composition comprising the construct such as a pharmaceutical composition), wherein the targeting construct comprises (a) an antibody or a fragment thereof, wherein the antibody or a fragment thereof specifically binds to a phospholipid (such as PE, CL, MDA, and/or PC); and (b) an active moiety (e.g., a therapeutic moiety or detectable moiety), wherein the antibody or fragment is a scFv having the sequence of SEQ ID NO:38. In some embodiments, the antibody or fragment thereof competitively inhibits the binding of a pathogenic antibody (such as monoclonal antibody C2) to phospholipid. In some embodiments, the antibody or antibody fragment thereof binds to the same epitope as a pathogenic antibody (such as monoclonal antibody C2) to phospholipid. In some embodiments, the phospholipid is present on the surface of a cell, a basement membrane (e.g., Bruch's membrane), or in a pathological structure (e.g., drusen) in an individual that is in or adjacent to a tissue undergoing (or is at risk of undergoing) tissue injury (such as non-ischemic injury) and/or oxidative damage. In some embodiments, the phospholipid is selected from the group consisting of phosphatidylethanolamine (PE), cardiolipin (CL), and phosphatidylcholine (PC). In some embodiments, the phospholipid is malondialdehyde (MDA). In some embodiments, the phospholipid is neutral. In some embodiments, the phospholipid is positively charged. In some embodiments, the phospholipid is oxidized. In some embodiments, the construct comprises an active moiety that comprises a therapeutic moiety (such as a complement inhibitor). In some embodiments, the construct comprises an active moiety that is a detectable moiety. In some embodiments, the construct is a fusion protein. In some embodiments, the targeting moiety and the active moiety are linked via a linker (such as a peptide linker).

In some embodiments, the targeting moiety and the active moiety are directly bonded, covalently bonded, or, reversibly bonded.

Targeting moieties (e.g., antibodies recognizing an injury-associated neoepitope)

The antibody or fragment thereof described herein (also referred to as the targeting moiety when provided in the context of a targeting construct) specifically bind to Annexin IV or a phospholipid.

The antibody or fragment thereof described herein (also referred to as the targeting moiety when provided in the context of a targeting construct) in some embodiments specifically bind to Annexin IV.

Annexin IV belongs to a family of proteins that are Ca2+ and phospholipid proteins. The structure of annexins consists of a conserved Ca2+ and membrane binding core of four annexin repeats (eight for annexin IV) and variable N-terminal regions. Annexins are soluble cytosolic proteins, but despite the lack of obvious signal sequences and the apparent inability to enter the classical secretory pathway, annexins have been identified in extracellular fluids or associated with the external cell surface through poorly understood binding sites. Annexin IV is predominantly produced by epithelial cells and is also found at high levels in lung, intestine, pancreas, liver, photoreceptors, and kidney. Rescher et al., J. Cell Sci., (2004), 117:2631-2639, Kulik et al., (2009) J Immunol. 182(9):5363-73, and Zernii et al., Biochemistry (Mosc)., (2003), 68(1):129-60. It is also present in drusen, the hallmarks of age-related macular degeneration (AMD) (Rayborn, M. E., Sakaguchi, S., Shadrach, K., Crabb, J. W., and Hollyfield, J. G. (2006) Ret Degen Dis Adv Exp Med Bio 572, 75-78).

In some embodiments, the Annexin IV is present on the surface of a cell (and/or in a pathological structure) in an individual that is in or adjacent to a tissue undergoing (or is at risk of undergoing) tissue injury. In some embodiments, the Annexin IV is present on the surface of a cell of an individual that is in or adjacent to a tissue undergoing (or is at risk of undergoing) non-ischemic injury. In some embodiments, the Annexin IV is present on the surface of a cell of an individual that is in or adjacent to a tissue undergoing (or is at risk of undergoing) oxidative damage. In some embodiments, the Annexin IV is present on the surface of a cell of an individual that is in or adjacent to a tissue undergoing (or is at risk of undergoing) ischemia-reperfusion injury. In some embodiments, the Annexin IV is produced by a nucleated cell (such as a mammalian cell). In some embodiments, the Annexin IV is recombinant protein.

In some embodiments, the Annexin IV is present on the surface of a cell, a basement membrane (e.g., Bruch's membrane), or in a pathological structure (e.g., drusen) in an individual that is in or adjacent to a tissue undergoing (or is at risk of undergoing) tissue injury. In some embodiments, the Annexin IV is present on the surface of a cell, a basement membrane (e.g., Bruch's membrane), or in a pathological structure (e.g., drusen) of an individual that is in or adjacent to a tissue undergoing (or is at risk of undergoing) non-ischemic injury. In some embodiments, the Annexin IV is present on the surface of a cell, a basement membrane (e.g., Bruch's membrane), or in a pathological structure (e.g., drusen) of an individual that is in or adjacent to a tissue undergoing (or is at risk of undergoing) oxidative damage. In some embodiments, the Annexin IV is present on the surface of a cell, a basement membrane (e.g., Bruch's membrane), or in a pathological structure (e.g., drusen) of an individual that is in or adjacent to a tissue undergoing (or is at risk of undergoing) ischemia-reperfusion injury. In some embodiments, the Annexin IV is produced by a nucleated cell (such as a mammalian cell). In some embodiments, the Annexin IV is recombinant protein.

In some embodiments, the epitope on Annexin IV for the antibody or fragment thereof is present on the surface of a cell (and/or in a pathological structure) in an individual that is in or adjacent to a tissue undergoing (or is at risk of undergoing) tissue injury but not on the surface of a cell that is in or adjacent to a tissue not undergoing (or is not at risk of undergoing) tissue injury. In some embodiments, the epitope on Annexin IV for the antibody or fragment thereof is present on the surface of a cell (and/or in a pathological structure) in an individual that is in or adjacent to a tissue undergoing (or is at risk of undergoing) non-ischemic injury but not on the surface of a cell that is in or adjacent to a tissue not undergoing (or is not at risk of undergoing) non-ischemic injury. In some embodiments, the epitope on Annexin IV for the antibody or fragment thereof is present on the surface of a cell that is in or adjacent to a tissue undergoing (or is at risk of undergoing) oxidative damage but not on the surface of a cell that is in or adjacent to a tissue not undergoing (or is not at risk of undergoing) oxidative damage. In some embodiments, the epitope on Annexin IV for the antibody or fragment thereof is present on the surface of a cell (and/or in a pathological structure) in an individual that is in or adjacent to a tissue undergoing (or is at risk of undergoing) ischemia-reperfusion injury but is not present on the surface of a cell that is in or adjacent to a tissue not undergoing (or is not at risk of undergoing) ischemia reperfusion injury.

In some embodiments, the epitope on Annexin IV for the antibody or fragment thereof is present on the surface of a cell, a basement membrane (e.g., Bruch's membrane), or in a pathological structure (e.g., drusen) in an individual that is in or adjacent to a tissue undergoing (or is at risk of undergoing) tissue injury but not on the surface of a cell, a basement membrane (e.g., Bruch's membrane), or in a pathological structure (e.g., drusen) that is in or adjacent to a tissue not undergoing (or is not at risk of undergoing) tissue injury. In some embodiments, the epitope on Annexin IV for the antibody or fragment thereof is present on the surface of a cell, a basement membrane (e.g., Bruch's membrane), or in a pathological structure (e.g., drusen) in an individual that is in or adjacent to a tissue undergoing (or is at risk of undergoing) non-ischemic injury but not on the surface of a cell, a basement membrane (e.g., Bruch's membrane), or in a pathological structure (e.g., drusen) that is in or adjacent to a tissue not undergoing (or is not at risk of undergoing) non-ischemic injury. In some embodiments, the epitope on Annexin IV for the antibody or fragment thereof is present on the surface of a cell, a basement membrane (e.g., Bruch's membrane), or in a pathological structure (e.g., drusen) that is in or adjacent to a tissue undergoing (or is at risk of undergoing) oxidative damage but not on the surface of a cell, a basement membrane (e.g., Bruch's membrane), or in a pathological structure (e.g., drusen) that is in or adjacent to a tissue not undergoing (or is not at risk of undergoing) oxidative damage. In some embodiments, the epitope on Annexin IV for the antibody or fragment thereof is present on the surface of a cell, a basement membrane (e.g., Bruch's membrane), or in a pathological structure (e.g., drusen) in an individual that is in or adjacent to a tissue undergoing (or is at risk of undergoing) ischemia-reperfusion injury but is not present on the surface of a cell, a basement membrane (e.g., Bruch's membrane), or in a pathological structure (e.g., drusen) that is in or adjacent to a tissue not undergoing (or is not at risk of undergoing) ischemia reperfusion injury.

In some embodiments, the antibody or fragment thereof described herein (also referred to as the targeting moiety when provided in the context of a targeting construct) specifically binds to a phospholipid, which include, but is not limited to, phosphatidylethanolamine (PE), cardiolipin (CL), phosphatidylcholine (PC), and malondialdehyde (MDA). PE, CL, and PC are classes of phospholipids found in biological membranes. Phosphatidylcholine is more commonly found in the exoplasmic or outer leaflet of a cell membrane. It is thought to be transported between membranes within the cell by phosphatidylcholine transfer protein (PCTP). The phospholipid is composed of a choline head group and glycerophosphoric acid with a variety of fatty acids, one being a saturated fatty acid and one being an unsaturated fatty acid. PE consists of a combination of glycerol esterified with two fatty acids and phosphoric acid. Whereas the phosphate group is combined with choline in phosphatidylcholine, it is combined with the ethanolamine in PE. The two fatty acids may be the same, or different, and are usually in the 1,2 positions (though they can be in the 1,3 positions). Cardiolipin (IUPAC name “1,3-bis(sn-3′-phosphatidyl)-sn-glycerol”) is an important component of the inner mitochondrial membrane, where it constitutes about 20% of the total lipid composition. Cardiolipin (CL) is a kind of diphosphatidylglycerol lipid, in which two phosphatidylglycerols connect with a glycerol backbone in the center to form a dimeric structure. In most animal tissues, cardiolipin contains 18-carbon fatty alkyl chains with 2 unsaturated bonds on each of them. It has been proposed that the (18:2)4 acyl chain configuration is an important structural requirement for the high affinity of CL to inner membrane proteins in mammalian mitochondria. Phospholipid accumulation has been shown in eyes with age-related macular degeneration (Lommatzsch, et al. (2008) Graefes Arch Clin Exp Ophthalmol. 246(6):803-10).

Malondialdehyde (MDA) is generated from reactive oxygen species (ROS), and as such is often assayed in vivo as a bio-marker of oxidative stress. Reactive oxygen species degrade polyunsaturated lipids, forming malondialdehyde. This compound is a reactive aldehyde and is one of the many reactive electrophile species that cause toxic stress in cells and form covalent protein adducts referred to as advanced lipoxidation end-products (ALE). The production of this aldehyde is also used as a biomarker to measure the level of oxidative stress in an organism. MDA modifications have been shown in eyes with age-related macular degeneration and in the mouse laser-induced CNV model of wet AMD (Weissman et al. (2011) Nature. 478(7367):76-81).

In some embodiments, the phospholipid (such as PE, CL, MDA, and/or PC) is present on the surface of a cell (or in a pathological structure, e.g., drusen) in an individual that is in or adjacent to a tissue undergoing (or is at risk of undergoing) tissue injury. In some embodiments, the phospholipid (such as PE, CL, MDA, and/or PC) is present on the surface of a cell (or in a pathological structure, e.g., drusen) of an individual that is in or adjacent to a tissue undergoing (or is at risk of undergoing) ocular disease. In some embodiments, the phospholipid (such as PE, CL, MDA, and/or PC) is present on the surface of a cell (or in a pathological structure, e.g., drusen) of an individual that is in or adjacent to a tissue undergoing (or is at risk of undergoing) oxidative damage. In some embodiments, the phospholipid is neutral. In some embodiments, the phospholipid is positively charged. In some embodiments, the phospholipid (such as PE, CL, MDA, and/or PC) is oxidized.

In some embodiments, the phospholipid (such as PE, CL, MDA, and/or PC) is present on the surface of a cell, a basement membrane (e.g., Bruch's membrane), or in a pathological structure (e.g., drusen) in an individual that is in or adjacent to an ocular tissue undergoing (or is at risk of undergoing) tissue injury. In some embodiments, the phospholipid (such as PE, CL, MDA, and/or PC) is present on the surface of a cell, a basement membrane (e.g., Bruch's membrane), or in a pathological structure (e.g., drusen) of an individual that is in or adjacent to an ocular tissue undergoing (or is at risk of undergoing) ocular disease. In some embodiments, the phospholipid (such as PE, CL, MDA, and/or PC) is present on the surface of a cell, a basement membrane (e.g., Bruch's membrane), or in a pathological structure (e.g., drusen) of an individual that is in or adjacent to a tissue undergoing (or is at risk of undergoing) oxidative damage. In some embodiments, the phospholipid is neutral. In some embodiments, the phospholipid is positively charged. In some embodiments, the phospholipid (such as PE, CL, MDA, and/or PC) is oxidized.

In some embodiments, the epitope of phospholipid (such as PE, CL, MDA, and/or PC) to which the antibody or fragment thereof binds is present on the surface of a cell or in a pathological structure (e.g., drusen) in an individual that is in or adjacent to a tissue undergoing (or is at risk of undergoing) tissue injury but not on the surface of a cell or in a pathological structure (e.g., drusen) that is in or adjacent to a tissue not undergoing (or is not at risk of undergoing) tissue injury. In some embodiments, the epitope of phospholipid (such as PE, CL, MDA, and/or PC) to which the antibody or fragment thereof binds is present on the surface of a cell or in a pathological structure (e.g., drusen) in an individual that is in or adjacent to a tissue undergoing (or is at risk of undergoing) ocular disease but not on the surface of a cell or in a pathological structure (e.g., drusen) that is in or adjacent to a tissue not undergoing (or is not at risk of undergoing) non-ocular disease. In some embodiments, the epitope on phospholipid (such as PE, CL, MDA, and/or PC) to which the antibody or fragment thereof binds is present on the surface of a cell or in a pathological structure (e.g., drusen) that is in or adjacent to a tissue undergoing (or is at risk of undergoing) oxidative damage but not on the surface of a cell or in a pathological structure (e.g., drusen) that is in or adjacent to a tissue not undergoing (or is not at risk of undergoing) oxidative damage.

In some embodiments, the epitope of phospholipid (such as PE, CL, MDA, and/or PC) to which the antibody or fragment thereof binds is present on the surface of a cell, a basement membrane (e.g., Bruch's membrane), or in a pathological structure (e.g., drusen) in an individual that is in or adjacent to a ocular tissue undergoing (or is at risk of undergoing) tissue injury but not on the surface of a cell, a basement membrane (e.g., Bruch's membrane), or in a pathological structure (e.g., drusen) that is in or adjacent to a ocular tissue not undergoing (or is not at risk of undergoing) tissue injury. In some embodiments, the epitope of phospholipid (such as PE, CL, MDA, and/or PC) to which the antibody or fragment thereof binds is present on the surface of a cell, a basement membrane (e.g., Bruch's membrane), or in a pathological structure (e.g., drusen) in an individual that is in or adjacent to a tissue undergoing (or is at risk of undergoing) ocular disease but not on the surface of a cell, a basement membrane (e.g., Bruch's membrane), or in a pathological structure (e.g., drusen) that is in or adjacent to a tissue not undergoing (or is not at risk of undergoing) non-ocular disease. In some embodiments, the epitope on phospholipid (such as PE, CL, MDA, and/or PC) to which the antibody or fragment thereof binds is present on the surface of a cell, a basement membrane (e.g., Bruch's membrane), or in a pathological structure (e.g., drusen) that is in or adjacent to a tissue undergoing (or is at risk of undergoing) oxidative damage but not on the surface of a cell, a basement membrane (e.g., Bruch's membrane), or in a pathological structure (e.g., drusen) that is in or adjacent to a tissue not undergoing (or is not at risk of undergoing) oxidative damage.

As described herein, a cell (and/or a pathological structure) that is in or adjacent to a particular tissue as described herein includes a cell (and/or a pathological structure, e.g., drusen) that is part of a tissue or organ, or adjacent to (near, directly next to, in the microenvironment of, bordering, flanking, adjoining) a tissue or organ, in which a certain event (such as non-ischemic injury or oxidative damage) is going to occur, is likely to occur, or is beginning to occur. As described herein, a cell, a basement membrane (e.g., Bruch's membrane), or in a pathological structure (e.g., drusen) that is in or adjacent to a particular tissue as described herein includes a cell that is part of a tissue or organ, or adjacent to (near, directly next to, in the microenvironment of, bordering, flanking, adjoining) a tissue or organ, in which a certain event (such as non-ischemic injury or oxidative damage) is going to occur, is likely to occur, or is beginning to occur. In the case of an adjacent cell, the cell is sufficiently within the microenvironment of the specific tissue or organ such that conditions of oxidative damage and/or inflammation affect the adjacent cell, as well as the specific tissue or organ. Such a cell may display signs of stress, including, but not limited to, the display of “stress proteins” (e.g., heat shock proteins and other proteins associated with a cellular stress response, including annexins) or other molecules on the cell surface (phospholipids, carbohydrate moieties), including the display of abnormal levels of proteins, modified proteins, or other molecules on the cell surface. Such a cell may be undergoing apoptosis or showing signs of apoptosis, such signs including morphological changes in the cell, chromatin condensation, changes in cellular signal transduction protein interactions, changes in intracellular calcium levels, externalization of phospholipids, cell detachment, loss of cell surface structures, etc.

As used herein, the term “selectively binds to” refers to the specific binding of one protein to another protein, to a lipid, or to a carbohydrate moiety (e.g., the binding of an antibody, a fragment thereof, or binding partner to an antigen), wherein the level of binding, as measured by any standard assay (e.g., an immunoassay), is statistically significantly higher than the background control for the assay. For example, when performing an immunoassay, controls typically include a reaction well/tube that contain antibody or antigen binding fragment alone (i.e., in the absence of antigen), wherein an amount of reactivity (e.g., non-specific binding to the well) by the antibody or antigen binding fragment thereof in the absence of the antigen is considered to be background. Binding can be measured using a variety of methods standard in the art, including, but not limited to: Western blot, immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, matrix-assisted laser desorption/ionization time-of-flight (MALDI-TOF) mass spectrometry, microcytometry, microarray, microscopy, fluorescence activated cell sorting (FACS), and flow cytometry.

According to the present invention, an “epitope” of a given protein or peptide or other molecule is generally defined, with regard to antibodies, as a part of or site on a larger molecule to which an antibody or antigen-binding fragment thereof will bind, and against which an antibody will be produced. The term epitope can be used interchangeably with the term “antigenic determinant”, “antibody binding site”, or “conserved binding surface” of a given protein or antigen. More specifically, an epitope can be defined by both the amino acid residues involved in antibody binding and also by their conformation in three-dimensional space (e.g., a conformational epitope or the conserved binding surface). An epitope can be included in peptides as small as about 4-6 amino acid residues, or can be included in larger segments of a protein, and need not be comprised of contiguous amino acid residues when referring to a three dimensional structure of an epitope, particularly with regard to an antibody-binding epitope. Antibody-binding epitopes are frequently conformational epitopes rather than a sequential epitope (i.e., linear epitope), or in other words, an epitope defined by amino acid residues arrayed in three dimensions on the surface of a protein or polypeptide to which an antibody binds. As mentioned above, the conformational epitope is not comprised of a contiguous sequence of amino acid residues, but instead, the residues are perhaps widely separated in the primary protein sequence, and are brought together to form a binding surface by the way the protein folds in its native conformation in three dimensions.

Competition assays can be performed using standard techniques in the art (e.g., competitive ELISA or other binding assays). For example, competitive inhibitors can be detected and quantitated by their ability to inhibit the binding of an antigen to a known, labeled antibody (e.g., the mAb B4) or to sera or another composition that is known to contain antibodies against the particular antigen (e.g., sera known to contain natural antibodies against the antigen).

According to the present invention, antibodies are characterized in that they comprise immunoglobulin domains and as such, they are members of the immunoglobulin superfamily of proteins. Generally speaking, an antibody molecule comprises two types of chains. One type of chain is referred to as the heavy or H chain and the other is referred to as the light or L chain. The two chains are present in an equimolar ratio, with each antibody molecule typically having two H chains and two L chains. The two H chains are linked together by disulfide bonds and each H chain is linked to an L chain by a disulfide bond. There are only two types of L chains referred to as lambda (λ) and kappa (κ) chains. In contrast, there are five major H chain classes referred to as isotypes. The five classes include immunoglobulin M (IgM or μ), immunoglobulin D (IgD or δ), immunoglobulin G (IgG or λ), immunoglobulin A (IgA or α), and immunoglobulin E (IgE or ε). The distinctive characteristics between such isotypes are defined by the constant domain of the immunoglobulin and are discussed in detail below. Human immunoglobulin molecules comprise nine isotypes, IgM, IgD, IgE, four subclasses of IgG including IgG1 (γ1), IgG2 (γ2), IgG3 (γ3) and IgG4 (γ4), and two subclasses of IgA including IgA1 (α1) and IgA2 (α2). In humans, IgG subclass 3 and IgM are the most potent complement activators (classical complement system), while IgG subclass 1 and to an even lesser extent, 2, are moderate to low activators of the classical complement system. IgG4 subclass does not activate the complement system (classical or alternative). The only human immunoglobulin isotype known to activate the alternative complement system is IgA. In mice, the IgG subclasses are IgG1, IgG2a, IgG2b and IgG3. Murine IgG1 does not activate complement, while IgG2a, IgG2b and IgG3 are complement activators.

Each H or L chain of an immunoglobulin molecule comprises two regions referred to as L chain variable domains (VL domains) and L chain constant domains (CL domains), and H chain variable domains (VH domains) and H chain constant domains (CH domains). A complete CH domain comprises three sub-domains (CH1, CH2, CH3) and a hinge region. Together, one H chain and one L chain can form an arm of an immunoglobulin molecule having an immunoglobulin variable region. A complete immunoglobulin molecule comprises two associated (e.g., di-sulfide linked) arms. Thus, each arm of a whole immunoglobulin comprises a VH+L region, and a CH+L region. As used herein, the term “variable region” or “V region” refers to a VH+L region (also known as an Fv fragment), a VL region or a VH region. Also as used herein, the term “constant region” or “C region” refers to a CH+L region, a CL region or a CH region.

The antigen specificity of an immunoglobulin molecule is conferred by the amino acid sequence of a variable, or V, region. As such, V regions of different immunoglobulin molecules can vary significantly depending upon their antigen specificity. Certain portions of a V region are more conserved than others and are referred to as framework regions (FR regions). In contrast, certain portions of a V region are highly variable and are designated hypervariable regions. When the VL and VH domains pair in an immunoglobulin molecule, the hypervariable regions from each domain associate and create hypervariable loops that form the antigen binding sites (antigen combining sites). Thus, the hypervariable loops determine the specificity of an immunoglobulin and are termed complementarity-determining regions (CDRs) because their surfaces are complementary to antigens.

Both an L chain and H chain V gene segment contain three regions of substantial amino acid sequence variability. Such regions are referred to as L chain CDR1, CDR2 and CDR3, and H chain CDR1, CDR2 and CDR3, respectively. The length of an L chain CDR1 can vary substantially between different VL regions. For example, the length of CDR1 can vary from about 7 amino acids to about 17 amino acids. In contrast, the lengths of L chain CDR2 and CDR3 typically do not vary between different VL regions. The length of an H chain CDR3 can vary substantially between different VH regions. For example, the length of CDR3 can vary from about 1 amino acid to about 20 amino acids. Each H and L chain CDR region is flanked by FR regions.

Limited digestion of an immunoglobulin with a protease may produce two fragments. An antigen binding fragment is referred to as an Fab, an Fab′, or an F(ab′)2 fragment. A fragment lacking the ability to bind to antigen is referred to as an Fc fragment. A Fab fragment comprises one arm of an immunoglobulin molecule containing a L chain (VL+CL domains) paired with the VH region and a portion of the CH region (CH1 domain). An Fab′ fragment corresponds to an Fab fragment with part of the hinge region attached to the CH1 domain. An F(ab′)2 fragment corresponds to two Fab′ fragments that are normally covalently linked to each other through a di-sulfide bond, typically in the hinge regions.

Isolated antibodies of the present invention can include serum containing such antibodies, or antibodies that have been purified to varying degrees. Whole antibodies of the present invention can be polyclonal or monoclonal. Alternatively, functional equivalents of whole antibodies, such as antigen binding fragments in which one or more antibody domains are truncated or absent (e.g., Fv, Fab, Fab′, or F(ab′)2 fragments), as well as genetically-engineered antibodies or antigen binding fragments thereof, including single chain antibodies (e.g., scFv), humanized antibodies, antibodies that can bind to more than one epitope (e.g., bi-specific antibodies), or antibodies that can bind to one or more different antigens (e.g., bi- or multi-specific antibodies), may also be employed in the invention.

In some embodiments, the targeting moiety of the targeting constructs provided herein comprises an antibody. In some embodiments, the targeting moiety is a scFv. In some embodiments, the targeting moiety is a scFv comprising a (i) a light chain variable domain of SEQ ID NO:13; and/or (ii) heavy chain variable domain of SEQ ID NO:15. In some embodiments, the targeting moiety is a scFv comprising (i) a light chain variable domain of SEQ ID NO:14; and/or (ii) heavy chain variable domain of SEQ ID NO:16. In some embodiments, the targeting moiety is a scFv having the sequence of SEQ ID NO:17. In some embodiments, the targeting moiety is a scFv having the sequence of SEQ ID NO:18.

In some embodiments, the targeting moiety is a scFv comprising a (i) a light chain variable domain of SEQ ID NO:34; and/or (ii) heavy chain variable domain of SEQ ID NO:36. In some embodiments, the targeting moiety is a scFv comprising (i) a light chain variable domain of SEQ ID NO:35; and/or (ii) heavy chain variable domain of SEQ ID NO:36. In some embodiments, the targeting moiety is a scFv having the sequence of SEQ ID NO:37. In some embodiments, the targeting moiety is a scFv having the sequence of SEQ ID NO:38.

In one embodiment, targeting constructs of the present invention include humanized antibodies or a fragment thereof (such as a humanized scFv). A humanized antibody or fragment thereof are molecules having an antigen binding site derived from an immunoglobulin from a non-human species, the remaining immunoglobulin-derived parts of the molecule being derived from a human immunoglobulin. The antigen binding site may comprise either complete variable regions fused onto human constant domains or only the complementarity determining regions (CDRs) grafted onto appropriate human framework regions in the variable domains. A humanized antibody or fragment thereof can be produced, for example, by modeling the antibody variable domains, and producing the antibodies using genetic engineering techniques, such as CDR grafting. A description various techniques for the production of humanized antibodies is found, for example, in Morrison et al. (1984) Proc. Natl. Acad. Sci. USA 81:6851-55; Whittle et al. (1987) Prot. Eng. 1:499-505; Co et al. (1990) J. Immunol. 148:1149-1154; Co et al. (1992) Proc. Natl. Acad. Sci. USA 88:2869-2873; Carter et al. (1992) Proc. Natl. Acad. Sci. 89:4285-4289; Routledge et al. (1991) Eur. J. Immunol. 21:2717-2725 and PCT Patent Publication Nos. WO 91/09967; WO 91/09968 and WO 92/113831.

In some embodiments, the antibody or fragment thereof does not activate complement activation. Methods of modifying antibodies or fragments thereof by reducing or eliminating their complement activation activities are known in the art (Tan et al. (1990) Proc Natl Acad Sci USA 87, 162-166).

In some embodiments, the antibody or fragment there of comprises: (i) a light chain variable domain comprising a sequence of SEQ ID NO:1, a sequence of SEQ ID NO:2, or a sequence of SEQ ID NO:3; and/or (ii) heavy chain variable domain comprising a sequence of SEQ ID NO:4, a sequence of SEQ ID NO:5, or a sequence of SEQ ID NO:6. In some embodiments, the antibody or fragment there of comprises: (i) a light chain variable domain comprising a sequence of SEQ ID NO:7, a sequence of SEQ ID NO:8, or a sequence of SEQ ID NO:9; and/or (ii) heavy chain variable domain comprising a sequence of SEQ ID NO:10, a sequence of SEQ ID NO:11, or a sequence of SEQ ID NO:12.

In some embodiments, the antibody or fragment there of comprises: (i) a light chain variable domain comprising a sequence of SEQ ID NO:1; (ii) a light chain variable domain comprising a sequence of SEQ ID NO:2; and (iii) a light chain variable domain comprising a sequence of SEQ ID NO:3. In some embodiments, the antibody or fragment there of comprises: (i) a light chain variable domain comprising a sequence of SEQ ID NO:7; (ii) a light chain variable domain comprising a sequence of SEQ ID NO:8; and (iii) a light chain variable domain comprising a sequence of SEQ ID NO:9.

In some embodiments, the antibody or fragment there of comprises: (i) heavy chain variable domain comprising a sequence of SEQ ID NO:4; (ii) heavy chain variable domain comprising a sequence of SEQ ID NO:5; and (iii) heavy chain variable domain comprising a sequence of SEQ ID NO:6. In some embodiments, the antibody or fragment there of comprises: (i) heavy chain variable domain comprising a sequence of SEQ ID NO:10; (ii) heavy chain variable domain comprising a sequence of SEQ ID NO:11; and (iii) heavy chain variable domain comprising a sequence of SEQ ID NO:12.

In some embodiments, the antibody or fragment there of comprises: (i) a light chain variable domain comprising a sequence of SEQ ID NO:1; (ii) a light chain variable domain comprising a sequence of SEQ ID NO:2; (iii) a light chain variable domain comprising a sequence of SEQ ID NO:3; (iv) heavy chain variable domain comprising a sequence of SEQ ID NO:4; (v) heavy chain variable domain comprising a sequence of SEQ ID NO:5; and (vi) heavy chain variable domain comprising a sequence of SEQ ID NO:6. In some embodiments, the antibody or fragment there of comprises: (i) a light chain variable domain comprising a sequence of SEQ ID NO:7; (ii) a light chain variable domain comprising a sequence of SEQ ID NO:8; (iii) a light chain variable domain comprising a sequence of SEQ ID NO:9; (iv) heavy chain variable domain comprising a sequence of SEQ ID NO:10; (v) heavy chain variable domain comprising a sequence of SEQ ID NO:11; and (vi) heavy chain variable domain comprising a sequence of SEQ ID NO:12.

In some embodiments, the antibody or fragment there of comprises: (i) a light chain CDR1 of SEQ ID NO:1; (ii) a light chain CDR2 of SEQ ID NO:2; (iii) a light chain CDR3 of SEQ ID NO:3; (iv) heavy chain CDR1 of SEQ ID NO:4; (v) heavy chain CDR2 of SEQ ID NO:5; and (vi) heavy chain CDR3 of SEQ ID NO:6. In some embodiments, the antibody or fragment there of comprises: (i) a light chain CDR1 of SEQ ID NO:7; (ii) a light chain CDR2 of SEQ ID NO: 8; (iii) a light chain CDR3 of SEQ ID NO:9; (iv) heavy chain CDR1 of SEQ ID NO:10; (v) heavy chain CDR2 of SEQ ID NO:11; and (vi) heavy chain CDR3 of SEQ ID NO:12.

In some embodiments, the antibody or fragment there of comprises a light chain variable domain of SEQ ID NO:13. In some embodiments, the antibody or fragment there of comprises a heavy chain variable domain of SEQ ID NO:15. In some embodiments, the antibody or fragment there of comprises a light chain variable domain of SEQ ID NO:14. In some embodiments, the antibody or fragment there of comprises a heavy chain variable domain of SEQ ID NO:16.

In some embodiments, the antibody or fragment there of comprises: (i) a light chain variable domain of SEQ ID NO:13; and (ii) heavy chain variable domain of SEQ ID NO:15. In some embodiments, the antibody or fragment there of comprises: (i) a light chain variable domain of SEQ ID NO:14; and (ii) heavy chain variable domain of SEQ ID NO:16.

In some embodiments, the antibody or fragment is a scFv having the sequence of SEQ ID NO:17. In some embodiments, the antibody or fragment is a scFv having the sequence of SEQ ID NO:18.

In some embodiments, the antibody or fragment thereof specifically binds to a phospholipid and comprises: (i) a light chain variable domain comprising a sequence of SEQ ID NO:25, a sequence of SEQ ID NO:26, or a sequence of SEQ ID NO:27; and/or (ii) heavy chain variable domain comprising a sequence of SEQ ID NO:28, a sequence of SEQ ID NO:29, or a sequence of SEQ ID NO:30. In some embodiments, the antibody or fragment thereof specifically binds to a phospholipid and comprises: (i) a light chain variable domain comprising a sequence of SEQ ID NO:31, a sequence of SEQ ID NO:32, or a sequence of SEQ ID NO:33; and/or (ii) heavy chain variable domain comprising a sequence of SEQ ID NO:28, a sequence of SEQ ID NO:29, or a sequence of SEQ ID NO:30.

In some embodiments, the antibody or fragment thereof specifically binds to a phospholipid and comprises: (i) a light chain variable domain comprising a sequence of SEQ ID NO:25; (ii) a light chain variable domain comprising a sequence of SEQ ID NO:26; and (iii) a light chain variable domain comprising a sequence of SEQ ID NO:27. In some embodiments, the antibody or fragment thereof specifically binds to a phospholipid and comprises: (i) a light chain variable domain comprising a sequence of SEQ ID NO:31; (ii) a light chain variable domain comprising a sequence of SEQ ID NO:32; and (iii) a light chain variable domain comprising a sequence of SEQ ID NO:33.

In some embodiments, the antibody or fragment thereof specifically binds to a phospholipid and comprises: (i) heavy chain variable domain comprising a sequence of SEQ ID NO:28; (ii) heavy chain variable domain comprising a sequence of SEQ ID NO:29; and (iii) heavy chain variable domain comprising a sequence of SEQ ID NO:30.

In some embodiments, the antibody or fragment thereof specifically binds to a phospholipid and comprises: (i) a light chain variable domain comprising a sequence of SEQ ID NO:25; (ii) a light chain variable domain comprising a sequence of SEQ ID NO:26; (iii) a light chain variable domain comprising a sequence of SEQ ID NO:27; (iv) heavy chain variable domain comprising a sequence of SEQ ID NO:28; (v) heavy chain variable domain comprising a sequence of SEQ ID NO:29; and (vi) heavy chain variable domain comprising a sequence of SEQ ID NO:30. In some embodiments, the antibody or fragment there of specifically binds to a phospholipid and comprises: (i) a light chain variable domain comprising a sequence of SEQ ID NO:31; (ii) a light chain variable domain comprising a sequence of SEQ ID NO:32; (iii) a light chain variable domain comprising a sequence of SEQ ID NO:33; (iv) heavy chain variable domain comprising a sequence of SEQ ID NO:28; (v) heavy chain variable domain comprising a sequence of SEQ ID NO:29; and (vi) heavy chain variable domain comprising a sequence of SEQ ID NO:30.

In some embodiments, the antibody or fragment there of specifically binds to a phospholipid and comprises: (i) a light chain CDR1 of SEQ ID NO:25; (ii) a light chain CDR2 of SEQ ID NO:26; (iii) a light chain CDR3 of SEQ ID NO:27; (iv) heavy chain CDR1 of SEQ ID NO:28; (v) heavy chain CDR2 of SEQ ID NO:29; and (vi) heavy chain CDR3 of SEQ ID NO:30. In some embodiments, the antibody or fragment there of specifically binds to a phospholipid and comprises: (i) a light chain CDR1 of SEQ ID NO:31; (ii) a light chain CDR2 of SEQ ID NO:32; (iii) a light chain CDR3 of SEQ ID NO:33; (iv) heavy chain CDR1 of SEQ ID NO:28; (v) heavy chain CDR2 of SEQ ID NO:29; and (vi) heavy chain CDR3 of SEQ ID NO:30.

In some embodiments, the antibody or fragment thereof comprises a light chain variable domain of SEQ ID NO:34. In some embodiments, the antibody or fragment thereof comprises a heavy chain variable domain of SEQ ID NO:36. In some embodiments, the antibody or fragment thereof comprises a light chain variable domain of SEQ ID NO:35.

In some embodiments, the antibody or fragment thereof comprises: (i) a light chain variable domain of SEQ ID NO:34; and (ii) heavy chain variable domain of SEQ ID NO:36. In some embodiments, the antibody or fragment thereof comprises: (i) a light chain variable domain of SEQ ID NO:35; and (ii) heavy chain variable domain of SEQ ID NO:36.

In some embodiments, the antibody or fragment is a scFv having the sequence of SEQ ID NO:37. In some embodiments, the antibody or fragment is a scFv having the sequence of SEQ ID NO:38.

In some embodiments, the antibody or fragment thereof does not activate complement activation. Methods of modifying antibodies or fragments thereof by reducing or eliminating their complement activation activities are known in the art (Tan et al. (1990) Proc Natl Acad Sci USA 87, 162-166).

In some embodiments, the antibody or fragment thereof specifically binds to Annexin IV and comprises: (i) a light chain variable domain comprising a sequence of SEQ ID NO:1, a sequence of SEQ ID NO:2, or a sequence of SEQ ID NO:3; and/or (ii) heavy chain variable domain comprising a sequence of SEQ ID NO:4, a sequence of SEQ ID NO:5, or a sequence of SEQ ID NO:6. In some embodiments, the antibody or fragment thereof specifically binds to Annexin IV and comprises: (i) a light chain variable domain comprising a sequence of SEQ ID NO:7, a sequence of SEQ ID NO:8, or a sequence of SEQ ID NO:9; and/or (ii) heavy chain variable domain comprising a sequence of SEQ ID NO:10, a sequence of SEQ ID NO:11, or a sequence of SEQ ID NO:12.

In some embodiments, the antibody or fragment thereof specifically binds to Annexin IV and comprises: (i) a light chain variable domain comprising a sequence of SEQ ID NO:1; (ii) a light chain variable domain comprising a sequence of SEQ ID NO:2; and (iii) a light chain variable domain comprising a sequence of SEQ ID NO:3. In some embodiments, the antibody or fragment thereof specifically binds to Annexin IV and comprises: (i) a light chain variable domain comprising a sequence of SEQ ID NO:7; (ii) a light chain variable domain comprising a sequence of SEQ ID NO:8; and (iii) a light chain variable domain comprising a sequence of SEQ ID NO:9.

In some embodiments, the antibody or fragment thereof specifically binds to Annexin IV and comprises: (i) heavy chain variable domain comprising a sequence of SEQ ID NO:4; (ii) heavy chain variable domain comprising a sequence of SEQ ID NO:5; and (iii) heavy chain variable domain comprising a sequence of SEQ ID NO:6. In some embodiments, the antibody or fragment thereof specifically binds to Annexin IV and comprises: (i) heavy chain variable domain comprising a sequence of SEQ ID NO:10; (ii) heavy chain variable domain comprising a sequence of SEQ ID NO:11; and (iii) heavy chain variable domain comprising a sequence of SEQ ID NO:12.

In some embodiments, the antibody or fragment thereof specifically binds to Annexin IV and comprises: (i) a light chain variable domain comprising a sequence of SEQ ID NO:1; (ii) a light chain variable domain comprising a sequence of SEQ ID NO:2; (iii) a light chain variable domain comprising a sequence of SEQ ID NO:3; (iv) heavy chain variable domain comprising a sequence of SEQ ID NO:4; (v) heavy chain variable domain comprising a sequence of SEQ ID NO:5; and (vi) heavy chain variable domain comprising a sequence of SEQ ID NO:6. In some embodiments, the antibody or fragment thereof specifically binds to Annexin IV and comprises: (i) a light chain variable domain comprising a sequence of SEQ ID NO:7; (ii) a light chain variable domain comprising a sequence of SEQ ID NO:8; (iii) a light chain variable domain comprising a sequence of SEQ ID NO:9; (iv) heavy chain variable domain comprising a sequence of SEQ ID NO:10; (v) heavy chain variable domain comprising a sequence of SEQ ID NO:11; and (vi) heavy chain variable domain comprising a sequence of SEQ ID NO:12.

In some embodiments, the antibody or fragment thereof specifically binds to Annexin IV and comprises: (i) a light chain CDR1 of SEQ ID NO:1; (ii) a light chain CDR2 of SEQ ID NO:2; (iii) a light chain CDR3 of SEQ ID NO:3; (iv) heavy chain CDR1 of SEQ ID NO:4; (v) heavy chain CDR2 of SEQ ID NO:5; and (vi) heavy chain CDR3 of SEQ ID NO:6. In some embodiments, the antibody or fragment thereof specifically binds to Annexin IV and comprises: (i) a light chain CDR1 of SEQ ID NO:7; (ii) a light chain CDR2 of SEQ ID NO:8; (iii) a light chain CDR3 of SEQ ID NO:9; (iv) heavy chain CDR1 of SEQ ID NO:10; (v) heavy chain CDR2 of SEQ ID NO:11; and (vi) heavy chain CDR3 of SEQ ID NO:12.

In some embodiments, the antibody or fragment thereof comprises a light chain variable domain of SEQ ID NO:13. In some embodiments, the antibody or fragment thereof comprises a heavy chain variable domain of SEQ ID NO:15. In some embodiments, the antibody or fragment thereof comprises a light chain variable domain of SEQ ID NO:14. In some embodiments, the antibody or fragment thereof comprises a heavy chain variable domain of SEQ ID NO:16.

In some embodiments, the antibody or fragment thereof comprises: (i) a light chain variable domain of SEQ ID NO:13; and (ii) heavy chain variable domain of SEQ ID NO:15. In some embodiments, the antibody or fragment thereof comprises: (i) a light chain variable domain of SEQ ID NO:14; and (ii) heavy chain variable domain of SEQ ID NO:16.

In some embodiments, the antibody or fragment thereof is an scFv. In some embodiments, the targeting moiety is an scFv comprising a (i) a light chain variable domain of SEQ ID NO: 34; and/or (ii) heavy chain variable domain of SEQ ID NO:36. In some embodiments, scFv comprises (i) a light chain variable domain of SEQ ID NO:35; and/or (ii) heavy chain variable domain of SEQ ID NO:36. In some embodiments, the antibody or fragment is a scFv having the sequence of SEQ ID NO:37. In some embodiments, the antibody or fragment is a scFv having the sequence of SEQ ID NO:38.

Therapeutic Moieties and Complement Modulators

In certain embodiments, the targeting construct comprises an active moiety that is a therapeutic moiety. In some embodiments, the therapeutic moiety is targeted or presented to the near space of the binding partner, e.g., Annexin IV, PE, PC, MDA, and/or CL, recognized by the targeting moiety.

In some embodiments the therapeutic moiety comprises an anti-VEGF drug, such as for treating macular edema, including, but not limited to, e.g., the monoclonal antibody bevacizumab (Avastin), derivatives of bevacizumab such as ranibizumab (Lucentis), orally-available small molecules that inhibit the tyrosine kinases stimulated by VEGF (e.g., lapatinib (Tykerb), sunitinib (Sutent), sorafenib (Nexavar), axitinib, and pazopanib), and aflibercept (EYLEA), a recombinant fusion protein consisting of portions of human VEGF receptors 1 and 2 extracellular domains fused to the Fc portion of human IgG1.

In certain embodiments, the therapeutic moiety in the targeting construct is an anti-viral agent, such as for the treatment of CMV retinitis, including, but not limited to, e.g., Ganciclovir, Foscarnet, Fomivirsen, Valganciclovir, and cidofovir. In certain embodiments, the therapeutic moiety in the targeting construct is a corticosteroid or an anti-TNFalpha agent, such as for the treatment of uveitis, including, but not limited to, e.g., prednisone, prednisolone, infliximab (Remicade), adalimumab (Humira), certolizumab pegol (Cimzia), and golimumab (Simponi), etanercept (Enbrel), xanthine derivatives (e.g., pentoxifylline) and Bupropion. In some embodiments, the therapeutic agent is an agent used for the treatment of glaucoma, including, but not limited to, e.g., a prostaglandins, a prostaglandin analog (such as misoprostol, lantanprost, bimaprost, or travoprost), a beta blocker (such as timolol, levobumomol, or betaxolol), a carbonic anhydrase inhibitor (such as dorzolamide (Trusopt), brinzolamide (Azopt), or acetazolamide (Diamox)), a miotic agent (such as pilocarpine), a cholinergic agent, and a neurotrophic factor to prevent retinal ganglion cell degeneration.

In certain embodiments, the therapeutic agent is an agent that is used in the treatment of diabetic retinopathy, including, but not limited to, e.g., an anti-VEGF drug or a Protein Kinase C inhibitor. In certain embodiments, the therapeutic agent is an agent that is used in the treatment of retinitis pigmentosa, e.g., ciliary neurotrophic factor (CNTF), the carbonic anhydrase inhibitor Acetazolamide, calcium channel blockers (such as Diltiazem), and immunosuppressive agents (if anti-retinal antibodies are present).

In certain embodiments, the therapeutic agent is an agent that is used in the treatment of proliferative vitreoretinopathy, including, but not limited to, e.g., minocyclin and Daunorubicin.

Methods of conjugating a targeting moiety, such as a B4 or C2 antibody or an antibody binding fragment thereof, to a drug, such a drug described herein, are known in the art and described in, e.g., McDonagh et al. (2006) Prot Engineer Design Select 19(7): 299-307; Hurwitz et al. (1975) Cancer Res 35:1175-1181; Garnett et al. (1983) Int J Cancer 31(5):661-670; Kovtun, et al. (2007) Cancer Letters 255(2): 232-40; Teicher et al. (2012) N Engl J Med 367(19):1847-8, and others.

In some embodiments, a therapeutic moiety is a complement modulator. An example of a complement modulator is a complement inhibitor. In some embodiments, such therapeutic moiety is a complement inhibitor. Accordingly, as used herein, the term “a therapeutic moiety” can encompass both a complement modulator and a complement inhibitor.

The constructs described herein in some embodiments comprise a complement modulator, such as a complement inhibitor.

As used herein, the term “complement inhibitor” refers to any compound, composition, or protein that reduces or eliminates complement activity. The reduction in complement activity may be incremental (e.g., a 10%, 20%, 30%, 40%, 50%, 60%, 70%, 80%, or 90% reduction in activity) or complete. For example, in some embodiments, a complement inhibitor can inhibit complement activity by at least 10 (e.g., at least 15, 20, 25, 30, 35, 40, 45, 50, 55, 60, 65, 70, 75, 80, 85, 90, or 95 or greater) % in a standard in vitro red blood cell hemolysis assay or an in vitro CH50eq assay. See, e.g., Kabat and Mayer (eds), “Experimental Immunochemistry, 2nd Edition,” 135-240, Springfield, Ill., C C Thomas (1961), pages 135-139, or a conventional variation of that assay such as the chicken erythrocyte hemolysis method as described in, e.g., Hillmen et al. (2004) N Engl J Med 350(6):552.

The CH50eq assay is a method for measuring the total classical complement activity in serum. This test is a lytic assay, which uses antibody-sensitized erythrocytes as the activator of the classical complement pathway and various dilutions of the test serum to determine the amount required to give 50% lysis (CH50). The percent hemolysis can be determined, for example, using a spectrophotometer. The CH50eq assay provides an indirect measure of terminal complement complex (TCC) formation, since the TCC themselves are directly responsible for the hemolysis that is measured.

The assay is well known and commonly practiced by those of skill in the art. Briefly, to activate the classical complement pathway, undiluted serum samples (e.g., human serum samples) are added to microassay wells containing the antibody-sensitized erythrocytes to thereby generate TCC. Next, the activated sera are diluted in microassay wells, which are coated with a capture reagent (e.g., an antibody that binds to one or more components of the TCC). The TCC present in the activated samples bind to the monoclonal antibodies coating the surface of the microassay wells. The wells are washed and, to each well, is added a detection reagent that is detectably labeled and recognizes the bound TCC. The detectable label can be, e.g., a fluorescent label or an enzymatic label. The assay results are expressed in CH50 unit equivalents per milliliter (CH50 U Eq/mL).

Additional methods for detecting and/or measuring complement activity in vitro are set forth and exemplified in the working examples.

The complement inhibitor described herein in some embodiments is a specific inhibitor of the lectin pathway. In some embodiments, the complement inhibitor is a specific inhibitor of the alternative pathway. In some embodiments, the complement inhibitor is a specific inhibitor of the classical pathway.

In some embodiments, the complement inhibitor is a soluble or membrane-bound protein such as, for example, membrane cofactor protein (MCP), decay accelerating factor (DAF/CD55), CD59, mouse complement receptor 1-related gene/protein y (Crry), human complement receptor 1 (CR1) or factor H, or Factor I, or an antibody specific for a component of a complement pathway such as, for example, eculizumab (an anti-CS antibody marketed under the trade name Soliris®), pexelizumab (the antigen-binding fragment of eculizumab), an anti-factor B antibody (such as the monoclonal antibody 1379 produced by ATCC Deposit No. PTA-6230), an anti-properdin antibody, an anti-factor D antibody, an anti-MASP antibody, an anti MBL-antibody, and the like (see below). Alternatively, a complement inhibitor may be a small molecule or a linear or cyclic peptide such as, for example, compstatin, N-acetylaspartylglutamic acid (NAAGA), and the like. In some embodiments, the complement inhibitor is selected from the group consisting of: an anti-C5 antibody, an Eculizumab, an pexelizumab, an anti-C3b antibody, an anti-C6 antibody, an anti-C7 antibody, an anti-factor B antibody, an anti-factor D antibody, and an anti-properdin antibody, a human membrane cofactor protein (MCP), a human decay accelerating factor (DAF), a mouse decay accelerating factor (DAF), a human CD59, a mouse CD59, a mouse CD59 isoform B, a mouse Crry, a human CR1, a Factor I, a human factor H, a mouse factor H, and a biologically active fragment of any the preceding.

As used herein, the term “membrane cofactor protein,” “MCP,” or “CD46” refers to a widely distributed C3b/C4b-binding cell surface glycoprotein which inhibits complement activation on host cells and serves as a cofactor for the factor I-mediated cleavage of C3b and C4b, including homologs thereof. T. J. Oglesby et al., J. Exp. Med. (1992) 175:1547-1551. MCP belongs to a family known as the regulators of complement activation (“RCA”). Family members share certain structural features, comprising varying numbers of short consensus repeat (SCR) domains, which are typically between 60 and 70 amino acids in length. Beginning at its amino-terminus, MCP comprises four SCRs, a serine/threonine/proline-enriched region, an area of undefined function, a transmembrane hydrophobic domain, a cytoplasmic anchor and a cytoplasmic tail. It is understood that species and strain variations exist for the disclosed peptides, polypeptides, and proteins, and that human MCP or biologically active fragments thereof encompass all species and strain variations.

SEQ ID NO:44 represents the full-length human MCP amino acid sequence (see, e.g., UniProtKB/Swiss-Prot. Accession No. P15529). Amino acids 1-34 correspond to the signal peptide, amino acids 35-343 correspond to the extracellular domain, amino acids 344-366 correspond to the transmembrane domain, and amino acids 367-392 correspond to the cytoplasmic domain. In the extracellular domain, amino acids 35-96 correspond to SCR 1, amino acids 97-159 correspond to SCR 2, amino acids 160-225 correspond to SCR 3, amino acids 226-285 correspond to SCR 4, and amino acids 302-326 correspond to the serine/threonine-rich domain. It is understood that species and strain variations exist for the disclosed peptides, polypeptides, and proteins, and that MCP or biologically active fragments thereof encompass all species and strain variations. As used herein, the term “biologically active” fragment of MCP refers to any soluble fragment lacking both the cytoplasmic domain and the transmembrane domain, including fragments comprising, consisting essentially of or consisting of 1, 2, 3, or 4 SCR domains, with or without the serine/threonine-rich domain, having some or all of the complement inhibitory activity of the full-length human MCP protein. In some embodiments, the complement inhibitor portion comprises full-length human MCP (amino acids 35-392 of SEQ ID NO:44), the extracellular domain of human MCP (amino acids 35-343 of SEQ ID NO:44), or SCRs 1-4 of human MCP (amino acids 35-285 of SEQ ID NO:44).

Decay accelerating factor, also referred to as CD55 (DAF/CD55) (SEQ ID NO:45 and SEQ ID NO:46), is an −70 kiloDalton (kDa) membrane-bound glycoprotein which inhibits complement activation on host cells. Like several other complement regulatory proteins, DAF comprises several approximately 60 amino acid repeating motifs termed short consensus repeats (SCR).

As used herein, the term “decay accelerating factor,” “DAF,” or “CD55” refers to a seventy kilodalton (“kDa”) membrane glycoprotein comprising four short consensus repeat (SCR) domains followed by a heavily O-glycosylated serine/threonine-rich domain at the C-terminus that elevates the molecule from the membrane surface, followed by a glycosylphosphatidylinositol (“GPI”) anchor. DAF protects the cell surface from complement activation by dissociating membrane-bound C3 convertases that are required to cleave complement protein C3 and to amplify the complement cascade. DAF prevents assembly or accelerates decay of both the C3- and C5-convertases of the alternative and classical complement pathways.

SEQ ID NO:45 represents the full-length human DAF amino acid sequence (see, e.g., UniProtKB/Swiss-Prot. Accession No. P08173); SEQ ID NO:46 represents the full-length mouse DAF amino acid sequence (see, e.g., UniProtKB/Swiss-Prot. Accession No. Q61475). In the human DAF sequence, amino acids 1-34 correspond to the signal peptide, amino acids 35-353 appear in the mature protein, and amino acids 354-381 are removed from the polypeptide after translation. Within the mature protein, amino acids 35-96 correspond to SCR 1, amino acids 96-160 correspond to SCR 2, amino acids 161-222 correspond to SCR 3, amino acids 223-285 correspond to SCR 4, and amino acids 287-353 correspond to the O-glycosylated serine/threonine-rich domain. The GPI anchor is attached to human DAF at a serine at position 353. In the mouse DAF sequence, amino acids 1-34 correspond to the signal peptide, amino acids 35-362 appear in the mature protein, and amino acids 363-390 are removed from the polypeptide after translation. Within the mature protein, amino acids 35-96 correspond to SCR 1, amino acids 97-160 correspond to SCR 2, amino acids 161-222 correspond to SCR 3, amino acids 223-286 correspond to SCR 4, and amino acids 288-362 correspond to the O-glycosylated serine/threonine-rich domain. The GPI anchor is attached to mouse DAF at a serine at position 362. It is understood that species and strain variations exist for the disclosed peptides, polypeptides, and proteins, and that DAF or biologically active fragments thereof encompass all species and strain variations. As used herein, the term “biologically active” fragment of DAF refers to any fragment of DAF lacking a GPI anchor and/or the amino acid to which it is attached (i.e., Ser-353), including any fragments of the full-length DAF protein comprising, consisting essentially of or consisting of 1, 2, 3, or 4 SCR domains, with or without the O-glycosylated serine/threonine-rich domain, having some or all the complement inhibitory activity of the full-length DAF protein.

As used herein, the term “CD59” refers to a membrane-bound 128 amino acid glycoprotein that potently inhibits the membrane attack complex (MAC) of complement. CD59 acts by binding to the C8 and/or C9 components of the MAC during assembly, ultimately preventing incorporation of the multiple copies of C9 required for complete formation of the osmolytic pore at the heart of the MAC. CD59 is both N- and O-glycosylated. The N-glycosylation comprises primarily bi- or tri-antennary structures with and without lactosamine and outer arm fucose residues, with variable sialylation present at some sites. Like DAF, CD59 is anchored in the cell membrane by a glycosylphosphatidylinositol (“GPI”) anchor, which is attached to an asparagine at amino acid 102. Soluble forms of CD59 (sCD59) have been produced, but they generally have low functional activity in vitro, particularly in the presence of serum, suggesting that unmodified sCD59 has little or no therapeutic efficacy. See, e.g., S. Meri et al., “Structural composition and functional characterization of soluble CD59: heterogeneity of the oligosaccharide and glycophosphoinositol (GPI) anchor revealed by laser-desorption mass spectrometric analysis,” Biochem. J. 316:923-935 (1996).

SEQ ID NO:47 represents the full-length human CD59 amino acid sequence (see, e.g., UniProtKB/Swiss-Prot. Accession No. P13987); SEQ ID NO:48 represents the full-length mouse CD59 sequence, isoform A (see, e.g., UniProtKB/Swiss-Prot. Accession No. 055186); SEQ ID NO:49 represents the full-length mouse CD59 sequence, isoform B (see, e.g., UniProtKB/SwissProt. Accession No. P58019). In the human CD59 sequence, amino acids 1-25 of SEQ ID NO:47 correspond to the leader peptide, amino acids 26-102 of SEQ ID NO:47 correspond to the mature protein, and amino acids 103-128 of SEQ ID NO:47 are removed after translation. The GPI anchor is attached to CD59 at an asparagine at position 102 of SEQ ID NO:47. In isoform A of the mouse CD59 sequence, amino acids 1-23 of SEQ ID NO:48 correspond to the leader peptide, amino acids 24-96 of SEQ ID NO: 48 correspond to the mature protein, and amino acids 97-123 of SEQ ID NO:48 are removed after translation. The GPI anchor is attached to CD59 at a serine at position 96 of SEQ ID NO: 48. In isoform B of the mouse CD59 sequence, amino acids 1-23 of SEQ ID NO: 49 correspond to the leader peptide, amino acids 24-104 of SEQ ID NO: 49 correspond to the mature protein, and amino acids 105-129 of SEQ ID NO:49 are removed after translation. The GPI anchor is attached to CD59 at an asparagine at position 104 of SEQ ID NO:49. It is understood that species and strain variations exist for the disclosed peptides, polypeptides, and proteins, and that CD59 or biologically active fragments thereof encompass all species and strain variations.

As used herein, the term “biologically active” fragment of human CD59 refers to any fragment of human CD59 lacking a GPI anchor and/or the amino acid to which it is attached (i.e., Asn-102), including any fragments of the full-length human CD59 protein having some or all the complement inhibitory activity of the full-length CD59 protein; and the term “biologically active” fragment of mouse CD59 refers to any fragment of mouse CD59 isoform A or isoform B lacking a GPI anchor and/or the amino acid to which it is attached (i.e., Ser-96 of isoform A, or Asp-104 of isoform B), including any fragments of either full-length mouse CD59 protein isoform having some or all the complement inhibitory activity of the full-length CD59 protein.

As used herein, the term “mouse complement receptor 1-related gene/protein y” or “Crry” refers to a membrane-bound mouse glycoprotein that regulates complement activation, including homologs thereof. Crry regulates complement activation by serving as a cofactor for complement factor I, a serine protease which cleaves C3b and C4b deposited on host tissue. Crry also acts as a decay-accelerating factor, preventing the formation of C4b2a and C3bBb, the amplification convertases of the complement cascade.

SEQ ID NO:50 represents the full-length mouse Crry protein amino acid sequence. Amino acids 1-40 correspond to the leader peptide, amino acids 41-483 of SEQ ID NO:50 correspond to the mature protein, comprising amino acids 41-405 of SEQ ID NO:50, corresponding to the extracellular domain, amino acids 406-426 of SEQ ID NO:50, corresponding to the transmembrane domain, and amino acids 427-483 of SEQ ID NO:50, corresponding to the cytoplasmic domain. In the extracellular domain, amino acids 83-143 of SEQ ID NO:50 correspond to SCR 1, amino acids 144-205 of SEQ ID NO:50 correspond to SCR 2, amino acids 206-276 of SEQ ID NO:50 correspond to SCR 3, amino acids 277-338 of SEQ ID NO:50 correspond to SCR 4, and amino acids 339-400 of SEQ ID NO:50 correspond to SCR 5. It is understood that species and strain variations exist for the disclosed peptides, polypeptides, and proteins, and that mouse Crry protein or biologically active fragments thereof encompasses all species and strain variations. As used herein, the term “biologically active” fragment of mouse Crry protein refers to any soluble fragment of mouse Crry lacking the transmembrane domain and the cytoplasmic domain, including fragments comprising, consisting essentially of or consisting of 1, 2, 3, 4, or 5 SCR domains, including any fragments of the full-length mouse Crry protein having some or all the complement inhibitory activity of the full-length Crry protein.

As used herein, the term “complement receptor 1,” “CR1,” or “CD35” refers to a human gene encoding a protein of 2039 amino acids, with a predicted molecular weight of 220 kilodaltons (“kDa”), including homologs thereof. The gene is expressed principally on erythrocytes, monocytes, neutrophils, and B cells, but is also present on some T lymphocytes, mast cells, and glomerular podocytes. CR1 protein is typically expressed at between 100 and 1000 copies per cell. CR1 is the main system for processing and clearance of complement-opsonized immune complexes. CR1 negatively regulates the complement cascade, mediates immune adherence and phagocytosis, and inhibits both the classic and alternative complement pathways. The full-length CR1 protein comprises a 42 amino acid signal peptide, an extracellular domain of 1930 amino acids, a 25 amino acid transmembrane domain, and a 43 amino acid C-terminal cytoplasmic domain. The extracellular domain of CR1 has 25 potential N-glycosylation signal sequences, and comprises 30 short consensus (“SCR”) domains, also known as complement control protein (CCP) repeats, or sushi domains, each 60 to 70 amino acids long. The sequence homology between SCRs ranges between 60-99 percent. The 30 SCR domains are further grouped into four longer regions termed long homologous repeats (“LHRs”), each encoding approximately 45 kDa segments of the CR1 protein, designated LHR-A, -B, -C, and -D. The first three comprise seven SCR domains each, while LHR-D comprises 9 SCR domains. The active sites on the extracellular domain of CR1 protein include a C4b-binding site with lower affinity for C3b in SCRs 1-4 comprising amino acids 42-295, a C3b-binding site with lower affinity for C4b in SCRs 8-11 comprising amino acids 490-745, a C3b-binding site with lower affinity for C4b in SCRs 15-18 comprising amino acids 940-1196, and a C1q-binding site in SCRs 22-28 comprising amino acids 1394-1842.

SEQ ID NO:51 represents the full-length human CR1 amino acid sequence (see, e.g., UniProtKB/Swiss-Prot. Accession No. P17927). Amino acids 1-41 correspond to the signal peptide, amino acids 42-2039 correspond to the mature protein, comprising amino acids 42-1971, corresponding to the extracellular domain, amino acids 1972-1996, corresponding to the transmembrane domain, and amino acids 1997-2039, corresponding to the cytoplasmic domain. In the extracellular domain, amino acids 42-101 correspond to SCR 1, 102-163 correspond to SCR2, amino acids 164-234 correspond to SCR3, amino acids 236-295 correspond to SCR4, amino acids 295-355 correspond to SCR5, amino acids 356-418 correspond to SCR6, amino acids 419-489 correspond to SCR7, amino acids 491-551 correspond to SCR8, amino acids 552-613 correspond to SCR9, amino acids 614-684 correspond to SCR10, amino acids 686-745 correspond to SCR11, amino acids 745-805 correspond to SCR12, amino acids 806-868 correspond to SCR13, amino acids 869-939 correspond to SCR14, amino acids 941-1001 correspond to SCR15, amino acids 1002-1063 correspond to SCR16, amino acids 1064-1134 correspond to SCR17, amino acids 1136-1195 correspond to SCR18, amino acids 1195-1255 correspond to SCR 19, amino acids 1256-1318 correspond to SCR 20, amino acids 1319-1389 correspond to SCR 21, amino acids 1394-1454 correspond to SCR 22, amino acids 1455-1516 correspond to SCR 23, amino acids 1517-1587 correspond to SCR 24, amino acids 1589-1648 correspond to SCR 25, amino acids 1648-1708 correspond to SCR 26, amino acids 1709-1771 correspond to SCR 27, amino acids 1772-1842 correspond to SCR 28, amino acids 1846-1906 correspond to SCR 29, amino acids 1907-1967 correspond to SCR 30. It is understood that species and strain variations exist for the disclosed peptides, polypeptides, and proteins, and that CR1 protein or biologically active fragments thereof encompass all species and strain variations. As used herein, the term “biologically active” fragment of CR1 protein refers to any soluble fragment of CR1 lacking the transmembrane domain and the cytoplasmic domain, including fragments comprising, consisting essentially of or consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 SCR domains, including any fragments of the full-length CR1 protein having some or all the complement inhibitory activity of the full-length CR1 protein.

As used herein, the term “complement factor H,” “factor H,” or “FH” refers to complement factor H, a single polypeptide chain plasma glycoprotein, including homologs thereof. The protein is composed of 20 conserved short consensus repeat (SCR) domains of approximately 60 amino acids, arranged in a continuous fashion like a string of beads, separated by short linker sequences of 2-6 amino acids each. Factor H binds to C3b, accelerates the decay of the alternative pathway C3-convertase (C3bBb), and acts as a cofactor for the proteolytic inactivation of C3b. In the presence of factor H, proteolysis by factor I results in the cleavage and inactivation of C3b. Factor H has at least three distinct binding domains for C3b, which are located within SCRs 1-4, SCRs 5-8, and SCRs 19-20. Each domain binds to a distinct region within the C3b protein: the N-terminal sites bind to native C3b; the second site, located in the middle region of factor H, binds to the C3c fragment and the site located within SCR19 and 20 binds to the C3d region. In addition, factor H also contains binding sites for heparin, which are located within SCR 7, SCRs 5-12, and SCR 20 of factor Hand overlap with those of the C3b binding sites. Structural and functional analyses have shown that the domains for the complement inhibitory activity of factor H are located within the first four N-terminal SCR domains.

SEQ ID NO:52 represents the full-length human factor H amino acid sequence (see, e.g., UniProtKB/Swiss-Prot. Accession No. P08603); SEQ ID NO:53 represents the full-length mouse factor H amino acid sequence (see, e.g., UniProtKB/Swiss-Prot. Accession No. P06909). In the human factor H sequence, amino acids 1-18 of SEQ ID NO:52 correspond to the signal peptide, and amino acids 19-1231 of SEQ ID NO:52 correspond to the mature protein. Within that protein, amino acids 21-80 of SEQ ID NO:52 correspond to SCR 1, amino acids 85-141 of SEQ ID NO:52 correspond to SCR 2, amino acids 146-205 of SEQ ID NO:52 correspond to SCR 3, amino acids 210-262 of SEQ ID NO:52 correspond to SCR 4, and amino acids 267-320 of SEQ ID NO:52 correspond to SCR 5. In the mouse factor H sequence, amino acids 1-18 of SEQ ID NO:53 correspond to the signal peptide, and amino acids 19-1234 of SEQ ID NO:53 correspond to the mature protein. Within that protein, amino acids 19-82 of SEQ ID NO:53 correspond to SCR 1, amino acids 83-143 of SEQ ID NO:53 correspond to SCR 2, amino acids 144-207 of SEQ ID NO:53 correspond to SCR 3, amino acids 208-264 of SEQ ID NO:53 correspond to SCR 4, and amino acids 265-322 of SEQ ID NO:53 correspond to SCR 5. It is understood that species and strain variations exist for the disclosed peptides, polypeptides, and proteins, and that factor H or biologically active fragments thereof encompass all species and strain variations.

As used herein, the term “biologically active” fragment of factor H refers to any portion of a factor H protein having some or all the complement inhibitory activity of the full-length factor H protein, and includes, but is not limited to, factor H fragments comprising SCRs 1-4, SCRs 1-5, SCRs 1-8, SCRs 1-18, SCRs 19-20, or any homolog of a naturally-occurring factor H or fragment thereof, as described in detail below. In some embodiments, the biologically active fragment of factor H has one or more of the following properties: (1) binding to C-reactive protein (CRP), (2) binding to C3b, (3) binding to heparin, (4) binding to sialic acid, (5) binding to endothelial cell surfaces, (6) binding to cellular integrin receptor, (7) binding to pathogens, (8) C3b co-factor activity, (9) C3b decay-acceleration activity, and (10) inhibiting the alternative complement pathway.

Thus, in some embodiments, the therapeutic moiety of a targeting construct described herein comprises a complement inhibitor or biologically active fragment thereof. In some embodiments, the complement inhibitor is selected from the group consisting of human MCP, human DAF, mouse DAF, human CD59, mouse CD59 isoform A, mouse CD59 isoform B, mouse Crry protein, human CR1, human factor H, or mouse factor H, a Factor I, or a biologically active fragment thereof.

In some embodiments, the complement inhibitor portion of the targeting construct comprises full-length human MCP (SEQ ID NO:44). In some embodiments, the complement inhibitor portion of the targeting construct comprises a biologically active fragment of human MCP (SEQ ID NO:44). In some embodiments, the biologically active fragment of human MCP is selected from the group consisting of SCRs 1-4 (amino acids 35-285 of SEQ ID NO:44), SCRs 1-4 plus the serine/threonine-rich domain (amino acids 35-326 of SEQ ID NO:44), and the extracellular domain of MCP (amino acids 35-343 of SEQ ID NO:44).’

In some embodiments, the complement inhibitor portion of the targeting construct comprises full-length human DAF. In some embodiments, the complement inhibitor portion of the construct comprises a biologically active fragment of human DAF (SEQ ID NO:45). In some embodiments, the biologically active fragment of human DAF is selected from the group consisting of SCRs 1-4 (amino acids 25-285 of SEQ ID NO:45) and SCRs 1-4 plus the O-glycosylated serine/threonine-rich domain (amino acids 25-353 of SEQ ID NO:45). In some embodiments, the complement inhibitor portion of the construct comprises full-length mouse DAF (SEQ ID N0:46). In some embodiments, the complement inhibitor portion of the construct comprises a biologically active fragment of mouse DAF. In some embodiments, the biologically active fragment of mouse DAF is selected from the group consisting of SCRs 1-4 (amino acids 35-286 of SEQ ID N0:46) and SCRs 1-4 plus the O-glycosylated serine/threonine-rich domain (amino acids 35-362 of SEQ ID N0:46).

In some embodiments, the complement inhibitor portion of the construct comprises full-length human CD59 (SEQ ID N0:47). In some embodiments, the complement inhibitor portion of the construct comprises a biologically active fragment of human CD59 (SEQ ID N0:47). In some embodiments, the biologically active fragment of human CD59 comprises the extracellular domain of human CD59 lacking its GPI anchor (amino acids 26-101 of SEQ ID N0:47). In some embodiments, the complement inhibitor portion of the construct comprises full-length mouse CD59, isoform A (SEQ ID N0:48). In some embodiments, the complement inhibitor portion of the construct comprises a biologically active fragment of mouse CD59, isoform A (SEQ ID N0:48). In some embodiments, the biologically active fragment of mouse CD59, isoform A comprises the extracellular domain of mouse CD59, isoform A lacking its GPI anchor (amino acids 24-95 of SEQ ID N0:48). In some embodiments, the complement inhibitor portion of the construct comprises full-length mouse CD59, isoform B (SEQ ID N0:49). In some embodiments, the complement inhibitor portion of the construct comprises a biologically active fragment of mouse CD59, isoform B (SEQ ID NO:49). In some embodiments, the biologically active fragment of mouse CD59, isoform B comprises the extracellular domain of mouse CD59, isoform Blacking its GPI anchor (amino acids 24-103 of SEQ ID NO:49).

In some embodiments, the complement inhibitor portion of the construct comprises full-length mouse Crry protein (SEQ ID NO:50). In some embodiments, the complement inhibitor portion of the construct comprises a biologically active fragment of mouse Crry protein (SEQ ID NO:50). In some embodiments, the biologically active fragment of mouse Crry protein is selected from the group consisting of SCRs 1-5 (amino acids 41-400 of SEQ ID NO:50) and the extracellular domain of mouse Crry protein (amino acids 41-405 of SEQ ID NO:50).

In some embodiments, the complement inhibitor portion of the construct comprises full-length human CR1 protein (SEQ ID NO:51). In some embodiments, the complement inhibitor portion of the construct comprises a biologically active fragment of human CR1 protein (SEQ ID NO:51). In some embodiments, the biologically active fragment of human CR1 protein is selected from the group consisting of SCRs 1-4 (amino acids 42-295 of SEQ ID NO:51), SCRs 1-10 (amino acids 42-684 of SEQ ID NO:51), SCRs 8-11 (amino acids 490-745 of SEQ ID NO:51), SCRs 15-18 (amino acids 940-1196 of SEQ ID NO:51), and SCRs 22-28 (amino acids 1394-1842 of SEQ ID NO:51).

In some embodiments, the complement inhibitor portion of the construct comprises full-length human (SEQ ID NO:52) or mouse (SEQ ID NO:53) factor H. In some embodiments, the complement inhibitor portion of the construct comprises a biologically active fragment of human (SEQ ID NO:52) or mouse (SEQ ID NO:53) factor H. In some embodiments, the biologically active fragment of human factor H (SEQ ID NO:52) is selected from the group consisting of SCRs 1-4 (amino acids 21-262 of SEQ ID NO:52), SCRs 1-5 of factor H (amino acids 21-320 of SEQ ID NO:52), SCRs 1-8 of factor H (amino acids 21-507 of SEQ ID NO:52), and SCRs 1-18 of factor H (amino acids 21-1104 of SEQ ID NO:52). In some embodiments, the biologically active fragment of mouse factor H (SEQ ID NO:53) is selected from the group consisting of SCRs 1-4 (amino acids 19-264 of SEQ ID NO:53), SCRs 1-5 of factor H (amino acids 19-322 of SEQ ID NO:53), SCRs 1-8 of factor H (amino acids 19-507 of SEQ ID NO:53), and SCRs 1-18 of factor H (amino acids 19-1109 of SEQ ID NO:53). In some embodiments, the biologically active fragment of human (SEQ ID NO:52) or mouse (SEQ ID NO:53) factor H comprises (and in some embodiments consists of or consists essentially of) at least the first four N-terminal SCR domains of factor H, including for example, at least any of the first 5, 6, 7, 8, 9, 10, 11, 12, 13, 14, 15, 16, 17, 18, or more N-terminal SCR domains of factor H.

In some embodiments, the complement inhibitor portion of the targeting construct is a homolog of any of the complement inhibitors described herein or a biologically active fragment thereof. Homologs of the complement inhibitors (or biologically active fragments thereof) include proteins which differ from a naturally occurring complement inhibitor (or biologically-active fragment thereof) in that at least one or a few, but not limited to one or a few, amino acids have been deleted (e.g., a truncated version of the protein, such as a peptide or fragment), inserted, inverted, substituted and/or derivatized (e.g., by glycosylation, phosphorylation, acetylation, myristoylation, prenylation, palmitation, amidation and/or addition glycosylphosphatidyl inositol). For example, homologue of a complement inhibitor may have an amino acid sequence that is at least about 70% identical to the amino acid sequence of a naturally complement inhibitor (e.g., SEQ ID NOs:44-53), for example at least about any of 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence of a naturally occurring complement inhibitor (e.g., SEQ ID NOs:44-53). Amino acid sequence identity can be determined in various ways, for example, using publicly available computer software such as BLAST, BLAST-2, ALIGN or MEGALIGNTM (DNAST AR) software. One skilled in the art can determine appropriate parameters for measuring alignment, including any algorithms needed to achieve maximal alignment over the full length of the sequences being compared.

In certain embodiments, a homologue of complement inhibitor (or a biologically active fragment thereof) retains all the alternative complement pathway inhibitory activity of the complement inhibitor (or a biologically active fragment thereof) from which it is derived. In certain embodiments, the homologue of a complement inhibitor (or a biologically-active fragment thereof) retains at least about 50%, for example, at least about any of 60%, 70%, 80%, 90%, or 95% of the complement inhibition activity the complement inhibitor (or a biologically-active fragment thereof) from which is derived.

In some embodiments, the complement inhibitor is an antibody (or an antigen binding fragment thereof) that binds to a complement component, e.g., a complement component selected from the group consisting of C1, C1q, Cis, C2, C2a, C3, C3a, C3b, C4, C4b, C5, C5a, C5b, C6, C7, C8, and C9. The complement polypeptides to which the antibodies or antigen binding fragments thereof bind can be, in some embodiments, human polypeptides, e.g., human C1, C1q, C1s, C2, C2a, C3, C3a, C3b, C4, C4b, C5, C5a, C5b, C6, C7, C8, C9, factor B, factor D, or properdin polypeptides. The amino acid sequences for the foregoing complement proteins are well-known in the art as are methods for preparing the proteins or fragments thereof for use in preparing an antibody (or antigen-binding fragment thereof) specific for one or more of the complement proteins. Suitable methods are also described and exemplified herein.

Exemplary anti-complement protein antibodies, which are suitable for incorporation into the targeting constructs described herein and for subsequent use in any of the methods described herein, are also well known in the art. For example, antibodies that bind to complement component C5 and inhibit the cleavage of C5 into fragments C5a and C5b include, e.g., eculizumab (Soliris®; Alexion Pharmaceuticals, Inc., Cheshire, Conn.) and pexelizumab (Alexion Pharmaceuticals, Inc., Cheshire, Conn.). See, e.g., Kaplan (2002) Curr Opin Investig Drugs 3(7):1017-23; Hill (2005) Clin Adv Hematol Oncol 3(11):849-50; Rother et al. (2007) Nature Biotechnol 25(11):1256-1488; Whiss (2002) Curr Opin Investig Drugs 3(6):870-7; Patel et al. (2005) Drugs Today (Barc) 41(3):165-70; and Thomas et al. (1996) Mol Immunol. 33(17-18):1389-401.

In some embodiments, the anti-C5 antibody can bind to an epitope in the alpha chain of the human complement component C5 protein. Antibodies that bind to the alpha chain of C5 are described in, for example, PCT application publication no. WO 2010/136311 and U.S. Pat. No. 6,355,245.

In some embodiments, the anti-C5 antibody can bind to an epitope in the beta chain of the human complement component C5 protein. Antibodies that bind to the C5 beta chain are described in, e.g., Moongkarndi et al. (1982) Immunobiol 162:397; Moongkarndi et al. (1983) Immunobiol 165:323; and Mollnes et al. (1988) Scand 1 Immunol 28:307-312.

Additional anti-C5 antibodies, and antigen-binding fragments thereof, suitable for use in the targeting constructs described herein are described in, e.g., PCT application publication no. WO 2010/015608, the disclosure of which is incorporated herein by reference in its entirety.

Antibodies that bind to C3b and, for example, inhibit the C3b convertase are also well known in the art. For example, PCT application publication nos. WO 2010/136311, WO b2009/056631, and WO 2008/154251, the disclosures of each of which are incorporated herein by reference in their entirety. Antagonistic anti-C6 antibodies and anti-C7 antibodies have been described in, e.g., Brauer et al. (1996) Transplantation 61(4):S88-S94 and U.S. Pat. No. 5,679,345.

In some embodiments, the complement inhibitor is an anti-factor B antibody (such as the monoclonal antibody 1379 produced by ATCC Deposit No. PTA-6230). Anti-factor B antibodies are also described in, e.g., Ueda et al. (1987) J Immunol 138(4):1143-9; Tanhehco et al. (1999) Transplant Proc 31(5):2168-71; U.S. patent application publication nos. 20050260198 and 2008029911; and PCT publication no. WO 09/029669.

In some embodiments, the complement inhibitor is an anti-factor D antibody, e.g., an antibody described in Pascual et al. (1990) 1 Immunol Methods 127:263-269; Sahu et al. (1993) Mol Immunol 30(7):679-684; Pascual et al. (1993) Eur 1 Immunol 23:1389-1392; Niemann et al. (1984) J Immunol 132(2):809-815; U.S. Pat. No. 7,439,331; or U.S. patent application publication no. 20080118506.

In some embodiments, the complement inhibitor is an anti-properdin antibody. Suitable anti-properdin antibodies are also well-known in the art and include, e.g., U.S. patent application publication nos. 20110014614 and PCT application publication no. WO2009110918.

In some embodiments, the complement inhibitor portion is an anti-MBL antibody. Mannose-binding mannan-binding lectin (MBL), a plasma protein, forms a complex with proteins known as MBL-associated serine proteases (MASPs). MBL binds to several monosaccharides that are uncharacteristic of mammalian proteins, e.g., mannose, N-acetylglucosamine, N-acetylmannoseamine, L-fucose and glucose, whereas sialic acid and galactose are not bound. When the MBL-MASP complex binds to microorganisms, the proenzymic forms of the serine proteases are activated and mediate the activation of complement components C4 and C2, thereby generating the C3 convertase C4b2b and leading to opsonization by the deposition of C4b and C3b fragments. MASP-2 has been shown to cleave C4 and C2, while MASP-1 may be responsible for direct cleavage of C3. The functions of MASP-3 and MAp19 are less well understood. Studies have shown a clear link between low levels of MBL and opsonic deficiency, as well as clinical manifestations such as severe diarrhea, chronic hepatitis and HIV infection, and autoimmune disease. See, Petersen et al., J. Immunological Methods, 257:107-16 (2001); Petersen et al., Molecular Immunology, 38:133-49 (2001). Anti-mannan-binding lectin antibodies are known in the art (see, e.g., Pradhan et al. (2012) Rheumatol. Int. epublished September, 2012) and commercially available (AbCam).

In some embodiments, the complement inhibitor portion is an anti-MASP antibody. The mannan-binding lectin-associated serine proteases (MASPs) are a family of at least three proteins (mannan-binding lectin-associated serine protease-1, -2 and -3 (MASP-1, MASP-2 and MASP-3, respectively)), which have been taught to play a significant role in modulation of the lectin pathway of complement activation. Petersen et al., Molecular Immunology 38:133-149 (2001).

MASP-1 has a histidine loop structure of the type found in trypsin and trypsin-like serine proteases. MASP-1 has been found to be involved in complement activation by MBL. A cDNA clone encoding MASP-1 has been reported that encodes a putative leader peptide of 19 amino acids followed by 680 amino acid residues predicted to form the mature peptide. MASP-2 (MBL-associated serine protease 2) is a serine protease also similar in structure to C1 r and C1 s of the complement pathway. Like these, and contrary to MASP-1, it has no histidine loop structure of the type found in trypsin and trypsin-like serine proteases. It has been theorized that MASP-1 can cleave C3, generating C3b, which may be deposited on an activated cell or tissue surface

It has been shown that MASP-2, cleaves C4 and C2, giving rise to the C3 convertase, C4b2b (Thiel et al., Nature, 386:506-10 (1997)). The MASP-2 protein comprises of a number of domains namely the CUB1, EGF, CUB2, CCP1, CCP2 and serine protease domains. It is believed that the domain responsible for association with MBL is situated in the N-terminus, whereas the serine protease domain is responsible for the serine protease activity of MASP-2. sMAP, also known as MAp19, is a 19 kd is derived from the same gene as MASP-2, which lacks the serine protease domain and a major part of the A chain. Skjoedt et al., Immunobiology, 215:921-31 (2010). Recently, a third member of the family, MASP-3 was identified, which shares a high degree of homology with MASP-1, such that it appears that MASP-1 and MASP-3 are generated as a result of alternative splicing of primary mRNA transcripts.

Antibodies against MBL, MASP-1, MASP-2, MASP-3 and the MBL/MASP complex, and their use for inhibiting the adverse effects of complement activation, such as ischemia-reperfusion injury, have been disclosed, for example, in WO04/075837; US 2009/0017031.

Other antibodies to MASP-2 have been described previously, as well. See, e.g., WO 02/06460, US2007/0009528, Peterson et al., Mol. Immunol. 37:803-11 (2000), Moller-Kristensen et al., J. of Immunol. Methods 282:159-67 (2003), Petersen et al., Mol. Immunol. 35:409, and WO 04/106384.

An additional related protein, MBL/Ficolin Associated Protein (MAP-1), which is present in low serum levels compared to MASP-1 and MASP-3, has been reported to function as a local lectin pathway specific complement inhibitor. Skjodt et al., Molecular Immunology, 47:2229-30 (2010). Accordingly MAP-1 itself, or fragments of MAP-1, may be useful in the present invention as an inhibitor of MASP, and accordingly, as a lectin-pathway-specific inhibitor of complement activation. Finally, the ficolin family of proteins are characterized by carbohydrate binding and opsonic activities, sharing a structure similar to MBL. Like MBL, the ficolins have been shown to associate with MASPs in serum and may mediate complement activation in response to pathogenic, necrotic, or apoptotic cell-specific carbohydrate markers. Accodingly, inhibitors of the ficolin family or functional fragments therof may be useful in the present invention as an inhibtor of MASPs and as a lectin-pathway specific inhibitor of complement activation. U.S. Pat. No. 6,333,034 and U.S. Pat. No. 7,423,128; see also, WO 2008/154018 and WO 2009/110918.

In some embodiments, the complement inhibitor portion is an antibody (or antigen binding fragment thereof) that specifically binds to a human complement component protein (e.g., human C5, C6, C7, C8, or C9). The terms “specific binding” or “specifically binds” refer to two molecules forming a complex (e.g., a complex between an antibody and a complement component protein) that is relatively stable under physiologic conditions. Typically, binding is considered specific when the association constant (Ka) is higher than 106 M-1. Thus, an antibody can specifically bind to a C5 protein with a Ka of at least (or greater than) 106 (e.g., at least or greater than 107, 108, 109, 1010, 1011, 1012, 1013, 1014, or 1015 or higher) M-1. Examples of antibodies that specifically bind to a human complement component C5 protein are described in, e.g., U.S. Pat. No. 6,355,245 and PCT application publication no. WO 2010/015608.

Methods for determining whether an antibody binds to a protein antigen and/or the affinity for an antibody to a protein antigen are known in the art and described herein. For example, the binding of an antibody to a protein antigen can be detected and/or quantified using a variety of techniques such as, but not limited to, Western blot, dot blot, surface plasmon resonance method (e.g., BIAcore system; Pharmacia Biosensor AB, Uppsala, Sweden and Piscataway, N.J.), or enzyme-linked immunosorbent assay (ELISA) assays. See, e.g., Harlow and Lane (1988) “Antibodies: A Laboratory Manual” Cold Spring Harbor Laboratory Press, Cold Spring Harbor, N.Y.; Benny K. C. Lo (2004) “Antibody Engineering: Methods and Protocols,” Humana Press (ISBN: 1588290921); Borrebaek (1992) “Antibody Engineering, A Practical Guide,” W.H. Freeman and Co., NY; Borrebaek (1995) “Antibody Engineering,” 2nd Edition, Oxford University Press, NY, Oxford; Johne et al. (1993) 1 Immunol Meth. 160:191-198; Jonsson et al. (1993) Ann Biol Clin 51:19-26; and Jonsson et al. (1991) Biotechniques 11:620-627. See also, U.S. Pat. No. 6,355,245.

In any of the embodiments described herein, the targeting construct also includes an amino acid linker sequence linking the targeting moiety and the therapeutic moiety (or a biologically active fragment thereof).

In some embodiments, the targeting moiety of the targeting construct is joined (e.g., directly or by way of a linker) to the amino-terminus of the therapeutic moiety. In some embodiments, the targeting moiety of the targeting construct is joined (e.g., directly or by way of a linker) to the carboxy-terminus of the therapeutic moiety (e.g., a complement inhibitor or drug described herein).

In some embodiments, a targeting construct described herein comprises more than one (e.g., two, three, four, five, six, or seven or more) therapeutic moiety, e.g., more than one complement inhibitor polypeptide or drug described herein. The two or more therapeutic moieties can be the same or different. For example, a targeting construct described herein can comprise, in some embodiments, two or more soluble CD59 portions (e.g., soluble human CD59 portions) or two or more beta blockers. In another example, a targeting construct described herein can contain two or more complement inhibitor polypeptide portions, wherein one is a soluble human CD59 and another is soluble human MCP. In another example, a targeting construct described herein can contain a complement inhibitor and a drug, e.g., one soluble CD59 portion and one corticosteroid. Thus, e.g., a targeting construct described herein can comprise: (a) a targeting moiety (e.g., a C2 antibody, a B4 antibody, or an antigen-binding fragment of either of the foregoing); (b) a first therapeutic moiety (e.g., a soluble form of CD59, e.g., human CD59); and (c) a second therapeutic moiety (e.g., a soluble form of DAF, e.g., a soluble form of human DAF, or a corticosteroid such as prednisone). The therapeutic moiety can be, e.g., any of those described herein including variants and biologically active fragments of the complement inhibitors described herein.

In some embodiments, the light chain of the targeting moiety of the targeting construct comprises at least one therapeutic moiety and the heavy chain comprises at least therapeutic moiety. The two or more complement inhibitor polypeptides can be the same or different. For example, in some embodiments, the targeting construct comprises the Fab fragment of a targeting moiety described herein, wherein: (i) the light chain of the Fab fragment comprises (at its C-terminal end) a complement inhibitor polypeptide such as DAF, CD59, or any of the complement inhibitor polypeptides described herein and (ii) the heavy chain of the Fab fragment comprises (at its C-terminal end) the same or a different therapeutic moiety as in (i), e.g., a complement inhibitor or a drug described herein. Appropriate pairing of the two chains can be expected to occur as an inherent property of the Fab. The complement inhibitor portion and the light chain or heavy chain of the Fab can be joined together directly or by way of a linker sequence (such as any of those described herein).

Detectable Moieties

In some embodiments, the targeting construct comprises a targeting moiety fused to an active moiety that is detectable moiety. In some embodiments, the detectable moiety can be a paramagnetic molecule, a paramagnetic nanoparticle, an ultrasmall superparamagnetic iron oxide (“USPIO”) nanoparticle, or a USPIO nanoparticle aggregate. In certain embodiments, the USPIO nanoparticle aggregate is between about 10 nm and about 150 nm in diameter, between about 65 nm and about 85 nm in diameter, or about 75 nm in diameter. In certain embodiments, the USPIO nanoparticle aggregate is about 150 nm in diameter. In certain embodiments, the USPIO nanoparticle aggregate is coated with dextran or an amphiphilic polymer, or the USPIO nanoparticle aggregate is encapsulated with phospholipid. In certain embodiments, the phospholipid is PEGylated. In certain embodiments, the PEGylated phospholipid is amine-functionalized or carboxylic acid-functionalized. In certain embodiments the PEGylated, amine-functionalized phospholipid is 1,2-distearoyl-sn-glycero-3-phosphoethanolamine-PEG2000.

In certain embodiments, the a paramagnetic nanoparticle is a superparamagnetic iron oxide (“SPIO”) nanoparticle, an SPIO nanoparticle aggregate, standard superparamagnetic iron oxide (“SSPIO”), an SSPIO nanoparticle aggregate, polydisperse superparamagnetic iron oxide (“PSPIO”), a PSPIO nanoparticle aggregate, monochrystalline SPIO, a monochrystalline SPIO aggregate, a monochrystalline iron oxide nanoparticle, monochrystalline iron oxide, or any other nanoparticle contrast agent known to one of skill in the art. Methods of conjugating such particles to a targeting moiety and detecting paramagnetic particle-conjugated targeting moieties are known in the art and described in, e.g., Richardson et al. (2001) Biosens and Bioelect 16:989-993; Boyaci et al. (2005) Anal Bioanal Chem 382(5):1234-41; Toma et al. (2005) Br J Cancer 93(1):131-6; and others.

In some embodiments, the detectable moiety is a liposome or another delivery vehicle containing Gadolinium chelate (“Gd-chelate”) molecules. In some embodiments, the detectable moiety is an electron-dense reagent, such as Gadolinium, an iodinated contrast agent, barium sulfate, thorium dioxide, gold, a gold nanoparticle, a gold nanoparticle aggregate. In some embodiments, the detectable moiety is a biocolloid, or a microbubble. In some embodiments, the detectable moiety is a radioisotope or a radionuclide, including, but not limited to, e.g., 32P, carbon-11, nitrogen-13, oxygen-15, fluorine-18, rubidium-82, fluorodeoxyglucose, a gamma ray emitting radionuclide, radiolabeled glucose, radiolabeled water, or radiolabeled ammonia. In certain embodiments, the detectable moiety is a positron-emitting radionuclide. In certain embodiments, the detectable moiety is a hapten, a protein (such as biotin), an enzyme, a digoxygenin, or a fluorophore, a two-photon fluorophore, a fluorescent dye, or a fluorescent moiety (e.g., a fluorescein, fluorescein isothiocyanate, or a fluorescein derivative). Methods of conjugating such detectable moieties to a targeting moiety are known in the art and are described elsewhere herein.

Targeting construct comprising a detectable moiety can be used in noninvasive methods of detecting complement-mediated inflammation or complement activation in the eye of an individual in need thereof. As noted elsewhere herein, the methods include administering to the individual a composition comprising an effective amount of a targeting construct comprising a detectable moiety and measuring the presence of the detectable moiety using an instrument and/or method (e.g. MRI, CT, SPECT, radiography, spectroscopy, microscopy, PET, ultrasound, or any other detection method described herein) capable of detecting the presence of the detectable moiety.

MRI can be used to non-invasively acquire tissue images with high resolution. Paramagnetic agents or USPIO nanoparticles or aggregates thereof enhance signal attenuation on T2-weighted magnetic resonance images, and conjugation of such nanoparticles to, e.g., an antibody described herein (or a fragment thereof) or a construct described herein, permits the detection of specific molecules at the cellular level. For example, MRI with nanoparticle detection agents can image cell migration (J. W. Bulte et al., 2001, Nat. Biotechnol. 19:1141-1147), apoptosis (M. Zhao et al., 2001, Nat. Med. 7:1241-1244), and can detect small foci of cancer. See e.g., Y. W. Jun et al., 2005, J. Am. Chem. Soc. 127:5732-5733; Y. M. Huh et al., 2005, J. Am. Chem. Soc. 127:12387-12391. Contrast-enhanced MRI is well-suited for the dynamic non-invasive imaging of macromolecules or of molecular events, but it requires ligands hat specifically bind to the molecule of interest. J. W. B 5 ulte et al., 2004, NMR Biomed. 17:484-499. Fluorescent dyes and fluorophores e.g. fluorescein, fluorescein isothiocyanate, and fluorescein derivatives) can be used to non-invasively acquire tissue images with high resolution, with for example spectrophotometry, two-photon fluorescence, two-photon laser microscopy, or fluorescence microscopy (e.g. of tissue biopsies). MRI can be used to non-invasively acquire tissue images with high resolution, with for example paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, other nanoparticle contrast agents. MRI can be used to non-invasively acquire tissue images with high resolution, with for example Gadolinium, including liposomes or other delivery vehicles containing Gadolinium chelate (“Gd-chelate”) molecules. Positron emission tomography (PET), PET/computed tomography (CT), single photon emission computed tomography (SPECT), and SPECT/CT can be used to non-invasively acquire tissue images with high resolution, with for example radionuclides (e.g. carbon-11, nitrogen-13, oxygen-15, fluorine-18, rubidium-82), fluorodeoxyglucose (e.g. fluorine-18 labeled), any gamma ray emitting radionuclides, positron-emitting radionuclide, radiolabeled glucose, radiolabeled water, radiolabeled ammonia. Ultrasound (ultrasonography) and contrast enhanced ultrasound (contrast enhanced ultrasonography) can be used to non-invasively acquire tissue images with high resolution, with for example biocolloids or microbubbles (e.g. including microbubble shells including albumin, galactose, lipid, and/or polymers; microbubble gas core including air, heavy gas(es), perfluorcarbon, nitrogen, octafluoropropane, perflexane lipid microsphere, perflutren, etc.). X-ray imaging (radiography) or CT can be used to non-invasively acquire tissue images with high resolution, with for example iodinated contrast agents (e.g. iohexol, iodixanol, ioversol, iopamidol, ioxilan, iopromide, diatrizoate, metrizoate, ioxaglate), barium sulfate, thorium dioxide, gold, gold nanoparticles, or gold nanoparticle aggregates. These detection methods and instruments and detectable moieties capable of measured or detected by the corresponding method are non-limiting examples.

As used herein, the term “ultrasmall superparamagnetic iron oxide nanoparticle” or “USPIO nanoparticle” refers to superparamagnetic iron oxide particles ranging from 1 to 50 nm in diameter, more typically between 5 and 40 nm in diameter (excluding 5 any coating applied after synthesis). USPIO nanoparticles are commonly made of maghemite (Fe2O3) or magnetite (Fe₃O₄) having crystal-containing regions of unpaired spins. Those magnetic domains are disordered in the absence of a magnetic field, but when a field is applied (i.e., while taking an MRI), the magnetic domains align to create a magnetic moment much greater than the sum of the individual unpaired electrons without resulting in residual magnetization of the particles. When injected into the blood stream, USPIO nanoparticles are taken up by macrophages and accumulate in inflamed tissues. Their iron moiety negatively enhances signal attenuation on T2-weighted images, and their relative concentrations can be assessed by decreased T2-signal intensity or, more precisely, by decreased spin-spin T2-relaxation time. The decreased T2-relaxation time (the transverse relaxation time) can thus be used to detect inflammation. The shortened T2 relaxation time results in a darkening of the magnetic resonance image where the particles are located, thereby generating “negative contrast.” This approach has been successfully utilized to detect renal inflammation in several models. In some cases, USPIO nanoparticles may be aggregated after synthesis to produce aggregates thereof (referred to herein as “ultrasmall superparamagnetic iron oxide (“USPIO”) nanoparticle aggregates” or “USPIO nanoparticle aggregates”) of 25 nm, 50 nm, 75 nm, 100 nm, or 150 nm, in diameter, or even larger.

The USPIO nanoparticles or aggregates thereof may be coated with a wide variety of materials, including natural or synthetic polymers, surfactants, phospholipids, or inorganic materials, any of which may be modified or derivatized to permit attachment of targeting groups, either directly or via different types of linkers, including peptides, polypeptides, proteins, or other chemical groups, or uncoated. Possible coatings include synthetic polymers, such as those based on poly(ethylene-co-vinyl acetate), polyvinylpyrrolidone (“PYP”), poly(lactic-co-glycolic acid) (“PLGA”), polyethylene glycol (“PEG”), polyvinyl alcohol (“PYA”), polyacrylic acid, and the like; natural polymers, such as gelatin, dextran, chitosan, pullulan, and the like; surfactants, such as sodium oleate, dodecylamine, sodium carboxymethylcellulose, and the like; inorganic materials, such as gold or silica; and biological materials, such as phospholipids.

Thus, a complement-mediated inflammation (such as in the eye) can be detected in an individual in a non-invasive manner by administering an the antibody-targeted USPIO nanoparticle or nanoparticle aggregate compositions and/or USPIO nanoparticle- or USPIO nanoparticle aggregate-conjugated targeting constructs provided herein, and taking a magnetic resonance taking a magnetic resonance image of the individual, or of the individual's eye. In some of the embodiments described herein, the composition administered to the individual is a pharmaceutical composition comprising any of the antibody (or antigen-binding fragment thereof) and/or construct described herein. In some of the embodiments described herein, the composition administered to the individual is a pharmaceutical composition comprising any of the antibody-targeted USPIO nanoparticle aggregate compositions described herein.

As used herein, the term “magnetic resonance imaging” or “MRI” refers to a non-invasive medical imaging technique commonly used to visualize the structure and function of the body that provides detailed images of the body in any plane. MRI provides much greater contrast between the different soft tissues of the body than other non-invasive imaging methods, such as computed tomography (CT), making it especially useful in neurological, musculoskeletal, cardiovascular, and oncological (cancer) imaging. Unlike CT, it does not require ionizing radiation, instead using a powerful magnetic field to align the nuclear magnetization of hydrogen atoms in water in the body. Radiofrequency fields are used to systematically alter the alignment of this magnetization, causing the hydrogen nuclei to produce a rotating magnetic field detectable by the scanner. This signal can be manipulated by additional magnetic fields to build up enough information to reconstruct an image of the body or a portion thereof, e.g., the eye.

When an individual lies in a scanner, the hydrogen nuclei (i.e., protons) found in abundance in water molecules throughout the individual's body, align with the strong main magnetic field. A second electromagnetic field, which oscillates at radiofrequencies and is perpendicular to the main field, is then pulsed to push a proportion of the protons out of alignment with the main field. These protons then drift back into alignment with the main field, emitting a detectable radiofrequency signal as they do so. Since protons in different body tissues (e.g., fat vs. muscle) realign at different speeds, different body structures can be imaged. Contrast agents may be injected intravenously to enhance the appearance of blood vessels, organs (e.g., the eye), tumors or sites of inflammation.

As used herein, an “effective amount” or “diagnostically effective amount” of an antibody-targeted ultrasmall superparamagnetic iron oxide (“USPIO”) nanoparticle or nanoparticle aggregate composition (including any of the pharmaceutical compositions described herein) is an amount sufficient to produce a clinically useful magnetic resonance image of complement-mediated inflammation such as in the eye. A clinically useful magnetic resonance image is one containing sufficient detail to enable an experienced clinician to assess the degree and/or extent of inflammation for purposes of diagnosis, monitoring the efficacy of a therapeutic intervention, and the like. As used herein, an “effective amount” or “diagnostically effective amount” of an antibody-targeted detectable moiety or a targeting construct comprising a detectable moiety (including any of the pharmaceutical compositions described herein) is an amount sufficient to produce a clinically useful characterization or measurement of complement-mediated inflammation or complement activation (e.g. in an individual, patient, human, mammal, clinical sample, tissue, biopsy) when coupled with a detection method capable of detecting the antibody (or fragment thereof) and/or the targeting moiety. A clinically useful characterization or measurement of complement-mediated inflammation or complement activation is one containing sufficient detail to enable an experienced clinician to assess the degree and/or extent of inflammation or complement activation for purposes of diagnosis, monitoring the efficacy of a therapeutic intervention, and the like.

Delivery of ultrasmall superparamagnetic iron oxide (“USPIO”) nanoparticles, USPIO nanoparticle aggregates, superparamagnetic iron oxide (“SPIO”) nanoparticles, SPIO nanoparticle aggregates or other nanoparticle contrast agents (examples of detectable moieties) to the sites of active inflammation via delivery of antibodies and/or targeting construct to sites of complement activation permits non-invasive magnetic resonance imaging of such inflammation, enabling the specific detection of complement activation throughout the body, and distinguishing complement-mediated inflammation from other types of inflammation.

Accordingly, in one aspect, the invention provides compositions comprising nanoparticle contrast agents conjugated to an antibody (or antigen binding fragment thereof) or a construct described herein for non-invasive medical or diagnostic imaging applications. In certain embodiments, the nanoparticle contrast agent-conjugated antibody (or antigen binding fragment thereof) or a construct comprises USPIO nanoparticles or aggregates thereof. In certain embodiments, the nanoparticle contrast agent-conjugated antibody (or antigen binding fragment thereof) or a construct comprise liposomes or other vehicles containing Gadolinium chelate (“Gdchelate”) molecules. Ultrasmall super paramagnetic iron oxide (“USPIO”) nanoparticles or aggregates are examples of detectable moieties that can be conjugated to a targeting construct described herein.

At least two physicochemical characteristics of ultrasmall super paramagnetic iron oxide (“USPIO”) nanoparticles or aggregates thereof vary with the size of the individual nanoparticles or nanoparticle aggregates. First, the ability of USPIO nanoparticle preparations to enhance contrast in MRI imaging and the degree of contrast enhancement both vary with nanoparticle diameter, because the magnetic moment of individual USPIO nanoparticles also varies with particle diameter. Iron oxide nanoparticles with diameters up to approximately 15 nm (preferably less than 10 nm) remain super paramagnetic, but larger iron oxide nanoparticles lose their superparamagnetic properties. Thus, there is an upper limit to the diameter of USPIO nanoparticles suitable for use as MRI contrast reagents. This limitation can be overcome by use of multiparticle aggregates of smaller individual USPIO nanoparticles. Such USPIO nanoparticle aggregates effectively enhance MRI contrast because the magnetic moments of the individual nanoparticles within each nanoparticle aggregate are additive. Unlike individual iron oxide nanoparticles, aggregates of ultra small super paramagnetic iron oxide nanoparticles do not lose their paramagnetic properties with increased size.

Second, the in vivo half-life (e.g., circulating plasma or blood half-life and tissue half-life) and biodistribution of USPIO nanoparticles or aggregates thereof varies with nanoparticle or aggregate size. For example, USPIO nanoparticles ˜10 nm or less in diameter (monochrystalline iron oxide nanoparticles) have a circulating blood half-life of ˜81 minutes (R. Weissleder et al., 1990, Radial. 175(2):489-493), USPIO nanoparticles ˜50 nm in diameter have a circulating half-life of ˜30 minutes (D. Pouliquen et al., 1991, Magnet. Resonance Imag. 9(3):275-283), USPIO nanoparticles ˜150 nm in diameter are thought to have a circulating half-life of less than ˜30 minutes, and USPIO nanoparticles ˜80 nm in diameter have a tissue half-life on the order of one to several days (e.g., 1, 2, 3, 4, 5, 6, 7, 8, 9, or more days) and a whole body half-life of ˜45 days (R. Weissleder et al., 1989, Am. J. Roentgenol. 152(1):167-173). Effective targeted MRI contrast-enhancing reagents must circulate in the vasculature long enough to recognize and bind the desired target (e.g., annexin IV or a phospholipid such as PE, PC, CL, or MDA) while still being cleared quickly enough to minimize any potential toxicity. Optimal USPIO nanoparticle or nanoparticle aggregate sizes for generating clinically useful magnetic resonance images vary depending on the organ (e.g., the kidney, eye, retina), tissue, and/or physiological phenomenon (e.g., complement-mediated inflammation) to be imaged.

The circulating half-life of USPIO nanoparticles or nanoparticle aggregates can also be altered (i.e., reduced or extended) by coating them with different materials. For instance, USPIO nanoparticles or nanoparticle aggregates can be coated with natural or synthetic polymers, surfactants, or phospholipids, among other materials, any of which may be modified or derivatized to permit attachment of an antibody (or antigen binding fragment thereof) and/or a construct described herein, either directly or indirectly via different types of linkers, including peptides, polypeptides, proteins, or other chemical groups. In some cases, the coatings may be further modified to incorporate synthetic polymers, natural polymers, amphiphilic polymers, or other molecules (e.g., polyvinylpyrrolidone (“PVP”), poly (lactic-coglycolic acid) (“PLGA”), polyethylene glycol (“PEG”), polyvinyl alcohol (“PYA”), polyacrylic acid, and the like) suitable for stabilizing the aggregates or minimizing their susceptibility to extravasation, opsonization, phagocytosis, endocytosis or other modes of physiological clearance. In some cases, USPIO nanoparticles or nanoparticle aggregates conjugated to an antibody (or antigen-binding fragment thereof) or construct can be phospholipid-encapsulated. As with USPIO nanoparticle or nanoparticle aggregate size, the particular coating, modification or derivatization suitable for targeting the nanoparticles or nanoparticle aggregates to a desired organ (e.g., the kidney, eye, retina), tissue, and/or physiological phenomenon (e.g., complement-mediated inflammation) may be determined empirically.

Variants of Targeting Constructs

Also encompassed are variants of the targeting constructs. A variant of the targeting construct described herein may be: (i) one in which one or more of the amino acid residues of the targeting moiety and/or the active moiety (i.e., wherein the active moiety comprises a protein) are substituted with a conserved or non-conserved amino acid residue (preferably a conserved amino acid residue) and such substituted amino acid residue may or may not be one encoded by the genetic code; or (ii) one in which one or more of the amino acid residues in the targeting and/or active moiety includes a substituent group, or (iii) one in which the targeting construct is fused with another compound, such as a compound to increase the half-life of the targeting construct (for example, polyethylene glycol), or (iv) one in which additional amino acids are fused to the targeting construct (such as the targeting moiety or the active moiety, wherein the active moiety comprises a protein), such as a leader or secretory sequence or a sequence which is employed for purification of the targeting construct, or (v) one in which the targeting construct is fused with a larger polypeptide, i.e., human albumin, an antibody or Fc, for increased duration of effect. Such variants are deemed to be within the scope of those skilled in the art from the teachings herein.

In some embodiments, the variant of the targeting construct contains conservative amino acid substitutions (defined further below) made at one or more predicted, preferably nonessential amino acid residues. A “nonessential” amino acid residue is a residue that is altered from the wild-type sequence of a protein without altering the biological activity, whereas an “essential” amino acid residue is required for biological activity. A “conservative amino acid substitution” is one in which the amino acid residue is replaced with an amino acid residue having a similar side chain.

Twenty amino acids are commonly found in proteins. Those amino acids can be grouped into nine classes or groups based on the chemical properties of their side chains. Substitution of one amino acid residue for another within the same class or group is referred to herein as a “conservative” substitution. Conservative amino acid substitutions can frequently be made in a protein without significantly altering the conformation or function of the protein. Substitution of one amino acid residue for another from a different class or group is referred to herein as a “non-conservative” substitution. In contrast, non-conservative amino acid substitutions tend to disrupt conformation and function of a protein. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). (See Table 1 below.)

TABLE 1
Example of amino acid classification

| | |
|---|---|
| Small/Aliphatic residues: | Gly, Ala, Val, Leu, Ile |
| Cyclic Imino Acid: | Pro |
| Hydroxyl-containing Residues: | Ser, Thr |
| Acidic Residues: | Asp, Glu |
| Amide Residues: | Asn, Gln |
| Basic Residues: | Lys, Arg |
| Imidazole Residue: | His |
| Aromatic Residues: | Phe, Tyr, Trp |
| Sulfur-containing Residues: | Met, Cys |

In some embodiments, the conservative amino acid substitution comprises substituting any of glycine (G), alanine (A), isoleucine (I), valine (V), and leucine (L) for any other of these aliphatic amino acids; serine (S) for threonine (T) and vice versa; aspartic acid (D) for glutamic acid (E) and vice versa; glutamine (Q) for asparagine (N) and vice versa; lysine (K) for arginine (R) and vice versa; phenylalanine (F), tyrosine (Y) and tryptophan (W) for any other of these aromatic amino acids; and methionine (M) for cysteine (C) and vice versa. Other substitutions can also be considered conservative, depending on the environment of the particular amino acid and its role in the three-dimensional structure of the protein. For example, glycine (G) and alanine (A) can frequently be interchangeable, as can alanine (A) and valine (V). Methionine (M), which is relatively hydrophobic, can frequently be interchanged with leucine and isoleucine, and sometimes with valine. Lysine (K) and arginine (R) are frequently interchangeable in locations in which the significant feature of the amino acid residue is its charge and the differing pKs of these two amino acid residues are not significant. Still other changes can be considered “conservative” in particular environments (see, e.g., Biochemistry at pp. 13-15, 2nd ed. Lubert Stryer ed. (Stanford University); Henikoff et al., Proc. Nat'l Acad. Sci. USA (1992) 89:10915-10919; Lei et al., J. Biol. Chem. (1995) 270(20):11882-11886).

Amino acid substitutions in the targeting moiety and/or the active moiety of the targeting construct is introduced to improve the functionality of the targeting construct. For example, amino acid substitutions can be introduced into the targeting moiety of targeting construct to increase binding affinity of the targeting moiety to its ligand(s), increase binding specificity of the targeting construct to its ligand(s), improve targeting of the targeting construct to desired sites, increase dimerization or multimerization of the targeting construct, and improve pharmacokinetics of the targeting construct. Similarly, amino acid substitutions can be introduced into the active moiety of the targeting construct to increase the functionality of the targeting construct molecule and improve pharmacokinetics of the targeting construct.

In some embodiments, the targeting construct is fused with another compound, such as a compound to increase the half-life of the targeting construct and/or to reduce potential immunogenicity of the targeting construct (for example, polyethylene glycol, “PEG”). The PEG can be used to impart increased stability, water solubility, size, slow rate of kidney clearance, and reduced immunogenicity to the targeting construct. See e.g., U.S. Pat. No. 6,214,966; Lee et al. (1999) Bioconjug Chem 10(6): 973-8; Kinstler et al. (2002) Advanced Drug Deliveries Reviews 54:477-485; and Roberts et al. (2002) Advanced Drug Delivery Reviews 54:459-476. The stabilization moiety can improve the stability, or retention of, the polypeptide by at least 1.5 (e.g., at least 2, 5, 10, 15, 20, 25, 30, 40, or 50 or more) fold. In the case of PEGylations, the fusion of a targeting construct described herein to PEG can be accomplished by any means known to one skilled in the art. For example, PEGylation can be accomplished by first introducing a cysteine mutation into the targeting moiety or the active moiety (i.e., wherein the active moiety comprises a protein), followed by site-specific derivatization with PEG-maleimide. The cysteine can be added to the C-terminus of the targeting construct. See, e.g., Tsutsumi et al. (2000) Proc. Natl. Acad. Sci. USA 97(15):8548-8553. Another modification which can be made to the targeting construct involves biotinylation. In certain instances, it may be useful to have the targeting construct biotinylated so that it can readily react with streptavidin. Methods for biotinylation of proteins are well known in the art. Additionally, chondroitin sulfate can be linked with the targeting construct.

In some embodiments, the targeting construct is fused to another moiety which further increases the targeting efficiency of the targeting construct. For example, a targeting construct comprising a B4 antibody can be fused to, e.g., a C2 antibody or another antibody that has the capability to bind or otherwise attach to an endothelial cell of a blood vessel (referred to as “vascular endothelial targeting amino acid ligand”). Exemplary vascular endothelial targeting ligands include, but are not limited to, VEGF, FGF, integrin, fibronectin, I-CAM, PDGF, or an antibody to a molecule expressed on the surface of a vascular endothelial cell.

In some embodiments, the targeting construct is conjugated (such as fused) to a ligand for intercellular adhesion molecules. For example, the target construct molecule can be conjugated to one or more carbohydrate moieties that bind to an intercellular adhesion molecule. The carbohydrate moiety facilitates localization of the target construct molecule to the site of injury. The carbohydrate moiety can be attached to the target construct molecule by means of an extracellular event such as a chemical or enzymatic attachment, or can be the result of an intracellular processing event achieved by the expression of appropriate enzymes. In some embodiments, the carbohydrate moiety binds to a particular class of adhesion molecules such as integrins or selectins, including E-selectin, L-selectin or P-selectin. In some embodiments, the carbohydrate moiety comprises an N-linked carbohydrate, for example the complex type, including fucosylated and sialylated carbohydrates. In some embodiments, the carbohydrate moiety is related to the Lewis X antigen, for example the sialylated Lewis X antigen.

For treatment of eye diseases such as AMD, the targeting construct can be conjugated (such as fused) to an antibody that recognizes an epitope of the drusen. Other targeting molecules such as small targeting peptide can also be used. Other modifications of the targeting construct include, for example, glycosylation, acetylation, phosphorylation, amidation, derivatization by known protecting/blocking groups, and the like.

The targeting construct may include the addition of an immunologically active domain, such as an antibody epitope or other tag, to facilitate targeting or purification of the polypeptide. The use of 6×His and GST (glutathione S transferase) as tags is well known. Inclusion of a cleavage site at or near the fusion junction will facilitate removal of the extraneous polypeptide from the targeting construct after purification. Other amino acid sequences that may be included in the targeting construct include functional domains, such as active sites from enzymes such as a hydrolase, glycosylation domains, and cellular targeting signals.

Variants of the targeting construct include polypeptides having an amino acid sequence sufficiently similar to the amino acid sequence of a targeting construct described herein. The term “sufficiently similar” means a first amino acid sequence that contains a sufficient or minimum number of identical or equivalent amino acid residues relative to a second amino acid sequence such that the first and second amino acid sequences have a common structural domain and/or common functional activity. For example, amino acid sequences that contain a common structural domain that is at least about 45%, preferably about 75% through 98%, identical are defined herein as sufficiently similar. Variants include variants of targeting constructs encoded by a polynucleotide that hybridizes to a polynucleotide of this invention or a complement thereof under stringent conditions. Such variants generally retain the functional activity of the targeting constructs of this invention. Libraries of fragments of the polynucleotides can be used to generate a variegated population of fragments for screening and subsequent selection. For example, a library of fragments can be generated by treating a double-stranded PCR fragment of a polynucleotide with a nuclease under conditions wherein nicking occurs only about once per molecule, denaturing the double-stranded DNA, renaturing the DNA to form double-stranded DNA which can include sense/antisense pairs from different nicked products, removing single-stranded portions from reformed duplexes by treatment with S1 nuclease, and ligating the resulting fragment library into an expression vector. By this method, one can derive an expression library that encodes N-terminal and internal fragments of various sizes of the targeting constructs of this invention.

Variants include targeting constructs that differ in amino acid sequence due to mutagenesis. In addition, bioequivalent analogs of the targeting constructs may also be constructed by making various substitutions on residues or sequences in the targeting moiety and/or the active moiety.

In some embodiments, targeting construct is fused at its N-terminus to a signal peptide. Such signal peptides are useful for the secretion of the targeting construct. Suitable signal peptides include, for example, the signal peptide of the CD5 protein (such as signal peptide of the human CD5 protein MPMGSLQPLATLYLLGMLVAS, SEQ ID NO:54). In some embodiments, the signal peptide of the CR2 protein is used. For example, in some embodiments, the signal peptide of the human CR2 protein (MGAAGLLGVFLALVAPG, SEQ ID NO:55 or MGAAGLLGVFLALVAPGVLG, SEQ ID NO:56) is used.

Targeting Construct Production Methods

The targeting construct described herein can be produced using a variety of techniques known in the art of molecular biology and protein chemistry. For example, a nucleic acid encoding a targeting construct described herein can be inserted into an expression vector that contains transcriptional and translational regulatory sequences, which include, e.g., promoter sequences, ribosomal binding sites, transcriptional start and stop sequences, translational start and stop sequences, transcription terminator signals, polyadenylation signals, and enhancer or activator sequences. The regulatory sequences include a promoter and transcriptional start and stop sequences. In addition, the expression vector can include more than one replication system such that it can be maintained in two different organisms, for example in mammalian or insect cells for e2xpression and in a prokaryotic host for cloning and amplification.

Several possible vector systems are available for the expression of targeting constructs from nucleic acids in mammalian cells. One class of vectors relies upon the integration of the desired gene sequences into the host cell genome. Cells which have stably integrated DNA can be selected by simultaneously introducing drug resistance genes such as E. coli gpt (Mulligan and Berg (1981) Proc Natl Acad Sci USA 78:2072) or Tn5 neo (Southern and Berg (1982) Mol Appl Genet 1:327). The selectable marker gene can be either linked to the DNA gene sequences to be expressed, or introduced into the same cell by co-transfection (Wigler et al. (1979) Ce1116:77). A second class of vectors utilizes DNA elements which confer autonomously replicating capabilities to an extrachromosomal plasmid. These vectors can be derived from animal viruses, such as bovine papillomavirus (Sarver et al. (1982) Proc Natl Acad Sci USA, 79:7147), polyoma virus (Deans et al. (1984) Proc Natl A cad Sci USA 81: 1292), or SV 40 virus (Lusky and Botchan (1981) Nature 293:79).

The expression vectors can be introduced into cells in a manner suitable for subsequent expression of the nucleic acid. The method of introduction is largely dictated by the targeted cell type, discussed below. Exemplary methods include CaPO4 precipitation, liposome fusion, lipofectin, electroporation, viral infection, dextran-mediated transfection, polybrene-mediated transfection, protoplast fusion, and direct microinjection.

Appropriate host cells for the expression of the targeting constructs include yeast, bacteria, insect, plant, and, as described above, mammalian cells. Of interest are bacteria such as E. coli, fungi such as Saccharomyces cerevisiae and Pichia pastoris, insect cells such as SF9, mammalian cell lines (e.g., human cell lines), as well as primary cell lines (e.g., primary mammalian cells). In some embodiments, the targeting constructs can be expressed in Chinese hamster ovary (CHO) cells or in a suitable myeloma cell line such as (NSO). Suitable cell lines also include, for example, BHK-21 (baby hamster kidney) cells; 293 (human embryonic kidney) cells; HMEpC (Human Mammary Epithelial cells; 3T3 (mouse embryonic fibroblast) cells.

The targeting moiety and the one or more active moieties may optionally be directly joined to each other, or may optionally be joined via a linker. Where the targeting and active moieties are directly joined, the hybrid vector is made where the DNA encoding the targeting and active moieties are themselves directly ligated to each other using known scientific methods. Where a linker is used, the hybrid vector is made where the DNA encoding the targeting moiety is ligated to DNA encoding one end of the linker; and the DNA encoding the active moiety is ligated to the other end of the linker. Methods are known for performing such ligations in proper orientation. Such ligation may be performed either in series, or as a three way ligation. Examples of sequences which may serve as the linker sequence in the present invention include short peptides of about 2 to about 16 amino acids in length. Among the peptide sequences useful as linkers in the present invention are (Gly-Ser)n, where n=1 to 8; (GlyGlyGlySer)n, where n=1 to 4; (GlySerSerGly)n, where n=1 to 4. Other examples of sequences useful as the linker sequence in the present invention include one or more short conserved region (SCR) domains from one or more of the following complement-related proteins: Factor H; complement receptor 1; complement receptor 2; Factor B; DAF; and others.

As will be recognized by the skilled artisan, many active moieties which may be used in the present invention occur in nature as secreted proteins in conjunction with a signal or leader peptide and/or as a pro-peptide which undergoes further intra- or extra-cellular processing. In such cases, the hybrid vectors of the present invention may include one or more DNA sequences encoding such signal or leader peptides and/or one or more DNA sequences encoding such propeptide sequence, depending upon whether such secretion and/or processing is desired. Alternatively, the hybrid vectors of the present disclosure may include DNA sequences encoding a different signal or leader peptide and/or pro-peptide sequence chosen to optimize the expression and localization of the targeting construct. In most cases, the signal peptide may be omitted, as the targeting moiety will supply sufficient information for targeting of the active moiety to the desired tissue and cells within the subject's body.

In some embodiments, a targeting construct described herein can be expressed in, and purified from, transgenic animals (e.g., transgenic mammals). For example, a targeting construct described herein can be produced in transgenic non-human mammals (e.g., rodents, sheep or goats) and isolated from milk as described in, e.g., Houdebine (2002) Curr Opin Biotechnol 13(6):625-629; van Kuik-Romeijn et al. (2000) Transgenic Res 9(2): 155-159; and Pollock et al. (1999) 1 Immunol Methods 231(1-2):147-157. Additional methods for producing proteins in mammalian milk products are described in, e.g., U.S. patent application publication nos. 200600105347 and 20040006776 and U.S. Pat. No. 7,045,676.

The targeting constructs described herein can be produced from cells by culturing a host cell transformed with the expression vector containing nucleic acid encoding the antibodies, under conditions, and for an amount of time, sufficient to allow expression of the proteins. Such conditions for protein expression will vary with the choice of the expression vector and the host cell, and will be easily ascertained by one skilled in the art through routine experimentation. For example, polypeptides expressed in E. coli can be refolded from inclusion bodies (see, e.g., Hou et al. (1998) Cytokine 10:319-30). Bacterial expression systems and methods for their use are well known in the art (see Current Protocols in Molecular Biology, Wiley & Sons, and Molecular Cloning—A Laboratory Manual—3rd Ed., Cold Spring Harbor Laboratory Press, New York (2001)). The choice of codons, suitable expression vectors and suitable host cells will vary depending on a number of factors, and may be easily optimized as needed. A targeting construct described herein can be expressed in mammalian cells or in other expression systems including but not limited to yeast, baculovirus, and in vitro expression systems (see, e.g., Kaszubska et al. (2000) Protein Expression and Purification 18:213-220).

Following expression, the targeting construct can be isolated. The term “purified” or “isolated” as applied to any of the proteins described herein (e.g., a targeting construct, a targeting moiety, and/or an active moiety) refers to a polypeptide that has been separated or purified from components (e.g., proteins or other naturally-occurring biological or organic molecules) which naturally accompany it, e.g., other proteins, lipids, and nucleic acid in a prokaryote expressing the proteins. Typically, a polypeptide is purified when it constitutes at least 60 (e.g., at least 65, 70, 75, 80, 85, 90, 92, 95, 97, or 99) %, by weight, of the total protein in a sample.

A targeting construct described herein can be isolated or purified in a variety of ways known to those skilled in the art depending on what other components are present in the sample. Standard purification methods include electrophoretic, molecular, immunological, and chromatographic techniques, including ion exchange, hydrophobic, affinity, and reverse-phase HPLC chromatography. For example, a targeting construct can be purified using a standard anti-targeting construct antibody affinity column. Ultrafiltration and diafiltration techniques, in conjunction with protein concentration, are also useful. See, e.g., Scopes (1994) “Protein Purification, 3rd edition,” Springer-Verlag, New York City, N.Y. The degree of purification necessary will vary depending on the desired use. In some instances, no purification of the expressed polypeptide thereof will be necessary.

Methods for determining the yield or purity of a purified polypeptide are known in the art and include, e.g., Bradford assay, UV spectroscopy, Biuret protein assay, Lowry protein assay, amido black protein assay, high pressure liquid chromatography (HPLC), mass spectrometry (MS), and gel electrophoretic methods (e.g., using a protein stain such as Coomassie Blue or colloidal silver stain).

In some embodiments, a targeting construct described herein can be synthesized de novo in whole or in part, using chemical methods well known in the art. For example, the component amino acid sequences can be synthesized by solid phase techniques, cleaved from the resin, and purified by preparative high performance liquid chromatography followed by chemical linkage to form a desired polypeptide. The composition of the synthetic peptides may be confirmed by amino acid analysis or sequencing.

Once expressed and/or purified, a targeting construct described herein can be assayed for any one of a numbered of desired properties using in vitro or in vivo assays such as any of those described herein. For example, a targeting construct described herein can be assayed for its ability to inhibit complement activity as described in.

In some embodiments, endotoxin can be removed from the targeting construct preparations. Methods for removing endotoxin from a protein sample are known in the art. For example, endotoxin can be removed from a protein sample using a variety of commercially available reagents including, without limitation, the ProteoSpin™ Endotoxin Removal Kits (Norgen Biotek Corporation), Detoxi-Gel Endotoxin Removal Gel (Thermo Scientific; Pierce Protein Research Products), MiraCLEAN® Endotoxin Removal Kit (Minis), or Acrodisc™-Mustang® E membrane (Pall Corporation).

Methods for detecting and/or measuring the amount of endotoxin present in a sample (both before and after purification) are known in the art and commercial kits are available. For example, the concentration of endotoxin in a protein sample can be determined using the QCL-1000 Chromogenic kit (BioWhittaker), the limulus amebocyte lysate (LAL)-based kits such as the Pyrotell®, Pyrotell®-T, Pyrochrome®, Chromo-LAL, and CSE kits available from the Associates of Cape Cod Incorporated.

Following expression and purification, the targeting constructs described herein can be modified. The modifications can be covalent or non-covalent modifications. Such modifications can be introduced into the targeting constructs by, e.g., reacting targeted amino acid residues in the targeting moiety and/or the active moiety with an organic derivatizing agent that is capable of reacting with selected side chains or terminal residues. Suitable sites for modification can be chosen using any of a variety of criteria including, e.g., structural analysis or amino acid sequence analysis of the targeting constructs described herein.

In some embodiments, a targeting construct described herein can be conjugated to a heterologous moiety. In embodiments where the heterologous moiety is a polypeptide, a targeting construct and a corresponding heterologous moiety described herein can be joined by way of fusion protein. The heterologous moiety can be, e.g., a heterologous polypeptide, a therapeutic agent (e.g., a toxin or a drug), or a detectable label such as, but not limited to, a radioactive label, an enzymatic label, a fluorescent label, or a luminescent label. Suitable heterologous polypeptides include, e.g., an antigenic tag (e.g., FLAG, polyhistidine, hemagglutinin (HA), glutathione-S-transferase (GST), or maltose-binding protein (MBP)) for use in purifying the targeting constructs. Heterologous polypeptides also include polypeptides that are useful as diagnostic or detectable markers, for example, luciferase, green fluorescent protein (GFP), or chloramphenicol acetyl transferase (CAT). Where the heterologous moiety is a polypeptide, the moiety can be incorporated into a fusion protein described herein, resulting in a fusion protein.

In some embodiments, a targeting construct described herein can be conjugated to a detectable moiety. In embodiments where the detectable moiety is a polypeptide (e.g., GFP), a targeting moiety and a corresponding detectable moiety described herein can be joined by way of fusion protein. The detectable moiety can be, e.g., a heterologous polypeptide, a therapeutic agent (e.g., a toxin or a drug), or a detectable label such as, but not limited to, a radioactive label, an enzymatic label, a fluorescent label, or a luminescent label. Suitable heterologous polypeptides include, e.g., an antigenic tag (e.g., FLAG, polyhistidine, hemagglutinin (HA), glutathione-S-transferase (GST), or maltose-binding protein (MBP)) for use in purifying the targeting constructs. Heterologous polypeptides also include polypeptides that are useful as diagnostic or detectable markers, for example, luciferase, green fluorescent protein (GFP), or chloramphenicol acetyl transferase (CAT).

Conjugates

In some embodiments, the fusion molecules described herein are created by linkage of two independently produced polypeptide fragments, e.g., an antibody (e.g., a Fab fragment of a B4 or C2 antibody) and a complement modulator polypeptide (e.g., a soluble form of CD59). In certain embodiments, the targeting moiety is conjugated to the active moiety through a lysine, cysteine, glutamate, aspartate, or arginine amino acid. A targeting moiety can be conjugated to an active moiety through, e.g., a reaction comprising a thiolated targeting moiety, and a maleoyl-activated amine of the active moiety; an EDC/NHS-activated targeting moiety, and an amine of the active moiety; or an EDC/NHS-activated carboxylic acid of the active moiety and an amine of the targeting moiety. Two proteins (e.g., a targeting construct described herein and a heterologous moiety or the two constituent parts of a targeting construct) can, in some embodiments, be chemically cross-linked using any of a number of known chemical cross linkers. Examples of such cross linkers are those which link two amino acid residues via a linkage that includes a “hindered” disulfide bond. In these linkages, a disulfide bond within the cross-linking unit is protected (by hindering groups on either side of the disulfide bond) from reduction by the action, for example, of reduced glutathione or the enzyme disulfide reductase. One suitable reagent, 4-succinimidyloxycarbonyla-methyl-a (2-pyridyldithio) toluene (SMPT), forms such a linkage between two proteins utilizing a terminal lysine on one of the proteins and a terminal cysteine on the other. Heterobifunctional reagents that cross-link by a different coupling moiety on each protein can also be used. Other useful cross-linkers include, without limitation, reagents which link two amino groups (e.g., N-5-azido-2-nitrobenzoyloxysuccinimide), two sulfhydryl groups (e.g., 1,4-bis-maleimidobutane), an amino group and a sulfhydryl group (e.g., m-maleimidobenzoyl-N-hydroxysuccinimide ester), an amino group and a carboxyl group (e.g., 4-[pazidosalicylamido]butylamine), and an amino group and a guanidinium group that is present in the side chain of arginine (e.g., p-azidophenyl glyoxal monohydrate).

In some embodiments, a fusion protein described herein can contain a heterologous moiety which is chemically linked to the fusion protein. For example, in some embodiments, a drug described herein, a fluorescent label, a paramagnetic label, a radioactive label, etc., can be directly conjugated to the amino acid backbone of the targeting construct and/or targeting moiety (e.g., for use of the labeled targeting construct for in vivo imaging studies).

In some embodiments, the targeting constructs can be modified, e.g., with a moiety that improves the stabilization and/or retention of the targeting constructs in circulation, e.g., in blood, serum, or other tissues. For example, a targeting construct described herein can be PEGylated as described in, e.g., Lee et al. (1999) Bioconjug Chem 10(6): 973-8; Kinstler et al. (2002) Advanced Drug Deliveries Reviews 54:477-485; and Roberts et al. (2002) Advanced Drug Delivery Reviews 54:459-476. The stabilization moiety can improve the stability, or retention of, targeting construct by at least 1.5 (e.g., at least 2, 5, 10, 15, 20, 25, 30, 40, or 50 or more) fold.

In some embodiments, the targeting constructs described herein can be glycosylated. In some embodiments, a targeting construct described herein can be subjected to enzymatic or chemical treatment, or produced from a cell, such that the targeting construct, targeting moiety, and/or active moiety has reduced or absent glycosylation. Methods for producing polypeptides with reduced glycosylation are known in the art and described in, e.g., U.S. Pat. No. 6,933,368; Wright et al. (1991) EMBO J 10(10):2717-2723; and Co et al. (1993) Mol Immunol 30:1361-1367.

Pharmaceutical Compositions

Also provided herein are pharmaceutical compositions comprising an antibody (or antigen-binding fragment thereof) and/or construct (e.g., a targeting construct) described herein and a pharmaceutically acceptable carrier. The pharmaceutical compositions may be suitable for a variety of modes of administration described herein, including for example systemic or localized administration. The pharmaceutical compositions can be in the form of eye drops, injectable solutions, or in a form suitable for inhalation (either through the mouth or the nose) or oral administration. The pharmaceutical compositions described herein can be packaged in single unit dosages or in multidosage forms.

In some embodiments, the pharmaceutical compositions comprise an antibody (or antigen-binding fragment thereof) and/or construct (e.g., a targeting construct) described herein and a pharmaceutically acceptable carrier suitable for administration to human. In some embodiments, the pharmaceutical compositions comprise a targeting construct and a pharmaceutically acceptable carrier suitable for intraocular injection. In some embodiments, the pharmaceutical compositions comprise a targeting construct and a pharmaceutically acceptable carrier suitable for topical application to the eye. In some embodiments, the pharmaceutical compositions comprise a targeting construct and a pharmaceutically acceptable carrier suitable for intravenous injection. In some embodiments, the pharmaceutical compositions comprise a targeting construct and a pharmaceutically acceptable carrier suitable for injection into the arteries (such as renal arteries).

The compositions are generally formulated as sterile, substantially isotonic, and in full compliance with all Good Manufacturing Practice (GMP) regulations of the U.S. Food and Drug Administration. In some embodiments, the composition is free of pathogen. For injection, the pharmaceutical composition can be in the form of liquid solutions, for example in physiologically compatible buffers such as Hank's solution or Ringer's solution. In addition, the targeting construct pharmaceutical composition can be in a solid form and redissolved or suspended immediately prior to use. Lyophilized compositions are also included.

For oral administration, the pharmaceutical compositions can take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate.

The present invention in some embodiments provides compositions comprising an antibody (or antigen-binding fragment thereof) and/or construct (e.g., a targeting construct) and a pharmaceutically acceptable carrier suitable for administration to the eye. Such pharmaceutical carriers can be sterile liquids, such as water and oil, including those of petroleum, animal, vegetable or synthetic origin, such as peanut oil, soybean oil, mineral oil, and the like. Saline solutions and aqueous dextrose, polyethylene glycol (PEG) and glycerol solutions can also be employed as liquid carriers, particularly for injectable solutions. Suitable pharmaceutical excipients include starch, glucose, lactose, sucrose, gelatin, malt, rice, sodium state, glycerol monostearate, glycerol, propylene, water, and the like. The pharmaceutical composition, if desired, can also contain minor amounts of wetting or emulsifying agents, or pH buffering agents. The targeting construct and other components of the composition may be encased in polymers or fibrin glues to provide controlled release of the targeting construct. These compositions can take the form of solutions, suspensions, emulsions, ointment, gel, or other solid or semisolid compositions, and the like. The compositions typically have a pH in the range of 4.5 to 8.0. The compositions must also be formulated to have osmotic values that are compatible with the aqueous humor of the eye and ophthalmic tissues. Such osmotic values will generally be in the range of from about 200 to about 400 milliosmoles per kilogram of water (“mOsm/kg”), but will preferably be about 300 mOsm/kg. The retina is considered to have an osmotic value of ˜283 mOsm/kg.

In some embodiment, the composition is formulated in accordance with routine procedures as a pharmaceutical composition adapted for injection intravenously, introperitoneally, or intravitreally. Typically, compositions for injection are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions may further comprise additional ingredients, for example preservatives, buffers, tonicity agents, antioxidants and stabilizers, nonionic wetting or clarifying agents, viscosity-increasing agents, and the like.

Suitable preservatives for use in a solution include polyquaternium-1, benzalkonium chloride, thimerosal, chlorobutanol, methyl paraben, propyl paraben, phenylethyl alcohol, edetate disodium, sorbic acid, benzethonium chloride, and the like. Typically (but not necessarily) such preservatives are employed at a level of from 0.001% to 1.0% by weight.

Suitable buffers include boric acid, sodium and potassium bicarbonate, sodium and potassium borates, sodium and potassium carbonate, sodium acetate, sodium biphosphate and the like, in amounts sufficient to maintain the pH at between about pH 6 and pH 8, and preferably, between about pH 7 and pH 7.5.

Suitable tonicity agents are dextran 40, dextran 70, dextrose, glycerin, potassium chloride, propylene glycol, sodium chloride, and the like, such that the sodium chloride equivalent of the ophthalmic solution is in the range 0.9 plus or minus 0.2%.

Suitable antioxidants and stabilizers include sodium bisulfite, sodium metabisulfite, sodium thiosulfite, thiourea and the like. Suitable wetting and clarifying agents include polysorbate 80, polysorbate 20, poloxamer 282 and tyloxapol. Suitable viscosity-increasing agents include dextran 40, dextran 70, gelatin, glycerin, hydroxyethylcellulose, hydroxmethylpropylcellulose, lanolin, methylcellulose, petrolatum, polyethylene glycol, polyvinyl alcohol, polyvinylpyrrolidone, carboxymethylcellulose and the like.

The use of viscosity enhancing agents to provide topical compositions with viscosities greater than the viscosity of simple aqueous solutions may be desirable to increase ocular absorption of the active compounds by the target tissues or increase the retention time in the eye. Such viscosity building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose or other agents know to those skilled in the art. Such agents are typically employed at a level of from 0.01% to 2% by weight.

In some embodiments, there is provided a pharmaceutical composition for delivery of a nucleotide encoding a targeting construct. The pharmaceutical composition for gene therapy can be in an acceptable diluent, or can comprise a slow release matrix in which the gene delivery vehicle or compound is imbedded. Alternatively, where the complete gene delivery system can be produced intact from recombinant cells, e.g., retroviral vectors, the pharmaceutical composition can comprise one or more cells which produce the gene delivery system.

In clinical settings, a gene delivery system for a gene therapeutic can be introduced into a subject by any of a number of methods. For instance, a pharmaceutical composition of the gene delivery system can be introduced systemically, e.g., by intravenous injection, and specific transduction of the protein in the target cells occurs predominantly from specificity of transfection provided by the gene delivery vehicle, cell-type or tissue-type expression due to the transcriptional regulatory sequences controlling expression of the receptor gene, or a combination thereof. In other embodiments, initial delivery of the recombinant gene is more limited with introduction into the animal being quite localized. For example, the gene delivery vehicle can be introduced by catheter, See U.S. Pat. No. 5,328,470, or by stereotactic injection, Chen et al. (1994), Proc. Natl. Acad. Sci., USA 91: 3054-3057. A polynucleotide encoding a targeting construct can be delivered in a gene therapy construct by electroporation using techniques described, Dev et al. (1994), Cancer Treat. Rev. 20:105-115.

In some embodiments, there is provided a pharmaceutical composition for gene delivery to the eye. Ophthalmic solutions useful for storing and/or delivering expression vectors have been disclosed, for example, in WO03077796A2.

Diseases to be Treated

The treatment and diagnosis methods described herein can be used for treating or diagnosing a variety of diseases, including, but not limited to, inflammatory diseases, transplant rejections, pregnancy-related diseases, adverse drug reactions, tissue damage resulting from ischemia-reperfusion injury, ocular diseases, kidney diseases, joint diseases, and autoimmune or immune complex disorders. In some embodiments, the disease to be treated or diagnosed include, but not limited to, systemic lupus erythematosus and glomerulonephritis, rheumatoid arthritis, cardiopulmonary bypass and hemodialysis, hyperacute rejection in organ transplantation, myocardial infarction, ischemia/reperfusion injury, antibody-mediated allograft rejection, for example, in the kidneys, and adult respiratory distress syndrome. Moreover, other inflammatory conditions and autoimmune/immune complex diseases are also closely associated with complement activation, including, but not limited to, thermal injury, severe asthma, anaphylactic shock, bowel inflammation, urticaria, angioedema, vasculitis, multiple sclerosis, myasthenia gravis, myocarditis, membranoproliferative glomerulonephritis, atypical hemolytic uremic syndrome, Sjogren's syndrome, renal and pulmonary ischemia/reperfusion, and other organ-specific inflammatory disorders. Accordingly, in some embodiments, the methods described herein are particularly useful for treating or diagnosing a complement-mediated disease including, but not limited to, inflammatory disease, a transplant rejection, pregnancy-related disease, adverse drug reaction, tissue damage resulting from ischemia-reperfusion injury, ocular disease, kidney disease, joint disease, or an autoimmune or immune complex disorder. In some embodiments, also provided herein are methods of treating or diagnosing a complement-mediated disease in an individual, comprising administering to the individual an effective amount of any of the compositions (such as composition comprising a targeting construct) described herein.

The methods described herein are particularly useful for treating or diagnosing inflammatory diseases including, but not limited to, burns, endotoxemia, septic shock, adult respiratory distress syndrome, cardiopulmonary bypass, hemodialysis, anaphylactic shock, asthma, angioedema, Crohn's disease, sickle cell anemia, poststreptococcal glomerulonephritis, membranous nephritis, pancreatitis, rheumatoid arthritis, inflammatory arthritis, inflammatory bowel disease, acute lung injury, and disseminated intravascular coagulation (DIC). In some embodiments, the inflammation (such as complement mediated inflammation) is associated with tissue damage resulting from inflammatory or autoinflammatory disorders, transplant rejection (cellular or antibody mediated), pregnancy-related diseases, adverse drug reactions, degenerative, neovascular, hemolytic, thrombotic, vasculitic, arthritic, regenerative, traumatic, autoimmune or immune complex disorders.

The compositions described herein are also useful for treating or diagnosing a transplant rejection including, but not limited to, a hyperacute transplant rejection, antibody-mediated transplant rejection, cellular-mediated transplant rejection, acute transplant rejection, and chronic transplant rejection. In some embodiments, the transplant is a xenograft, an allograft, or an isograft. In some embodiments, the transplant is a fluid, a cell, a tissue or an organ. In some embodiments, the transplant is selected from the group consisting of: a heart, liver, kidney, lung, pancreas, intestine, stomach, testis, hand, arm, leg, uterus, ovary, and thymus. In some embodiments, the transplant is selected from the group consisting of: a bone, tendons, cornea, skin, heart valve, islets of Langerhans, bone marrow, hematopoietic stem cell, blood transfusion, or vein. In some embodiments, the transplant is a heart, liver or kidney. Transplant rejections can result in several complications such as graft-versus-host disease. In some embodiments, a complement-mediated disease is graft-versus-host disease.

The methods described herein are also particularly useful for treating or diagnosing a pregnancy-related disease including, but not limited to, HELLP (Hemolytic anemia, elevated liver enzymes, and low platelet count), recurrent fetal loss, atypical hemolytic uremic syndrome, fetal hypoxia syndrome, hypertensive disease, and pre-eclampsia.

In addition, the methods described herein are useful for treating or diagnosing an adverse drug reaction including, but not limited to, a drug allergy, a radiographic contrast media allergy, and IL-2 induced vascular leakage.

The methods described herein are also useful for treating or diagnosing tissue damage resulting from ischemia-reperfusion injury following, but not limited to, acute myocardial infarction, aneurysm, aneurysm repair, deep hypothermic circulatory arrest, tourniquet use, solid organ transplant, stroke including perinatal stroke, hemorrhagic shock, crush injury, multiple organ failure, hemodialysis, hypovolemic shock, spinal cord injury, traumatic brain injury, intestinal ischemia, retinal ischemia, cardiopulmonary bypass, emergency coronary surgery for failed percutaneous transluminal coronary angioplasty (PCTA), and any vascular surgery with blood vessel cross clamping, pancreatitis after manipulation of pancreatic or bile duct. In some embodiments, tissue damage can be treated before, during, or after the ischemic event (such as intestinal ischemia) that triggers ischemia-reperfusion injury.

In some embodiments, tissue damage is treated or diagnosed with any of the methods disclosed herein by administering a targeting construct (or a composition comprising the targeting construct) disclosed herein before reperfusion. In some embodiments, tissue damage is treated or diagnosed with any of the methods disclosed herein by administering a targeting construct (or a composition comprising the targeting construct) disclosed herein after reperfusion. In some embodiments, the ischemia-reperfusion injury is selected from the group consisting of: myocardial ischemia-reperfusion, renal ischemia-reperfusion injury, gastrointestinal ischemia-reperfusion injury, hepatic ischemia-reperfusion injury, skeletal muscle ischemia-reperfusion injury, cerebral ischemia-reperfusion injury, pulmonary ischemia-reperfusion injury, intestine ischemia-reperfusion injury, retinal ischemia-reperfusion injury, and joint ischemia-reperfusion injury. In some embodiments, tissue damage is caused by oxidative damage.

There are instances when a therapy or surgery induces a reperfusion but not an ischemia (referred herein as non-ischemia reperfusion injury). Such therapy or surgery includes, but is not limited to, pharmacological thrombolysis, including intravenous and endovascular therapies for stroke, acute coronary syndromes, peripheral arterial occlusion, pulmonary embolus, renal artery occlusion, mechanical thrombolysis, e.g. percutaneous coronary intervention, peripheral arterial angioplasty, visceral arterial angioplasty, coronary artery bypass grafting, carotid endarterectomy, mesenteric ischemia, shock including hemorrhagic, cardiogenic, neurogenic, analphylactic, flap-failure, e.g. plastic surgery, re-implantation of digits and limbs, and strangulated bowel. Accordingly, in some embodiments, tissue damage resulting from non-ischemia reperfusion injury is treated or diagnosed with any of the methods disclosed herein by administering a targeting construct (or a composition comprising the targeting construct) disclosed herein.

The methods described herein are also particularly useful for treating or diagnosing a kidney disease including, but not limited to, acute kidney injury, hemolytic uremic syndrome, glomerulonephritis, membranous glomerulonephritis, mesangioproliferative glomerulonephritis, acute postinfectious glomerulonephritis (such as poststreptococcal glomerulonephritis), cryoglobulinemic glomerulonephritis, lupus nephritis, membranoproliferative glomerulonephritis (such as mesangiocapillary glomerulonephritis), dense deposit disease, minimal change disease, diabetic nephropathy, Henoch-Schonlein purpura nephritis, IgA nephropathy, chronic kidney disease, delayed graft function of a kidney transplant, acute and chronic renal transplant rejection, proteinuric renal disease and nephrotic syndrome, hypertensive kidney disease, and focal segmental glomerulosclerosis. In some embodiments, the kidney disease is a glomerular disease. For example, the methods are useful for treating or diagnosing glomerular disease that leads to binding of natural IgM to damaged glomerulus. In some embodiments, damaged glomerulus can be a result of mechanical, metabolic, chemical, oxidative or immunologic stress. In some embodiments, damaged glomerulus can be a result of ischemia, diabetes, hypertension, and secondary focal segmental glomerulosclerosis. Symptoms of damaged glomerulus include an inflammatory response such as cytokine release and fibrosis such as collagen mesangial matrix deposition, tubular cell damage, and tubulointerstitial fibrosis. The methods are also useful for treating or diagnosing kidney disease such a glomerulonephritis which is inflammation of the glomerulus. Glomerulonephritis is commonly associated with deposition of electron dense material in the glomerulus which contains complement components, including C3. The methods are also useful for treating or diagnosing acute kidney injury associated with renal ischemia. Ischemia is the leading cause of acute kidney injury. Ischemia and subsequent reperfusion elicit acute kidney injury through endothelial dysfunction, leukocyte-mediated inflammation and decreased microvascular blood flow that can lead to rarefaction of the peritubular capillaries, shifting the fragile balance of oxygen supply and demand to the corticomedullary junction toward a negative oxygen balance. The shift in balance causes a hypoxic environment and can lead to accumulation of fibrosis and subsequent development of chronic kidney disease. In some embodiments, the kidney disease is due to a factor H deficiency.

The methods described herein are also useful for treating or diagnosing a joint disease including, but not limited to, arthritis (such as rheumatoid arthritis) and joint inflammation associated with infection (such as hepatitis B infection), inflammatory disease (such as inflammatory bowel disease) or autoimmune disease (such as systemic lupus erythematosus). In some embodiments, methods provided herein are useful for treating or diagnosing a joint disease including, but not limited to, arthritis, amyloid arthropathy, amyloidosis, ankylosing spondylitis, carpal tunnel syndrome, temporal arteritis, polymyalgia rheumatica, polyarthralgia, tendinitis, Whipple's disease, bursitis, trigeminal neuralgia, fibromyoma, fibrositis, autoimmune arthritis, rheumatoid arthritis, juvenile arthritis, psoriatic arthritis, lupus arthritis, polyarthritis, inflammatory arthritis not resulting from an autoimmune disease or disorder, such as an infectious arthritis, i.e., joint pain, soreness, stiffness and swelling caused by an infectious agent such as bacteria (including mycoplasma), viruses, fungi, septic arthritis, or osteoarthritis. Joint disease can be associated with symptoms such as joint stiffness, pain, weakness, joint fatigue, tenderness and swelling. Accordingly, in some embodiments, symptoms of joint disease can be treated or diagnosed with any of the methods disclosed herein by administering a targeting construct (or a composition comprising the targeting construct) disclosed herein. For example, the compositions are useful for treating or diagnosing arthritis or symptoms of arthritis. In some embodiments, the arthritis is selected from the group consisting of: rheumatoid arthritis, juvenile onset rheumatoid arthritis, psoriatic arthritis, and lupus arthritis. In some embodiments the arthritis is osteoarthritis. In some embodiments, the arthritis is infectious arthritis caused by a bacterial pathogen, such as Haemophilus influenzae, Gonoccous spp., Mycoplasma spp. Meingococcus spp., Pneumococcus spp., Streptococcus spp., Staphyloccus spp., Salmonella spp., Brucella spp., Neisseria spp., Streptobacillus moniliformis (Haverhill fever), Mycobacterium tuberculosis, Treponema pallidum (syphilis), Treponema pertenue (yaws), or Rickettsia spp. In some embodiments, the arthritis is infectious arthritis caused by a viral pathogen, such as a rubella virus, a mumps virus, a varicella-zoster virus, an adenovirus, an echovirus, a herpes simplex virus, a cytomegalovirus, a parvovirus, a retrovirus, and alphavirus, or a hepatitis virus. In some embodiments, the arthritis is infectious arthritis caused by a fungus, such as Coccidioides spp., Histoplasmoa spp., Blastomyces spp., Cryptococcus spp., Candida spp., or Sporothrix spp. As another example, the compositions are useful for treating or diagnosing a joint disease or symptoms of a joint disease. In some embodiments, the joint disease is arthritis, amyloid arthropathy, amyloidosis, ankylosing spondylitis, carpal tunnel syndrome, temporal arteritis, polymyalgia rheumatica, polyarthralgia, tendinitis, Whipple's disease, bursitis, trigeminal neuralgia, fibromyoma, and fibrositis. In some embodiments, the joint disease is associated with arthritis. In some embodiments, the joint disease precedes the development of arthritis. In some embodiments, the joint disease develops due to the onset of arthritis.

Rheumatoid arthritis affects approximately 1% of the population, with women affected three times more commonly than men. Rheumatoid arthritis and juvenile onset rheumatoid arthritis are systemic diseases with numerous pathologic manifestations in addition to their joint inflammatory aspects. In rheumatoid arthritis, these manifestations include vasculitis (inflammation of the blood vessels), which can affect nearly any organ system and can cause numerous pathologic sequelae including polyneuropathy, cutaneous ulceration, and visceral infarction. Pleuropulmonary manifestations include pleuritis, interstitial fibrosis, pleuropulmonary nodules, pneumonitis, and arteritis. Other manifestations include the development of inflammatory rheumatoid nodules on a variety of periarticular structures such as extensor surfaces, as well as on pleura and meninges. Weakness and atrophy of skeletal muscle are common. Many patients with systemic lupus erythematosis also develop joint inflammation referred to as lupus arthritis. Systemic lupus erythematosis is an autoimmune disease of unknown cause in which numerous different cells, tissues, and organs are damaged by pathogenic autoantibodies and immune complexes. Clinical manifestations of systemic lupus erythematosis are numerous and include a variety of maculopapular rashes, nephritis, cerebritis, vasculitis, hematologic abnormalities including cytopenias and coagulopathies, pericarditis, myocarditis, pleurisy, gastrointestinal symptoms, and the aforementioned joint inflammation. Osteoarthritis represents the most common chronic joint disease. It is manifested by pain, stiffness, and swelling of the involved joints. Articular cartilage, responsible for the most critical mechanical functions of the joint, is the major target tissue of osteoarthritis and the breakdown of articular cartilage in osteoarthritis is mediated by various enzymes such as metalloproteinases, plasmin, and cathepsin, which are in turn stimulated by various factors that can also act as inflammatory mediators. These factors include cytokines such as interleukin-1, which is known to activate the pathogenic cartilage and synovial proteases. Synovial inflammation becomes more frequent as the disease progresses. Psoriatic arthritis is a chronic inflammatory joint disorder that affects 5 to 8% of people with psoriasis. A significant percentage of these individuals (one-fourth) develop progressive destructive disease. Twenty five percent of psoriasis patients with joint inflammation develop symmetric joint inflammation resembling the joint inflammation manifestations of rheumatoid arthritis, and over half of these go on to develop varying degrees of j oint destruction.

The methods described herein are useful for treating or diagnosing an autoimmune or immune complex including, but not limited to, but is not limited to, myasthenia gravis, Alzheimer's disease, multiple sclerosis, emphysema, obesity, neuromyelitis optica, rheumatoid arthritis, osteoarthritis, systemic lupus erythematosus, lupus nephritis, IgG4 associated diseases, insulin-dependent diabetes mellitus, acute disseminated encephalomyelitis, Addison's disease, antiphospholipid antibody syndrome, thrombotic thrombycytopenic purpura, autoimmune hepatitis, Crohn's disease, Goodpasture's syndromes, Graves' disease, Guillain-Barre syndrome, Hashimoto's disease, idiopathic thrombocytopenic purpura, pemphigus, Sjogren's syndrome, Takayasu's arteritis, autoimmune glomerulonephritis, dense deposit disease (also known as membranoproliferative glomerulonephritis type II), membranous disease, paroxysmal nocturnal hemoglobinuria, age-related macular degeneration, diabetic maculopathy, uveitis, retinal degeneration disorders, diabetic nephropathy, focal segmental glomerulosclerosis, ANCA associated vasculitis, hemolytic uremic syndrome, Shiga-toxin-associated hemolytic uremic syndrome, atypical hemolytic uremic syndrome, and inflammation associated cardiopulmonary bypass and hemodialysis. In some embodiments, the disease to be treated or diagnosed is an autoimmune glomerulonephritis, which includes, but is not limited to, immunoglobulin A nephropathy or membranoproliferative glomerularnephritis type I. In some embodiments, an autoimmune or immune complex disorder is an inflammatory disease.

The methods described herein are particularly useful for treating or diagnosing ocular diseases including, but not limited to, age-related macular degeneration (“AMD”), including wet AMD and dry AMD, CMV retinitis, macular edema, uveitis, glaucoma, diabetic retinopathy, retinitis pigmentosa, retinal detachment, proliferative vitreoretinopathy and ocular melanoma. For example, the methods are useful for treating or diagnosing age-related macular degeneration (AMD). AMD is clinically characterized by progressive loss of central vision which occurs as a result of damage to the photoreceptor cells in an area of the retina called the macula. AMD has been broadly classified into two clinical states: a wet form and a dry form, with the dry form making up to 80-90% of total cases. The dry form is characterized clinically by the presence of macular drusen, which are localized deposits between the retinal pigment epithelium (RPE) and the Bruch's membrane, and by geographic atrophy characterized by RPE cell death with overlying photoreceptor atrophy. Wet AMD, which accounts for approximately 90% of serious vision loss, is associated with neovascularization in the area of the macular and leakage of these new vessels. The accumulation of blood and fluid can cause retinal detachment followed by rapid photoreceptor degeneration and loss of vision. It is generally accepted that the wet form of AMD is preceded by and arises from the dry form.

Analysis of the contents of drusen in AMD patients has shown a large number of inflammatory proteins including amyloid proteins, coagulation factors, and a large number of proteins of the complement pathway. A genetic variation in the complement factor H substantially raises the risk of age-related macular degeneration (AMD), suggesting that uncontrolled complement activation underlies the pathogenesis of AMD. Edward et al., Science 2005, 308:421; Haines et al., Science 2005, 308:419; Klein et al., Science 308:385-389; Hageman et al., Proc. Natl. Acad. Sci. USA 2005, 102:7227.

In some embodiments, the methods described herein can be used to treat or diagnose cytomegalovirus (CMV) retinitis. CMV retinitis is an infection that causes inflammation of the photoreceptor cells in the retina. CMV is typically rare in immunocompetent individuals. However, individuals who are immunocompromised, e.g., by diseases, transplants, or chemotherapy, are particularly susceptible to CMV retinitis. Retinitis usually begins in one eye, but often progresses to the other eye. Without treatment, progressive damage to the retina can lead to blindness in 4-6 months or less.

In some embodiments, the methods described herein can be used to treat or diagnose macular edema. Macular edema occurs when fluid and protein deposits collect on or under the macula of the eye, causing it to thicken and swell. The swelling may distort an individual's central vision, as the macula holds tightly packed cones that provide sharp, clear central vision to enable a person to see detail, form, and color that is directly in the direction of gaze. Macular edema can be classified into two types. Cystoid macular edema (CME) involves fluid accumulation in the outer plexiform layer secondary to abnormal perifoveal retinal capillary permeability. Diabetic macular edema (DME) is similarly caused by leaking macular capillaries. DME is the most common cause of visual loss in both proliferative, and non-proliferative diabetic retinopathy.

In certain embodiments, the methods described herein can be used to treat or diagnose uveitis, i.e., inflammation of the uvea (the iris, ciliary body, and choroid of the eye beneath the sclera). Uveitis is typically associated with eye infections, eye injuries, and/or autoimmune disorders. However, in many cases, the cause is unknown. The most common form of uveitis is anterior uveitis, which involves inflammation in iris. Posterior uveitis affects the choroid, a layer of blood vessels and connective tissue in the middle part of the eye. Another form of uveitis is pars planitis. This inflammation affects the narrowed area (pars plana) between the iris and the choroid.

In certain embodiments, the methods described herein can be used to treat or diagnose glaucoma, a group of eye conditions that lead to damage to the optic nerve, and loss of vision. The nerve damage involves loss of retinal ganglion cells in a characteristic pattern. The many different subtypes of glaucoma can all be considered to be a type of optic neuropathy. Raised intraocular pressure (above 21 mmHg or 2.8 kPa) is the most important and only modifiable risk factor for glaucoma. Intraocular pressure is a function of production of liquid aqueous humor by the ciliary processes of the eye, and its drainage through the trabecular meshwork. Aqueous humor flows from the ciliary processes into the posterior chamber, bounded posteriorly by the lens and the zonules of Zinn, and anteriorly by the iris. It then flows through the pupil of the iris into the anterior chamber, bounded posteriorly by the iris and anteriorly by the cornea. From here, the trabecular meshwork drains aqueous humor via Schlemm's canal into scleral plexuses and general blood circulation.

In open/wide-angle glaucoma, flow is reduced through the trabecular meshwork, due to the degeneration and obstruction of the trabecular meshwork, whose original function is to absorb the aqueous humor. Loss of aqueous humor absorption leads to increased resistance and thus a chronic, painless buildup of pressure in the eye. In close/narrow-angle, the iridocorneal angle is completely closed because of forward displacement of the final roll and root of the iris against the cornea, resulting in the inability of the aqueous fluid to flow from the posterior to the anterior chamber and then out of the trabecular network. This accumulation of aqueous humor causes an acute increase of pressure and pain.

In some embodiments, the methods described herein can be used to treat or diagnose diabetic retinopathy, a complication of diabetes that causes damage that results from microvascular retinal changes. Small blood vessels, such as those in the eye, are especially vulnerable to poor blood sugar control. An over accumulation of glucose and/or fructose damages the tiny blood vessels in the retina. Hyperglycemia-induced pericyte death and thickening of the basement membrane lead to increased permeability of the vascular walls, which changes the formation of the blood-retinal barrier. In some individuals, diabetic retinopathy is accompanied by macular edema. As diabetic retinopathy progresses, the lack of oxygen in the retina causes fragile, new, blood vessels to grow along the retina and in the vitreous humour. Without timely treatment, these new blood vessels can bleed, cloud vision, and destroy the retina and/or cause tractional retinal detachment.

In certain embodiments, the methods described herein can be used to treat or diagnose retinitis pigmentosa (RP), a group of inherited, degenerative eye diseases that cause severe vision impairment and blindness. Mutations in more than 60 genes are known to cause retinitis pigmentosa. Approximately 20% of RP is autosomal dominant (ADRP), 20% is autosomal recessive (ARRP), and 10% is X linked (XLRP), while the remaining 50% is found in patients without any known affected relatives. The genes associated with retinitis pigmentosa play essential roles in the structure and function of photoreceptors in the retina, and the progressive degeneration of these cells causes vision loss.

In certain embodiments, the methods described herein can be used to treat or diagnose proliferative vitreoretinopathy, i.e., the formation of scar tissue within the eye that is often a complication of rhegmatogenous retinal detachment. During rhegmatogenous retinal detachment, fluid from the vitreous humor enters a retinal hole. The accumulation of fluid in the subretinal space and the tractional force of the vitreous on the retina result in rhegmatogenous retinal detachment. During this process the retinal cell layers come in contact with vitreous cytokines, which trigger the proliferation and migration of retinal pigmented epithelium (RPE). The RPE cells undergo epithelial-mesenchymal transition (EMT) and develop the ability to migrate out into the vitreous. During this process the RPE cell layer-neural retinal adhesion and RPE-ECM (extracellular matrix) adhesions are lost. The RPE cells lay down fibrotic membranes while they migrate and these membranes contract and pull at the retina, and this can lead to secondary retinal detachment after primary retinal detachment surgery.

In certain embodiments, the treatment methods described herein can be used in conjunction with, e.g., surgery for the repair of a retinal tear, hole or detachment, or with, e.g., radiation therapy for the treatment of ocular melanoma.

In certain embodiments, the compositions and methods described herein can be used to treat and/or improve the outcome of corneal wound healing and/or corneal transplantation. The corneal wound healing response is a complex cascade involving cytokine mediated interactions between the epithelial cells, stromal keratocytes, corneal nerves, lacrimal glands, tear film and cells of the immune system. The response of the tissue changes depends on the inciting injury. For example, incisional, lamellar and surface scrape injuries, like the ones used in keratorefractive surgery procedures, are followed by typical wound healing responses that are similar in some respects, but different in others. For example, elsewhere in the body, wound healing culminates in scar formation and vascularisation whereas one of the most crucial aspects of corneal wound healing is how the healing processes aim to minimize these end results, which would otherwise have serious visual consequences. Causes of corneal scarring include almost any disruption to normal corneal structure and function, whether from infection, laser refractive surgery, corneal transplantation, ocular trauma (chemical or physical) or corneal dystrophies.

Corneal transplantation, also known as corneal grafting, is a surgical procedure where a damaged or diseased cornea is replaced by donated corneal tissue (the graft) in its entirety (penetrating keratoplasty) or in part (lamellar keratoplasty). The graft is taken from a recently deceased individual with no known diseases or other factors that may affect the viability of the donated tissue or the health of the recipient. Since the cornea has no blood vessels (it takes its nutrients from the aqueous humor) it heals much more slowly than a cut on the skin. The risks are similar to other intraocular procedures, but additionally include graft rejection (lifelong), detachment or displacement of lamellar transplants and primary graft failure. There is also a risk of infection.

The present invention provides methods of treating or diagnosing an ocular disease described herein by administering an effective amount of a composition comprising a targeting construct. In some embodiments, the invention provides methods of treating or diagnosing one or more aspects or symptoms of the ocular diseases described herein, including, but not limited to, formation of ocular drusen, inflammation in the eye or eye tissue, loss of photoreceptor cells, loss of vision (including for example visual acuity and visual field), neovascularization (such as choroidal neovascularization or CNV), and retinal detachment. Other related aspects, such as photoreceptor degeneration, RPE degeneration, retinal degeneration, chorioretinal degeneration, cone degeneration, retinal dysfunction, retinal damage in response to light exposure (such as constant light exposure), damage of the Bruch's membrane, loss of RPE function, gain or RPE function, loss of integrity of the histoarchitecture of the cells and/or extracellular matrix of the normal macular, loss of function of the cells in the macula, photoreceptor dystrophy, mucopolysaccharidoses, rod-cone dystrophies, cone-rod dystrophies, anterior and posterior uvitis, and diabetic neuropathy, are also included.

In some embodiments, there are provided methods of treating or diagnosing a drusen-associated disease. The term “drusen-associated disease” refers to any disease in which formation of drusen or drusen-like extracellular disease plaque takes place, and for which drusen or drusen-like extracellular disease plaque causes or contributes to thereto or represents a sign thereof. For example, AMD, characterized by the formation of macular drusen, is considered as a drusen-associated disease. Non-ocular drusen-related disease include, but are not limited to, amyloidosis, elastosis, dense deposit disease, and/or atherosclerosis.

### Ocular Diseases

The compositions and methods described herein are particularly useful for treating ocular diseases. For example, the compositions and methods can be useful in detecting and/or treating ischemia/reperfusion (I/R) injury to the eye. As used herein, the term “ischemia/reperfusion injury” refers to inflammatory injury to the endothelium and underlying parenchymal tissues following reperfusion of hypoxic tissues. It is a general syndrome that is responsible for both acute and chronic injury to various tissues including, for example, myocardium, central nervous system, hind limb, intestine, and eye. Ischemia reperfusion injury can result in necrosis and irreversible cell injury. The complement pathway (including the alternative complement pathway) is a major mediator of I/R injury. The noninvasive methods provided herein are thus useful for detection of complement-mediated inflammation associated with ischemia reperfusion that occurs in eye.

The compositions and methods provided herein may also be used to detect and/or treat complement-mediated inflammation in drusen-associated diseases. For example, age-related macular degeneration (AMD), characterized by the formation of macular drusen, is considered a drusen-associated disease. AMD is clinically characterized by progressive loss of central vision which occurs as a result of damage to the photoreceptor cells in an area of the retina called the macula. AMD has been broadly classified into two clinical states: a wet form and a dry form, with the dry form making up to 80-90% of total cases. The dry form is characterized clinically by the presence of macular drusen, which are localized deposits between the retinal pigment epithelium (RPE) and the Bruch's membrane, and by geographic atrophy characterized by RPE cell death with overlying photoreceptor atrophy. Wet AMD, which accounts for approximately 90% of serious vision loss, is associated with neovascularization in the area of the macular and leakage of these new vessels. The accumulation of blood and fluid can cause retinal detachment followed by rapid photoreceptor degeneration and loss of vision. It is generally accepted that the wet form of AMD is preceded by and arises from the dry form.

Analysis of the contents of drusen in AMD patients has shown a large number of inflammatory proteins including amyloid proteins, coagulation factors, and a large number of proteins of the complement pathway. A genetic variation in the complement factor H substantially raises the risk of age-related macular degeneration (AMD), suggesting that uncontrolled complement activation underlies the pathogenesis of AMD. Edward et al., Science 2005, 308:421; Haines et al., Science 2005, 308:419; Klein et al., Science 308:385-389; Hageman et al., Proc. Natl. Acad. Sci. USA 2005, 102:7227. In addition, lipid accumulation and modifications, as well as the presence of Annexin II, has been reported in pathological structures associated with AMD (see, e.g., references cited above). Animal models of AMD respond favorably to complement therapeutics such as those described here (Rohrer, et al. (2009) Invest Ophthalmol Vis Sci. 50(7):3056-64; Rohrer, et al. (2012) J Ocul Pharmacol Ther. 28(4):402-9).

In some embodiments, the compositions and methods described herein can be used to detect and/or treat cytomegalovirus (CMV) retinitis. CMV retinitis is an infection that causes inflammation of the photoreceptor cells in the retina. CMV is typically rare in immunocompetent individuals. However, individuals who are immunocompromised, e.g., by diseases, transplants, or chemotherapy, are particularly susceptible to CMV retinitis. Retinitis usually begins in one eye, but often progresses to the other eye. Without treatment, progressive damage to the retina can lead to blindness in 4-6 months or less.

In some embodiments, the compositions and methods described herein can be used to detect and/or treat macular edema. Macular edema occurs when fluid and protein deposits collect on or under the macula of the eye, causing it to thicken and swell. The swelling may distort an individual's central vision, as the macula holds tightly packed cones that provide sharp, clear central vision to enable a person to see detail, form, and color that is directly in the direction of gaze. Macular edema can be classified into two types. Cystoid macular edema (CME) involves fluid accumulation in the outer plexiform layer secondary to abnormal perifoveal retinal capillary permeability. Diabetic macular edema (DME) is similarly caused by leaking macular capillaries. DME is the most common cause of visual loss in both proliferative, and non-proliferative diabetic retinopathy.

In certain embodiments, the compositions and methods described herein can be used to detect and/or treat uveitis, i.e., inflammation of the uvea (the iris, ciliary body, and choroid of the eye beneath the sclera). Uveitis is typically associated with eye infections, eye injuries, and/or autoimmune disorders. However, in many cases, the cause is unknown. The most common form of uveitis is anterior uveitis, which involves inflammation in iris. Posterior uveitis affects the choroid, a layer of blood vessels and connective tissue in the middle part of the eye. Another form of uveitis is pars planitis. This inflammation affects the narrowed area (pars plana) between the iris and the choroid.

In certain embodiments, the compositions and methods described herein can be used to detect and/or treat glaucoma, a group of eye conditions that lead to damage to the optic nerve, and loss of vision. The nerve damage involves loss of retinal ganglion cells in a characteristic pattern. The many different subtypes of glaucoma can all be considered to be a type of optic neuropathy. Raised intraocular pressure (above 21 mmHg or 2.8 kPa) is the most important and only modifiable risk factor for glaucoma. Intraocular pressure is a function of production of liquid aqueous humor by the ciliary processes of the eye, and its drainage through the trabecular meshwork. Aqueous humor flows from the ciliary processes into the posterior chamber, bounded posteriorly by the lens and the zonules of Zinn, and anteriorly by the iris. It then flows through the pupil of the iris into the anterior chamber, bounded posteriorly by the iris and anteriorly by the cornea. From here, the trabecular meshwork drains aqueous humor via Schlemm's canal into scleral plexuses and general blood circulation.

In open/wide-angle glaucoma, flow is reduced through the trabecular meshwork, due to the degeneration and obstruction of the trabecular meshwork, whose original function is to absorb the aqueous humor. Loss of aqueous humor absorption leads to increased resistance and thus a chronic, painless buildup of pressure in the eye. In close/narrow-angle, the iridocorneal angle is completely closed because of forward displacement of the final roll and root of the iris against the cornea, resulting in the inability of the aqueous fluid to flow from the posterior to the anterior chamber and then out of the trabecular network. This accumulation of aqueous humor causes an acute increase of pressure and pain.

In some embodiments, the compositions and methods described herein can be used to detect and/or treat diabetic retinopathy, a complication of diabetes that causes damage that results from microvascular retinal changes. Small blood vessels, such as those in the eye, are especially vulnerable to poor blood sugar control. An overaccumulation of glucose and/or fructose damages the tiny blood vessels in the retina. Hyperglycemia-induced pericyte death and thickening of the basement membrane lead to increased permeability of the vascular walls, which changes the formation of the blood-retinal barrier. In some individuals, diabetic retinopathy is accompanied by macular edema. As diabetic retinopathy progresses, the lack of oxygen in the retina causes fragile, new, blood vessels to grow along the retina and in the vitreous humour. Without timely treatment, these new blood vessels can bleed, cloud vision, and destroy the retina and/or cause tractional retinal detachment.

In certain embodiments, the compositions and methods described herein can be used to detect and/or treat retinitis pigmentosa (RP), a group of inherited, degenerative eye diseases that cause severe vision impairment and blindness. Mutations in more than 60 genes are known to cause retinitis pigmentosa. Approximately 20% of RP is autosomal dominant (ADRP), 20% is autosomal recessive (ARRP), and 10% is X linked (XLRP), while the remaining 50% is found in patients without any known affected relatives. The genes associated with retinitis pigmentosa play essential roles in the structure and function of photoreceptors in the retina, and the progressive degeneration of these cells causes vision loss.

In certain embodiments, the compositions and methods described herein can be used to detect and/or treat proliferative vitreoretinopathy, i.e., the formation of scar tissue within the eye that is often a complication of rhegmatogenous retinal detachment. During rhegmatogenous retinal detachment, fluid from the vitreous humor enters a retinal hole. The accumulation of fluid in the subretinal space and the tractional force of the vitreous on the retina result in rhegmatogenous retinal detachment. During this process the retinal cell layers come in contact with vitreous cytokines, which trigger the proliferation and migration of retinal pigmented epithelium (RPE). The RPE cells undergo epithelial-mesenchymal transition (EMT) and develop the ability to migrate out into the vitreous. During this process the RPE cell layer-neural retinal adhesion and RPE-ECM (extracellular matrix) adhesions are lost. The RPE cells lay down fibrotic membranes while they migrate and these membranes contract and pull at the retina, and this can lead to secondary retinal detachment after primary retinal detachment surgery.

In certain embodiments, the compositions described herein can be used in conjunction with, e.g., surgery for the repair of a retinal tear, hole or detachment, or with, e.g., radiation therapy for the treatment of ocular melanoma.

In certain embodiments, the compositions and methods described herein can be used to treat and/or improve the outcome of corneal wound healing and/or corneal transplantation. The corneal wound healing response is a complex cascade involving cytokine mediated interactions between the epithelial cells, stromal keratocytes, corneal nerves, lacrimal glands, tear film and cells of the immune system. The response of the tissue changes depends on the inciting injury. For example, incisional, lamellar and surface scrape injuries, like the ones used in keratorefractive surgery procedures, are followed by typical wound healing responses that are similar in some respects, but different in others. For example, elsewhere in the body, wound healing culminates in scar formation and vascularisation whereas one of the most crucial aspects of corneal wound healing is how the healing processes aim to minimize these end results, which would otherwise have serious visual consequences. Causes of corneal scarring include almost any disruption to normal corneal structure and function, whether from infection, laser refractive surgery, corneal transplantation, ocular trauma (chemical or physical) or corneal dystrophies.

Corneal transplantation, also known as corneal grafting, is a surgical procedure where a damaged or diseased cornea is replaced by donated corneal tissue (the graft) in its entirety (penetrating keratoplasty) or in part (lamellar keratoplasty). The graft is taken from a recently deceased individual with no known diseases or other factors that may affect the viability of the donated tissue or the health of the recipient. Since the cornea has no blood vessels (it takes its nutrients from the aqueous humor) it heals much more slowly than a cut on the skin. The risks are similar to other intraocular procedures, but additionally include graft rejection (lifelong), detachment or displacement of lamellar transplants and primary graft failure. There is also a risk of infection.

The present invention provides methods of detecting and/or treating an ocular disease described herein by administering an effective amount of a composition comprising a targeting construct. In some embodiments, the invention provides methods of treating or preventing one or more aspects or symptoms of the ocular diseases described herein, including, but not limited to, formation of ocular drusen, inflammation in the eye or eye tissue, loss of photoreceptor cells, loss of vision (including for example visual acuity and visual field), neovascularization (such as choroidal neovascularization or CNV), and retinal detachment. Other related aspects, such as photoreceptor degeneration, RPE degeneration, retinal degeneration, chorioretinal degeneration, cone degeneration, retinal dysfunction, retinal damage in response to light exposure (such as constant light exposure), damage of the Bruch's membrane, loss of RPE function, gain or RPE function, loss of integrity of the histoarchitecture of the cells and/or extracellular matrix of the normal macular, loss of function of the cells in the macula, photoreceptor dystrophy, mucopolysaccharidoses, rod-cone dystrophies, cone-rod dystrophies, anterior and posterior uvitis, and diabetic neuropathy, are also included. In some embodiments, the invention provides methods of improving corneal wound healing and/or improving the outcome of corneal transplantations.

In some embodiments, there are provided methods of treating an ocular disease in an individual, e.g., an ocular disease described herein, comprising administering to the individual an effective amount of a composition comprising a targeting construct: a) a targeting moiety comprising a B4 or C2 antibody or a fragment thereof, and b) an active moiety comprising a therapeutic moiety or a fragment thereof. In certain embodiments, the ocular disease is AMD. In certain embodiments, the AMD is wet AMD. In certain embodiments, the AMD is dry AMD. In addition to macular degeneration, other eye diseases that can be treated by methods of the present invention include, for example, retinitis pigmentosa, diabetic retinopathy, and other eye diseases that involve a local inflammatory process. In some embodiments, the eye disease is uveitis (anterior and posterior). In some embodiments, the eye disease is retinitis pigmentosa. In some embodiments, the eye disease involves the cornea. In some embodiments, the eye disease is proliferative vitreoretinopathy, retinal detachment, corneal wound healing, corneal transplants, or ocular melanoma.

In some embodiments, there are provided methods of treating (such as reducing, delaying, eliminating, or preventing) formation of drusen and other extracellular deposits in the eye of an individual, comprising administering to the individual an effective amount of a composition comprising a targeting construct comprising: a) a targeting moiety comprising a B4 or C2 antibody or a fragment thereof, and b) an active moiety, e.g., one or more therapeutic moieties described herein. In some embodiments, there are provided methods of treating (such as reducing, delaying, eliminating, or preventing) inflammation in the eye of an individual, comprising administering to the individual an effective amount of a composition comprising targeting construct comprising: a) a targeting moiety comprising a B4 or C2 antibody or a fragment thereof, and b) an active moiety, e.g., one or more therapeutic moieties described herein. In some embodiments, there are provided methods of treating (such as reducing, delaying, eliminating, or preventing) loss of photoreceptors cells in an individual, comprising administering to the individual an effective amount of a composition comprising a targeting construct comprising: a) a targeting moiety comprising a B4 or C2 antibody or a fragment thereof, and b) an active moiety, e.g., one or more therapeutic moieties described herein. In some embodiments, there are provided methods of treating (such as reducing, delaying, eliminating, or preventing) loss of photoreceptors cells in an individual, comprising administering to the individual an effective amount of a composition comprising a targeting construct comprising: a) a targeting moiety comprising a B4 or C2 antibody or a fragment thereof, and b) an active moiety, e.g., one or more therapeutic moieties described herein. In some embodiments, there are provided methods of treating (such as reducing, delaying, eliminating, or preventing) neovascularization associated with AMD, comprising administering to the individual an effective amount of a composition comprising a targeting construct comprising: a) a targeting moiety comprising a B4 or C2 antibody or a fragment thereof, and b) an active moiety, i.e., one or more therapeutic moieties described herein. In some embodiments, there are provided methods of treating (such as reducing, delaying, eliminating, or preventing) retinal detachment, comprising administering to the individual an effective amount of a composition comprising a targeting construct comprising: a) a targeting moiety comprising a B4 or C2 antibody or a fragment thereof, and b) an active moiety, e.g., one or more therapeutic moieties described herein. In some embodiments, there are provided methods of improving (including for example decreasing, delaying, or blocking loss of) visual acuity or visual field in the eye of an individual, comprising administering to the individual an effective amount of a composition comprising a targeting construct comprising: a) a targeting moiety comprising a B4 or C2 antibody or a fragment thereof, and b) an active moiety, i.e., one or more therapeutic moieties described herein. In some embodiments, there are provided methods of improving corneal wound healing or the outcome of corneal transplantations in the eye of an individual, comprising administering to the individual an effective amount of a targeting construct described herein.

In some embodiments, there are provided methods of treating a drusen-associated disease. The term “drusen-associated disease” refers to any disease in which formation of drusen or drusen-like extracellular disease plaque takes place, and for which drusen or drusen-like extracellular disease plaque causes or contributes to thereto or represents a sign thereof. For example, AMD, characterized by the formation of macular drusen, is considered as a drusen-associated disease. Non-ocular drusen-related disease include, but are not limited to, amyloidosis, elastosis, dense deposit disease, and/or atherosclerosis.

### Modes of Administration

The compositions described herein can be administered to an individual via any route, including, but not limited to, intravenous (e.g., by infusion pumps), intraperitoneal, intraocular, intra-arterial, intrapulmonary, oral, inhalation, intravesicular, intramuscular, intra-tracheal, subcutaneous, intraocular, intrathecal, transdermal, transpleural, intraarterial, topical, inhalational (e.g., as mists of sprays), mucosal (such as via nasal mucosa), subcutaneous, transdermal, gastrointestinal, intraarticular, intracisternal, intraventricular, rectal (i.e., via suppository), vaginal (i.e., via pessary), intracranial, intraurethral, intrahepatic, and intratumoral. In some embodiments, the compositions are administered systemically (for example by intravenous injection). In some embodiments, the compositions are administered locally (for example by intraarterial or intraocular injection).

In some embodiments, the compositions are administered directly to the eye or the eye tissue. As used herein, the term “eye” refers to any and all anatomical tissues and structures associated with an eye. The eye has a wall composed of three distinct layers: the outer sclera, the middle choroid layer, and the inner retina. The chamber behind the lens is filled with a gelatinous fluid referred to as the vitreous humor. At the back of the eye is the retina, which detects light. The cornea is an optically transparent tissue, which conveys images to the back of the eye. The cornea includes one pathway for the permeation of drugs into the eye. Other anatomical tissue structures associated with the eye include the lacrimal drainage system, which includes a secretory system, a distributive system and an excretory system. The secretory system comprises secretors that are stimulated by blinking and temperature change due to tear evaporation and reflex secretors that have an efferent parasympathetic nerve supply and secrete tears in response to physical or emotional stimulation. The distributive system includes the eyelids and the tear meniscus around the lid edges of an open eye, which spread tears over the ocular surface by blinking, thus reducing dry areas from developing.

In some embodiments, the compositions are administered directly to the eye or the eye tissue. In some embodiments, the compositions are administered topically to the eye, for example, in eye drops. In some embodiments, the compositions are administered by injection to the eye (intraocular injection) or to the tissues associated with the eye. The compositions can be administered, for example, by intraocular injection, periocular injection, subretinal injection, intravitreal injection, trans-septal injection, subscleral injection, intrachoroidal injection, intracameral injection, subconjunctival injection, subconjuntival injection, sub-Tenon's injection, retrobulbar injection, peribulbar injection, or posterior juxtascleral delivery. These methods are known in the art. For example, for a description of exemplary periocular routes for retinal drug delivery, see Periocular routes for retinal drug delivery, Raghava et al. (2004), Expert Opin. Drug Deliv. 1(1):99-114. The compositions may be administered, for example, to the vitreous, aqueous humor, sclera, conjunctiva, the area between the sclera and conjunctiva, the choroid tissues, the retina choroids tissues, macula, or other area in or proximate to the eye of an individual. The compositions can also be administered to the individual as an implant. Preferred implants are biocompatible and/or biodegradable sustained release formulations which gradually release the compounds over a period of time. Ocular implants for drug delivery are well-known in the art. See, e.g., U.S. Pat. Nos. 5,501,856, 5,476,511, and 6,331,313. The compositions can also be administered to the individual using iontophoresis, including, but are not limited to, the ionophoretic methods described in U.S. Pat. No. 4,454,151 and U.S. Pat. App. Pub. No. 2003/0181531 and 2004/0058313.

In some embodiments, the compositions are administered intravascularly, such as intravenously (IV) or intraarterially. In some embodiments (for example for the treatment of renal diseases), the compositions are administered directly into arteries (such as renal arteries).

In some embodiments, the compositions are administered directly into the joint tissue. In some embodiments, the compositions are administered to the synovium.

The optimal effective amount of the compositions can be determined empirically and will depend on the type and severity of the disease, route of administration, disease progression and health, mass and body area of the individual. Such determinations are within the skill of one in the art. The effective amount can also be determined based on in vitro complement activation assays. Examples of dosages of antibodies (or antigen-binding fragments thereof) and/or constructs (e.g., targeting constructs) which can be used for methods described herein include, but are not limited to, an effective amount within the dosage range of any of about 0.01 μg/kg to about 300 mg/kg, or within about 0.1 μg/kg to about 40 mg/kg, or with about 1 μg/kg to about 20 mg/kg, or within about 1 μg/kg to about 10 mg/kg. For example, when administered intraocularly, the composition may be administered at low microgram ranges, including for example about 0.1 μg/kg or less, about 0.05 μg/kg or less, or 0.01 μg/kg or less. In some embodiments, the amount of an antibody (or antigen-binding fragment thereof) and/or construct (e.g., a targeting construct) administered to an individual is about 10 μg to about 500 mg per dose, including for example any of about 10 μg to about 50 μg, about 50 μg to about 100 μg, about 100 μg to about 200 μg, about 200 μg to about 300 μg, about 300 μg to about 500 μg, about 500 μg to about 1 mg, about 1 mg to about 10 mg, about 10 mg to about 50 mg, about 50 mg to about 100 mg, about 100 mg to about 200 mg, about 200 mg to about 300 mg, about 300 mg to about 400 mg, or about 400 mg to about 500 mg per dose.

The antibody (or antigen-binding fragment thereof) and/or construct (e.g., targeting construct) compositions may be administered in a single daily dose, or the total daily dose may be administered in divided dosages of two, three, or four times daily. The compositions can also be administered less frequently than daily, for example, six times a week, five times a week, four times a week, three times a week, twice a week, once a week, once every two weeks, once every three weeks, once a month, once every two months, once every three months, or once every six months. The compositions may also be administered in a sustained release formulation, such as in an implant which gradually releases the composition for use over a period of time, and which allows for the composition to be administered less frequently, such as once a month, once every 2-6 months, once every year, or even a single administration. The sustained release devices (such as pellets, nanoparticles, microparticles, nanospheres, microspheres, and the like) may be administered by injection or surgical implanted in various locations in the eye or tissue associated with the eye, such as intraocular, intravitreal, subretinal, periocular, subconjunctival, or sub-tenons.

The antibody (or antigen-binding fragment thereof) and/or construct (e.g., a targeting construct) compositions (e.g, pharmaceutical compositions) can be administered alone or in combination with other molecules known to have a beneficial effect on retinal attachment or damaged retinal tissue, including molecules capable of tissue repair and regeneration and/or inhibiting inflammation. Examples of useful cofactors include anti-VEGF agents (such as an antibody against VEGF), basic fibroblast growth factor (bFGF), ciliary neurotrophic factor (CNTF), axokine (a mutein of CNTF), leukemia inhibitory factor (LIF), neutrotrophin 3 (NT-3), neurotrophin-4 (NT-4), nerve growth factor (NGF), insulin-like growth factor II, prostaglandin E2, 30 kD survival factor, taurine, and vitamin A. Other useful cofactors include symptom-alleviating cofactors, including antiseptics, antibiotics, antiviral and antifungal agents and analgesics and anesthetics.

Gene Therapy

The targeting constructs can also be delivered by expression of the targeting construct fusion protein in vivo, which is often referred to as “gene therapy”. For example, cells may be engineered with a polynucleotide (DNA or RNA) encoding for the fusion protein ex vivo, the engineered cells are then provided to an individual to be treated with the fusion protein. Such methods are well-known in the art. For example, cells may be engineered by procedures known in the art by use of a retroviral particle containing RNA encoding for the fusion protein of the present invention.

Local delivery of the targeting construct of the present invention using gene therapy may provide the therapeutic agent to the target area, for example to the eye or the eye tissue.

The an antibodies (or antigen-binding fragments thereof) and/or constructs (e.g., a targeting constructs) can also be delivered by expression of the antibody and/or targeting construct in vivo, which is often referred to as “gene therapy”. For example, cells may be engineered with a polynucleotide (DNA or RNA) encoding for the antibody and/or targeting construct ex vivo, the engineered cells are then provided to an individual to be treated with the antibody and/or targeting construct. Such methods are well-known in the art. For example, cells may be engineered by procedures known in the art by use of a retroviral particle containing RNA encoding for the antibody and/or targeting construct of the present invention.

Local delivery of the antibody (or antigen-binding fragment thereof) and/or construct (e.g., a targeting construct) of the present invention using gene therapy may provide the therapeutic agent to the target area, for example to the eye or the eye tissue.

Methods of gene delivery are known in the art. These methods include, but are not limited to, direct DNA transfer, see, e.g., Wolff et al. (1990) Science 247: 1465-1468; 2) Liposome-mediated DNA transfer, see, e.g., Caplen et al. (1995) Nature Med. 3:39-46; Crystal (1995) Nature Med. 1:15-17; Gao and Huang (1991) Biochem. Biophys. Res. Comm. 179:280-285; 3) Retrovirus-mediated DNA transfer, see, e.g., Kay et al. (1993) Science 262:117-119; Anderson (1992) Science 256:808-813; 4) DNA Virus-mediated DNA transfer. Such DNA viruses include adenoviruses (preferably Ad2 or Ad5 based vectors), herpes viruses (preferably herpes simplex virus based vectors), and parvoviruses (preferably “defective” or non-autonomous parvovirus based vectors, more preferably adeno-associated virus based vectors, most preferably AAV-2 based vectors). See, e.g., Ali et al. (1994) Gene Therapy 1:367-384; U.S. Pat. No. 4,797,368, incorporated herein by reference, and U.S. Pat. No. 5,139,941.

Retroviruses from which the retroviral plasmid vectors hereinabove mentioned may be derived include, but are not limited to, Moloney Mouse Leukemia Virus, spleen necrosis virus, retroviruses such as Rous Sarcoma Virus, Harvey Sarcoma Virus, avian leukosis virus, gibbon ape leukemia virus, human immunodeficiency virus, adenovirus, Myeloproliferative Sarcoma Virus, and mammary tumor virus. In one embodiment, the retroviral plasmid vector is derived from Moloney Mouse Leukemia Virus.

Adenoviruses have the advantage that they have a broad host range, can infect quiescent or terminally differentiated cells, such as neurons or hepatocytes, and appear essentially non-oncogenic. See, e.g., Ali et al. (1994), supra, p. 367. Adenoviruses do not appear to integrate into the host genome. Because they exist extrachromosomally, the risk of insertional mutagenesis is greatly reduced. Ali et al. (1994), supra, p. 373.

Adeno-associated viruses exhibit similar advantages as adenoviral-based vectors. However, AAVs exhibit site-specific integration on human chromosome 19 (Ali et al. (1994), supra, p. 377).

The gene therapy vectors include one or more promoters. In some embodiments, the vector has a promoter that drives expression in multiple cell types. In some embodiments, the vector has a promoter that drives expression in specific cell types (such as cells of retina or cells in the kidney). Suitable promoters which may be employed include, but are not limited to, the retroviral LTR; the SV40 promoter; and the human cytomegalovirus (CVM) promoter described in Miller et al. (1989) Biotechniques 7(9):980-990, or any other promoter (e.g., cellular promoters such as eukaryotic cellular promoters including, but not limited to, the histone, pol III, and .beta.-actin promoters). Other viral promoters which may be employed include, but are not limited to, adenovirus promoters, thymidine kinase (TK) promoters, and B19 parvovirus promoters. The selection of a suitable promoter will be apparent to those skilled in the art from the teachings contained herein.

The nucleic acid sequence encoding an antibody (or antigen-binding fragment thereof) and/or construct (e.g., a targeting construct) is under the control of a suitable promoter. Suitable promoters which may be employed include, but are not limited to, adenoviral promoters, such as the adenoviral major late promoter; or heterologous promoters, such as the cytomegalovirus (CMV) promoter; the respiratory syncytial virus (RSV) promoter; inducible promoters, such as the MMT promoter, the metallothionein promoter; heat shock promoters; the albumin promoter; the ApoAl promoter; human globin promoters; viral thymidine kinase promoters, such as the Herpes Simplex thymidine kinase promoter; retroviral LTRs (including the modified retroviral LTRs hereinabove described); the (3-actin promoter; and human growth hormone promoter.

Retroviral plasmid vectors can be employed to transduce packaging cell lines to form producer cell lines. Examples of packaging cells which may be transfected are described in Miller (1990) Human Gene Therapy 1:5-14. The vectors may transduce the packaging cells through any means known in the art. Such means include, but are not limited to, electroporation, the use of liposomes, and CaPO4 precipitation. In one alternative, the retroviral plasmid vector may be encapsulated into a liposome, or coupled to a lipid, and then administered to a host. The producer cell line generates infectious retroviral vector particles which include the nucleic acid sequence(s) encoding the polypeptides. Such retroviral vector particles then may be employed, to transduce eukaryotic cells, either in vitro or in vivo. The transduced eukaryotic cells will express the nucleic acid sequence(s) encoding the polypeptide. Eukaryotic cells which may be transduced include, but are not limited to, embryonic stem cells, embryonic carcinoma cells, as well as hematopoietic stem cells, hepatocytes, fibroblasts, myoblasts, keratinocytes, endothelial cells, and bronchial epithelial cells.

In some embodiments, gene delivery vectors which direct expression of an antibody (or antigen-binding fragment thereof) and/or construct (e.g., a targeting construct) in the eye are used. Vectors for gene delivery to the eye are known in the art, and have been disclosed, for example, in U.S. Pat. No. 6,943,153, and U.S. Patent Application Publication Nos. US20020194630, US20030129164, US200600627165.

In some embodiments, the complement activation is inhibited by contacting a body fluid with a composition comprising an antibody (or antigen-binding fragment thereof) and/or construct (e.g., a targeting construct) ex vivo under conditions that permit the antibody (or antigen-binding fragment thereof) and/or construct (e.g., targeting construct) to function to inhibit complement activation. Suitable body fluids include those that can be returned to the individual, such as blood, plasma, or lymph. Affinity adsorption apheresis is described generally in Nilsson et al. (1988) Blood 58(1):38-44; Christie et al. (1993) Transfusion 33:234-242; Richter et al. (1997) ASAIO J. 43(1):53-59; Suzuki et al. (1994) Autoimmunity 19: 105-112; U.S. Pat. No. 5,733,254; Richter et al. (1993) Metabol. Clin. Exp. 42:888-894; and Wallukat et al. (1996) Int'l J. Card. 54:1910195.

Accordingly, the invention include methods of treating one or more diseases described herein in an individual comprising treating the individual's blood extracorporeally (i.e., outside the body or ex vivo) with a composition comprising a targeting construct under conditions that permit the molecule to function to inhibit complement activation, and returning the blood to the individual.

Unit Dosages, Articles of Manufacture, and Kits

Also provided are unit dosage forms of an antibody (or antigen-binding fragment thereof) and/or construct (e.g., targeting construct) compositions, each dosage containing from about 0.01 mg to about 50 mg, including for example any of about 0.1 mg to about 50 mg, about 1 mg to about 50 mg, about 5 mg to about 40 mg, about 10 mg to about 20 mg, or about 15 mg of the targeting construct. In some embodiments, the unit dosage forms of an antibody (or antigen-binding fragment thereof) and/or construct (e.g., a targeting construct) composition comprises about any of 0.01 mg-0.1 mg, 0.1 mg-0.2 mg, 0.2 mg-0.25 mg, 0.25 mg-0.3 mg, 0.3 mg-0.35 mg, 0.35 mg-0.4 mg, 0.4 mg-0.5 mg, 0.5 mg-1.0 mg, 10 mg-20 mg, 20 mg-50 mg, 50 mg-80 mg, 80 mg-100 mg, 100 mg-150 mg, 150 mg-200 mg, 200 mg-250 mg, 250 mg-300 mg, 300 mg-400 mg, or 400 mg-500 mg targeting construct. In some embodiments, the unit dosage form comprises about 0.25 mg targeting construct. The term “unit dosage form” refers to a physically discrete unit suitable as unitatry dosages for an individual, each unit containing a predetermined quantity of active material calculated to produce the desired therapeutic effect, in association with a suitable pharmaceutical carrier, diluent, or excipient. These unit dosage forms can be stored in a suitable packaging in single or multiple unit dosages and may also be further sterilized and sealed.

Also provided are articles of manufacture comprising the compositions described herein in suitable packaging. Suitable packaging for compositions (such as ophthalmic compositions) described herein are known in the art, and include, for example, vials (such as sealed vials), vessels, ampules, bottles, jars, flexible packaging (e.g., sealed Mylar or plastic bags), and the like. These articles of manufacture may further be sterilized and/or sealed.

The present invention also provides kits comprising compositions (or unit dosages forms and/or articles of manufacture) described herein and may further comprise instruction(s) on methods of using the composition, such as uses described herein. The kits described herein may further include other materials desirable from a commercial and user standpoint, including other buffers, diluents, filters, needles, syringes, and package inserts with instructions for performing any methods described herein.

### EXAMPLES

### Example 1

### Generation of Targeting Constructs

To identify self-reactive monoclonal antibodies (mAbs) that recognize neo-epitopes on ischemic tissues, fresh isolated intestinal epithelial cells (IECs) were used to screen hybridomas obtained by the fusion of peritoneal, lymph node, and spleen cells from wild-type C57BL/6 mice with the Sp2/0-Ag14 myeloma cell line as described in Kulik et al., J Immunol., (2009), 182:5363-5373. Briefly, hybridoma fusions were screened based on reactivity by Western blot analysis on IEC lysates and positive surface staining of IECs as detected by flow cytometric analysis. For flow cytometry analysis, isolated IECs were washed in staining buffer (2% FCS/0.01% NaN3/PBS) then resuspended in staining buffer containing hybridoma supernatant, and incubated for 30 min at room temperature. After incubation, cells were washed in staining buffer three times and then incubated with secondary goat anti-mouse IgM (μ-chain specific) antibodies (Jackson ImmunoResearch Laboratories) for 30 minutes at room temperature. Following incubation, cells were washed as described above and then resuspended in the staining buffer. Flow cytometry was performed using a BD Biosciences FACSCalibur. For Western blot analysis, IECs were lysed on ice for 20 min in a buffer containing 0.5% Triton X-100, 0.5% Chaps, 20 mM Tris-HCl (pH 7.5), 1 mM EDTA, 10 μg/ml leupeptin, and protease inhibitor mixture (Roche Molecular Biochemicals). Lysates were cleared by centrifugation at 8000×g for 5 minutes. After separation by 8% Tris-glycine SDS-PAGE, the proteins were transferred to a polyvinylidene difluoride membrane. The membrane was blocked overnight with 5% nonfat milk dissolved in PBS. The membrane was washed in PBS and then probed with an antibody from hybridoma supernatant for 1-2 hours in 2% milk/PBS, washed, and then incubated with HRP-conjugated secondary antibodies. A positive signal was visualized using the ECL system (PerkinElmer). Candidate hybridomas were subsequently serially recloned to obtain monoclonal cell lines stably producing a single mAb. To purify candidate mAbs, antibodies from the exhausted supernatants of cultured hybridomas were affinity purified on a column of agarose beads with goat anti-human IgM (Sigma-Aldrich). Bound antibody was eluted with a buffer containing 0.1 M glycine (pH 2.3) and collected into a buffer containing 1.5 M Tris (pH 8.8). Eluted mAb was dialyzed against PBS (pH 7.4) for 48 hours and concentrated using centrifugal filtration on Centricon Plus-20 (Millipore). Antibody concentration was determined by measuring the A280 nm of the sample, and purity was confirmed by analysis on a 10% SDS-PAGE gel.

A hybridoma of interest produced an IgM κ isotype antibody designated as mAb B4. Flow cytometric analysis demonstrated that mAb B4 bound to a surface epitope on IEC but not to freshly isolated splenocytes or thymocytes. By Western blot analysis, mAb B4 recognized a protein with a molecular weight of 37 kDa in IEC lysates but not lysates from freshly isolated splenocytes or thymocytes. When other tissue lysates were probed by Western blot with mAb B4, the epitope was found to be widely distributed, including in the lung, liver, and kidney. To identify the protein recognized by mAb B4 on IEC, 2D gel separation of proteins according to their molecular weight and charge was conducted based on the method described in Vossenaar et al., Arthritis Res. Ther., (2004), 6:R142-R150 and Kulik et al., J Immunol., (2009), 182:5363-5373. After 2D separation, proteins reactive with mAb B4 were characterized by electrospray liquid chromatography MS analysis as described in Kulik et al., J Immunol., (2009), 182:5363-5373, and it was determined that mouse annexin IV was a protein recognized by mAb B4.

Another hybridoma of interest produced an IgM antibody designated as mAb C2 which did not react with Western blots of IECs. To determine reactivity of mAb C2 to phospholipids, which have been suggested to be exposed in apoptotic and ischemic cells, ELISA analysis was performed using phospholipid as a binding partner as described in Elvington et al., J Immunol., (2012), 188:1460-1468. Briefly, microtiter plates (Immulon 1B; Dynatech Laboratories, Chatilly, Va.) coated with 100 μl/well of 50 μg/ml phospholipid in methanol were dried under blowing air to allow the organic solvent to evaporate, and the wells were then washed with PBS and blocked with 1% BSA. Supernatant from the mAb C2 hybridoma was added to wells and bound antibody was detected by alkaline phosphatase-conjugated goat anti-mouse IgM (Jackson ImmunoResearch Laboratories, West Grove, Pa.). Phospholipids assayed included phosphatidylserine (PS)-1,2-distearoyl-sn-glycerol-3-[phospho-L-serine] (Avanti Polar-lipids, Alabaster, Ala.) (referred to as PS), cardiolipin from bovine heart (referred to as CL), phosphatidylethanolamine (PE)-1,2-diacyl-sn-glycero-3-phosphoethanolamine (referred to as PE), phosphotidylglycerol (PG)-1,2-diacyl-sn-glycero-3-phospho-(1-rac-glycerol) from yolk lecithin (referred to as PG), and phosphorylcholine (PC)(10)-BSA (Biosearch Technologies, Novato, Calif.) (referred to as PC-BSA). mAb C2 was shown to recognize a subset of phospholipids that included PC-BSA, PE, and CL, but not PG or PS.

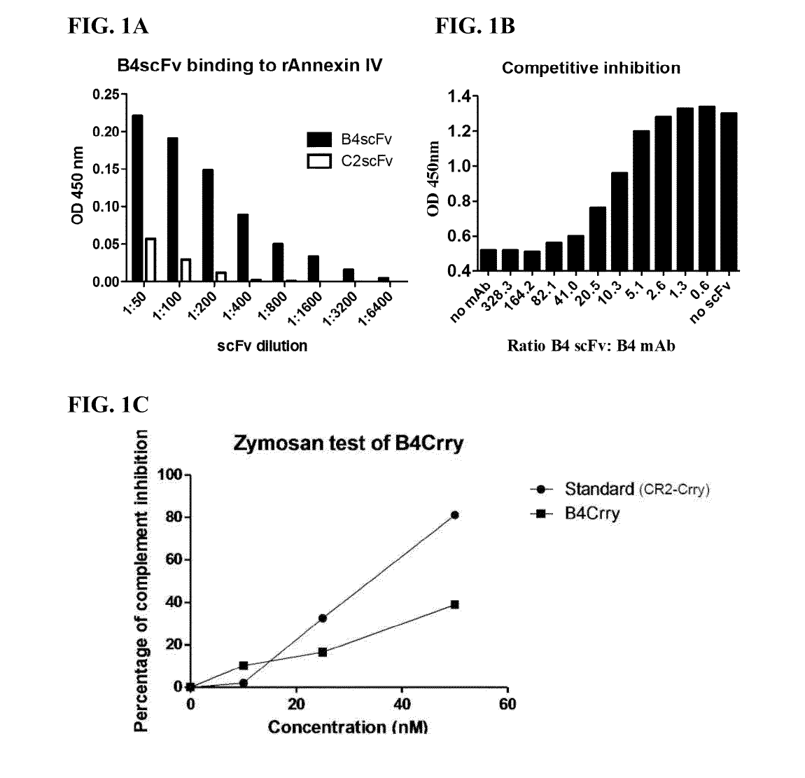

Since B4 and C2 mAbs were identified as antibodies that recognize neo-epitopes of ischemic tissue that can contribute to complement pathway mediated injury, targeting constructs comprising a targeting moiety, in this case scFv isolated from the IgM-B4 antibody or IgM-C2 antibody, and an active moiety, in this case a complement modulator, were generated and tested for their ability to protect ischemic tissue from injury. For generation of targeting constructs a scFv from both IgM-B4 antibody and IgM-C2 antibody was prepared by amplifying the isolated VH and VL genes from cDNA and by linking using overlap extension PCR and expressed as a scFv with an N-terminal His-tag. Purified scFv had the expected molecular weight by SDS-PAGE. The purified scFv retained parent IgM binding specificity as demonstrated by its ability to directly bind to its binding partner, which in the case of B4scFv, was the ability to directly bind to recombinant annexin IV in vitro as well as to competitively inhibit binding of B4 mAb to annexin IV (FIGS. 1A and 1B). The targeting moiety B4scFv or C2scFv encoding sequences were linked with the artificial linker (G4S1)2 to an active moiety encoding sequence selected from the complement modulators: complement receptor 1-like protein (Crry), complement factor H (fH), or CD59 molecule complement regulatory protein (CD59). For Crry and CD59, the sequence encoding the extracellular domain of the proteins was used. For fH, the sequence encoding the N-terminal 5 short consensus repeat domains (the active region) was used. The targeting construct encoding sequences were inserted into an expression vector and expression plasmids were transfected into Chinese hamster ovary (CHO) cells. Positive high expressing clones were selected by a limiting dilution assay and targeting construct proteins, specifically B4scFv-Crry, B4scFv-fH, B4scFv-CD59, C2scFv-Crry, C2scFv-fH, and C2scFv-CD59 proteins were produced, recovered from culture supernatant, and purified by affinity chromatography using either antibodies to the active moiety or to the His tag. The purified targeting construct had the expected molecular weight by SDS-PAGE and the activity of the complement modulator was retained. In the case of B4scFv-Crry, in vitro complement modulatory activity was tested in a standard assay that measured C3 deposition on zymosan particles and compared to positive control CR2-Crry (FIG. 1C).

Mouse monoclonal antibody B4 was deposited at the ATCC with ATCC Deposit No. PTA-13522 (B4/14/12). Mouse monoclonal antibody C2 was deposited at the ATCC with ATCC Deposit No. PTA-13523 (C2/19/8).

### Example 2

### Reduction of Spinal Cord Injury by Treatment with a Targeting Construct

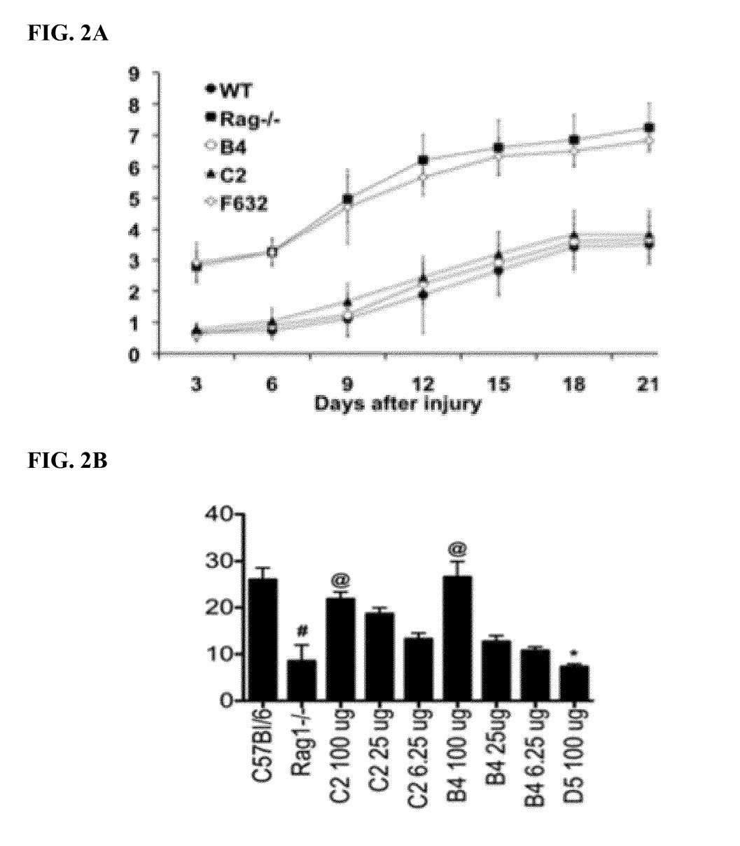

The role of self-reactive C2 or B4 mAbs in spinal cord injury (SCI) and cerebral ischemia reperfusion injury (ischemic stroke) was investigated. Rag1−/− mice, which produce no mature T-cells or B-cells and are therefore antibody-deficient, have been previously reported to be protected from ischemia reperfusion (IR) injury. See Williams et al., J. Appl. Physiol. (1999), 86:938-942. For these studies, mouse models of SCI and middle cerebral artery occlusion (MCAo) (with 60 minutes ischemia and 24 hours reperfusion) were used as described in Qiao et al, Am. J. Pathol, (2006), 169:1039-1047 and Atkinson et al, J Immunol., (2006), 177:7266-7274. Antibodies were administered intravenously 60 minutes after SCI or at time of reperfusion for MCAo model. Locomotor function was assessed after SCI using the Basso, Beattie and Bresnahan (BBB) rating scale developed for rats, but adapted for mice (Noble et al., J. Neuroscience, (2002), 22:7526-7535). To measure infract volume after MCAo, coronal section from isolated brains were stained with 2% triphenyltetrazolium (TTC) and infracted area (excluding dye) was determined using NIH image analysis software. It was determined that IgM antibody deficient Rag1−/− mice were protected from SCI as measured by locomotor activity, but injury was reconstituted in Rag1−/− mice to wild-type mouse levels by intravenous administration of C2 or B4 mAbs, but not with control F632 mAb (FIG. 2A). Furthermore, B4 mAb and C2 mAb, but not control antibody, reconstituted cerebral ischemia and reperfusion injury in Rag1−/− mice (FIG. 2B).

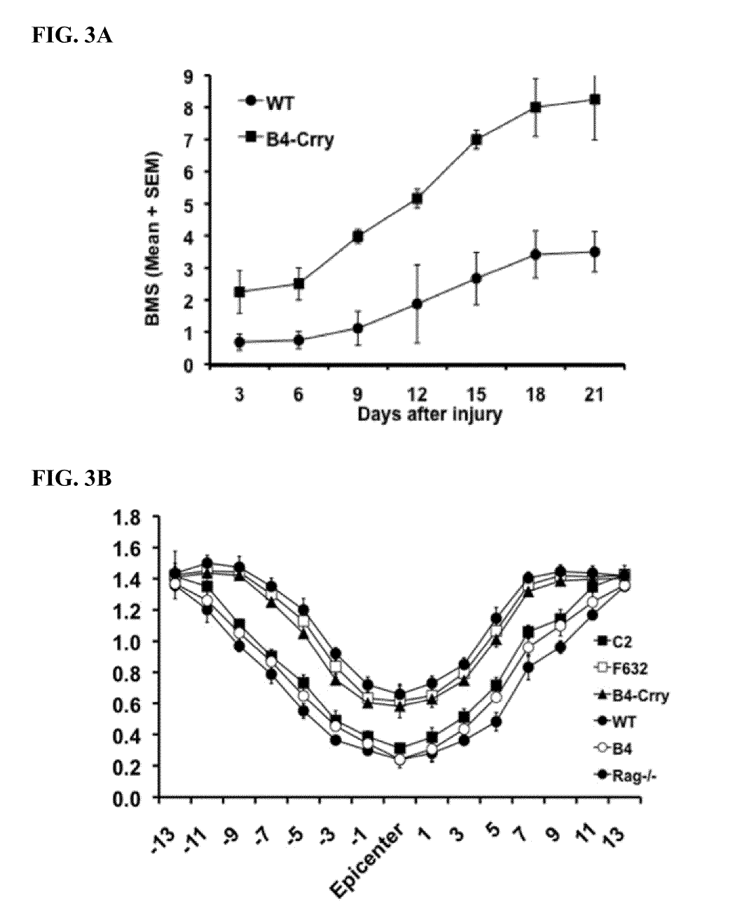

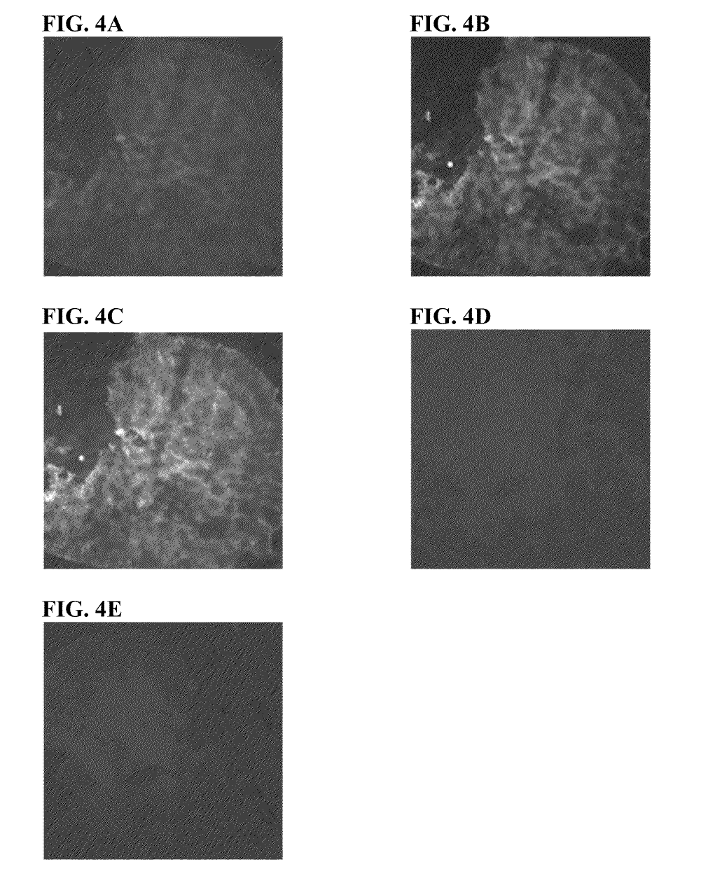

C57Bl/6 wild-type mice were administered targeting construct B4scFV-Crry, to test a possible protective effect of this targeting construct against self-reactive antibodies in an ischemic injury model of SCI. In this SCI model, 0.2 mg B4-Crry or phosphate buffered saline (PBS) was injected intravenously 60 minutes post-injury. Assessment of locomotor activity showed that B4-Crry treated mice had significantly improved recovery and protected mice from SCI as compared to PBS treated mice (FIG. 3A). Tissue sparing was also analyzed at 3 days post SCI, and there was significantly increased tissue sparing in B4scFv-Crry treated C57Bl/6 wild-type mice (FIG. 3B, filled triangles) as compared to control C57Bl/6 wild-type mice administered PBS (FIG. 3B, filled circles). Tissue sparing of B4scFv-Crry treated C57Bl/6 wild-type mice was comparable to tissue sparing in Rag1−/− mice treated with PBS (FIG. 3B, filled circles, upper line) or control F632 mAb (FIG. 3B, empty squares) but was significantly increased as compared to Rag1−/− mice reconstituted with C2 mAb (FIG. 3B, filled squares) or B4 mAb (FIG. 3B, open circles). Furthermore, IgM binding to the spinal cord following SCI in C57Bl/6 wild type mice was confirmed, and IgM and C3 was shown to colocalize in spinal cords from wild-type mice 24 hours after injury (FIGS. 4A-4C). Administration of B4scFv-Crry reduced IgM and C3 deposition 24 hours after injury (FIGS. 4D-4E).

### Example 3

### Administration of a Targeting Construct Did not Increase Host Susceptibility to Infection

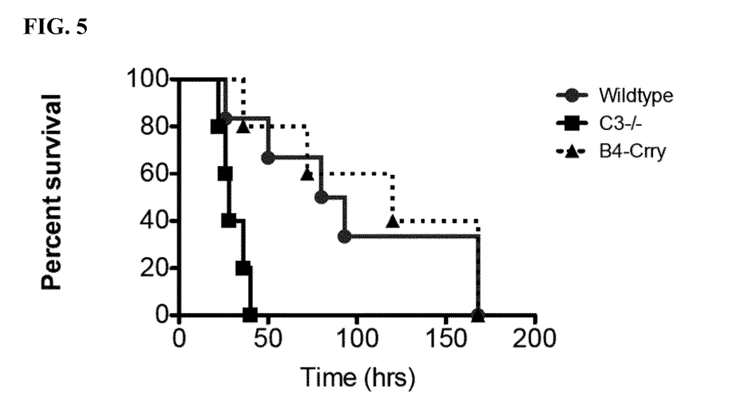

A major potential advantage of targeted versus systemic complement inhibition is that a targeted approach is less likely to impact physiologically important roles of complement. One such important role, and one that has some relevance to a transplant patient, is host defense against infection. The effect of B4scFv-Crry on susceptibility to infection using a well characterized model of polymicrobial sepsis was investigated with a 0.2 mg dose that was therapeutic in animal models of SCI as well as animal models of hepatic and cardiac ischemia reperfusion injury. Cecal ligation and puncture was performed in C3 deficient mice or in wild-type mice treated with either B4scFv-Crry or PBS, and survival was monitored. B4scFv-Crry had no effect on host susceptibility to infection in a model of acute septic peritonitis, and mice treated with B4scFv-Crry survived significantly longer following cecal ligation puncture, and survival was not significantly different compared to PBS treated wild-type mice (FIG. 5). In contrast, all mice deficient in C3 died within 48 hours, a similar result that was previously obtained for both C3 deficient mice and mice treated with a therapeutic dose of Crry-Ig, a systemic counterpart of B4scFv-Crry.

### Example 4

### Reduction of Cardiac Ischemia Reperfusion Injury by Treatment with a Targeting Construct

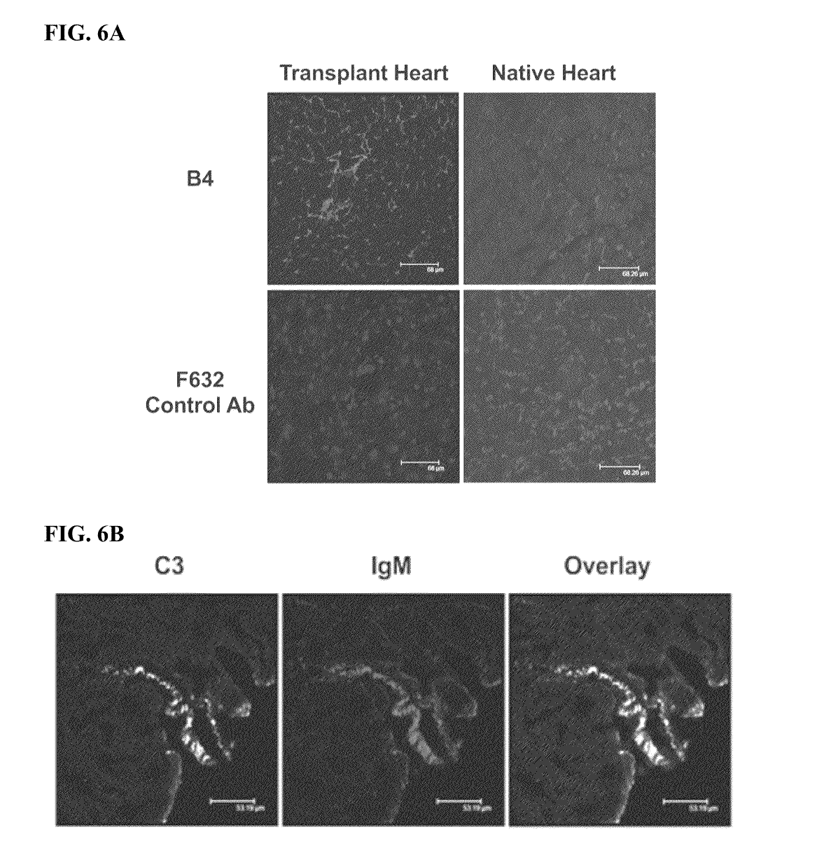

The role of self-reactive natural IgM in post-transplant cardiac ischemia reperfusion injury was investigated in isografts after heart transplantation from C57BL/6 wild-type donors to either antibody deficient Rag1−/− recipients or Rag1−/− recipients reconstituted with C2 mAb or B4 mAb. mAbs were administered intravenously to recipient immediately following reperfusion of transplanted heart. Immunofluorescence for endothelial markers, IgM and C3 binding was performed on 8 micron cryosections stained with appropriate fluorescently labeled antibodies, and imaged with a confocal microscope. For histopathology, sections were stained with H&E and scored by an observer blinded to experimental groups. Sections were scored on a scale of 0-3 as described in Atkinson et al., J. Immunol., (2010), 185:7007-7013. Compared to grafts in wild-type recipients (Balb/C to C57BL/6), hearts transplanted into antibody-deficient Rag1−/− recipient mice by heterotopic abdominal heart transplantation were protected from post-transplant cardiac IR injury as evidenced by reduced histologic injury and inflammation and decreased serum levels of cardiac troponin I, an index of cardiac cell damage. However, reconstitution with either B4 mAb or C2 mAb, but not control F632 IgM, in Rag1−/− recipients restored cardiac IR injury in the transplanted hearts to that seen in wild-type recipients. The graft specificity and binding characteristics of B4 mAb and C2 mAb was investigated by immunofluorescence analysis of graft sections. In B4 mAb (FIG. 6A) and C2 mAb treated heart recipient Rag1−/− mice, both mAbs bound to endothelial cells of arterioles, capillaries, and microvessels within the mycocardium of the transplanted heart, but not the native heart. Additional mAb binding in grafts was seen on myocytes, with a preferential localization to injured myocytes in epicardium. No IgM immunostaining could be detected in the graft or native heart in animals reconstituted with F623 control mAb (FIG. 6A). Analysis of IgM binding and complement activation showed that B4 IgM co-localized with C3d (complement activation product) in hearts transplanted in Rag1−/− recipients and that endogenous IgM co-localized with C3d in grafts of wild-type recipients (FIG. 6B).

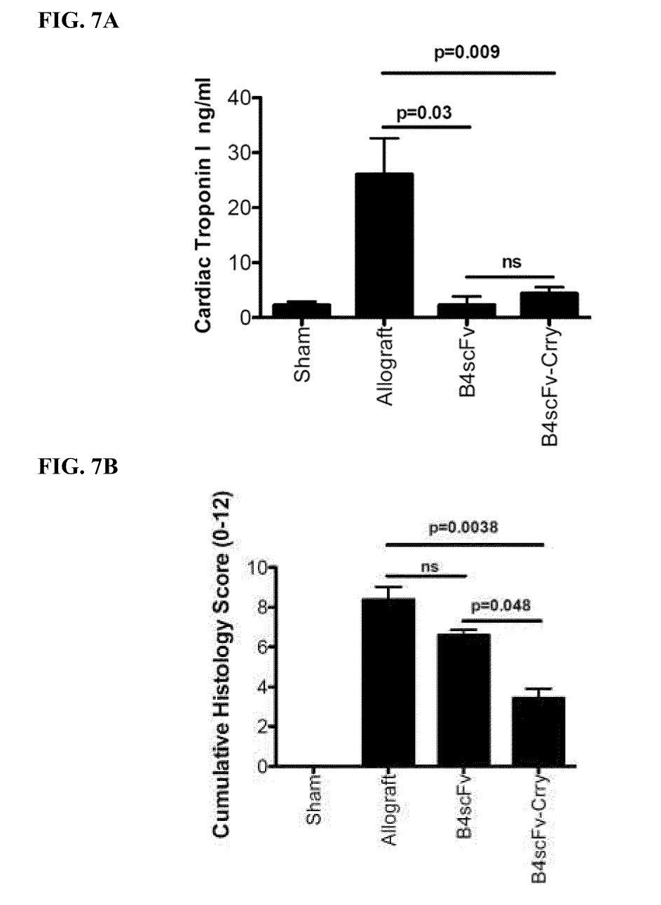

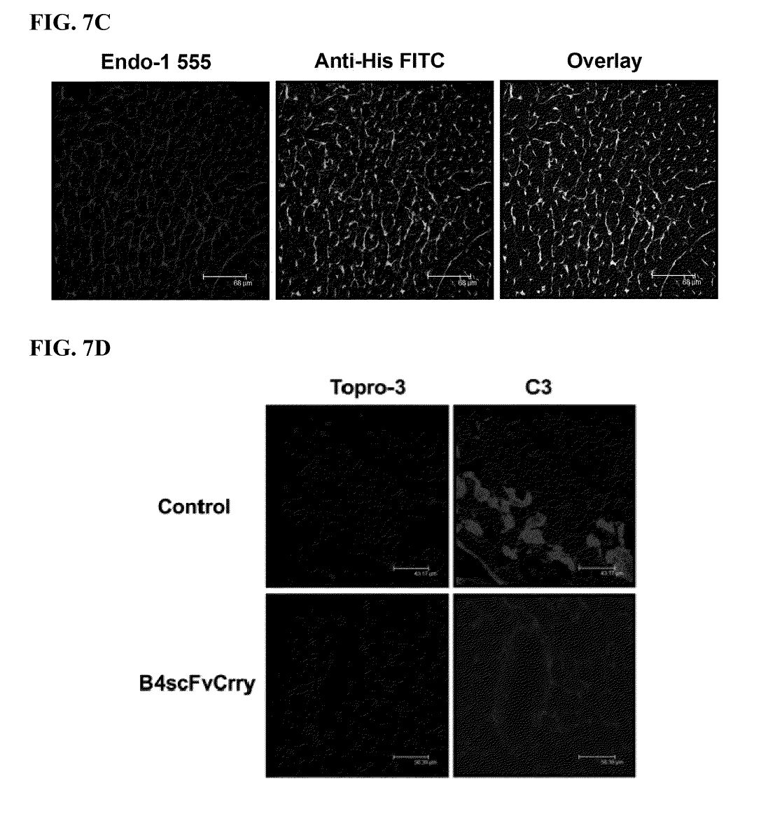

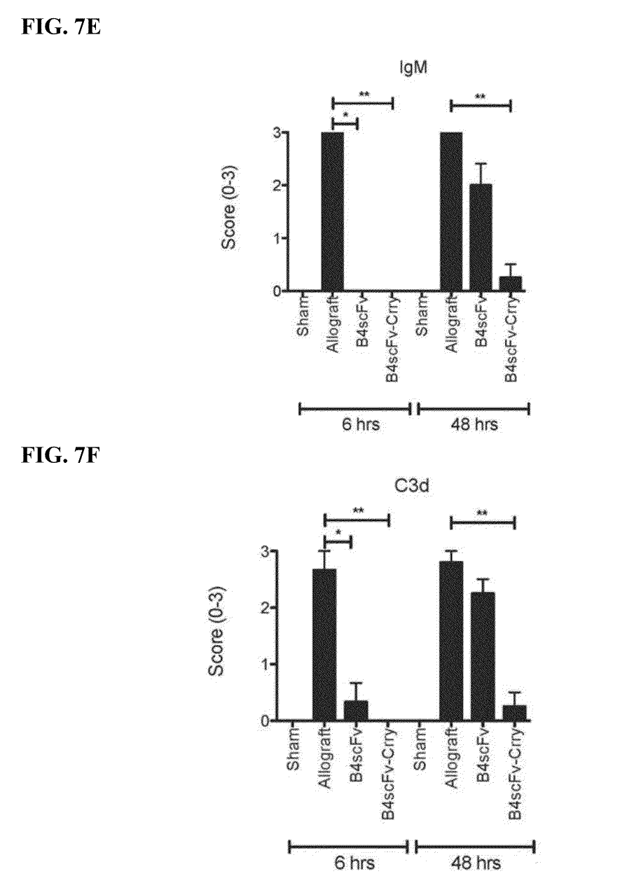

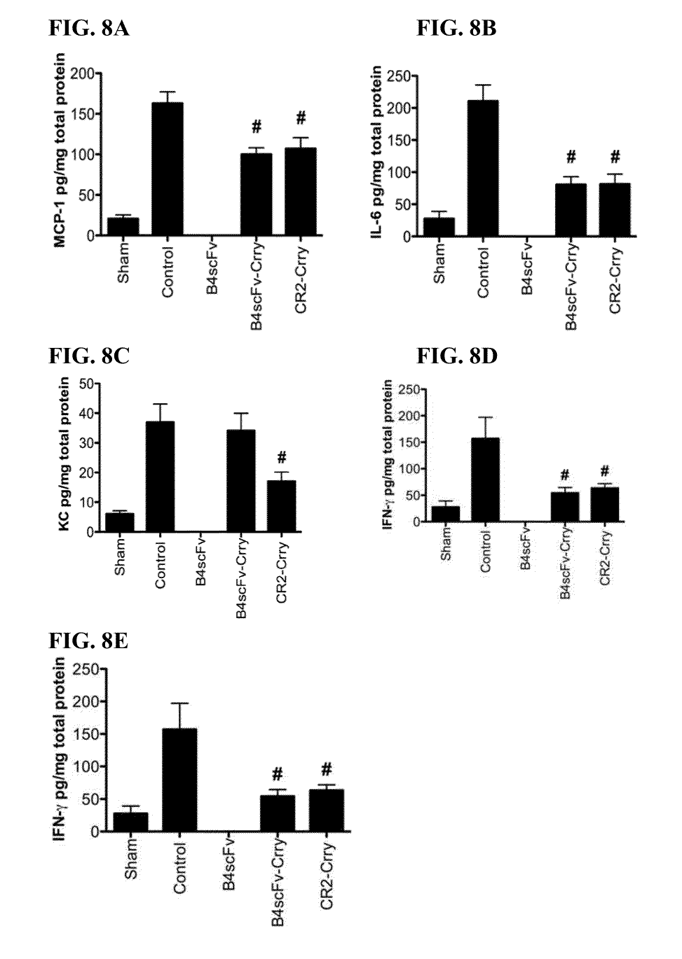

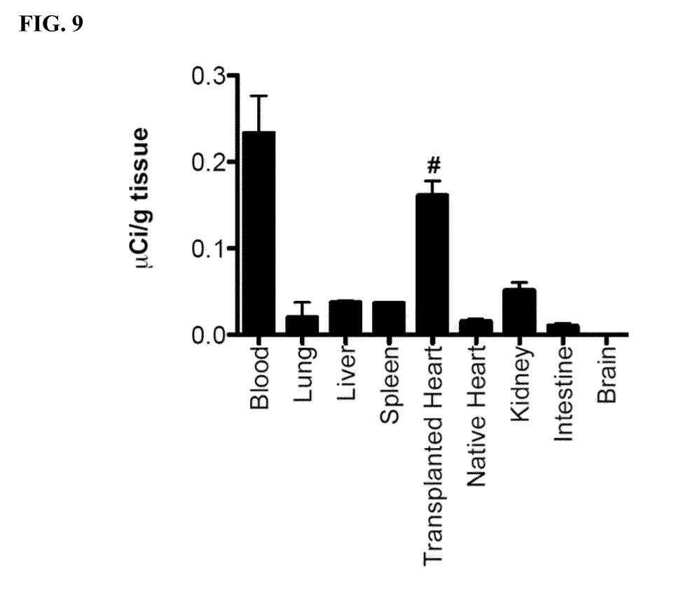

In a therapeutic protocol, a 0.2 mg dose of B4scFV-Crry or B4scFv was administered immediately post-transplantation in a wild-type allograft transplant model (Balb/C to C57BL/6). Allografts were harvested at 6 hours (after intravenous B4scFv treatment) or 48 hours (after intravenous B4scFV-Crry treatment) after reperfusion and analyzed. When administered immediately after reperfusion, administration of B4scFv or B4scFv-Crry resulted in a significant reduction in mycocardial IRI as evidenced by decreased cardiac troponin I, an index of cardiac cell damage levels (FIG. 7A). Grafts were also assessed for histological evidence of injury and inflammatory cell infiltration. In accordance with cardiac troponin I levels, grafts from B4scFv-Crry treated recipients had a significantly lower injury and inflammation histology score than grafts from vehicle treated recipients (FIG. 7B). Reduced graft injury and inflammation in B4scFv treated recipients was also observed as compared to recipients receiving PBS vehicle (FIG. 7B). In His-tagged B4scFv treated allografts, graft sections stained positive with anti-His antibodies (FIG. 7C), and B4scFv co-localized with a pan-endothelial marker (Endo-1) with B4scFv binding seen in post-ischemic vessels of all sizes. In addition, B4scFv-Crry reduced C3 deposition as shown by a reduced C3 immunostaining (FIG. 7D). Quantitative analysis of the images demonstrated that IgM binding and C3d deposition occurred in grafts from recipients treated with PBS, with staining predominantly in the microvasculature at 6 hours post-transplantation, and in the microvasculature and on myocytes at 48 hours post-transplantation (FIGS. 7E-7F). Treatment of recipients with either B4scFv or B4scFv-Crry significantly reduced or eliminated both IgM and C3d deposition in grafts at 6 hours post-transplantation. At 48 hours post-transplantation, however, significant reductions in IgM binding and C3d deposition was seen only in grafts from B4scFv-Crry treated recipients (FIGS. 7E-7F). To further investigate the inflammatory environment modulated by B4scFv-Crry, the effect of this molecule on graft levels of specific cytokines and chemokines was assessed. B4scFv-Crry treatment of recipients significantly reduced graft levels of the cardiotoxic cytokine IL-6 and MCP-1 as compared to PBS vehicle treatment. Graft levels of KC at 48 hours post-transplantation were not altered by treatment with either B4scFv-Crry (FIG. 8). These molecules also reduced inflammatory cell infiltration as compared to PBS control treated mice.

To determine the circulatory half-life (t1/2) of B4scFV-Crry, 100 μg B4scFv-Crry was injected intravenously into C57BL/6 recipient mice, and plasma concentration of the protein was determined at different time points by ELISA using an anti-Crry antibody. There was a two-phase elimination profile; an initial rapid phase with a t1/2 of 9.7 minutes, and a second prolonged phase with a t1/2 of 5.5 hours. A similar two-phase elimination profile has been shown for other biologics, including untargeted Crry (Crry-Ig), although the second phase t1/2 for Crry-Ig was considerably longer (40 hrs). Significant therapeutic efficacy was seen with a 0.2 mg dose of B4scFv-Crry, and since preferential accumulation at the target site would be expected to translate to a lower dose requirement for therapeutic benefit, a biodistribution study was performed. 125I-labeled B4scFv-Crry was injected into recipient mice immediately after heart transplantation, and tissue distribution of radiolabel determined 6 hours later. In addition to being present in the circulation, 125I-B4 scFv-Crry localized primarily to the transplanted graft (FIG. 9).

### Example 5

### Species Cross-Reactivity of Self-Reactive IgM

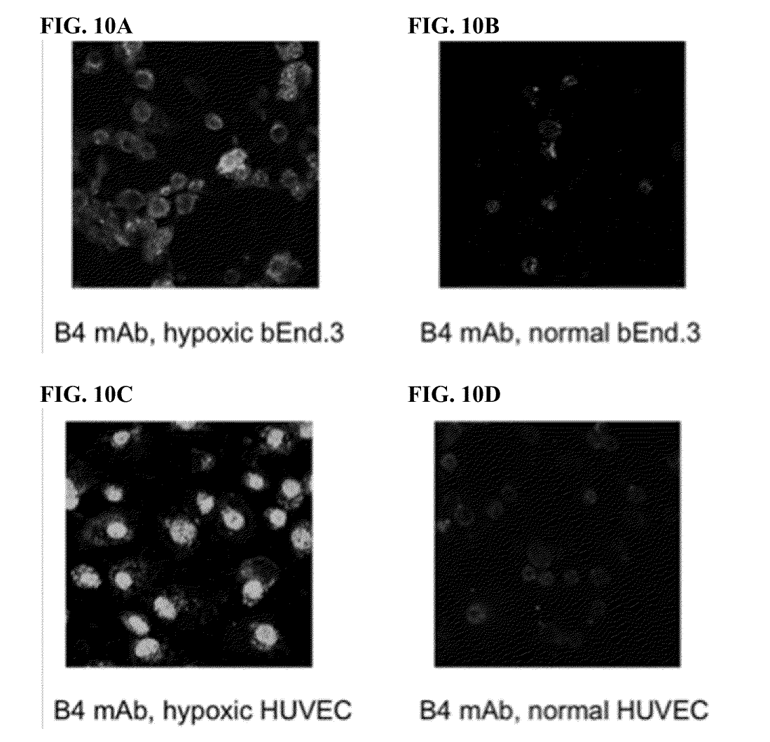

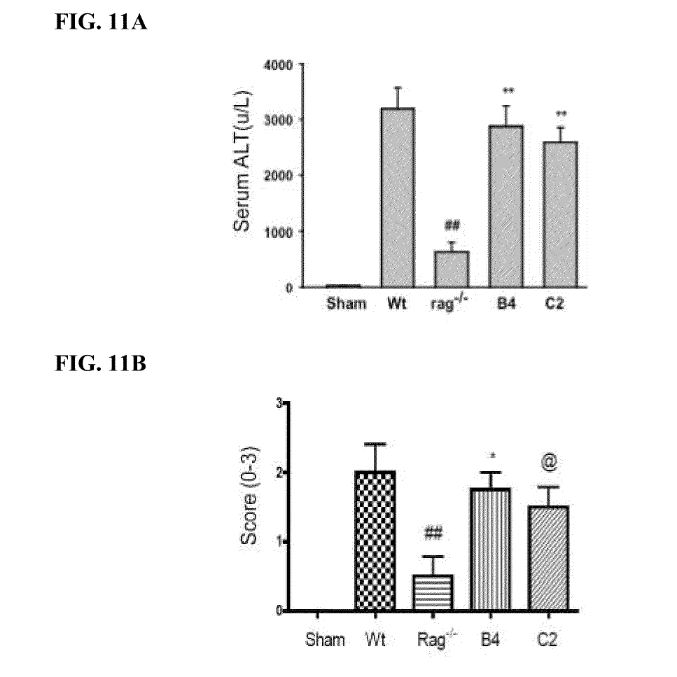

Reconstitution of Rag1−/− mice with human natural IgM reconstituted ischemia reperfusion injury (IRI) via IgM mediated complement activation, demonstrating species cross reactivity. Furthermore, there were high levels of anti-annexin IV Abs (B4 mAb specificity) present in human serum. Cross species reactivity of mouse B4 mAb and C2 mAb was further investigated by exposing human umbilical vein endothelial cells and a mouse brain endothelial cell line to 3 hours of hypoxia followed by 12 hours normoxia in the presence of either B4 mAb or C2 mAb in vitro. Flow cytometry and immunofluorescence microscopy demonstrated that both mAbs bound to mouse (FIG. 10A) and human cells (FIG. 10C) exposed to hypoxia, but not to control normoxic cells (FIGS. 10B and 10D), indicating that B4 and C2 show cross specificity for human neoepitopes.

### Example 6

### Reduction of Hepatic Ischemia Reperfusion Injury by Treatment with a Targeting Construct

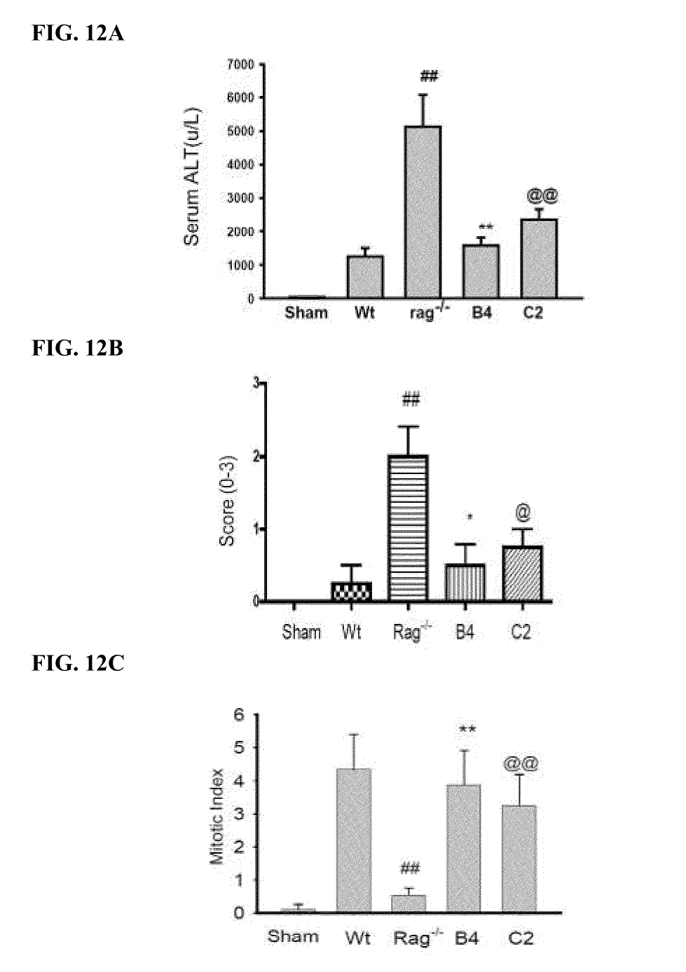

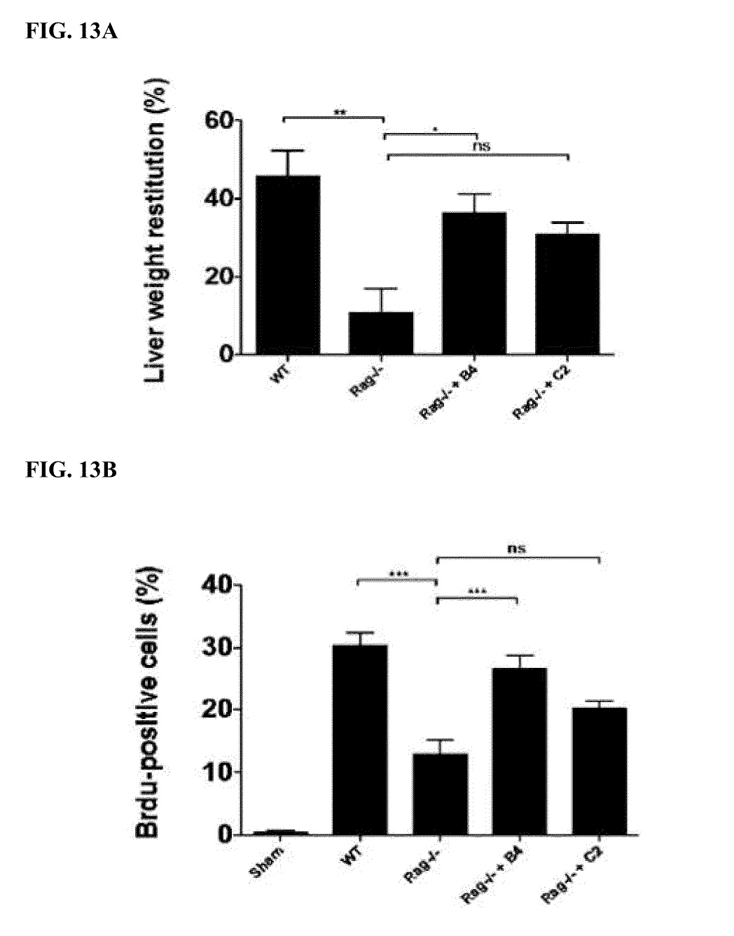

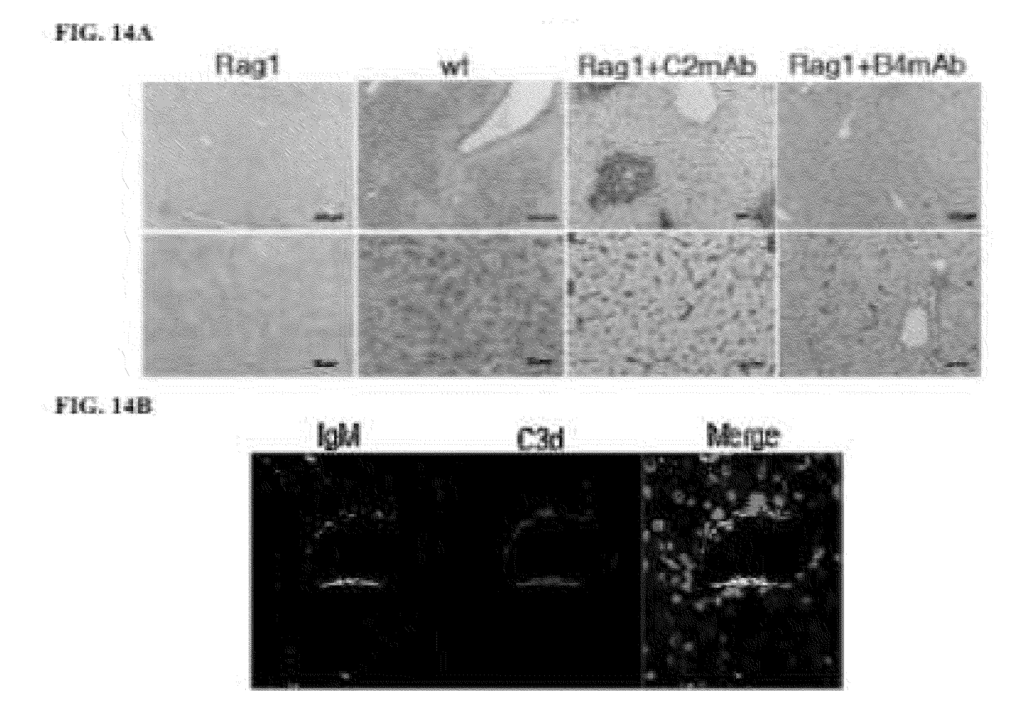

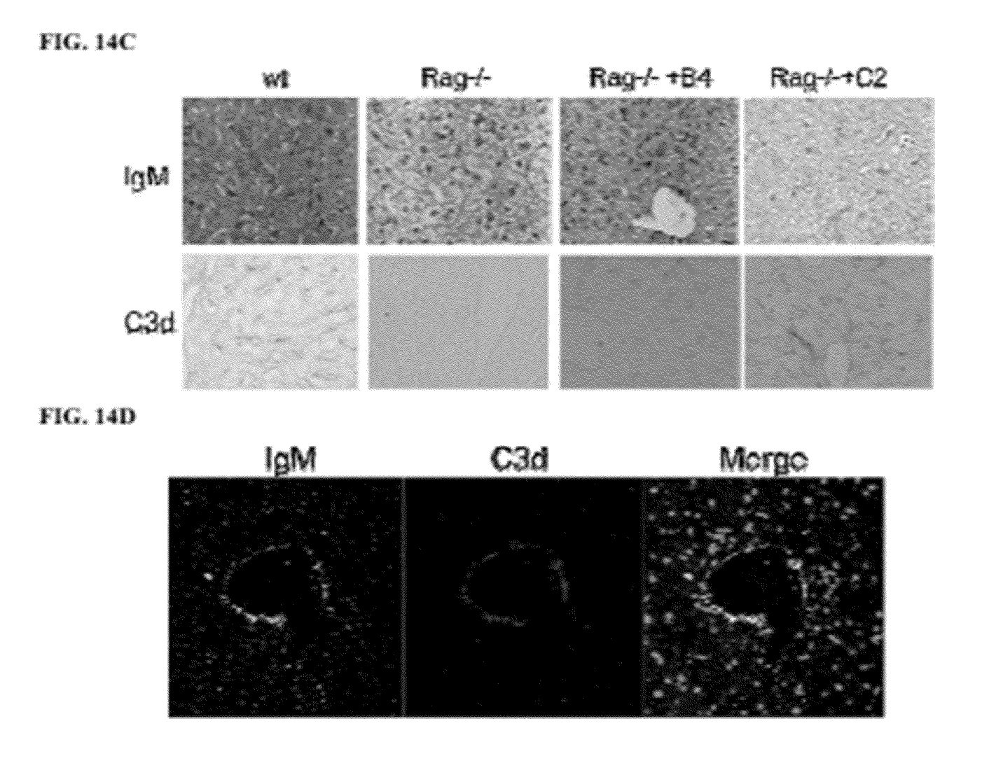

Complement activation plays an important role in both hepatic ischemia/reperfusion injury (IRI) and in the priming phase of liver regeneration. Utilizing separate models of hepatic IRI and 70% partial hepatectomy (Phx), the role of IgM in both hepatic IRI and liver regeneration was investigated. For IRI experiments, saline or a 25 μg dose of C2 mAb or B4 mAb was injected into antibody deficient (Rag1−/−) mice following 30 minutes total warm ischemia and just prior to reperfusion. Samples were taken after 6 hours of reperfusion. Serum ALT levels were measured by ELISA and histological necrosis was quantified in H&E stained sections (scale 0-3). For 70% partial hepatectomy experiments, saline or a 10 μg dose of C2 mAb or B4 mAb was injected into antibody deficient (Rag1−/−) mice immediately following 70% Phx. Samples were taken 48 hours post Phx. Mitotic index was calculated as a measure of hepatic regeneration (Mitotic figures/total cells in 10 hpf). Serum ALT levels were measured and histological necrosis was quantified (scale 0-3). The results showed that antibody deficient Rag1−/− mice were protected from hepatic IRI, and reconstitution of Rag1−/− mice with either B4 mAb or C2 mAb restored IRI to a level close to that seen in wild-type mice as determined by serum ALT levels (FIG. 11A) and histological score (FIG. 11B). Following 70% PHx in Rag1−/− mice, there was increased injury in the remnant liver compared to wild-type mice, and reconstitution of Rag1−/− mice with either B4 mAb or C2 mAb was protective as determined by serum ALT levels (FIG. 12A) and histological score (FIG. 12B). Rag1−/− mice had an impaired regenerative response after 70% PHx compared to wild-type mice, but reconstitution with B4 mAb or C2 mAb restored regeneration as assessed by mitotic index (FIG. 12C), liver weight restitution (FIG. 13A), and increase of Brdu-positive cells (FIG. 13B). Analysis of liver sections showed that IgM was deposited in the liver of wild-type mice after ischemia and reperfusion, and both B4 mAb and C2 mAb were deposited in livers of Rag1−/− mice after IR (FIG. 14A) and after PHx (FIG. 14C). Furthermore, analysis of liver sections after either IR injury (FIG. 14B) or PHx (FIG. 14D) showed IgM deposition co-localized with C3 deposition and there was no detectable IgM or C3 in sections from untreated Rag1−/− mice.

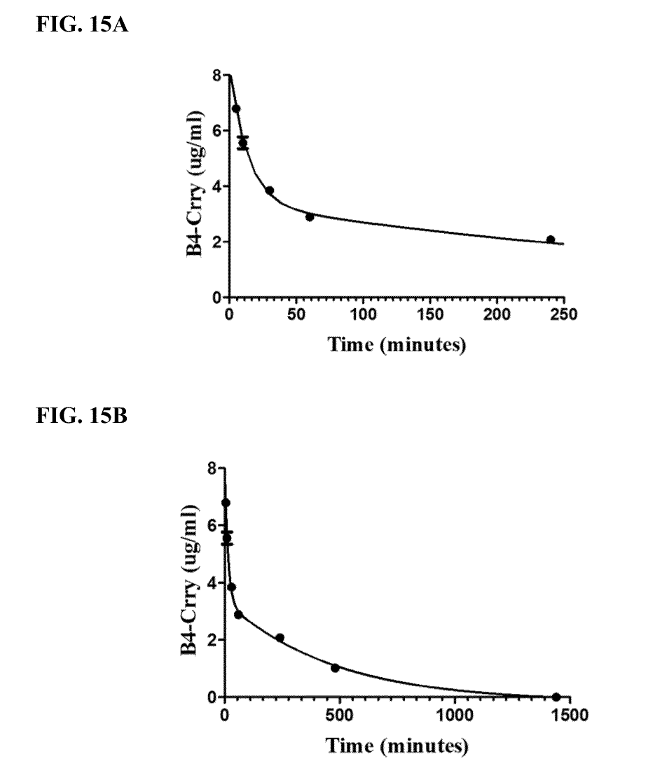

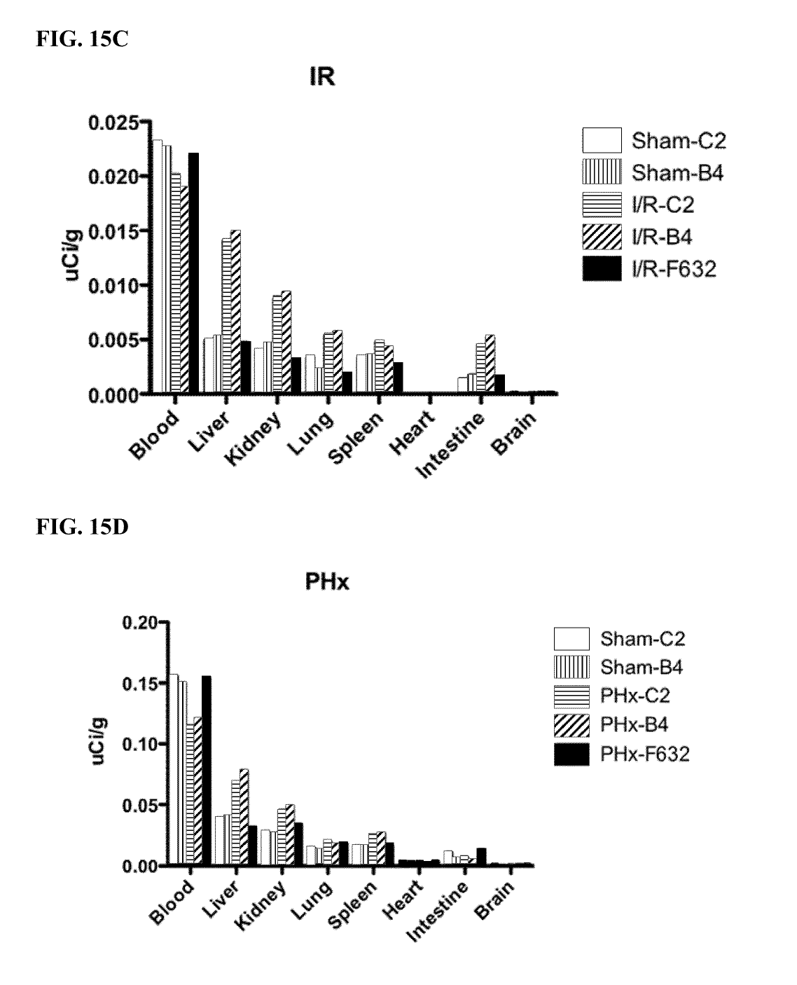

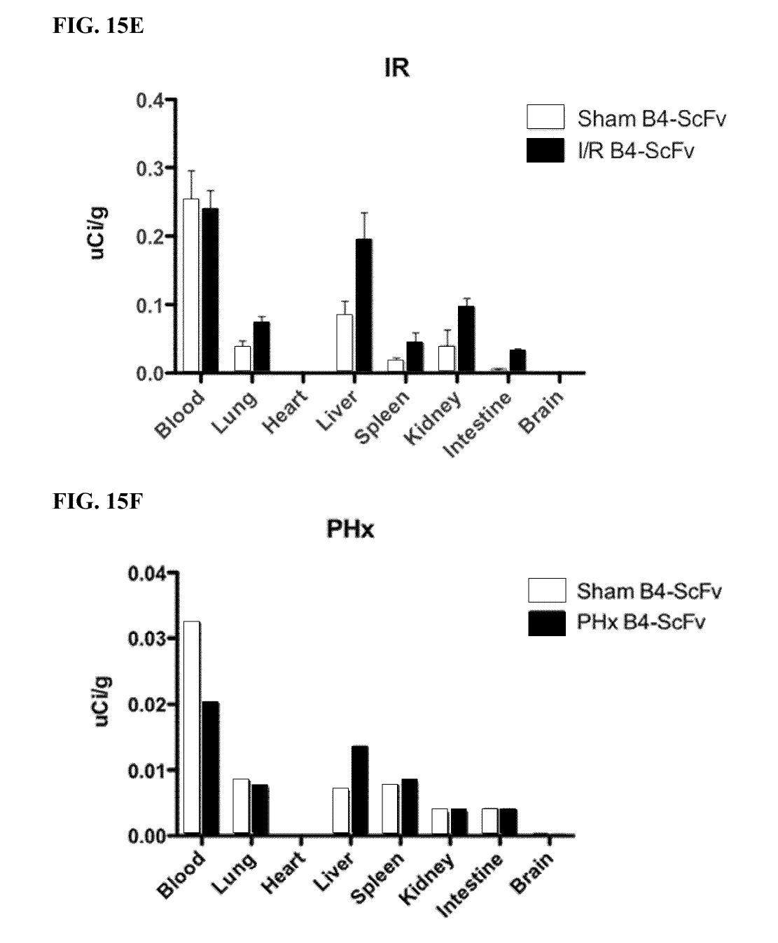

To determine the circulatory half-life (t1/2) of B4scFV-Crry, 100 μg B4scFv-Crry was injected intravenously into mice, and plasma concentration of the protein was determined at different time points by ELISA using an anti-Crry antibody. There was a two-phase elimination profile; an initial rapid phase with a t1/2 of 27 minutes, and a second prolonged phase with a t1/2 of 6.5 hours (FIGS. 15A-15B). To further determine the specificity of the C2 and B4 IgMs for injured tissues, biodistribution studies were performed in Rag1−/− mice. Rag1−/− mice were injected intravenously with 125Iodine radiolabeled C2, B4, or isotype control antibody F632 after one of the following procedures: sham, 70% PHx, or IR. Organs and blood were harvested 6 hours after surgical procedures. Blood was removed by cardiac puncture and the animals were perfused with PBS before the heart, brain, liver, intestine, lung, kidney, and spleen were removed. Tissues were rinsed with PBS, shredded, weighed and then radioactivity was measured with a Hewlett-Packard 5780 γ counter. Results were recorded in μCi/g of tissue. Both C2 and B4 antibodies targeted mainly to the liver following either IR or PHx (FIGS. 15C-15D). Additionally, some binding of both C2 and B4 was seen in the kidneys following IR or PHx and in the intestines following IR. Isotype control antibody F632 was not found in higher quantities in any organ as compared to sham animals. These findings suggested that similar epitopes were expressed following both IR and PHx in the liver, and that these epitopes were only found on injured tissues. Given the specificity of these epitopes for injured tissues they may serve as a ligand to specifically target a therapy to liver following either PHx or IRI. To confirm the role of IgM, B4 scFv was administered to the animals. B4 scFv had the same biodistribution profile following IRI or Phx as the B4 IgM mAb used in the above studies (FIGS. 15E-15F).

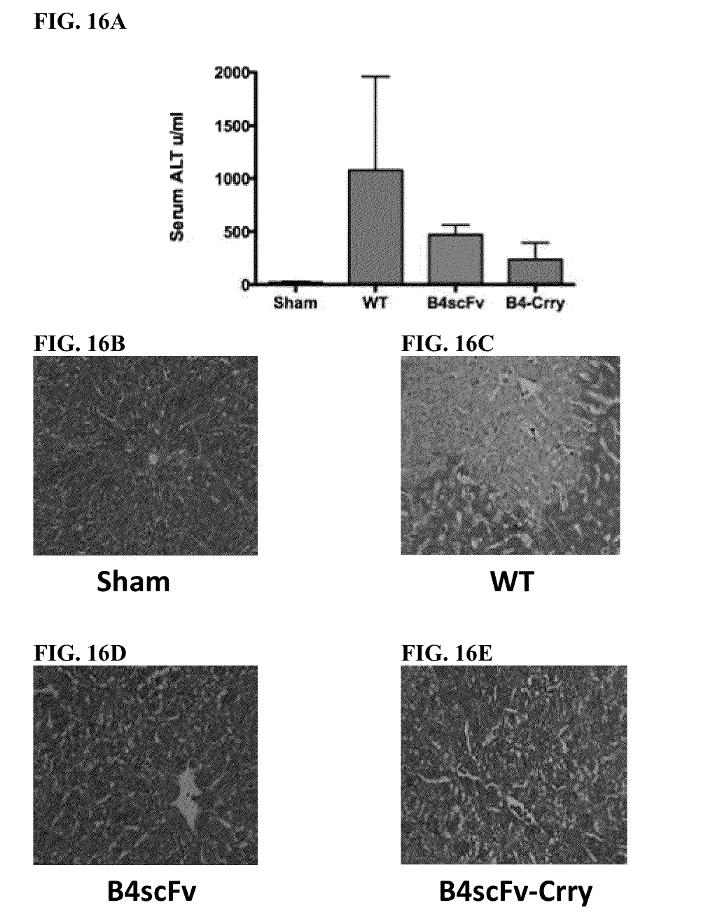

The in vivo activity of the targeting moiety B4scFv and the targeting construct B4scFv-Crry was assessed in the mouse model of hepatic ischemia reperfusion injury. Administration of B4scFv or B4scFv-Crry in mice subjected to 35 minutes of ischemia followed with 24 hours of reperfusion protected mice against hepatic ischemia reperfusion injury as demonstrated by serum ALT levels (FIG. 16A) and immunohistochemistry (FIGS. 16B-16E).

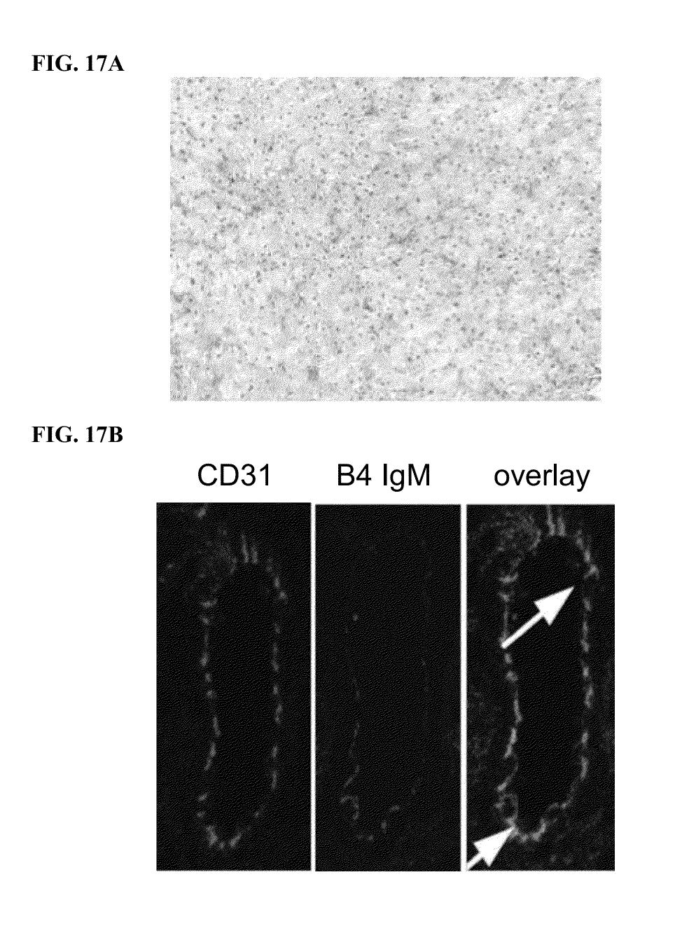

To determine if B4 mAb bound to human ischemic liver tissues, frozen human tissue samples were harvested just prior to transplantation. These livers were therefore ischemic but not yet reperfused. Sectioned liver was also stained for IgM post-reperfusion (FIG. 17A). It was observed that B4 mAb bound to human ischemic tissue and colocalized with the vascular endothelial marker CD31 demonstrating that B4 mAb bound to ischemic tissue in both humans and mice (FIG. 17B).

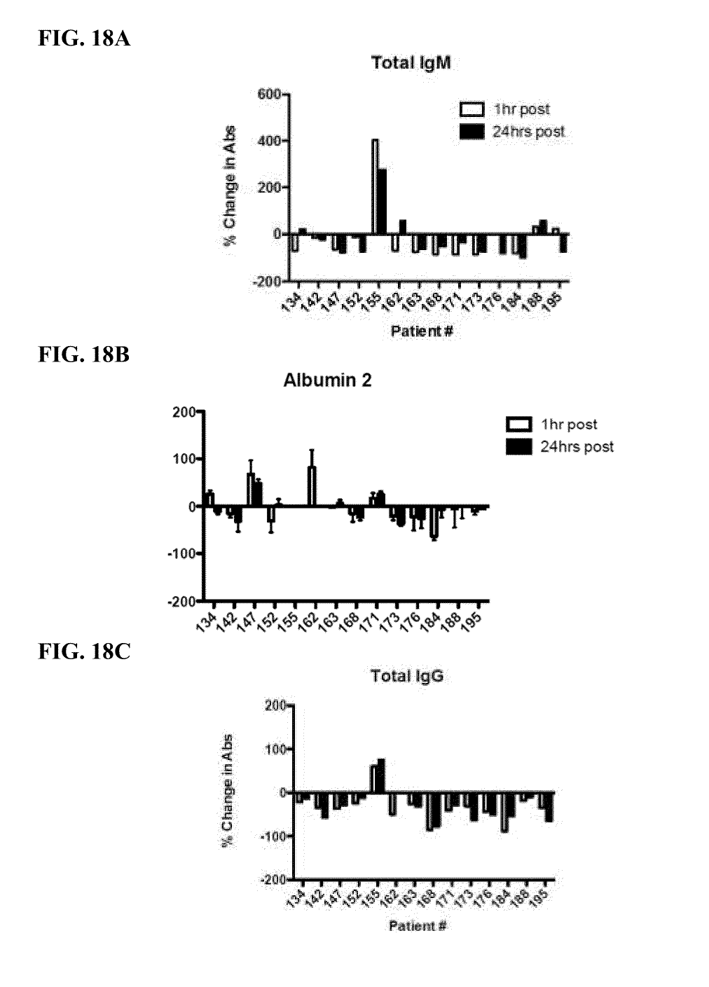

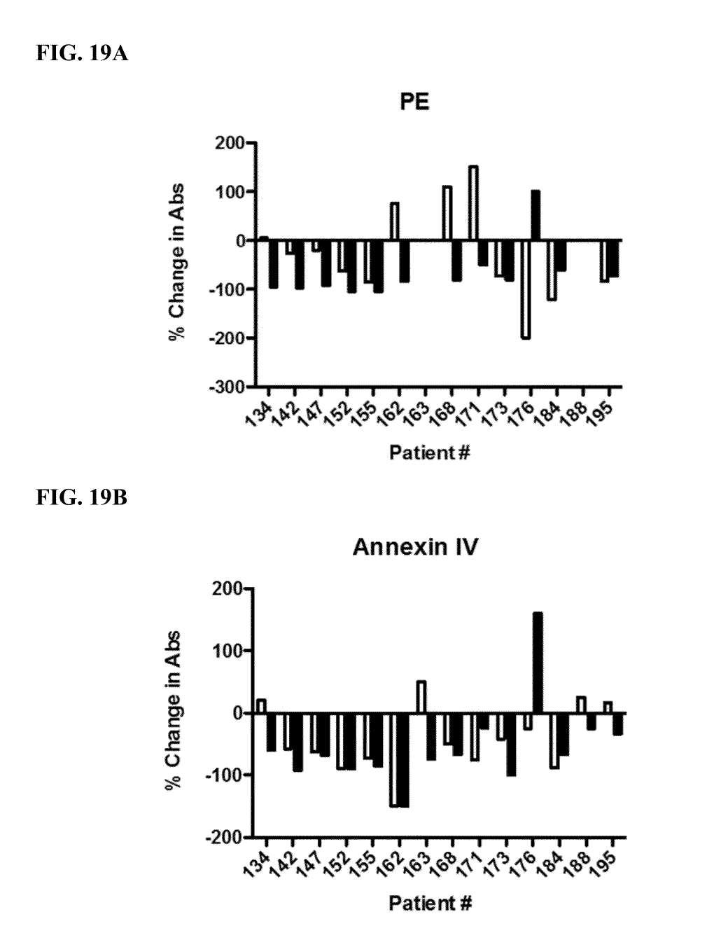

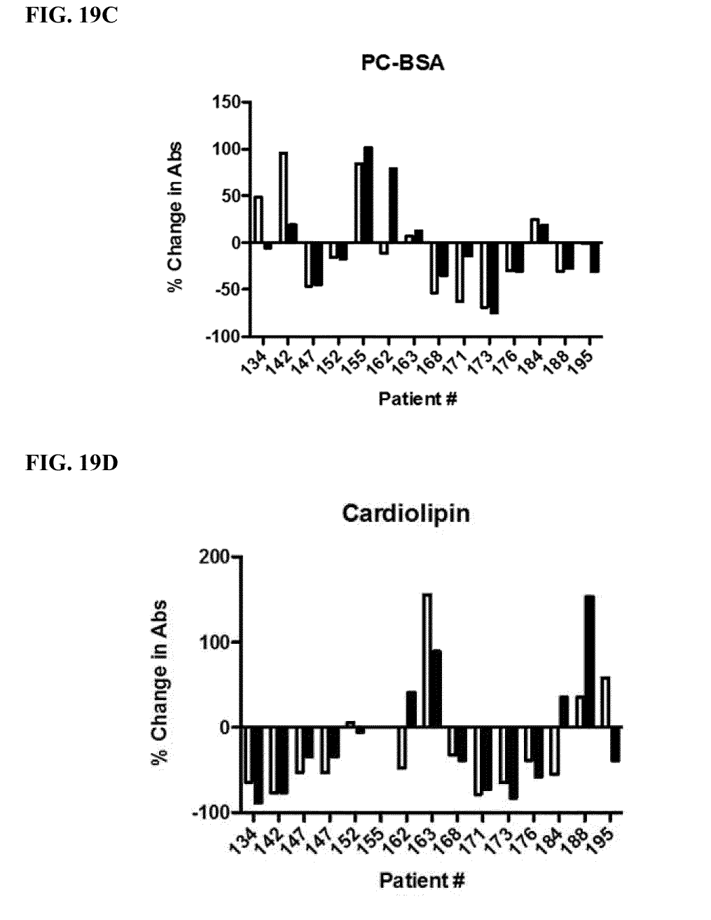

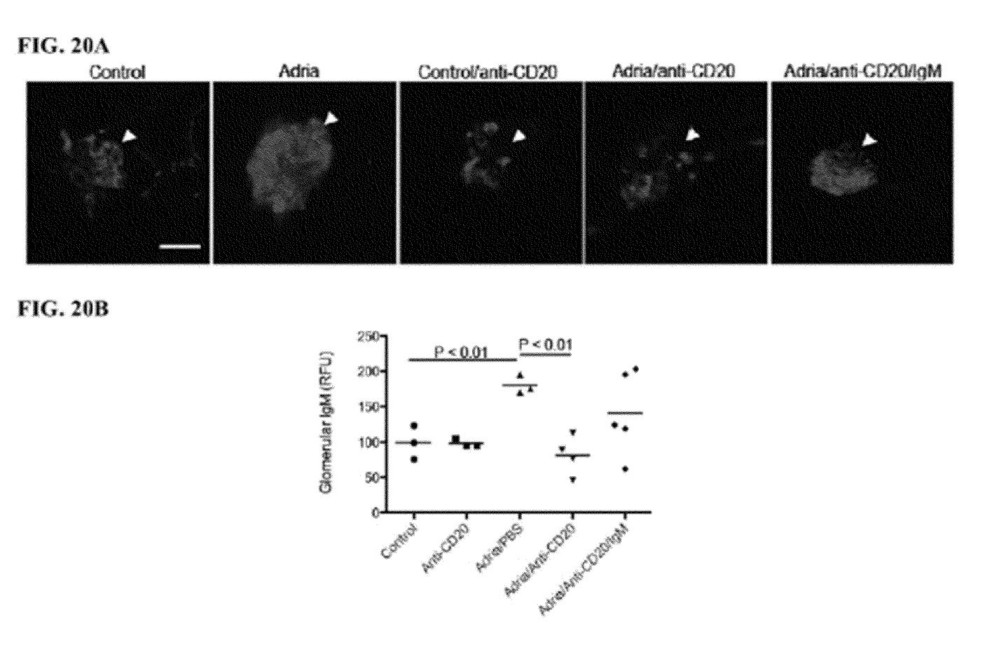

To determine if serum levels of natural IgMs were decreased following liver transplantation in human, serum samples were taken from patients undergoing liver transplantation at three time points: just prior to transplantation, 1 hours post-transplantation and 24 hours post-transplantation. Using ELISA to measure natural IgMs, it was observed that patient serum levels of IgMs specific for phospholipids and annexin IV were decreased at 1 and 24 hrs after transplantation as compared to the levels seen just prior to transplantation (FIGS. 19A-19D). Total IgM levels were not significantly different between the time points (FIGS. 18A-18C). The antigen specificities of the antibodies depleted in human serum were the same as the specificity of the mouse mAbs B4 and C2. To test if the depletion of these antibodies from the serum was a result of them binding to neoepitopes exposed in the post ischemic liver, levels of bound IgM in the liver were measured prior to ischemia and then following reperfusion. There was little IgM detected in normally perfused livers. However, livers that were ischemic and then reperfused had high levels of IgM bound in a characteristic sinusoidal pattern. These data indicate that similar events occur in murine and human hepatic IRI, in that specific circulating natural IgMs recognized and bound to neoepitotpes expressed following ischemia.

### Example 7

### In Vivo Study of IgM Effects in a Chemical and Ischemic Reperfusion Mouse Model of Renal Injury

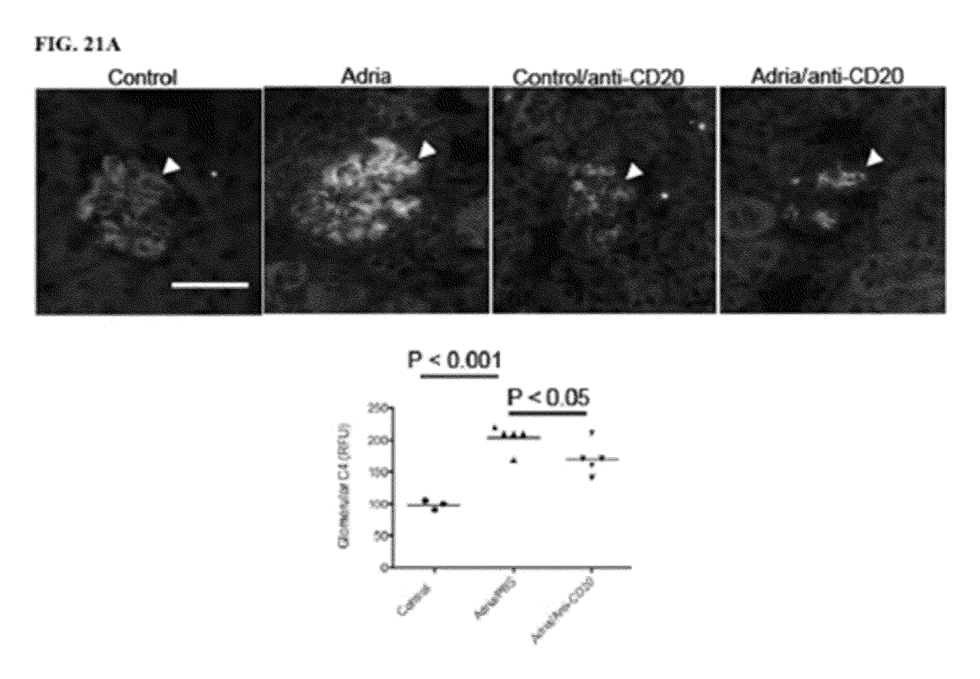

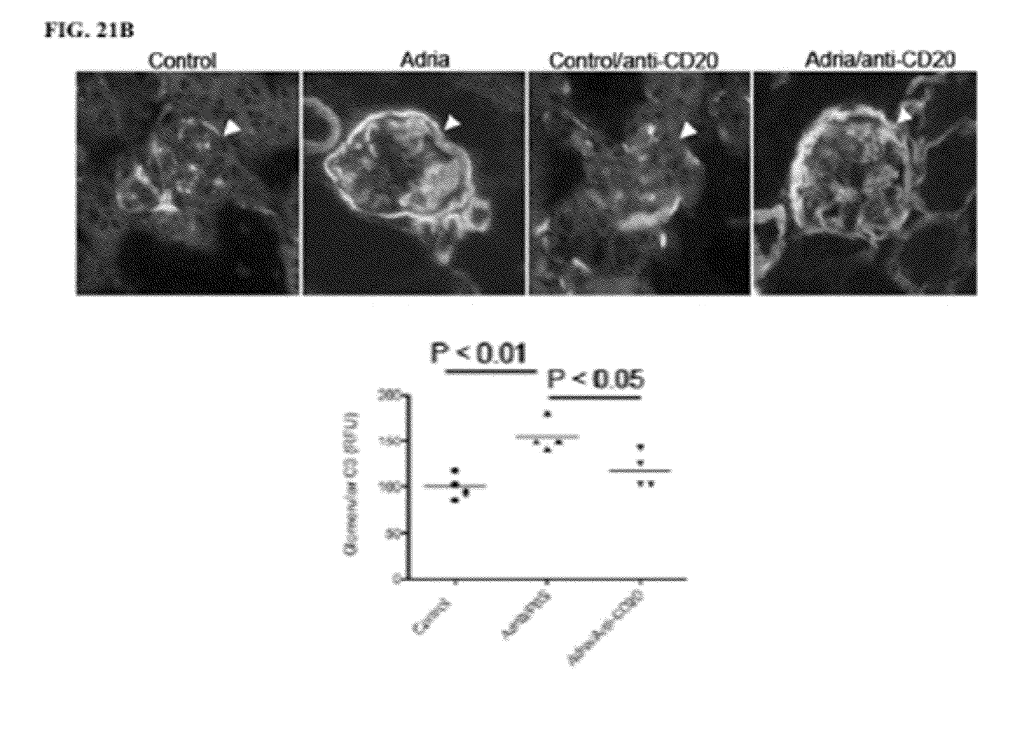

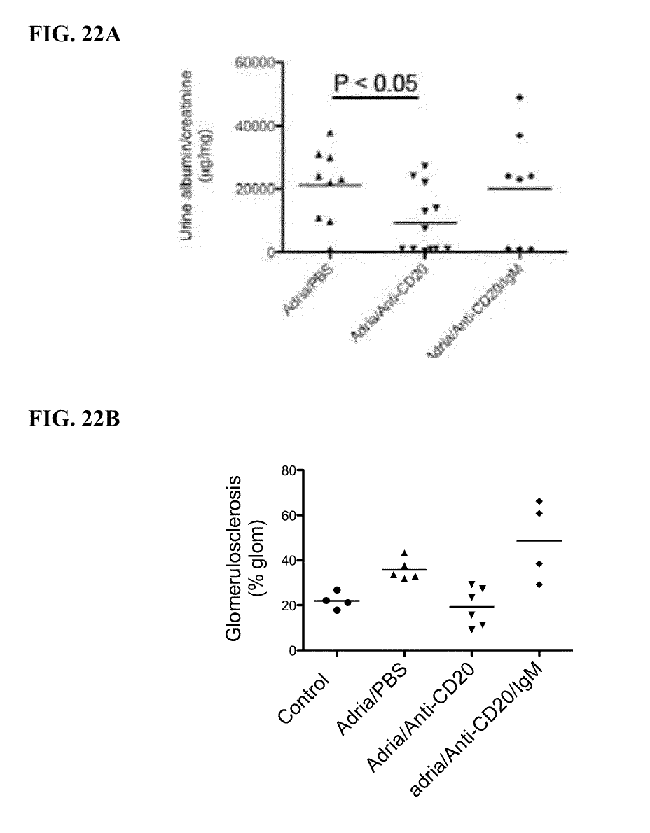

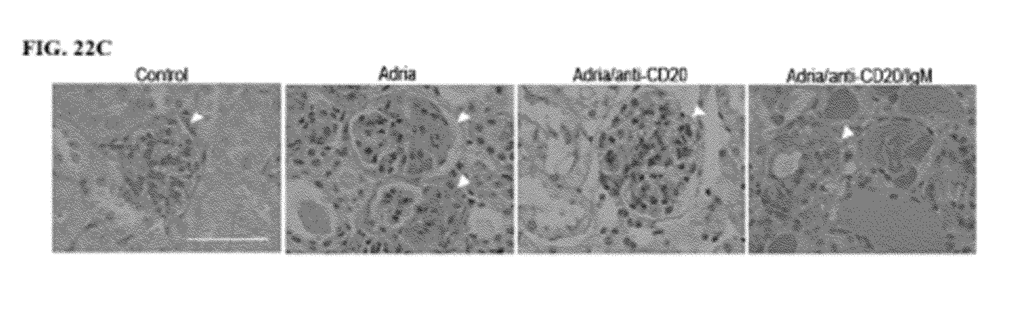

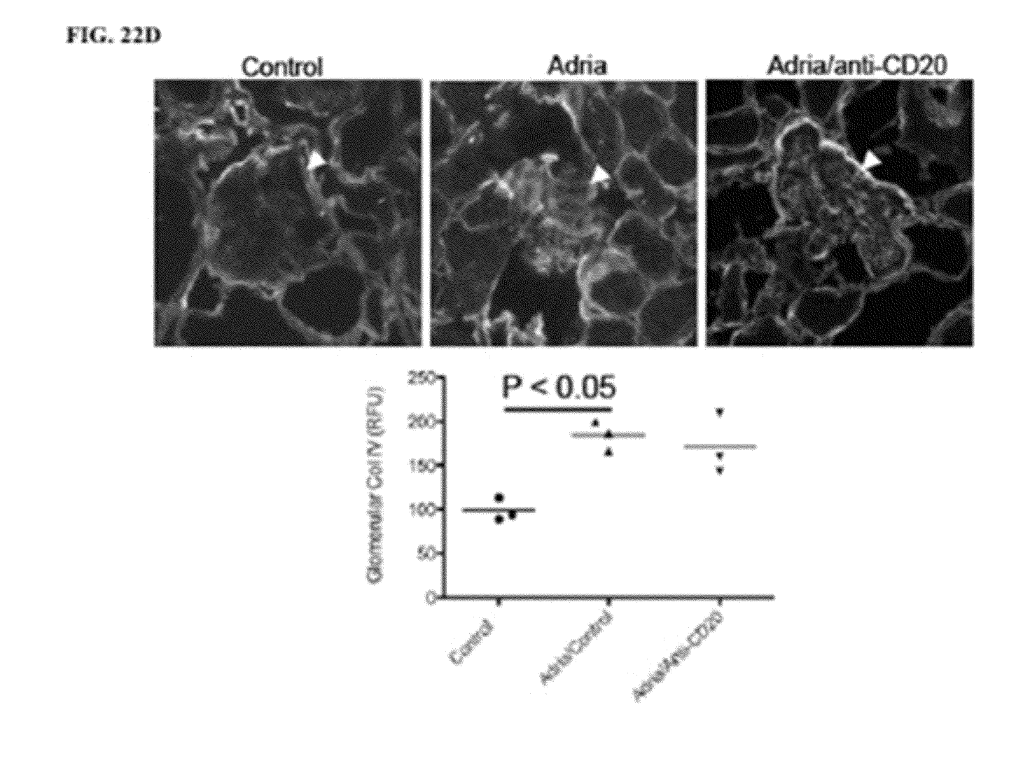

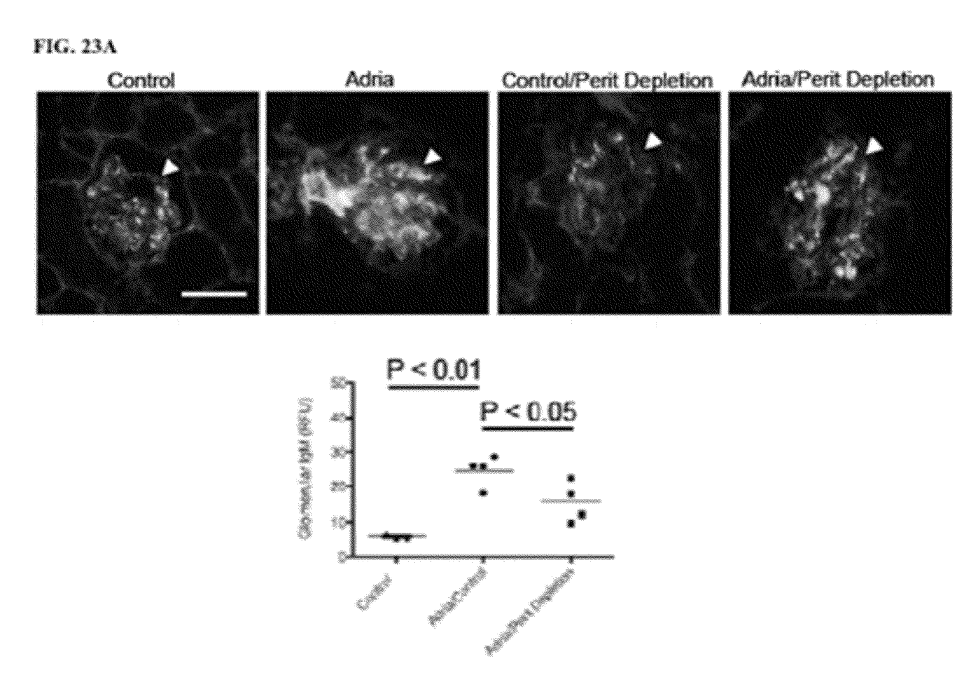

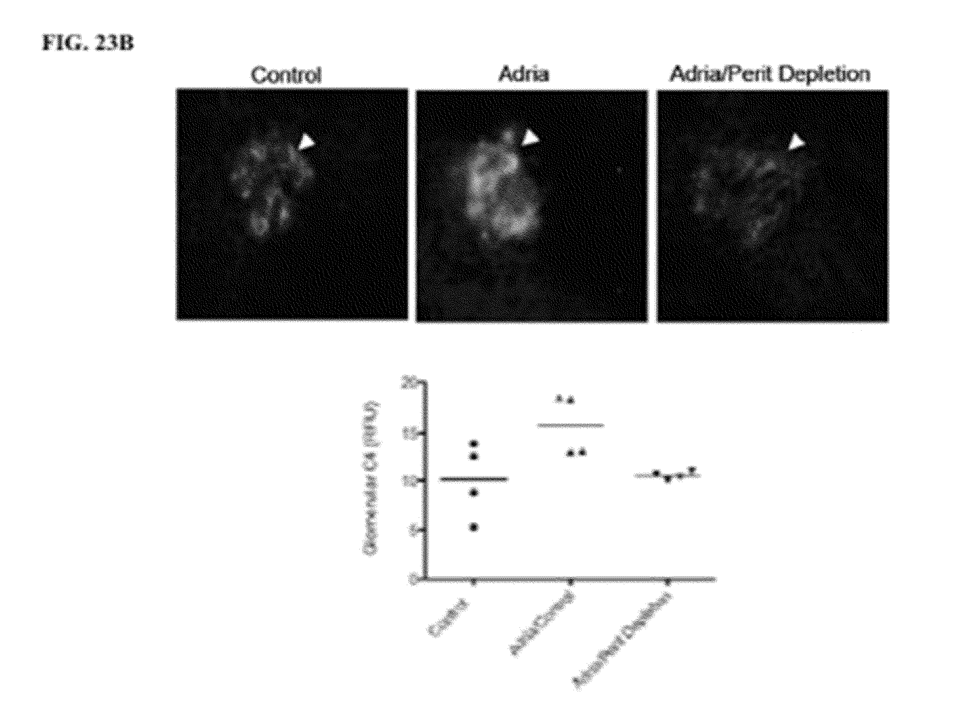

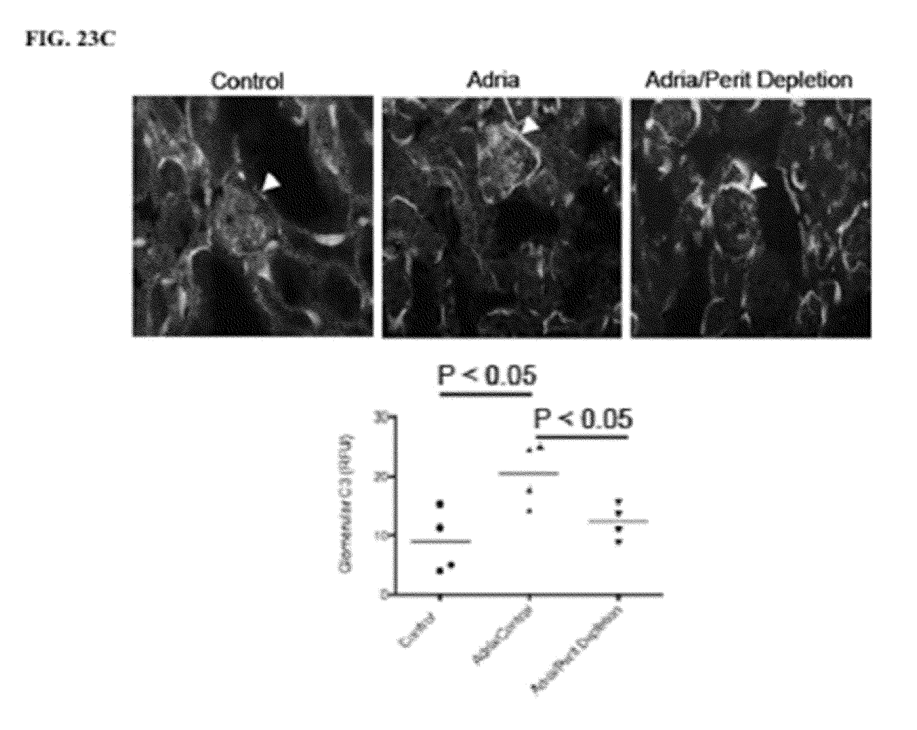

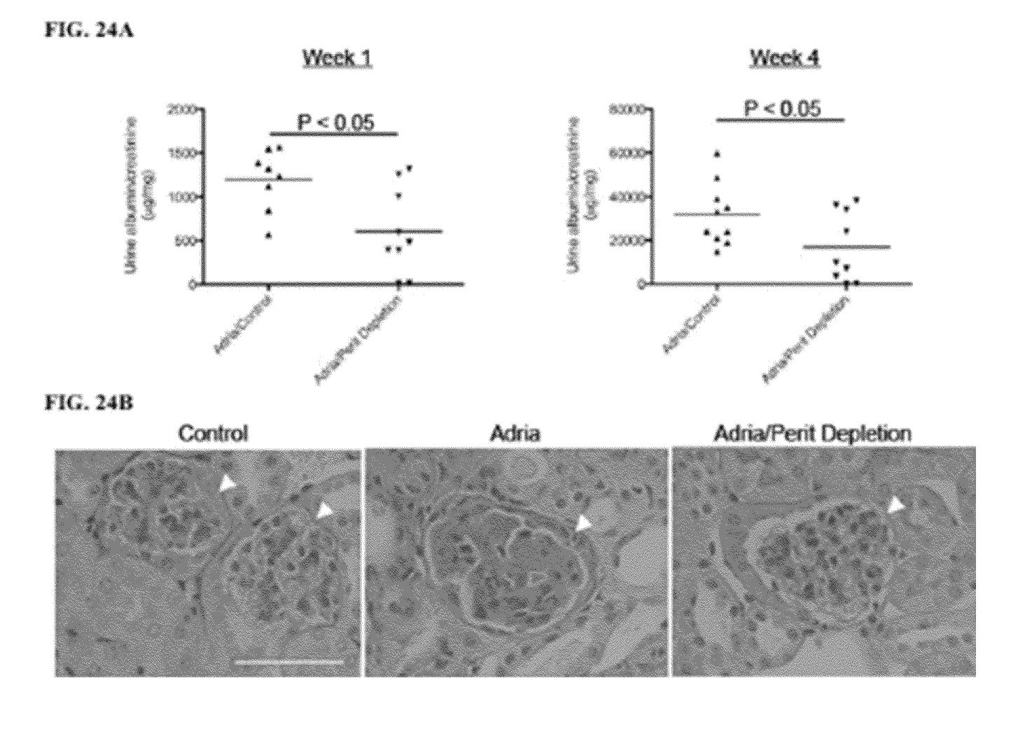

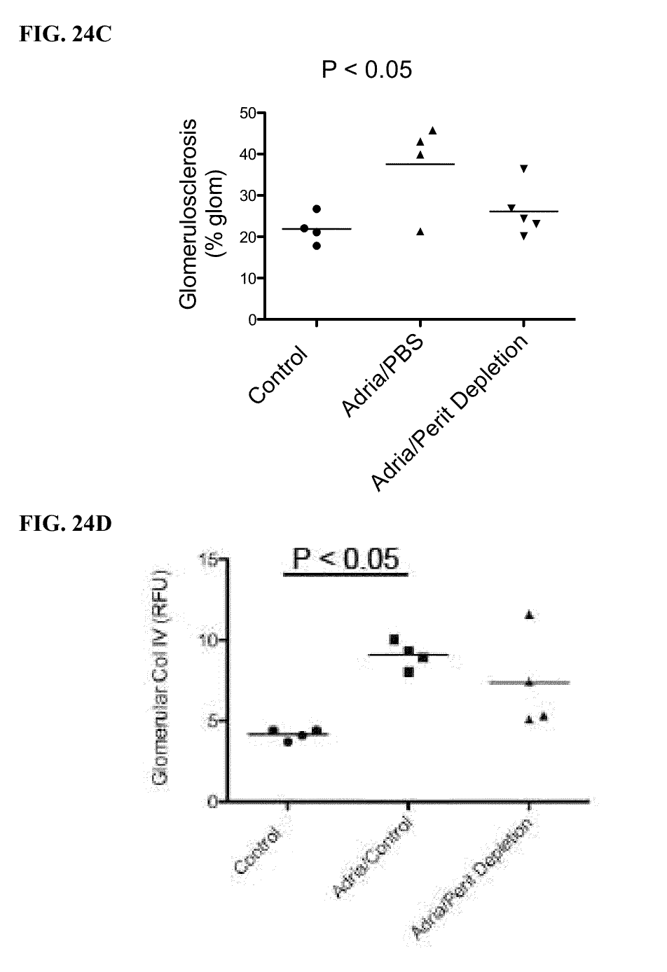

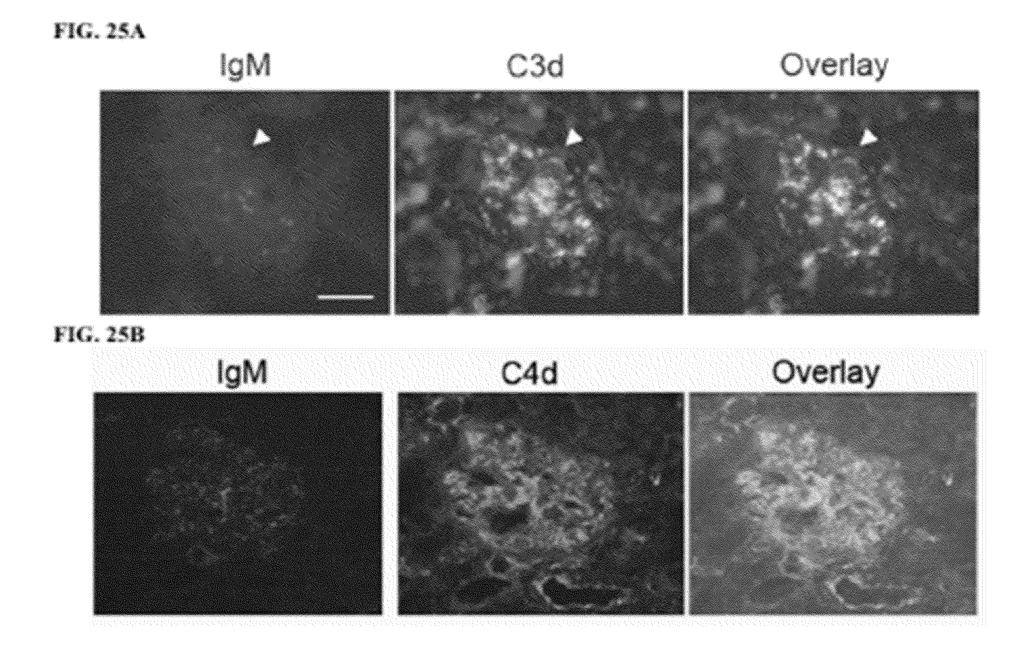

The role of natural IgM antibodies in a chemically induced mouse model of renal injury was investigated. Adriamycin nephropathy was induced in Balb/c mice in 8 to 10 week old male mice with a single intravenous injection of 11 mg/kg adriamycin and the abundance of glomerular IgM was examined (FIGS. 20A-20B). Injection of the mice with adriamycin caused a significant increase in the abundance of glomerular IgM. Pre-treatment of the mice with anti-CD20 prevented an increase in glomerular IgM after injection with adriamycin, and levels of glomerular IgM in mice that received both anti-CD20 and adriamycin were similar to those seen in healthy controls. To determine whether IgM eluted specifically from kidneys with adriamycin nephropathy would bind to glomerular epitopes when re-injected into mice, IgM was purified from the kidneys of adriamycin treated mice and then injected into mice with adriamycin nephropathy that had previously undergone B cell depletion with the anti-CD20. This was repeated every week during the course of the study. A trend towards greater glomerular IgM was seen in the reconstituted mice (FIGS. 20A and 20B). In mice with adriamycin nephropathy, low levels of glomerular C4d deposition were detected in control mice but significantly increased in mice treated with adriamycin (FIG. 21A). Treatment of mice with anti-CD20 reduced glomerular C4d, confirming that the glomerular C4d deposits were caused by immunoglobulin-mediated complement activation. Immunostaining of kidneys for C3 deposits showed a similar pattern. C3 deposition increased in mice after treatment with adriamycin, and treatment of the mice with anti-CD20 prevented this increase (FIG. 21B). Urine albumin/creatinine ratios were measured as a marker of glomerular injury. Mice with adriamycin nephropathy were grossly albuminuric (FIG. 22A), and treatment of the mice with anti-CD20 reduced the degree of albuminuria in mice with adriamycin nephropathy. When anti-CD20 treated mice were treated with adriamycin and then re-injected with purified glomerular IgM, however, the degree of albuminuria was comparable to that seen in mice that received adriamycin alone. This demonstrated that IgM is an important mediator of injury in this model. There was less glomerulosclerosis in mice treated with the anti-CD20 antibody (FIGS. 22B-22C). Mice with adriamycin demonstrated greater glomerular collagen IV deposition than control mice, but treatment with anti-CD20 did not significantly reduce glomerular collagen IV deposition (FIG. 22D). Natural antibody IgM is predominantly produced by peritoneal B-1a cells. Treatment of mice with anti-CD20 depletes peritoneal B cells, although less effectively than it depletes splenic B cells. In order to effectively reduce the peritoneal B cells without affecting splenic B cell function, peritoneal cells were lysed by hypotonic shock. This treatment caused lysis of all peritoneal cells, including macrophages, but it did not affect splenic B cells. Reduction in circulating B cells was observed using this strategy, suggesting that the peritoneal compartment is the source of some circulating B cells. Although all peritoneal cells were reduced by this method, the treatment did not reduce the number of B-1a cells as a percentage of total peritoneal cells. Consequently, the effects of this treatment many not have been specific to depletion of B-1a cells. The level of total IgM in the serum was unaffected by this treatment. In order to deplete the peritoneal B cells in mice with adriamycin nephropathy, the peritoneal cells were lysed by hypotonic shock every three days for two weeks prior to injection with adriamycin in order to give sufficient time for pre-formed IgM to turn over. Depletion of the cells was maintained by intra-peritoneal injections of distilled water every three days during the course of the study. Although this treatment is very effective at immediately reducing the peritoneal B cell numbers, the cells re-accumulated and the depletion at day 14 was not complete. As controls, another group of mice received peritoneal injections with PBS according to the same schedule. When reported as a percentage of total peritoneal cells the B-1a cells were not reduced by this treatment because the treatment reduced all peritoneal cells equally. In mice that underwent peritoneal B cell depletion, the accumulation of glomerular IgM was significantly attenuated after injection of the mice with adriamycin (FIG. 23A). Depletion of the peritoneal cells showed a trend towards reduction of glomerular C4 deposition, and glomerular C3 deposition was reduced by this treatment (FIGS. 23B-23C). Tubulointerstitial C3 deposition was unaffected by this treatment. These results demonstrated that peritoneal B cells generated a significant proportion of the IgM deposited in the glomeruli in this model. Levels of circulating IgM were not affected by the peritoneal cell depletion even though glomerular deposition was reduced, suggesting that the glomerular IgM deposits were comprised of IgM that bound to specific glomerular antigens. As with treatment of mice with anti-CD20, depletion of the peritoneal B cells reduced glomerular complement activation. Urine albumin/creatinine ratios were measured in samples collected 1 or 4 weeks after injection with adriamycin (FIG. 24A). The level of albuminuria was significantly lower in mice that had undergone peritoneal cell depletion at both the early and the later time-point. The reduction in albuminuria at the 1-week time-point indicated that glomerular IgM contributed to injury at an early phase of disease in this model. Some glomeruli in the mice that underwent peritoneal cell depletion appeared normal (FIG. 24B). As was seen in mice treated with anti-CD20, however, depletion of the peritoneal B cells did not significantly attenuate the overall degree of collagen IV accumulation in adriamycin treated mice, however there was less glomerulosclerosis (FIGS. 24B-24D). Previous series have reported that glomerular IgM and/or C3 may be detected in up to 90% of patients with FSGS. The biopsy reports of 174 cases of focal segmental glomerulosclerosis (FSGS) evaluated over the past eight years was reviewed. Of those biopsies, approximately 23% demonstrated glomerular IgM without C3, approximately 2% demonstrated glomerular C3 without IgM, and 7% of the biopsies demonstrated glomerular IgM and C3. Dual staining for IgM and for C3d on tissue in which both factors were detectable was performed (FIG. 25A). C3d and IgM showed a similar pattern of distribution throughout the glomerulus. Dual staining for IgM and C4 was also performed (FIG. 25B). This staining demonstrated similar co-localization of these factors within the glomerulus.

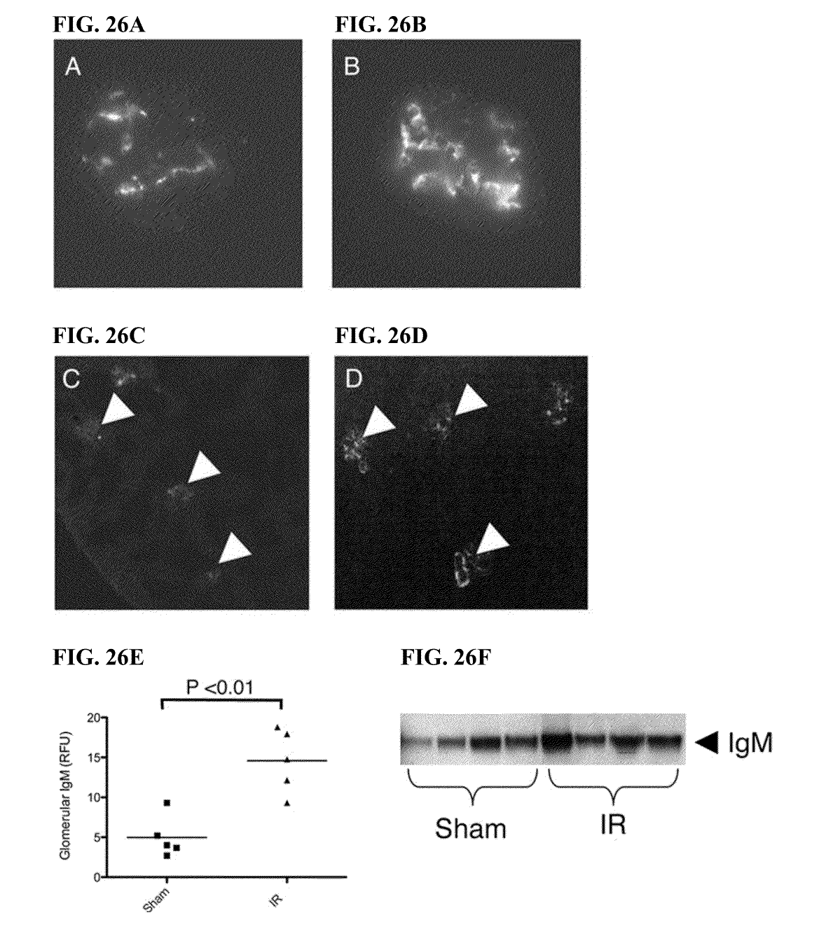

Sham-treated mice and mice subjected to renal IR injury were examined for tissue deposition of IgG and IgM. See Renner et al., J. Immunol., (2010), 185:4393-440 for a description for the renal IR injury mouse model. Briefly, mice were anesthetized with 300 μl 2,2,2-Tribromoethanol (Sigma-Aldrich) by intraperitoneal injection. Laparotomies were performed, and the renal pedicles were located and isolated by blunt dissection. The pedicles were clamped with surgical clips (Miltex Instrument, Bethpage, N.Y.), and occlusion of blood flow was confirmed by visual inspection of the kidneys. The clamps were left in place for 24 min and then released. The kidneys were observed for ˜1 min to ensure blood reflow, and then fascia and skin were sutured with 4-0 silk (U.S. Surgical, Norwalk, Conn.). Sham surgery was performed in an identical manner, except that the renal pedicles were not clamped. The mice were volume resuscitated with 0.5 ml normal saline subcutaneous injection. After 8, 24, 48, or 72 h of reperfusion, the mice were anesthetized, and blood was obtained by cardiac puncture. Laparotomy was performed, and the kidneys were harvested. IgG was not seen in any of the kidneys, but IgM was seen in the mesangium of sham-treated kidneys (FIGS. 26A and 26C) and kidney that underwent IR (FIGS. 26B and 26D). The level of mesangial IgM increased after renal I/R (FIGS. 26E-26F), suggesting that circulating IgM bound to the mesangium during reperfusion. IgM was not seen along the tubular basement membrane. Peritoneal B cells in wild-type mice were depleted for 2 weeks by injecting distilled water into the peritoneum, and control mice were injected with an equal volume of PBS. Depletion of the B-1 population was confirmed by flow-cytometry analysis of B220, CD5, and CD19. Lysis of the peritoneal B-1 cells did not alter the overall levels of circulating IgM, but it did reduce levels of mesangial IgM after sham treatment and after renal I/R compared with control mice. Depletion of peritoneal B-1 cells was also associated with a significantly attenuated increase in serum urea nitrogen (SUN) after 24 h of reperfusion. SUN levels were not significantly different from control mice by 48 hours of reperfusion. Although the decrease in mesangial IgM was associated with protection of renal function, mice that underwent peritoneal B cell depletion still demonstrated tubular necrosis comparable to that seen in wild-type mice.

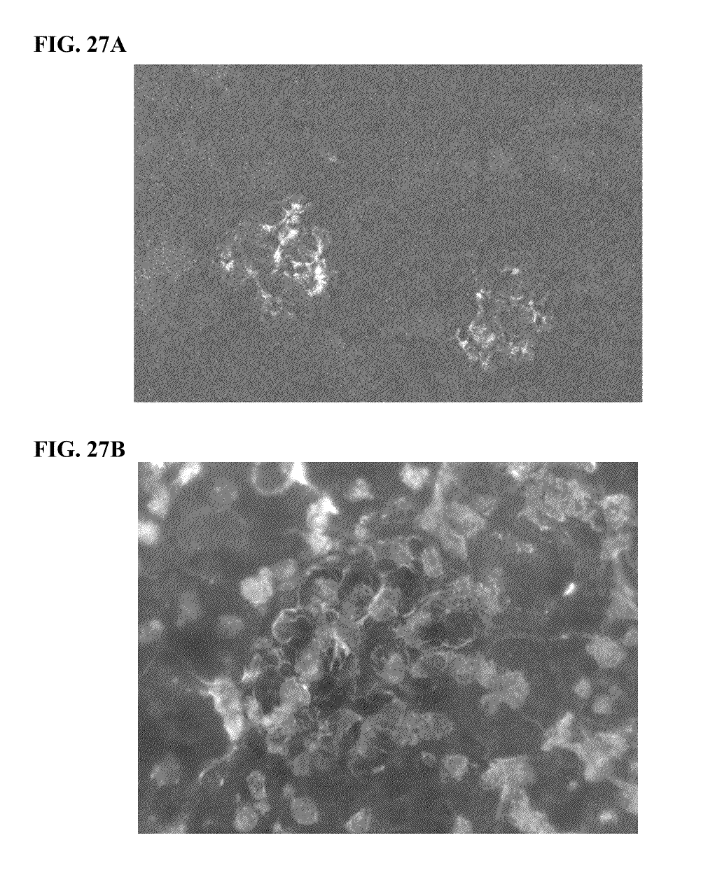

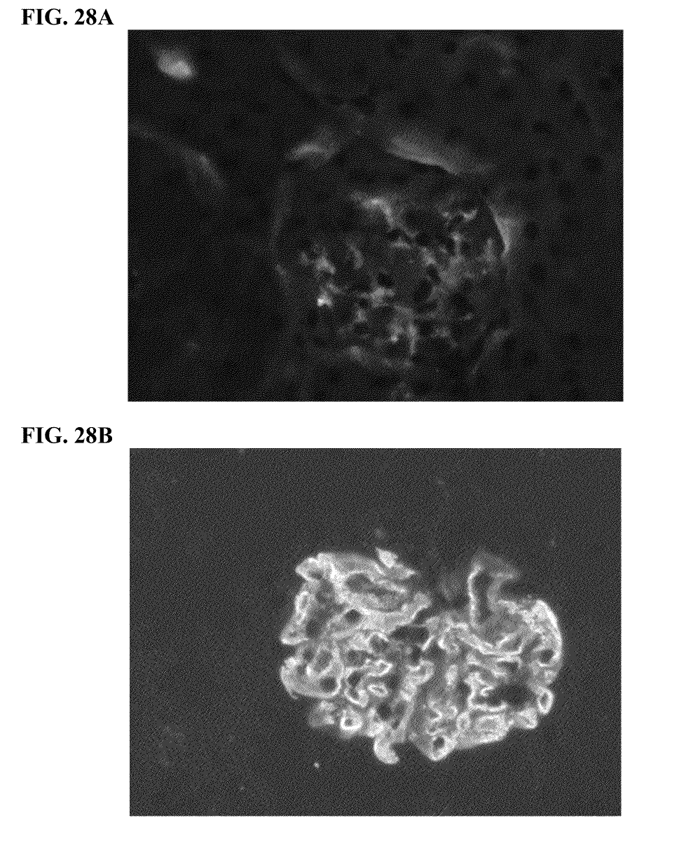

The role of self-reactive C2 or B4 mAbs in a renal IR injury model was investigated. See Renner et al., J. Immunol., (2010), 185:4393-440 for a description for the renal IR injury mouse model. Briefly, mice were anesthetized with 300 μl 2,2,2-Tribromoethanol (Sigma-Aldrich) by intraperitoneal injection. Laparotomies were performed, and the renal pedicles were located and isolated by blunt dissection. The pedicles were clamped with surgical clips (Miltex Instrument, Bethpage, N.Y.), and occlusion of blood flow was confirmed by visual inspection of the kidneys. The clamps were left in place for 24 min and then released. The kidneys were observed for ˜1 min to ensure blood reflow, and then fascia and skin were sutured with 4-0 silk (U.S. Surgical, Norwalk, Conn.). Sham surgery was performed in an identical manner, except that the renal pedicles were not clamped. The mice were volume resuscitated with 0.5 ml normal saline subcutaneous injection. After 8, 24, 48, or 72 h of reperfusion, the mice were anesthetized, and blood was obtained by cardiac puncture. A 100 mcg dose of B4 mAb was injected into a B cell deficient (Mu) mouse subjected to unilateral renal ischemia reperfusion and kidneys were harvested for immunohistochemistry analysis. In this model, B4 mAb and C2 mAb localized in glomeruli consistent with the ability of B4 mAb to recognize neoepitopes due to ischemia (FIGS. 27 AND 27B).

### Example 8

### In Vivo Study of IgM Effects in a Systemic Factor H Deficiency Mouse Model of Renal Disease

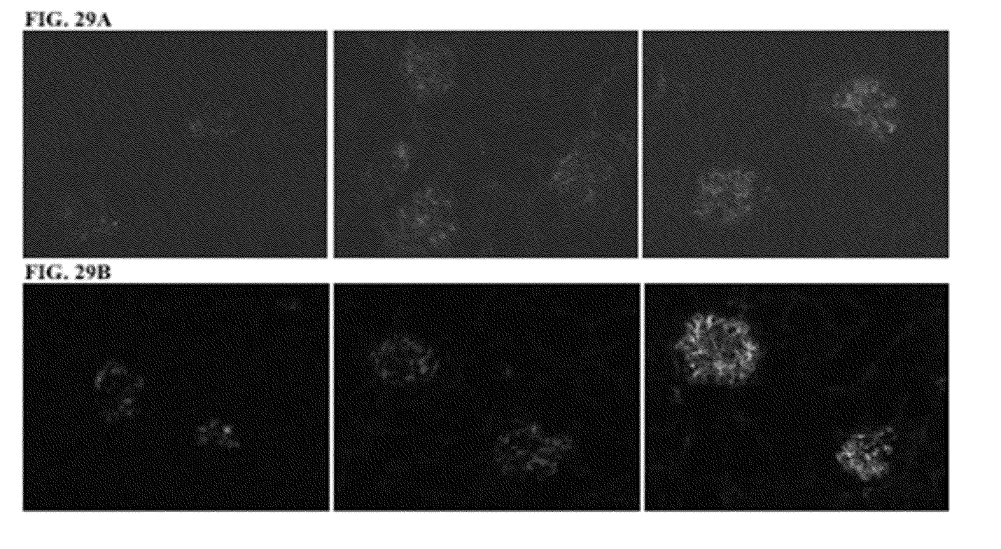

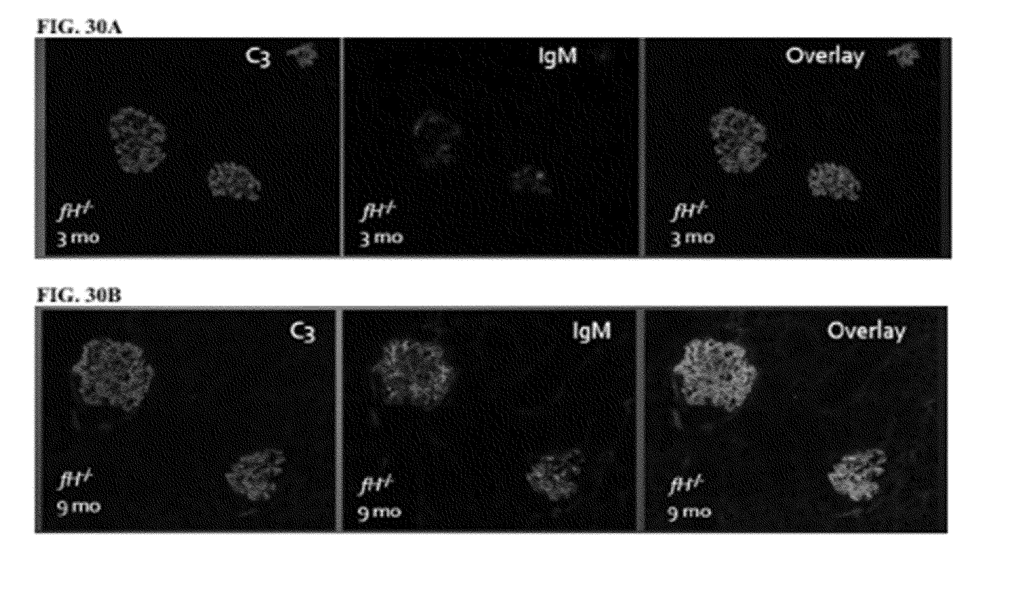

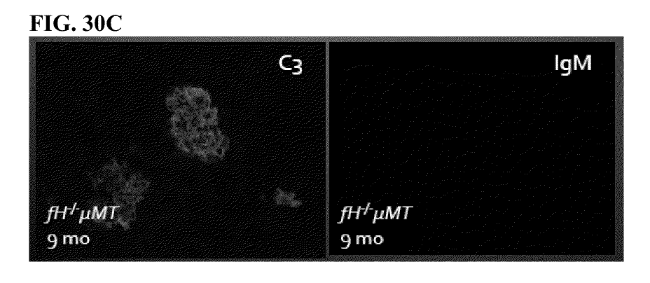

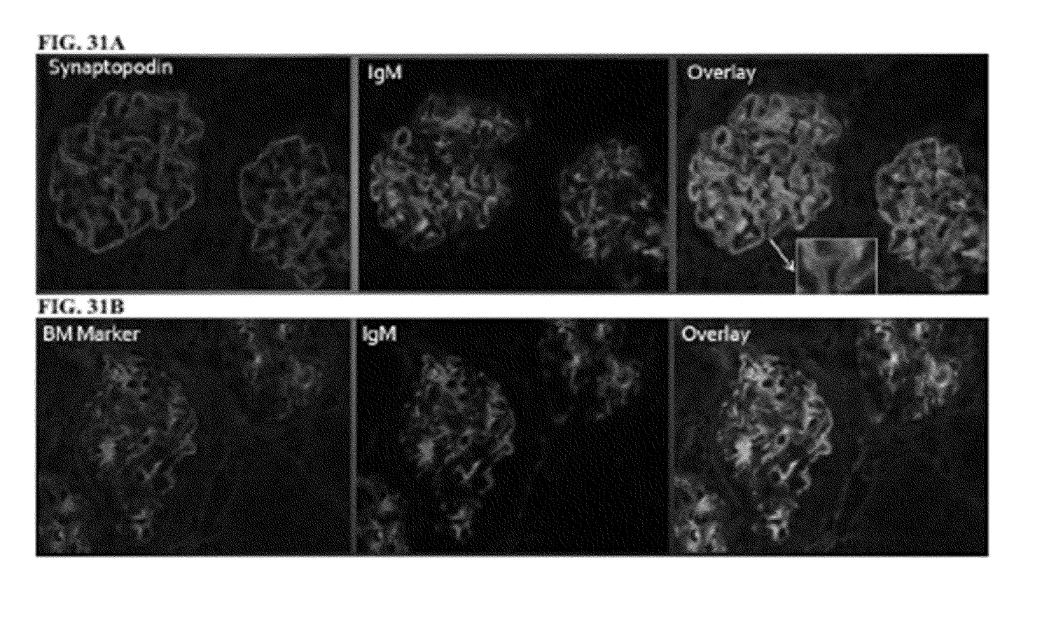

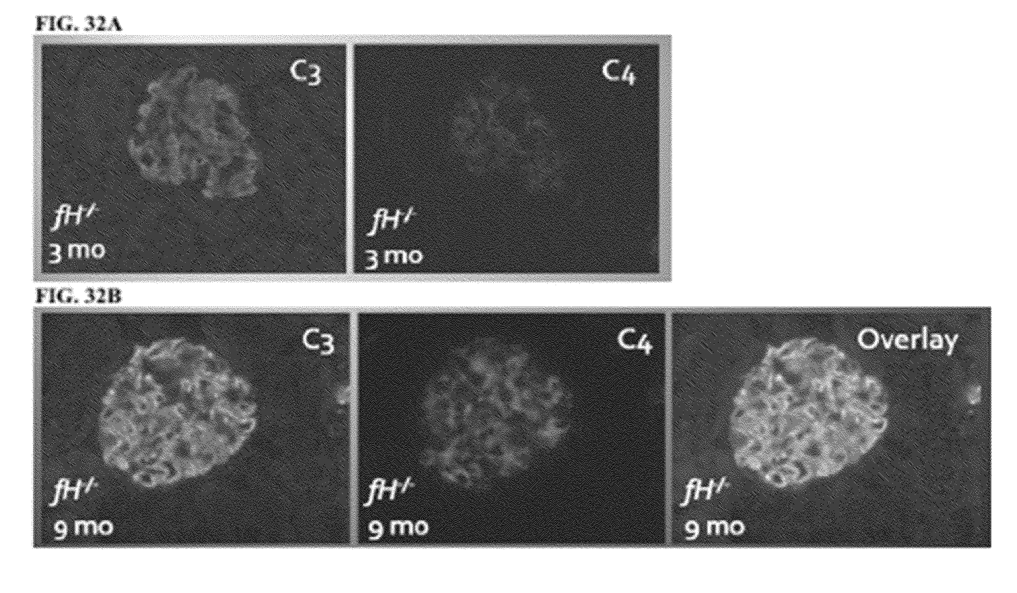

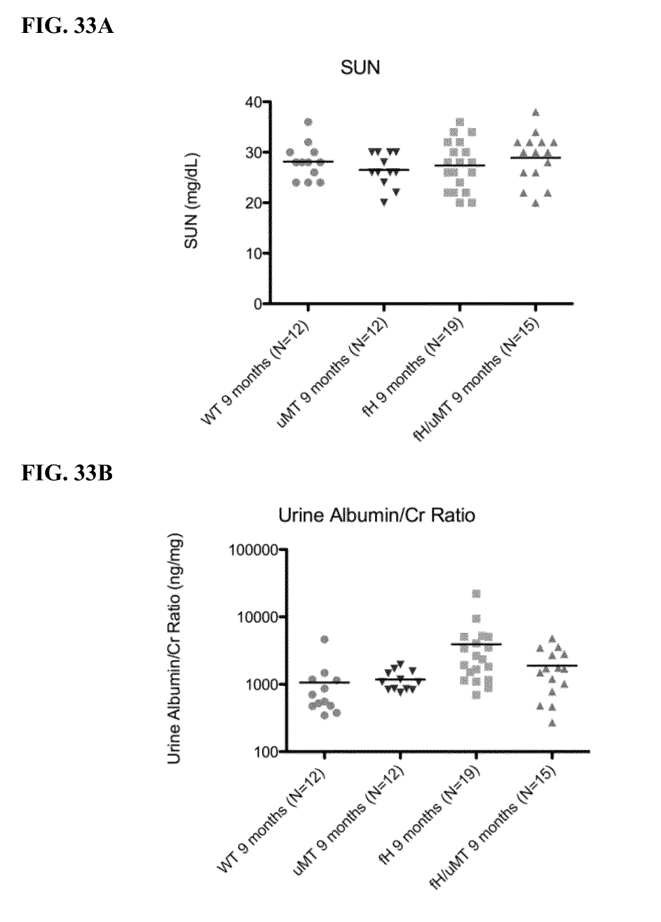

Patients with systemic factor H deficiency primarily develop renal disease with complement deposition within the glomerulus. Factor H knockout mice develop similar disease with glomerular deposition of C3 and albuminuria. See Pickering et al., Nature Genetics., (2002)., 31:424-28. The role of natural IgM deposition and complement activation in the fH knockout model (fH−/− mice), which is not an immune complex model, was investigated. Double knockout mice that lacked factor H and B (fH/μMT mice) were also generated and studied. The kidneys were harvested from wild-type, fH−/−, and fH/μMT mice for immunohistochemistry analysis. Immunofluorescence staining demonstrated increased glomerular C3 deposition in fH/μMT and fH−/− mice as compared to wild-type mice (FIGS. 28A and 28B). Immunofluorescence staining also demonstrated increased glomerular IgM deposition in fH−/− mice as compared to wild-type mice which increased with age (FIGS. 29A and 29B) which followed C3 deposition (FIGS. 30A and 30B). Immunofluorescence staining of glomerulus from fH/μMT showed C3 deposition but no IgM deposition (FIG. 30C). Ultrastructural analysis of immunofluorescence images of glomerulus from fH−/− mice showed co-localization with a marker for the glomerular basement membrane (FIGS. 31A-31B). Additional immunofluorescence staining of glomerulus from fH−/− mice showed that both C3 and C4 were deposited (FIGS. 32A-32B). Histopathology section of the kidneys from fH−/− mice showed basement membrane thickening and foot process effacement. Serum urea nitrogen (SUN) and creatinine (Cr) were similar among the wild-type, fH−/−, and fH/μMT mice (FIG. 33A). However, fH−/− mice developed albuminuria while fH/μMT mice showed protection from albuminuria (FIG. 33B).

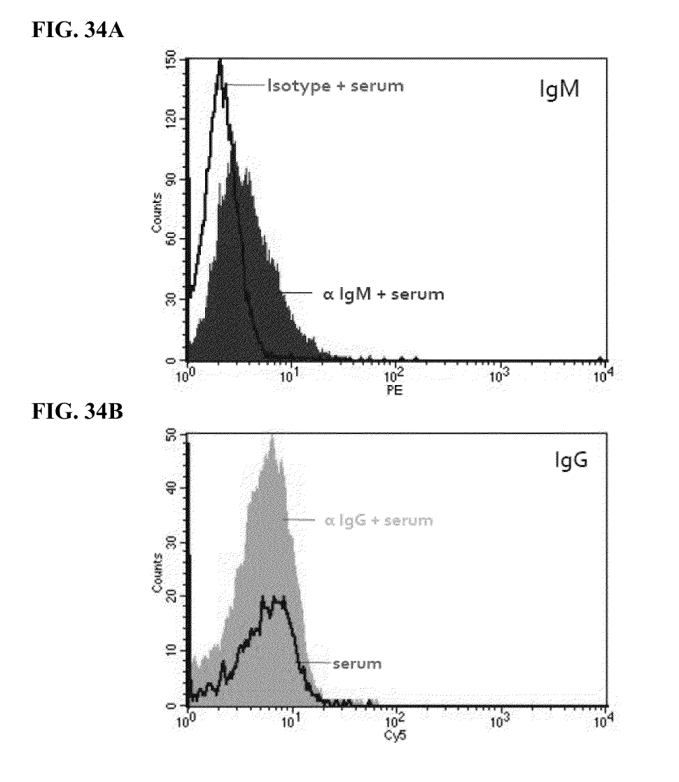

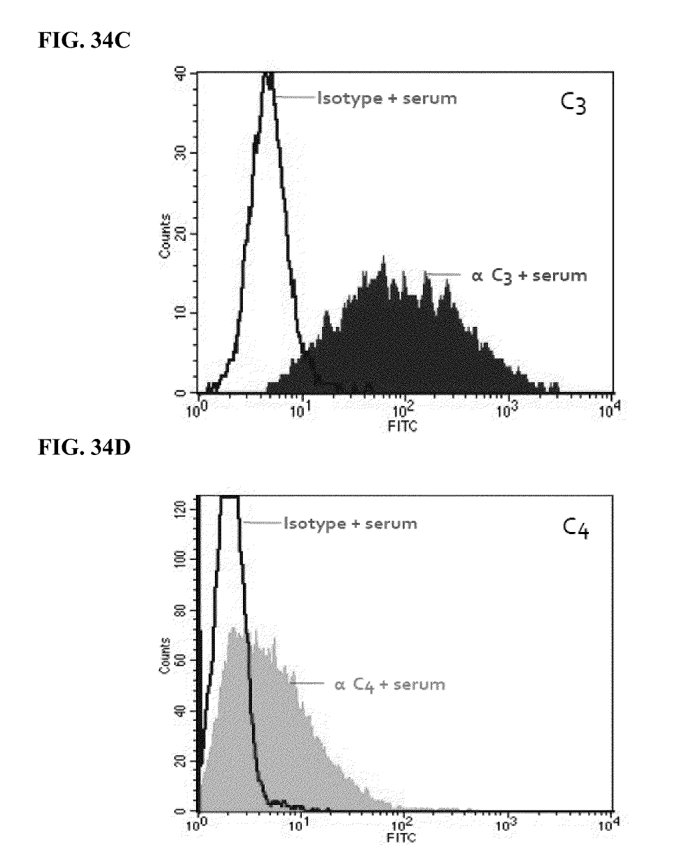

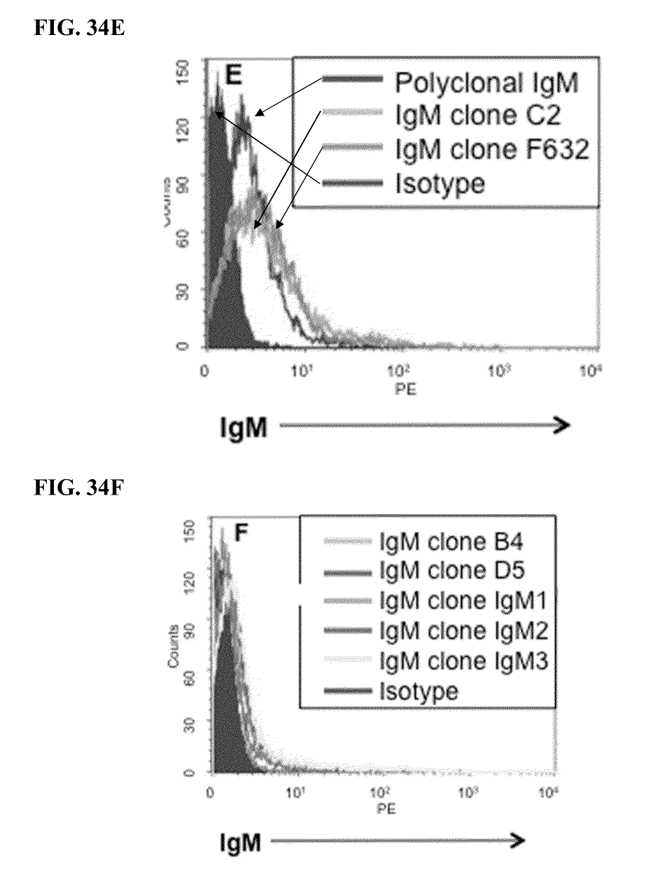

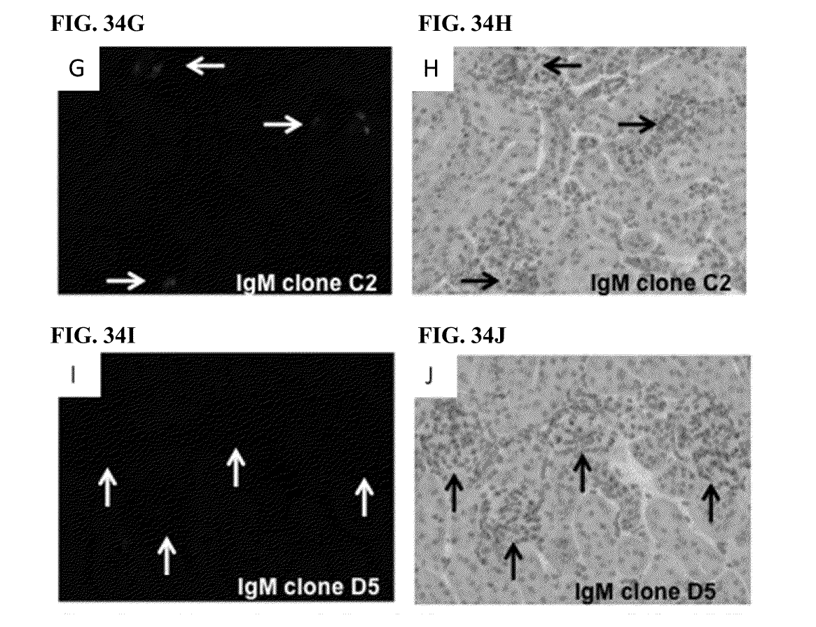

To identify the deposition sites of IgM, an in vitro experiment was conducted by incubating a murine mesangial cell line with wild-type mouse serum. The cells were then analyzed by flow cytometry for the presence of C3, C4, IgM, and IgG. Flow cytometric analysis demonstrated that IgM but not IgG bound to mesangial cells (FIGS. 34A and 34B). Furthermore, both C3 and C4 were bound by mesangial cells (FIGS. 34C and 34D).

In order to characterize the glomerular epitopes, monoclonal natural IgM antibodies were screened for their ability to bind to mesangial cells. Two of the monoclonal antibodies tested, C2 and F632, bound to mesangial cells in vitro (FIG. 34E). The five other clones that were tested did not bind to the mesangial cells (FIG. 34F). The binding of these mAbs to mesangial cells from C57BL/6 mice that were grown in primary culture and to conditionally immortalized mesangial cells that were developed from H-2Kb-tsA58 transgenic mice was also tested. C2 and F632 also bound to these mesangial cells lines, whereas the other IgM clones did not. Next, the ability of these antibodies to bind in vivo was tested. Three μMT mice were each injected with 100 μg of mAb C2, F632, or D5 (D5 did not bind to mesangial cells in vitro). Deposits of IgM were seen in the kidneys of all of the mice injected with C2 (FIGS. 34G and 34H) and F632, but no deposits were seen in the mice injected with D5 (FIGS. 34I and 34J). The identical pattern of mAb binding seen with all three mesangial cell types and with the in vivo experiment supported the specificity of IgM binding in these assays. It also suggested that the target antigen may be the same in all of these assays.

### Example 9

### In Vivo Study of IgM Effects in an Ischemic Reperfusion Mouse Model of Arthritis

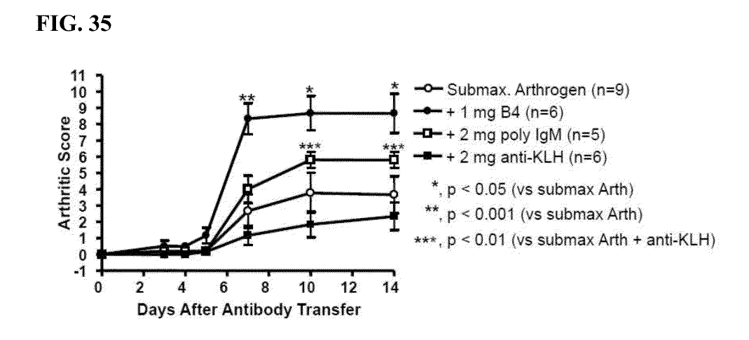

The role of natural IgM antibodies in an ischemic reperfusion mouse model of arthritis was investigated. In these experiments, passive arthritis was induced by intravenous transfer of a submaximal dose of a cocktail of monoclonal antibodies to CII (Arthrogen-CIA®, Chemicon) and/or the monoclonal antibody B4 mAb as well as purified total IgM from wild-type mice. An IgM monoclonal antibody to trinitrophenol-KLH (anti-TNP; BD PharMingen) was administered as a negative control. Arthrogen was titrated to determine the dose that would yield submaximal disease in animals for use in combination with test and control antibodies. An intraperitoneal injection of 50 micrograms/mouse of LPS followed three days after administration of each antibody. From days 1 through 14 after the initial transfer, mice were scored daily by an individual blinded to their treatments for signs of arthritis in the paws based on the following scale: 0=no redness or swelling, 1=one digit swollen, 2=two digits swollen, 3=three digits affected, and 4=entire paw swollen with ankylosis. The scores for each of four paws of a mouse were totaled to give a final score with a maximal severity of 16. Analysis of arthritic signs demonstrated that administration of B4 mAb resulted in a significantly greater arthritic score than poly IgM or a submaximal dose of the cocktail of arthritis-inducing monoclonal antibodies, Arthrogen (FIG. 35).

### Example 10

### In Vitro and In Vivo Study of IgM Effects in Mouse Model of Age-Related Macular Degeneration

Methods and Materials

Reagents

Pooled normal human serum (NHS) from Quidel Corporation (Santa Clara, Calif.) was used as a source of complement. To probe the involvement of the classical pathway, complement C2- and C4-depleted serum as well as complement proteins C2 and C4 were purchased from Complement Technology, Inc. (CompTech; Tyler, Tex.), and C1q-depleted serum was contributed by Deborah Fraser, (University of California Irvine, Irvine, Calif.) (Fraser, D. A., Laust, A. K., Nelson, E. L., and Tenner, A. J. (2009) J Immunol 183, 6175-6185). To probe the involvement of the lectin pathway, recombinant M-ficolin, H-ficolin, and mannan-binding lectin (MBL)-associated serine protease 2 (MASP-2) (Frederiksen, P. D., Thiel, S., Larsen, C. B., and Jensenius, J. C. (2005) Scand J Immunol 62, 462-473; Thiel, S., Kolev, M., Degn, S., Steffensen, R., Hansen, A. G., Ruseva, M., and Jensenius, J. C. (2009) J Immunol 182, 2939-2947; Zacho, R. M., Jensen, L., Terp, R., Jensenius, J. C., and Thiel, S. (2012) J Biol Chem 287, 8071-8081), purified L-ficolin (Lacroix, M., Dumestre-Perard, C., Schoehn, G., Houen, G., Cesbron, J. Y., Arlaud, G. J., and Thielens, N. M. (2009) J Immunol 182, 456-465) and recombinant MBL (Jensenius, J. C., Jensen, P. H., McGuire, K., Larsen, J. L., and Thiel, S. (2003) Biochem Soc Trans 31, 763-767) were used. Finally, to determine the requirement for the alternative pathway, factor B-depleted serum was purchased from CompTech. To analyze the involvement of immunoglobulins (Ig) in triggering the lectin pathway, Ig-depleted serum was obtained from Sunnylab (Sittingbourne, UK) or collected from rag1−/− mice; and reconstitution experiments were performed by adding back antigen-specific (IgM-C2) (Elvington, A., Atkinson, C., Kulik, L., Zhu, H., Yu, J., Kindy, M. S., Holers, V. M., and Tomlinson, S. (2012) J Immunol 188, 1460-1468) or control IgMs (F1102; raised against 4-Hydroxy-3-nitrophenylacetyl hapten conjugated to KLH and purified in the same way as IgM-C2). Primary antibodies included a mouse monoclonal anti-MBL from Millipore (Billerica, Mass.), a rabbit polyclonal anti-MBL from Abcam (Cambridge, Mass.), a rabbit polyclonal MASP-2, a goat antibody against human C3 (CompTech), and monoclonal antibodies to human H-Ficolin, L-Ficolin and M-Ficolin from Santa Cruz Biotechnology (Santa Cruz, Calif.). Species-specific secondary antibodies were from Zymed Laboratories (Invitrogen; Carlsbad, Calif.).

Mice and Collection of Serum

C57BL/6 (B6) and B6 rag1−/− mice were generated from breeding pairs (Jackson Laboratory; Bar Harbor, Me.). For collection of serum, mice were deeply anesthetized (ketamine/xylazine, 80/10 mg/kg). Blood was collected in BD vacutainer tubes by cardiac puncture and serum was collected after clot formation (2 hours on ice) and centrifugation (1000-1400 rcf, 4° C. for 10 min).

Cell Culture

ARPE-19 cells were expanded in Dulbecco's modified Eagle's medium F12 (Invitrogen) with 10% fetal bovine serum (FBS) and antibiotics as described before (Thurman, J. M., Renner, B., Kunchithapautham, K., Ferreira, V. P., Pangbum, M. K., Ablonczy, Z., Tomlinson, S., Holers, V. M., and Rohrer, B. (2009) J Biol Chem 284, 16939-16947). Human fetal retinal pigment epithelium (RPE) cells were prepared and expanded in Minimum Essential Medium (MEM; Sigma-Aldrich, St. Louis, Mo.) with 15% FBS, following our published protocol (Bandyopadhyay, M., and Rohrer, B. (2012) Invest Ophthalmol Vis Sci 53, 1953-1961). Globes were supplied by Advanced Bioscience Recourses (Alameda, Calif.), and experiments adhered to the Declaration of Helsinki on the ethical principles for medical research involving human materials.

Transepithelial Resistance (TER) Measurements

ARPE-19 cells or human fetal RPE were grown as mature monolayers on 6-well Transwell inserts (Corning, 0.4 μm PET, 24 mm insert) in the presence of 5% FBS for 2-3 weeks (Ablonczy, Z., and Crosson, C. E. (2007) Exp Eye Res 85, 762-771). For the final 2-3 days prior to the experiments, cells were changed to serum-free media. Complement activation was induced as reported previously (Thurman, J. M., Renner, B., Kunchithapautham, K., Ferreira, V. P., Pangbum, M. K., Ablonczy, Z., Tomlinson, S., Holers, V. M., and Rohrer, B. (2009) J Biol Chem 284, 16939-16947), exposing cells to 0.5 mM H2O2 in the presence of 10% normal human serum (NHS). It was previously shown that sublytic complement activation results in VEGF release, which in turn reduces barrier function (Thurman, J. M., Renner, B., Kunchithapautham, K., Ferreira, V. P., Pangburn, M. K., Ablonczy, Z., Tomlinson, S., Holers, V. M., and Rohrer, B. (2009) J Biol Chem 284, 16939-16947), TER measurements are a convenient readout for the level of activity in the complement cascade. TER was determined by measuring the resistance across the monolayer with an EVOM volt-ohmmeter (World Precision Instruments, Sarasota, Fla.). The value for cell monolayers was determined by subtracting the TER for filters without cells and the percentage calculated using the starting value as reference.

Binding Assays

For testing the binding of ficolin, non-specific IgM or antigen-specific IgM (IgMC2) binding, ARPE-19 cells were grown as monolayers in 96-well plates. Cells were changed to serum-free media 2 days before the experiments. To identify ficolin binding, normal human serum was used as the source of ficolins. Cells were incubated with serial dilutions of serum for 1 hour at 37° C., washed and fixed in PBS containing 4% paraformaldehyde, and non-specific binding sites were blocked with 1% BSA in PBS. Bound ficolins were detected with corresponding antibodies followed by alkaline phosphatase conjugated secondary antibody and color development using the pNPP phosphatase substrate system (KPL; Gaithersburg, Md.). To characterize non-specific IgM or antigen-specific IgM (IgM-C2) binding to control and oxidatively-stressed ARPE-19 cells, neo-epitopes were generated by exposing cells to 0.5 mM H2O2 for 10 minutes prior to incubation with serial dilutions of serum (for detection of IgM binding) or C2 antibody (in PBS). Bound IgMs were detected with alkaline phosphatase conjugated secondary antibody and color development using the pNPP phosphatase substrate system.

Depletion of MBL

MBL-depleted human serum was prepared using mannan-agarose (Sigma-Aldrich, St. Louis, Mo.) as depleting beads according to published protocols (Rajagopalan, R., Salvi, V. P., Jensenius, J. C., and Rawal, N. (2009) Immunol Lett 123, 114-124). A 2.0 mL column of mannan-agarose was prepared and equilibrated with Veronal buffer (Lonza; Allendale, N.J.) containing calcium chloride (3 mM) and magnesium chloride (10 mM). Normal human serum was passed through the column and the flow through was collected. The depletion of MBL was confirmed by ELISA and Western blot.

MBL ELISA

Microtiter (Immulon2; Dynatech Laboratories, Chatilly, Va.) plates were first coated with 10 μg/mL polyclonal rabbit anti-MBL capture antibody overnight at 4° C. The plates were then washed three-times with PBS and blocked with 3% milk in PBS for 1 hr at room temperature, followed by exposure to the antigen (normal human serum or MBL-depleted human serum) for 2 hrs at 37° C. The plates were again washed and incubated with monoclonal antibody to MBL followed by peroxidase conjugated secondary antibody and color development using Turbo-TMB ELISA (Pierce; Thermo Scientific, Rockford, Ill.).

Characterization of IgM-C2 Epitopes

ELISAs to determine reactivity of the IgM-C2 to phospholipids were performed as described (Elvington, A., Atkinson, C., Kulik, L., Zhu, H., Yu, J., Kindy, M. S., Holers, V. M., and Tomlinson, S. (2012) J Immunol 188, 1460-1468). In short, microtiter plates (Immulon 1B) were coated with 100 μL/well 50 μg/mL phospholipid in methanol. After the plates were air-dried, the wells were washed with PBS and blocked with 1% BSA. IgM was added to wells and bound-Ab detected by alkaline-phosphatase-conjugated goat anti-mouse IgM (Jackson ImmunoResearch Laboratories, West Grove, Pa.). Relative units of Ab were calculated by comparing OD at 405 nm for individual titrated serum with a standard curve of OD measurements established previously (Elvington, A., Atkinson, C., Kulik, L., Zhu, H., Yu, J., Kindy, M. S., Holers, V. M., and Tomlinson, S. (2012) J Immunol 188, 1460-1468). Binding of IgM-C2 was compared against a control IgM known to cross-react with annexin IV (IgM-B4) (Elvington, A., Atkinson, C., Kulik, L., Zhu, H., Yu, J., Kindy, M. S., Holers, V. M., and Tomlinson, S. (2012) J Immunol 188, 1460-1468). Two phospholipids were assayed; phosphorylcholine (PC)(10)-BSA (Biosearch Technologies, Novato, Calif.) and malondialdehyde (MDA)-BSA (Cell Biolabs, San Diego, Calif.).

SDS-Polyacrylamide Gel Electrophoresis and Western Blotting

Protein concentration was determined by BCATM protein assay according to the manufacturer's instructions (Pierce, Rockford, Ill.). For each sample, 40 μg of protein was denatured, subjected to SDS-polyacrylamide gel electrophoresis and analyzed by immunoblotting with appropriate antibodies.

Immunofluorescence Staining

Surface exposure of phospholipid-specific epitopes was examined by immunofluorescence microscopy. ARPE-19 cells were grown on 35-mm lysine-coated glass-bottom culture dishes (MatTek Corporation; Ashland, Mass.), treated with H2O2 for 10 minutes, fixed in PBS containing 4% paraformaldehyde and nonspecific binding sites were blocked with 1% normal goat serum and 3% BSA in PBS (preabsorption buffer) for 2 hours. The cells were incubated overnight at 4° C. with either rabbit anti-MDA polyclonal antibody (1:200 in PBS) followed by incubation for 1 hr at room temperature with FITC-conjugated goat anti-rabbit IgG (1:200; Zymed Laboratories, Invitrogen); or with IgM natural antibody (IgM-C2) followed by goat anti-mouse IgM (1:200; Zymed Laboratories, Invitrogen). As a negative control, primary antibodies were omitted. Staining was also performed ex vivo on eyes with CNV lesions (see below). Eyecups were fixed in 4% paraformaldehyde, washed, preabsorbed and incubated over night at 4° C. with C2-IgM (1:200), followed by anti-mouse IgM (1:200) as described above. Omission of primary antibody staining served as the negative control. Staining of cells and flatmounts was examined by confocal microscopy (Oympus FluoView).

In Vivo CNV Induction and Assessment

B6 rag1−/− and C57BL/6 mice were housed in the Medical University of South Carolina animal care facility under a 12:12 hour light:dark cycle with access to food and water ad libitum. All experiments were performed in accordance with the Association for Research in Vision and Ophthalmology and were approved by the Institutional Animal Care and Use Committee. CNV lesions (four spots in each eye surrounding the optic nerve) were generated as described previously using argon laser photocoagulation (532 nm; 100 μm spot size; 0.1 s duration; 100 mW) (Rohrer, B., Long, Q., Coughlin, B., Wilson, R. B., Huang, Y., Qiao, F., Tang, P. H., Kunchithapautham, K., Gilkeson, G. S., and Tomlinson, S. (2009) Invest Ophthalmol Vis Sci 50, 3056-3064). Animals (n=6-8 per treatment group) were treated on days 0, 2 and 4 with C2-IgM or control F1102-IgM (100 μg diluted in 400 μL PBS) using intraperitoneal (IP) injections. IP injections have been shown to be effective for antibody delivery to CNV lesions (Campa, C., Kasman, I., Ye, W., Lee, W. P., Fuh, G., and Ferrara, N. (2008) Invest Ophthalmol Vis Sci 49, 1178-1183). Relative CNV size was determined in flatmount preparations of RPE-choroid stained with ICAM2 (Campa, C., Kasman, I., Ye, W., Lee, W. P., Fuh, G., and Ferrara, N. (2008) Invest Ophthalmol Vis Sci 49, 1178-1183). Staining, flatmounting, imaging and analysis of fluorescence measurements by confocal microscopy were performed as reported previously (Rohrer, B., Long, Q., Coughlin, B., Wilson, R. B., Huang, Y., Qiao, F., Tang, P. H., Kunchithapautham, K., Gilkeson, G. S., and Tomlinson, S. (2009) Invest Ophthalmol Vis Sci 50, 3056-3064). Data are expressed as mean±SEM per eye.

Statistics

For data consisting of multiple groups, one-way ANOVA followed by Fisher's post hoc test (P<0.05) was used; single comparisons were analyzed by Student t test analysis (P<0.05).

Sublytic complement activation as a function of complement status.

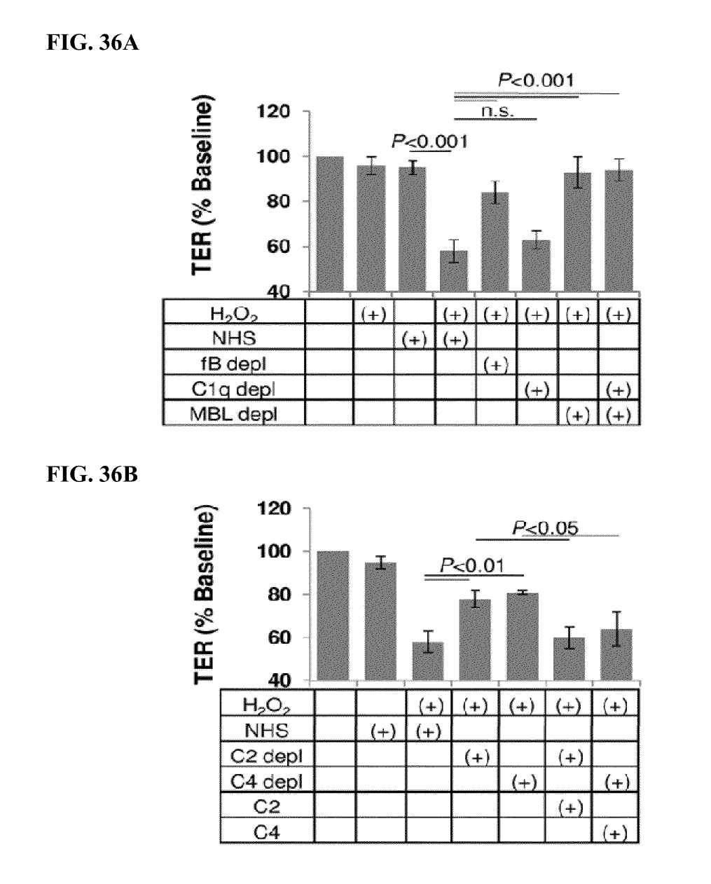

Using a combination of serum-depletion strategies, complement activation pathways involved in TER reduction were analyzed (FIG. 36A). ARPE-19 cells grown as monolayers on Transwell filters develop TER levels of 40-45 □/cm2, a value that is not affected over the course of a 4-hour exposure to 0.5 mM of H2O2 or 10% of normal human serum (NHS). However, the co-treatment with H2O2+NHS reduced TER by >40% (i.e., resulting in <60% baseline TER values; P<0.001). TER reduction in control serum did not differ significantly from that elicited in the presence of C1q-depleted serum, whereas both MBL- or factor B-depleted serum were found to be ineffective in reducing TER (i.e., resulting in ˜95% baseline TER values after 4 hours of exposure; n.s.). Taken together, these results allow the conclusion that the lectin pathway is responsible for triggering the complement attack on oxidatively-stressed RPE cells, followed by the amplification by the alternative pathway.

The typical activity of the lectin pathway serine protease MASP-2 is to split complement C2 and C4 into their respective a-(C2a and C4a) and b-components (C2b and C4b), resulting in the formation of the C3 convertase (C4b2a complex) (Takahashi, M., Mori, S., Shigeta, S., and Fujita, T. (2007) Adv Exp Med Biol 598, 93-104). However, recently, bypass mechanisms have been observed; MASP-2 has been shown to activate the alternative pathway via a C2-bypass pathway (Tateishi, K., and Matsushita, M. (2011) Microbiol Immunol 55, 817-821), and Schwaeble and colleagues have described a lectin pathway-dependent C4-bypass (Schwaeble, W. J., Lynch, N. J., Clark, J. E., Marber, M., Samani, N. J., Ali, Y. M., Dudler, T., Parent, B., Lhotta, K., Wallis, R., Farrar, C. A., Sacks, S., Lee, H., Zhang, M., Iwaki, D., Takahashi, M., Fujita, T., Tedford, C. E., and Stover, C. M. (2011) Proc Natl Acad Sci USA 108, 7523-7528). Removing C2 or C4 from normal human serum attenuated the effect of H2O2+serum on TER (˜80% baseline TER values) (FIG. 36B), although not to the level of MBL-depleted serum (˜95% baseline TER values). Addition of physiological levels of C2 (10 μg/mL) and C4 (400 μg/mL) to their respective depleted serum reconstituted the effect of serum on TER in both C2- and C4-depleted sera (FIG. 36B). Based on the partial effect on TER reduction of the C2- and C4-depleted sera, the results suggest that while activity in the lectin pathway involves the generation of the regular C4b2a complex, a contribution by MASP-2 mediated activation of the alternative pathway cannot be excluded as has been described for MASP-1 and MASP-3 (Banda, N. K., Takahashi, M., Takahashi, K., Stahl, G. L., Hyatt, S., Glogowska, M., Wiles, T. A., Endo, Y., Fujita, T., Holers, V. M., and Arend, W. P. (2011) Mol Immunol 49, 281-289).

Pattern Recognition Molecules in the Lectin Pathway

Complement activation requires the binding of a ligand by a pattern recognition molecule. For the lectin pathway, those entry molecules are mannan-binding lectin (MBL) and the ficolins (H-ficolin, L-ficolin and M-ficolin), which then activate the MBL-associated serine protease, MASP-2. Here, a combination of binding assays and reconstitution experiments was employed to determine which pattern recognition molecule could be employed to recognize ligands on oxidatively-stressed RPE cells to activate the lectin pathway.

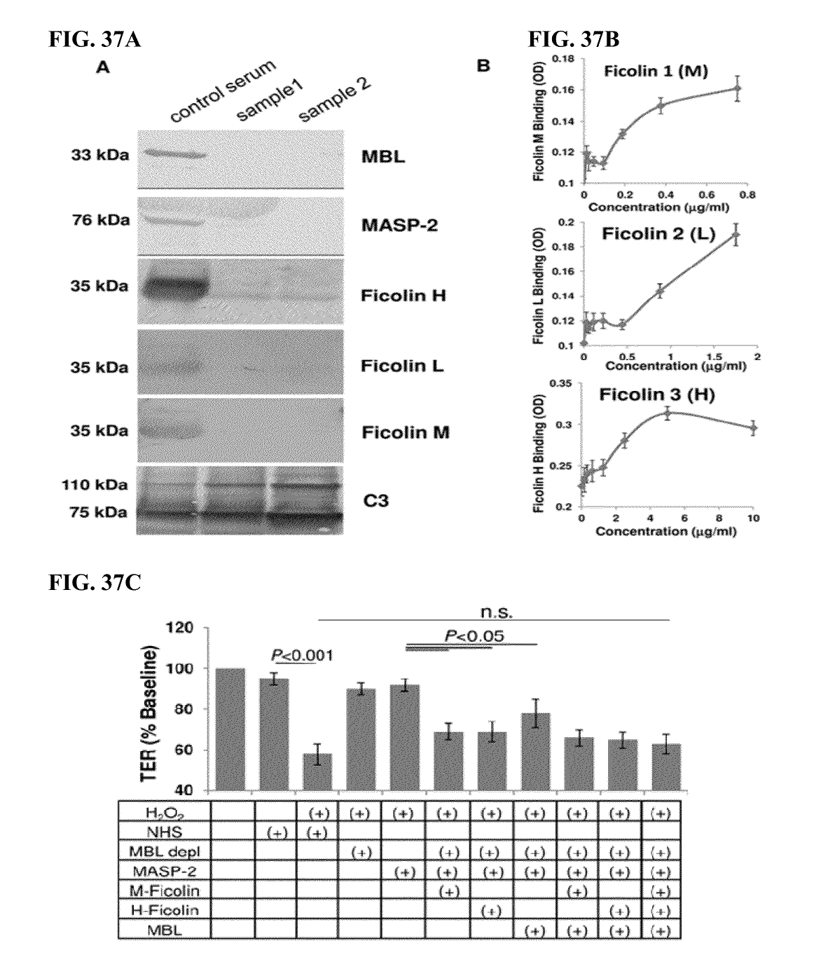

In the blood, MBL or ficolin are both complexed with inactive MASP; thus, if MBL is removed from serum using a mannan-agarose column, MASP levels might also be affected. In addition L-ficolin has been shown to bind directly to cyanogen-activated Sepharose beads (Tan, S. M., Chung, M. C., Kon, O. L., Thiel, S., Lee, S. H., and Lu, J. (1996) Biochem J 319 (Pt 2), 329-332), a problem that might also apply to H-ficolin and Mficolin. Western blot analysis confirmed that serum passed over a mannan-agarose column is depleted of MBL, MASP-2, M-ficolin and L-ficolin, or levels are below detection level, whereas H ficolin levels were drastically reduced (FIG. 37A). As a positive control, complement C3 levels were unaffected by the depletion process (FIG. 37A). To narrow down which ficolins recognize binding sites on ARPE-19 cells, monolayers were exposed to serial dilutions of serum in the presence 0.5 mM H2O2 (1 hr at 37° C.) and bound ficolins were detected using subtype-specific antibodies (FIG. 37B). In oxidatively-stressed cells, M- and H-ficolin binding was found to be saturable at physiological concentrations (mean serum concentrations of ficolins are M-ficolin 1.1, L-ficolin 3.3, and H-ficolin 18.4 μg/mL; (Zacho, R. M., Jensen, L., Terp, R., Jensenius, J. C., and Thiel, S. (2012) J Biol Chem 287, 8071-8081)), whereas L-ficolin binding was not saturable, but did appear to bind. However, since L-ficolin has been shown to bind non-specifically to BSA in solid-phase binding assays, a possible contribution of L-ficolin cannot be ruled out completely (Faro, J., Chen, Y., Jhaveri, P., Oza, P., Spear, G. T., Lint, T. F., and Gewurz, H. (2008) Clin Exp Immunol 151, 275-283).

Thus, the flow-through from the mannan-agarose column was utilized for reconstitution experiments, comparing MBL with M- and H-ficolin for their ability to reconstitute activity in the TER assay (FIG. 37C). Exposure of monolayers to H2O2+MBL-depleted serum resulted in ˜95% baseline TER values after 4 hours of exposure, whereas H2O2+NHS reduced TER to <60% of baseline values. Addition of MASP-2 (50 ng/mL) in combination with either one of the three pattern recognition molecules at physiological levels increased activity in the TER assay (P<0.05) to levels that were not significantly different from those of complete normal human serum. Adding MBL to either M- or H-ficolin or both did not further increase the activity. Thus, the lectin pathway can be activated in oxidatively-stressed cells by either MBL/MASP or ficolin/MASP.

Activation of the Lectin Pathway by Natural IgM

The lectin pathway has historically been recognized as a pathway that is activated by ficolin/MASP or MBL/MASP recognizing specific carbohydrates or acetylated molecules on pathogen surfaces; however, more recently, it has been shown that IgM molecules recognizing epitopes generated during ischemia reperfusion injury can activate the lectin pathway (Zhang, M., Takahashi, K., Alicot, E. M., Vorup-Jensen, T., Kessler, B., Thiel, S., Jensenius, J. C., Ezekowitz, R. A., Moore, F. D., and Carroll, M. C. (2006) J Immunol 177, 4727-4734). This experiment demonstrates that ficolin/MASP or MBL/MASP requires immunoglobulins, and specifically natural IgM to initiate complement activation on the cell surface of RPE cells.

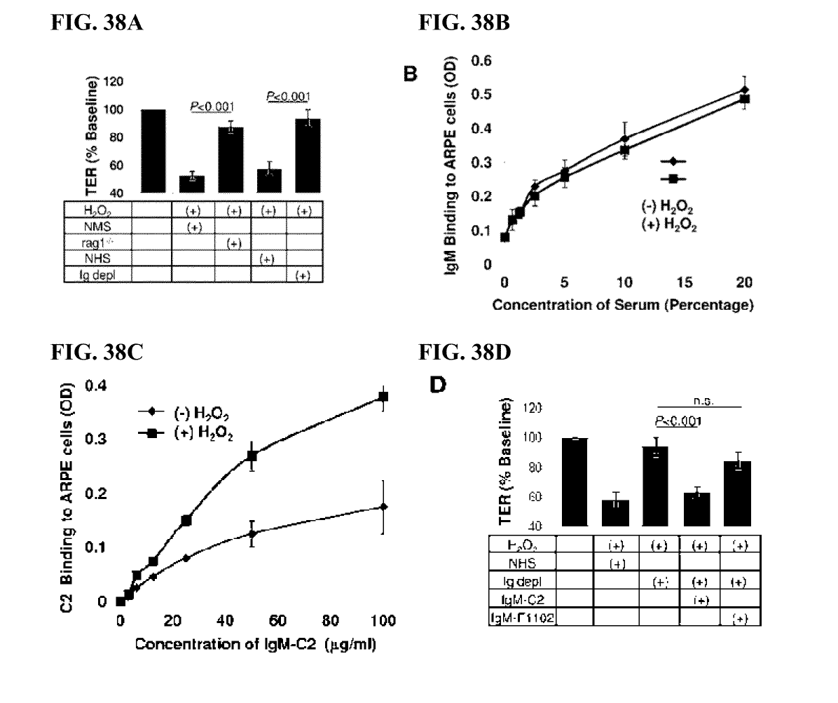

TER reduction was tested in complement-sufficient serum (mouse or human) and compared to serum from which Igs had been eliminated either genetically (rag1−/− mice) or by depletion (human Ig-depleted serum) (FIG. 38A). Both human and mouse complement-sufficient sera reduced TER by 40-50%, whereas Ig-depleted serum was ineffective. To characterize non-specific IgM binding to control and oxidatively-stressed ARPE-19 cells, cells were exposed to serial dilutions of serum followed by detection of bound IgMs using an IgM-specific secondary antibody coupled to alkaline phosphatase. However, just like for the ficolins and MASP, binding under both conditions was indistinguishable (FIG. 38B). When epitope-specific IgM-C2 binding to control and oxidatively-stressed ARPE-19 cells was compared, exposing cells to serial dilutions of IgM-C2 antibody followed by colorimetic detection of bound IgMs, ˜2.5-fold higher binding of IgM-C2 could be documented when comparing control and oxidative-stress conditions (FIG. 38C). Addition of the IgM-C2, but not a control antibody (IgM F1102, raised against dinitrophenol) was able to reconstitute activity to levels indistinguishable from normal human serum (FIG. 38D).

Identification of Ligands for Natural IgMs on RPE Cells

Cell binding assays have shown that IgM-C2 binding and injury is augmented under oxidative-stress conditions (FIG. 38C). Oxidative damage of membrane phospholipids results in the formation of malondialdehyde (MDA), an end-product of lipid peroxidation. The natural ligand of IgM-C2 on oxidatively-stressed RPE cells was further characterized.

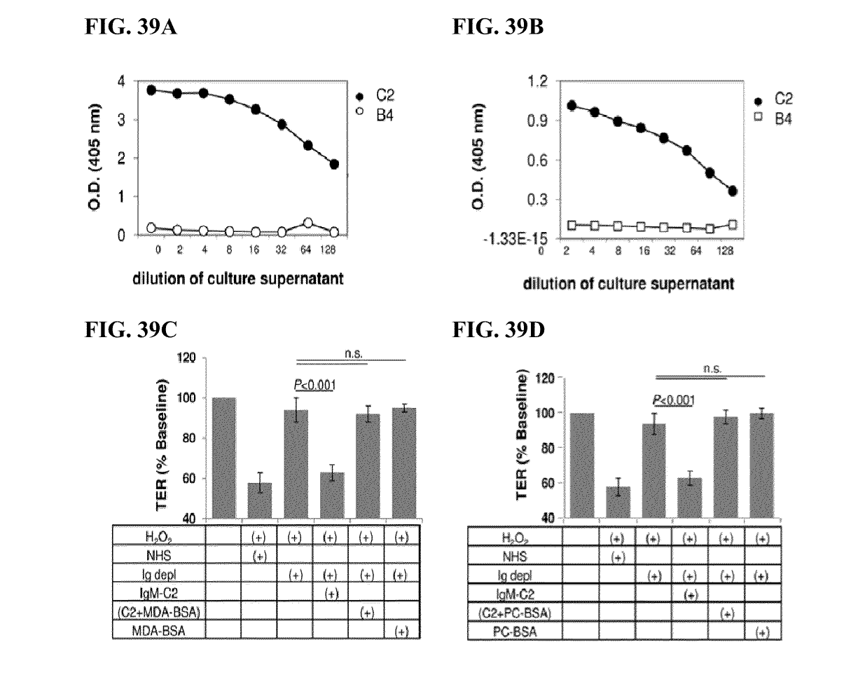

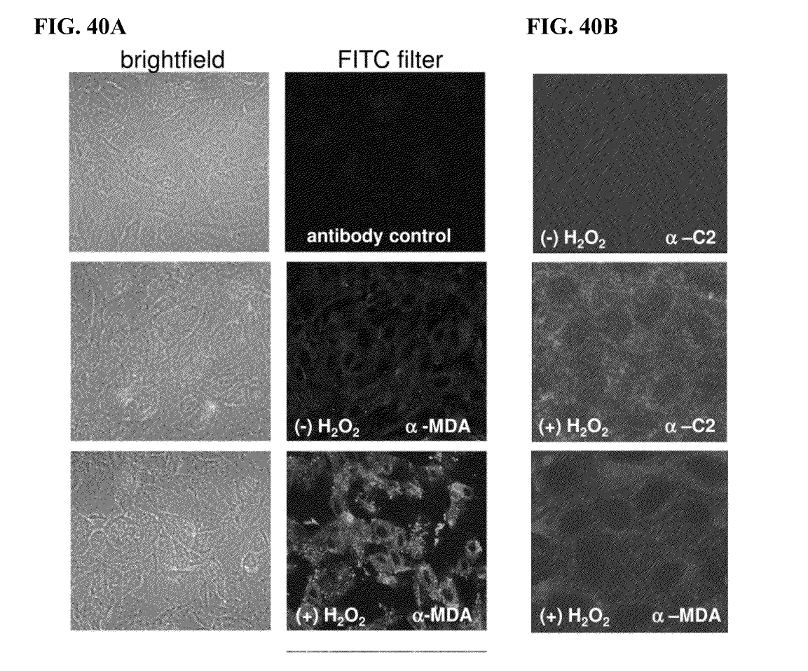

ELISAs to determine the reactivity of IgM-C2 to ligands were performed using microtiter plates coated with phosphorylcholine (PC)-BSA or malondialdehyde (MDA)-BSA in methanol. IgMC2 recognized both PC and MDA (FIGS. 39A, 39B). To determine which ligand is relevant for IgM-C2 binding to oxidatively-stressed RPE cells, IgM-C2 was preabsorbed with either MDA-BSA or PCBSA. IgM-C2 preabsorbed with either MDA-BSA or PC-BSA completely abolished its ability to reconstitute Ig-depleted serum (FIGS. 39C,39D). MDA and C2-IgM neoepitopes could be identified in a punctate fashion on H2O2-treated cells when compared to control cells. Both the anti-MDA antibody and the IgM-C2 antibody recognize epitopes present in puncta across the apical surface of the ARPE-19 cells (FIGS. 40A-40B).

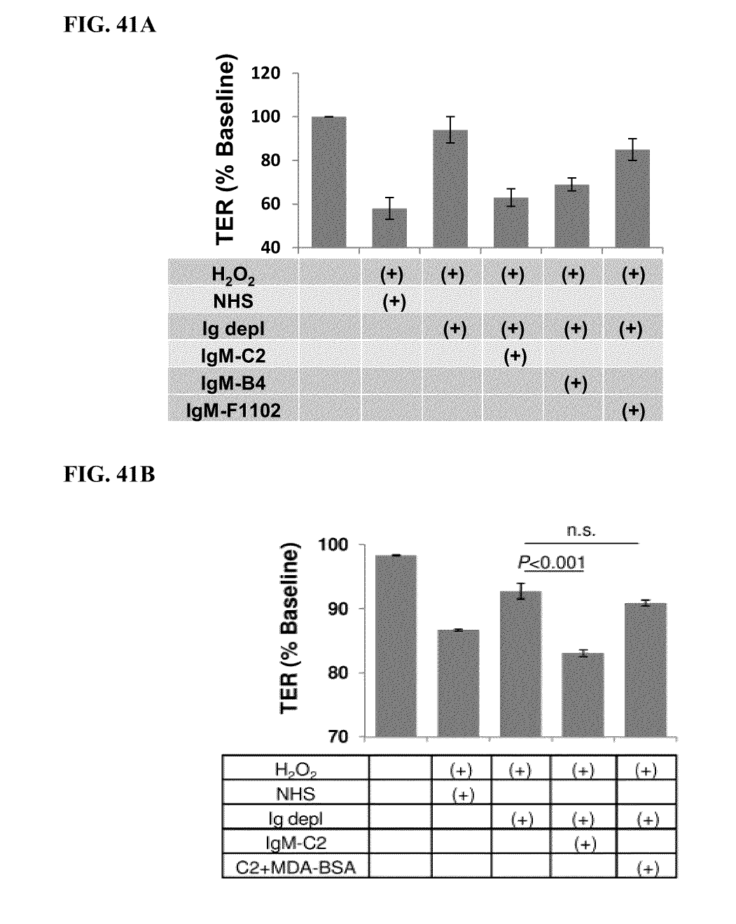

To confirm that the same neoepitopes are generated in primary human RPE cells, primary fetal RPE cells were grown into monolayers with a stable TER level of 250-300 Ω/cm2. These monolayers are susceptible to complement attack (FIGS. 41A-41B), albeit not to the same degree as ARPE-19 cells. The combined treatment of H2O2+10% NHS significantly decreased TER (P<0.001), which was attenuated by the elimination of all immunoglobulins (Ig-depleted serum; P<0.01). IgM-C2 (P<0.01), but not IgM-F1102 was able to reconstitute Ig-depleted serum, an effect that could be eliminated by preabsorption with MDA-BSA.

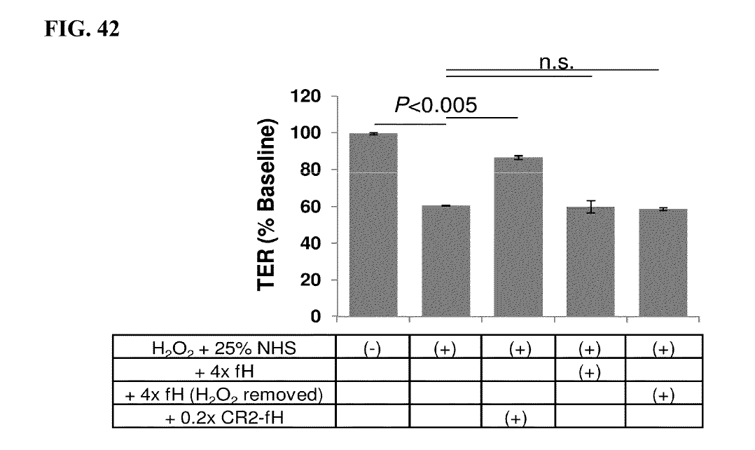

MDA has recently been shown to bind CFH. In a human acute monocytic leukemia cell line, malondialdehyde-acetaldehyde-BSA elicited a proinflammatory response as determined by IL-8 secretion, which was inhibited by physiological concentrations of CFH. If MDA represents a ligand for CFH in ARPE-19 cells, physiological concentrations of CFH might inhibit complement activation to prevent TER reduction. Since all the experiments thus far have been executed with 10% NHS, the experiments were repeated with higher NHS concentrations as well as in the presence of exogenous CFH. Average CFH concentration in serum is ˜500 μg/mL. Hence, TER experiments were performed in the presence of 25% NHS (FIG. 42) or 25% NHS supplemented with 375 μg of CFH. Both combinations were unable to prevent TER reduction by H2O2+NHS. To ensure that potential modification of CFH by H2O2 does not impair its binding to its ligand on ARPE-19 cells, the supernatant containing H2O2 was removed after 5 minutes of stimulation and NHS+exogenous CFH was added, which also did not prevent TER deterioration. However, CR2-fH (at 10 μg/mL), a CFH mimetic that consists of the complement receptor-2 binding domain for C3bi and C3d coupled to the inhibitory domain of CFH was able to inhibit TER deterioration induced by H2O2+NHS as previously reported (Thurman, J. M., Renner, B., Kunchithapautham, K., Ferreira, V. P., Pangburn, M. K., Ablonczy, Z., Tomlinson, S., Holers, V. M., and Rohrer, B. (2009) J Biol Chem 284, 16939-16947). Thus, on ARPE-19 cells, using H2O2 as the oxidant stimulus, MDA does not appear to serve as a ligand for CFH.

Identification of Roles for IgM-C2 In Vivo

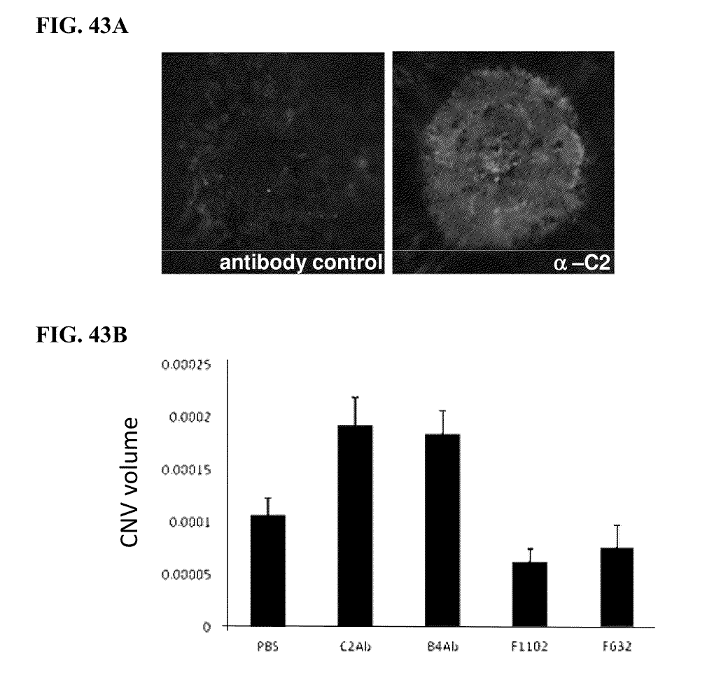

Mouse CNV laser lesions were examined by immunohistochemistry for labeling with the C2-IgM antibody. Specific labeling could be identified in CNV lesions as opposed to the area surrounding the lesion, when compared to the secondary antibody-only control (FIG. 43A). The MDA-specific antibody labeling was indistinguishable from that shown previously (Weismann, D., Hartvigsen, K., Lauer, N., Bennett, K. L., Scholl, H. P., Charbel Issa, P., Cano, M., Brandstatter, H., Tsimikas, S., Skerka, C., Superti-Furga, G., Handa, J. T., Zipfel, P. F., Witztum, J. L., and Binder, C. J. (2011) Nature 478, 76-81).

To address the physiological relevance of the C2-IgM autoantibodies in CNV lesions, it was examined whether C2-IgM reconstitution experiments would alter injury in rag1−/− mice. CNV lesions were examined in rag 1−/− mice after 3 administrations every 48 hours of either PBS, the F1102-IgM, the F632-IgM, the B4-IgM, or the C2-IgM (100 μg/mouse in 100 μL of PBS). While the control IgM antibodies (F1102-IgM and F632-IgM) had no effect on the size of the CNV lesion when compared to PBS-injected animals, CNV lesions were approximately twice the size after C2-IgM injections (P<0.01) or B4-IgM. Neither C2-IgM or B4-IgM injections had any effect in wildtype mice (FIG. 43B).

The principal findings obtained here, studying complement activation in oxidatively stressed RPE monolayers, can be summarized as follows: (1) oxidative stress generates neoepitopes on RPE cell surfaces that contain phospholipids, including MDA; (2) specific autoantibodies present in normal serum recognize these surface epitopes, and consequently trigger the activation of the complement cascade using the lectin pathway; (3) both MBL and ficolin can serve as the pattern recognition receptors for the lectin pathway; (4) this basal activity generated by the lectin pathway is subsequently amplified by the alternative pathway to generate the maximal effect; and finally, (5) the C2-IgM antibody recognizes neoepitopes in mouse CNV lesions and augments CNV development in antibody-deficient, rag1−/− mice.

In the present example, TER was used as a convenient, rapid and sensitive readout of complement activation. The availability of complement- and Ig-depleted serum as well as purified protein and individual IgMs allowed us to dissect the activation pathway for the terminal complement cascade on these oxidatively-stressed RPE cells. Since the 3 complement pathways require unique entry or activator molecules, pathway-specific serum can be generated (C1q-depeted serum, no CP; MASP-2-depleted serum, no LP; factor B-depleted serum, no AP; and C1q-MASP-2-double-depleted serum, AP-only) (FIGS. 36A-36B). Both the LP and the AP are necessary for generating the loss of TER. AP plays a role in amplifying the complement cascade on oxidatively stressed RPE cells. Since the serum depleted for the LP pathway components was found to be depleted for MASP-2, MBL and all three ficolins, (FIG. 37A), reconstitution studies could be performed with the individual pattern recognition molecules. Roles for MBL, M-ficolin and H-ficolin could be demonstrated utilizing the TER assay. Since specific binding of M- and H-ficolin to RPE cells could be demonstrated in the context of complete serum, LP can be activated by the presence of a specific antibody/antigen complex. While Ig-dependence was demonstrated utilizing Ig-depleted serum (FIG. 38A), H2O2-dependent changes in total IgM (FIG. 38B) or total IgG (data not shown) could not be demonstrated. However, H2O2-dependent changes in binding of a phospholipid antigen-specific IgM (IgM-C2) was revealed (FIG. 38C), and in reconstitution assays, IgM-C2 antibodies, but not a control antibody, F1102, resulted in the restoration of the effect on TER in Ig-depleted serum (FIG. 38D). Control antibody, F1102, when tested in binding assays, also showed no saturable binding to RPE cells (data not shown). Finally, antigen-specificity for C2-IgM was further refined by ELISA (FIG. 39A) and TER experiments (FIGS. 39B,39C). Since the original characterization of the ligands (Elvington, A., Atkinson, C., Kulik, L., Zhu, H., Yu, J., Kindy, M. S., Holers, V. M., and Tomlinson, S. (2012) J Immunol 188, 1460-1468) was performed in the absence of H2O2, but phospholipids can undergo peroxidation, binding was compared between phosphatidylcholine (non-oxidized) and malondialdehyde (MDA; oxidized) coupled to bovine serum albumin (BSA). Specific binding could be documented by ELISA, and preabsorption of the antibody with either MDA-BSA or PC-BSA interfered with activity in the TER assay. H2O2-dependent binding could be documented for both a MDA-specific antibody, as well as the neoepitope-specific IgM on RPE cell surfaces (FIGS. 40A-40B). Finally, the neoepitopes recognizable by IgM-C2 and IgM-B4 are also present on oxidatively-stressed primary fetal human RPE cells (FIGS. 41A-41B).

The results of the Examples show that alterations in barrier function produced by oxidative stress-mediated complement activation requires a phospholipid as a ligand, LP initiation molecules, and alternative pathway amplification, followed by activation of the terminal pathway, including transient membrane attack complex activation. This is the first report that identifies a potential ligand, the pattern recognition receptor, and the pathway required for activation in a model relevant for AMD.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

### Sequence Chart

TABLE 2
Summary of sequences

| SEQ | | |
|---|---|---|
| ID | | Type of |
| NO: | Function | Sequence |
| 1. | B4 CDR-L1 | Amino acid |
| 2. | B4 CDR-L2 | Amino acid |
| 3. | B4 CDR-L3 | Amino acid |
| 4. | B4 CDR-H1 | Amino acid |
| 5. | B4 CDR-H2 | Amino acid |
| 6. | B4 CDR-H3 | Amino acid |
| 7. | B4 CDR-L1 | Amino acid |
| 8. | B4 CDR-L2 | Amino acid |
| 9. | B4 CDR-L3 | Amino acid |
| 10. | B4 CDR-H1 | Amino acid |
| 11. | B4 CDR-H2 | Amino acid |
| 12. | B4 CDR-H3 | Amino acid |
| 13. | B4 light chain variable domain | Amino acid |
| 14. | B4 light chain variable domain | Amino acid |
| 15. | B4 heavy chain variable domain | Amino acid |
| 16. | B4 heavy chain variable domain | Amino acid |
| 17. | B4 scFV | Amino acid |
| 18. | B4 scFV | Amino acid |
| 19. | B4 light chain variable domain | Nucleic acid |
| 20. | B4 light chain variable domain | Nucleic acid |
| 21. | B4 heavy chain variable domain | Nucleic acid |
| 22. | B4 heavy chain variable domain | Nucleic acid |
| 23. | B4 scFV | Nucleic acid |
| 24. | B4 scFV, optimized sequence for CHO expression | Nucleic acid |
| 25. | C2 CDR-L1 | Amino acid |
| 26. | C2 CDR-L2 | Amino acid |
| 27. | C2 CDR-L3 | Amino acid |
| 28. | C2 CDR-H1 | Amino acid |
| 29. | C2 CDR-H2 | Amino acid |
| 30. | C2 CDR-H3 | Amino acid |
| 31. | C2 CDR-L1 | Amino acid |
| 32. | C2 CDR-L2 | Amino acid |
| 33. | C2 CDR-L3 | Amino acid |
| 34. | C2 light chain variable domain | Amino acid |
| 35. | C2 light chain variable domain | Amino acid |
| 36. | C2 heavy chain variable domain | Amino acid |
| 37. | C2 scFV | Amino acid |
| 38. | C2 scFV | Amino acid |
| 39. | C2 light chain variable domain | Nucleic acid |
| 40. | C2 light chain variable domain | Nucleic acid |
| 41. | C2 heavy chain variable domain | Nucleic acid |
| 42. | C2 scFV | Nucleic acid |
| 43. | C2 scFV, optimized sequence for CHO expression | Nucleic acid |
| 44. | Full-length human membrane cofactor protein | Amino acid |
| | (MCP) | |
| 45. | Full-length human decay accelerating factor (DAF) | Amino acid |
| 46. | Full-length mouse decay accelerating factor (DAF) | Amino acid |
| 47. | Full-length human CD59 | Amino acid |
| 48. | Full-length mouse CD59 | Amino acid |
| 49. | Full-length mouse CD59, isoform B | Amino acid |
| 50. | Full-length mouse Crry | Amino acid |
| 51. | Full-length human CR1 | Amino acid |
| 52. | Full-length human factor H | Amino acid |
| 53. | Full-length mouse factor H | Amino acid |
| 54. | Signal peptide of the human CD5 protein | Amino acid |
| 55. | Signal peptide of the human CR2 protein | Amino acid |
| 56. | Signal peptide of the human CR2 protein | Amino acid |
| 57. | B4 scFV | Nucleic acid |
| 58. | C2 scFV | Nucleic acid |

### Sequences

B4 CDR-L1 amino acid sequence
(SEQ ID NO: 1)
SSISSNY
B4 CDR-L2 amino acid sequence
(SEQ ID NO: 2)
RTS
B4 CDR-L3 amino acid sequence
(SEQ ID NO: 3)
QQGSSIPRTRSEGAPSWK
B4 CDR-H1 amino acid sequence
(SEQ ID NO: 4)
GYTFTSYW
B4 CDR-H2 amino acid sequence
(SEQ ID NO: 5)
IGPNSGGT
B4 CDR-H3 amino acid sequence
(SEQ ID NO: 6)
ARRMVKGCYGLLGPRDHGHRLL
B4 CDR-L1 amino acid sequence
(SEQ ID NO: 7)
QSIVHSNGNTY
B4 CDR-L2 amino acid sequence
(SEQ ID NO: 8)
KVS
B4 CDR-L3 amino acid sequence
(SEQ ID NO: 9)
FQGSHVPYT
B4 CDR-H1 amino acid sequence
(SEQ ID NO: 10)
GYTFTDYY
B4 CDR-H2 amino acid sequence
(SEQ ID NO: 11)
INPNNGGT
B4 CDR-H3 amino acid sequence
(SEQ ID NO: 12)
ARYDYAWYFDV
B4 VL amino acid sequence
(SEQ ID NO: 13)
DIELTQSPTTMAASPGEKITITCSASSSISSNYLHWYQQKPGFSPKLLI
YRTSNLASGVPARFSGSGSGTSYSLTIGTMEAEDVATYYCQQGSSIPRT
RSEGAPSWK
B4 VL amino acid sequence
(SEQ ID NO: 14)
DVLMTQTPLSLPVSLGDQASISCRSSQSIVHSNGNTYLEWYLQKPGQSP
KLLIYKVSNRFSGVPDRFSGSGSGTDFTLKISRVEAEDLGVYYCFQGSH
VPYTFGGGTKLEIK
B4 VH amino acid sequence
(SEQ ID NO: 15)
VKLQESGAELVKPGASVKLSCKASGYTFTSYWMHWVKQRPGRGLEWIGR
IGPNSGGTKYNEKFKSKATLTVDKPSSTAYMQLSSLTSEDSAVYYCARR
MVKGCYGLLGPRDHGHRLL
B4 VH amino acid sequence
(SEQ ID NO: 16)
VKLQESGPELVKPGASVKISCKASGYTFTDYYMNWVKQSHGKSLEWIGD
INPNNGGTSYNQKFKGKATLTVDKSSSTAYMELRSLTSEDSAVYYCARY
DYAWYFDVWGQGTTVTVSS
B4 scFV amino acid sequence
(SEQ ID NO: 17)
HHHHHHVKLQESGAELVKPGASVKLSCKASGYTFTSYWMHWVKQRPGRG
LEWIGRIGPNSGGTKYNEKFKSKATLTVDKPSSTAYMQLSSLTSEDSAV
YYCARRMVKGCYGLLGPRDHGHRLLKGRIPAHWRPLLVDPSSVPSLASG
GGGGSGGGGSWISAEFALDIELTQSPTTMAASPGEKITITCSASSSISS
NYLHWYQQKPGFSPKLLIYRTSNLASGVPARFSGSGSGTSYSLTIGTME
AEDVATYYCQQGSSIPRTRSEGAPSWK
B4 scFV amino acid sequence
(SEQ ID NO: 18)
MSVPTQVLGLLLLWLTDARCVKLQESGPELVKPGASVKISCKASGYTFT
DYYMNWVKQSHGKSLEWIGDINPNNGGTSYNQKFKGKATLTVDKSSSTA
YMELRSLTSEDSAVYYCARYDYAWYFDVWGQGTTVTVSSGGGGSGGGGS
GGGGDVLMTQTPLSLPVSLGDQASISCRSSQSIVHSNGNTYLEWYLQKP
GQSPKLLIYKVSNRFSGVPDRFSGSGSGTDFTLKISRVEAEDLGVYYCF
QGSHVPYTFGGGTKLEIKRIEGRHHHHHH
B4 VL nucleic acid sequence
(SEQ ID NO: 19)
GACATTGAGCTCACCCAGTCTCCAACCACCATGGCTGCATCTCCCGGGG
AGAAGATCACTATCACCTGCAGTGCCAGCTCAAGTATAAGTTCCAATTA
CTTGCATTGGTATCAGCAGAAGCCAGGATTCTCCCCTAAACTCTTGATT
TATAGGACATCCAATCTGGCTTCTGGAGTCCCAGCTCGCTTCAGTGGCA
GTGGGTCTGGGACCTCTTACTCTCTCACAATTGGCACCATGGAGGCTGA
AGATGTTGCCACTTACTACTGCCAGCAGGGTAGTAGTATACCACGTACA
CGTTCGGAGGGGGCACCAAGCTGGAAA
B4 VL nucleic acid sequence
(SEQ ID NO: 20)
GATGTTTTGATGACCCAAACTCCACTCTCCCTGCCTGTCAGTCTTGGAG
ATCAAGCCTCCATCTCTTGCAGATCTAGTCAGAGCATTGTACATAGTAA
TGGAAACACCTATTTAGAATGGTACCTGCAGAAACCAGGCCAGTCTCCA
AAGCTCCTGATCTACAAAGTTTCCAACCGATTTTCTGGGGTCCCAGACA
GGTTCAGTGGCAGTGGATCAGGGACAGATTTCACACTCAAGATCAGCAG
AGTGGAGGCTGAGGATCTGGGAGTTTATTACTGCTTTCAAGGTTCACAT
GTTCCGTACACGTTCGGAGGGGGGACCAAGCTGGAAATAAAACG
B4 VH nucleic acid sequence
(SEQ ID NO: 21)
GTGAAACTGCAGGAGTCAGGGGCTGAGCTTGTGAAGCCTGGGGCTTCAG
TGAAGCTGTCCTGCAAGGCTTCTGGCTACACCTTCACCAGCTACTGGAT
GCACTGGGTGAAGCAGAGGCCTGGACGAGGCCTTGAGTGGATTGGAAGG
ATTGGTCCTAATAGTGGTGGTACTAAGTACAATGAGAAGTTCAAGAGCA
AGGCCACACTGACTGTAGACAAACCCTCCAGCACAGCCTACATGCAGCT
CAGCAGCCTGACATCTGAGGACTCTGCGGTCTATTATTGTGCAAGAAGA
ATGGTAAAGGGGTGCTATGGACTACTGGGGCCAAGGGACCACGGTCACC
GTCTCCTCA
B4 VH nucleic acid sequence
(SEQ ID NO: 22)
GTGAAGCTGCAGGAGTCTGGACCTGAGCTGGTGAAGCCTGGGGCTTCAG
TGAAGATATCCTGTAAGGCTTCTGGATACACGTTCACTGACTACTACAT
GAACTGGGTGAAGCAGAGCCATGGAAAGAGCCTTGAGTGGATTGGAGAT
ATTAATCCTAACAATGGTGGTACTAGCTACAACCAGAAGTTCAAGGGCA
AGGCCACATTGACTGTAGACAAGTCCTCCAGCACAGCCTACATGGAGCT
CCGCAGCCTGACATCTGAGGACTCTGCAGTCTATTACTGTGCAAGATAT
GATTACGCTTGGTACTTCGATGTCTGGGGCCAAGGGACCACGGTCACCG
TCTCCTCA
B4 scFV nucleic acid sequence
(SEQ ID NO: 23)
GCCGCCACCATGAGTGTGCCCACTCAGGTCCTGGGGTTGCTGCTGCTGT
GGCTTACAGATGCCAGATGTGTGAAGCTGCAGGAGTCTGGACCTGAGCT
GGTGAAGCCTGGGGCTTCAGTGAAGATATCCTGTAAGGCTTCTGGATAC
ACGTTCACTGACTACTACATGAACTGGGTGAAGCAGAGCCATGGAAAGA
GCCTTGAGTGGATTGGAGATATTAATCCTAACAATGGTGGTACTAGCTA
CAACCAGAAGTTCAAGGGCAAGGCCACATTGACTGTAGACAAGTCCTCC
AGCACAGCCTACATGGAGCTCCGCAGCCTGACATCTGAGGACTCTGCAG
TCTATTACTGTGCAAGATATGATTACGCTTGGTACTTCGATGTCTGGGG
CCAAGGGACCACGGTCACCGTCTCCTCAGGCGGAGGTGGGTCGGGTGGC
GGCGGATCTGGCGGAGGTGGGGATGTTTTGATGACCCAAACTCCACTCT
CCCTGCCTGTCAGTCTTGGAGATCAAGCCTCCATCTCTTGCAGATCTAG
TCAGAGCATTGTACATAGTAATGGAAACACCTATTTAGAATGGTACCTG
CAGAAACCAGGCCAGTCTCCAAAGCTCCTGATCTACAAAGTTTCCAACC
GATTTTCTGGGGTCCCAGACAGGTTCAGTGGCAGTGGATCAGGGACAGA
TTTCACACTCAAGATCAGCAGAGTGGAGGCTGAGGATCTGGGAGTTTAT
TACTGCTTTCAAGGTTCACATGTTCCGTACACGTTCGGAGGGGGGACCA
AGCTGGAAATAAAACGGATCGAAGGCCGGCATCACCATCATCACCACTG
ATAG
CHO optimized B4 scFV nucleic acid sequence
(SEQ ID NO: 24)
ATGTCCGTGCCTACCCAGGTGCTCGGACTCCTGCTGCTGTGGCTCACCG
ACGCCAGGTGTGTGAAGCTGCAGGAGAGCGGACCCGAGCTGGTGAAGCC
TGGAGCCTCCGTGAAGATCAGCTGCAAGGCTTCCGGATACACCTTCACC
GACTACTATATGAACTGGGTGAAGCAGAGCCACGGCAAGAGCCTGGAGT
GGATCGGCGACATCAACCCTAACAACGGCGGCACCTCCTACAACCAGAA
GTTCAAGGGCAAGGCTACACTGACCGTGGACAAGTCCTCCAGCACCGCC
TACATGGAGCTCAGGAGCCTGACCTCCGAGGATTCCGCCGTCTATTACT
GTGCCCGGTACGACTACGCCTGGTATTTCGACGTGTGGGGCCAGGGCAC
AACCGTCACAGTCTCCAGCGGAGGAGGAGGAAGCGGCGGCGGAGGATCC
GGAGGCGGAGGCGATGTCCTGATGACACAGACACCTCTGAGCCTCCCCG
TGAGCCTGGGAGACCAAGCCTCCATCTCCTGCAGGTCCTCCCAGTCCAT
CGTGCACAGCAATGGCAACACCTACCTGGAGTGGTATCTGCAGAAGCCT
GGCCAGTCCCCCAAGCTGCTGATCTACAAGGTGTCCAACCGGTTCAGCG
GCGTCCCTGACAGGTTCTCCGGATCCGGAAGCGGCACAGATTTCACCCT
GAAGATCAGCAGGGTCGAGGCCGAGGACCTGGGAGTGTACTACTGCTTC
CAGGGCTCCCATGTCCCTTACACCTTCGGCGGCGGCACCAAACTGGAGA
TCAAGCGGATCGAGGGCAGGCATCACCACCATCACCACTGA
C2 CDR-L1 amino acid sequence
(SEQ ID NO: 25)
KSVSTSGYSY
C2 CDR-L2 amino acid sequence
(SEQ ID NO: 26)
LVS
C2 CDR-L3 amino acid sequence
(SEQ ID NO: 27)
QHIRELTRSEGGPSWK
C2 CDR-H1 amino acid sequence
(SEQ ID NO: 28)
GYTFTSYW
C2 CDR-H2 amino acid sequence
(SEQ ID NO: 29)
INPSNGGT
C2 CDR-H3 amino acid sequence
(SEQ ID NO: 30)
ARRGIRLRHFDY
C2 CDR-L1 amino acid sequence
(SEQ ID NO: 31)
QDVGTA
C2 CDR-L2 amino acid sequence
(SEQ ID NO: 32)
WAS
C2 CDR-L3 amino acid sequence
(SEQ ID NO: 33)
QQYSSYPLT
C2 VL amino acid sequence
(SEQ ID NO: 34)
DIVMTQSPASLAVSLGQRATISYRASKSVSTSGYSYMHWNQQKPGQPPR
LLIYLVSNLESGVPARFSGSGSGTDFTLNIHPVEEEDAATYYCQHIREL
TRSEGGPSWK
C2 VL amino acid sequence
(SEQ ID NO: 35)
DIQMTQSPKFMSTSVGDRVSITCKASQDVGTAVAWYQQKPGQSPKLLIY
WASTRHTGVPDRFTGSGSGTDFTLTISNVQSEDLADYFCQQYSSYPLTF
GAGTKLELK
C2 VH amino acid sequence
(SEQ ID NO: 36)
VKLQESGTELVKPGASVKLSCKASGYTFTSYWMHWVKQRPGQGLEWIGN
INPSNGGTNYNEKFKSKATLTVDKSSSTAYMQLSSLTSEDSAVYYCARR
GIRLRHFDYWGQGTTVTVS
C2 scFV amino acid sequence
(SEQ ID NO: 37)
VKLQESGTELVKPGASVKLSCKASGYTFTSYWMHWVKQRPGQGLEWIGN
INPSNGGTNYNEKFKSKATLTVDKSSSTAYMQLSSLTSEDSAVYYCARR
GIRLRHFDYWGQGTTVTVSSRANSADIHHTGGRSSMHLEGPIRPIVSRI
SGGGGGSGGGGSWISAEFALDIVMTQSPASLAVSLGQRATISYRASKSV
STSGYSYMHWNQQKPGQPPRLLIYLVSNLESGVPARFSGSGSGTDFTLN
IHPVEEEDAATYYCQHIRELTRSEGGPSWK
C2 scFV amino acid sequence
(SEQ ID NO: 38)
MSVPTQVLGLLLLWLTDARCVKLQESGTELVKPGASVKLSCKASGYTFT
SYWMHWVKQRPGQGLEWIGNINPSNGGTNYNEKFKSKATLTVDKSSSTA
YMQLSSLTSEDSAVYYCARRGIRLRHFDYWGQGTTVTVSSGGGGSGGGG
SGGGGSDIQMTQSPKFMSTSVGDRVSITCKASQDVGTAVAWYQQKPGQS
PKLLIYWASTRHTGVPDRFTGSGSGTDFTLTISNVQSEDLADYFCQQYS
SYPLTFGAGTKLELKRIEGRHHHHHH
C2 VL nucleic acid sequence
(SEQ ID NO: 39)
GACATTGTGATGACACAGTCTCCTGCTTCCTTAGCTGTATCTCTGGGGC
AGAGGGCCACCATCTCATACAGGGCCAGCAAAAGTGTCAGTACATCTGG
CTATAGTTATATGCACTGGAACCAACAGAAACCAGGACAGCCACCCAGA
CTCCTCATCTATCTTGTATCCAACCTAGAATCTGGGGTCCCTGCCAGGT
TCAGTGGCAGTGGGTCTGGGACAGACTTCACCCTCAACATCCATCCTGT
GGAGGAGGAGGATGCTGCAACCTATTACTGTCAGCACATTAGGGAGCTT
ACACGTTCGGAGGGGGGACCAAGCTGGAAA
C2 VL nucleic acid sequence
(SEQ ID NO: 40)
GACATCCAGATGACCCAGTCTCCCAAATTCATGTCCACATCAGTAGGAG
ACAGGGTCAGCATCACCTGCAAGGCCAGTCAGGATGTGGGTACTGCTGT
AGCCTGGTATCAACAGAAACCAGGGCAATCTCCTAAACTACTGATTTAC
TGGGCATCCACCCGGCACACTGGAGTCCCTGATCGCTTCACAGGCAGTG
GATCTGGGACAGATTTCACTCTCACCATTAGCAATGTGCAGTCTGAAGA
CTTGGCAGATTATTTCTGTCAGCAATATAGCAGCTATCCTCTCACGTTC
GGTGCTGGGACCAAGCTGGAGCTGAAAC
C2 VH nucleic acid sequence
(SEQ ID NO: 41)
GTGAAACTGCAGGAGTCTGGGACTGAACTGGTGAAGCCTGGGGCTTCAG
TGAAGCTGTCCTGCAAGGCTTCTGGCTACACCTTCACCAGCTACTGGAT
GCACTGGGTGAAGCAGAGGCCTGGACAAGGCCTTGAGTGGATTGGAAAT
ATTAATCCTAGCAATGGTGGTACTAACTACAATGAGAAGTTCAAGAGCA
AGGCCACACTGACTGTAGACAAATCCTCCAGCACAGCCTACATGCAGCT
CAGCAGCCTGACATCTGAGGACTCTGCGGTCTATTATTGTGCAAGAAGA
GGCATACGGTTACGACACTTTGACTACTGGGGCCAAGGGACCACGGTCA
CCGTCTCC
C2 scFV nucleic acid sequence
(SEQ ID NO: 42)
GCCGCCACCATGAGTGTGCCCACTCAGGTCCTGGGGTTGCTGCTGCTGT
GGCTTACAGATGCCAGATGTGTGAAACTGCAGGAGTCTGGGACTGAACT
GGTGAAGCCTGGGGCTTCAGTGAAGCTGTCCTGCAAGGCTTCTGGCTAC
ACCTTCACCAGCTACTGGATGCACTGGGTGAAGCAGAGGCCTGGACAAG
GCCTTGAGTGGATTGGAAATATTAATCCTAGCAATGGTGGTACTAACTA
CAATGAGAAGTTCAAGAGCAAGGCCACACTGACTGTAGACAAATCCTCC
AGCACAGCCTACATGCAGCTCAGCAGCCTGACATCTGAGGACTCTGCGG
TCTATTATTGTGCAAGAAGAGGCATACGGTTACGACACTTTGACTACTG
GGGCCAAGGGACCACGGTCACCGTCTCCTCTGGCGGAGGTGGGTCGGGT
GGCGGCGGATCTGGCGGAGGTGGGTCGGACATCCAGATGACCCAGTCTC
CCAAATTCATGTCCACATCAGTAGGAGACAGGGTCAGCATCACCTGCAA
GGCCAGTCAGGATGTGGGTACTGCTGTAGCCTGGTATCAACAGAAACCA
GGGCAATCTCCTAAACTACTGATTTACTGGGCATCCACCCGGCACACTG
GAGTCCCTGATCGCTTCACAGGCAGTGGATCTGGGACAGATTTCACTCT
CACCATTAGCAATGTGCAGTCTGAAGACTTGGCAGATTATTTCTGTCAG
CAATATAGCAGCTATCCTCTCACGTTCGGTGCTGGGACCAAGCTGGAGC
TGAAACGGATCGAAGGCCGGCATCACCATCATCACCACTGATAG
CHO optimized C2 scFV nucleic acid sequence
(SEQ ID NO: 43)
ATGAGCGTGCCTACACAGGTGCTCGGCCTGCTGCTCCTCTGGCTGACAGA
CGCCCGGTGTGTGAAGCTGCAGGAGTCCGGAACCGAGCTGGTGAAACCTG
GCGCCAGCGTGAAACTGAGCTGCAAAGCCAGCGGATACACCTTCACCTCC
TACTGGATGCACTGGGTGAAACAGAGGCCTGGCCAGGGCCTGGAATGGAT
TGGCAACATCAACCCCAGCAACGGCGGCACCAACTACAATGAGAAGTTCA
AGAGCAAGGCCACCCTGACCGTGGATAAGTCCTCCTCCACCGCCTACATG
CAGCTGTCCTCCCTCACCTCCGAGGACAGCGCCGTCTATTACTGTGCCAG
GCGGGGCATCAGGCTGAGGCACTTCGACTACTGGGGCCAAGGCACAACCG
TCACCGTGAGCTCCGGAGGAGGAGGCAGCGGAGGCGGAGGCTCCGGCGGA
GGCGGAAGCGACATTCAGATGACCCAGAGCCCCAAGTTCATGTCCACCTC
CGTCGGCGACAGGGTGAGCATCACCTGTAAGGCCAGCCAGGATGTCGGCA
CAGCTGTGGCCTGGTACCAGCAGAAGCCCGGCCAGTCCCCCAAGCTGCTG
ATCTACTGGGCTTCCACAAGGCATACCGGCGTCCCCGATAGGTTCACAGG
CTCCGGCTCCGGCACCGACTTCACACTCACCATCAGCAACGTCCAGTCCG
AGGACCTGGCCGACTACTTCTGCCAGCAGTACTCCAGCTACCCCCTCACC
TTCGGCGCTGGCACCAAGCTGGAACTCAAGCGGATCGAGGGCAGGCATCA
CCACCATCACCACTGATAG
Full-length human membrane cofactor protein (MCP)
(SEQ ID NO: 44)
MEPPGRRECPFPSWRFPGLLLAAMVLLLYSFSDACEEPPTFEAMELIGKP
KPYYEIGERVDYKCKKGYFYIPPLATHTICDRNHTWLPVSDDACYRETCP
YIRDPLNGQAVPANGTYEFGYQMHFICNEGYYLIGEEILYCELKGSVAIW
SGKPPICEKVLCTPPPKIKNGKHTFSEVEVFEYLDAVTYSCDPAPGPDPF
SLIGESTIYCGDNSVWSRAAPECKVVKCRFPVVENGKQISGFGKKFYYKA
TVMFECDKGFYLDGSDTIVCDSNSTWDPPVPKCLKVLPPSSTKPPALSH
SVSTSSTTKSPASSASGPRPTYKPPVSNYPGYPKPEEGILDSLDVWVIAV
IVIAIVVGVAVICVVPYRYLQRRKKKGTYLTDETHREVKFTSL
Full-length human decay accelerating factor (DAF)
(SEQ ID NO: 45)
MTVARPSVPAALPLLGELPRLLLLVLLCLPAVWGDCGLPPDVPNAQPAL
EGRTSFPEDTVITYKCEESFVKIPGEKDSVICLKGSQWSDIEEFCNRSCE
VPTRLNSASLKQPYITQNYFPVGTVVEYECRPGYRREPSLSPKLTCLQNL
KWSTAVEFCKKKSCPNPGEIRNGQIDVPGGILFGATISFSCNTGYKLFGS
TSSFCLISGSSVQWSDPLPECREIYCPAPPQIDNGIIQGERDHYGYRQSV
TYACNKGFTMIGEHSIYCTVNNDEGEWSGPPPECRGKSLTSKVPPTVQKP
TTVNVPTTEVSPTSQKTTTKTTTPNAQATRSTPVSRTTKHFHETTPNKGS
GTTSGTTRLLSGHTCFTLTGLLGTLVTMGLLT
Full-length mouse decay accelerating factor (DAF)
(SEQ ID NO: 46)
MIRGRAPRTRPSPPPPLLPLLSLSLLLLSPTVRGDCGPPPDIPNARPILG
RHSKFAEQSKVAYSCNNGFKQVPDKSNIVVCLENGQWSSHETFCEKSCVA
PERLSFASLKKEYLNMNFFPVGTIVEYECRPGFRKQPPLPGKATCLEDLV
WSPVAQFCKKKSCPNPKDLDNGHINIPTGILFGSEINFSCNPGYRLVGVS
STFCSVTGNTVDWDDEFPVCTEIHCPEPPKINNGIMRGESDSYTYSQVVT
YSCDKGFILVGNASIYCTVSKSDVGQWSSPPPRCIEKSKVPTKKPTINVP
STGTPSTPQKPTTESVPNPGDQPTPQKPSTVKVSATQHVPVTKTTVRHPI
RTSTDKGEPNTGGDRYIYGHTCLITLTVLHVMLSLIGYLT
Full-length human CD59
(SEQ ID NO: 47)
MGIQGGSVLFGLLLVLAVFCHSGHSLQCYNCPNPTADCKTAVNCSSDFDA
CLITKAGLQVYNKCWKFEHCNFNDVTTRLRENELTYYCCKKDLCNFNEQL
ENGGTSLSEKTVLLLVTPFLAAAWSLHP
Full-length mouse CD59
(SEQ ID NO: 48)
MRAQRGLILLLLLLAVFCSTAVSLTCYHCFQPVVSSCNMNSTCSPDQDSC
LYAVAGMQVYQRCWKQSDCHGEIIMDQLEETKLKFRCCQFNLCNKSDGSL
GKTPLLGTSVLVAILNLCFLSHL
Full-length mouse CD59, isoform B
(SEQ ID NO: 49)
MRAQRGLILLLLLLAVFCSTAVSLKCYNCFQFVSSCKINTTCSPNLDSCL
YAVAGRQVYQQCWKLSDCNSNYIMSRLDVAGIQSKCCQWGLCNKNLDGLE
EPNNAETSSLRKTALLGTSVLVAILKFCF
Full-length mouse complement receptor 1-related
gene/protein y (Crry)
(SEQ ID NO: 50)
MEVSSRSSEPLDPVWLLVAFGRGGVKLEVLLLFLLPFTLGELRGGLGKHG
HTVHREPAVNRLCADSKRWSGLPVSAQRPFPMGHCPAPSQLPSAKPINLT
DESMFPIGTYLLYECLPGYIKRQFSITCKQDSTWTSAEDKCIRKQCKTPS
DPENGLVHVHTGIQFGSRINYTCNQGYRLIGSSSAVCVITDQSVDWDTEA
PICEWIPCEIPPGIPNGDFFSSTREDFHYGMVVTYRCNTDARGKALFNLV
GEPSLYCTSNDGEIGVWSGPPPQCIELNKCTPPPYVENAVMLSENRSLFS
LRDIVEFRCHPGFIMKGASSVHCQSLNKWEPELPSCFKGVICRLPQEMSG
FQKGLGMKKEYYYGENVTLECEDGYTLEGSSQSQCQSDGSWNPLLAKCVS
RSISGLIVGIFIGIIVFILVIIVFIWMILKYKKRNTTDEKYKEVGIHLNY
KEDSCVRLQSLLTSQENSSTTSPARNSLTQEVS
Full-length human complement receptor 1(CR1)
(SEQ ID NO: 51)
MGASSPRSPEPVGPPAPGLPFCCGGSLLAVVVLLALPVAWGQCNAPEWL
PFARPTNLTDEFEFPIGTYLNYECRPGYSGRPFSIICLKNSVWTGAKDR
CRRKSCRNPPDPVNGMVHVIKGIQFGSQIKYSCTKGYRLIGSSSATCII
SGDTVIWDNETPICDRIPCGLPPTITNGDFISTNRENFHYGSVVTYRCN
PGSGGRKVFELVGEPSIYCTSNDDQVGIWSGPAPQCIIPNKCTPPNVEN
GILVSDNRSLFSLNEVVEFRCQPGFVMKGPRRVKCQALNKWEPELPSCS
RVCQPPPDVLHAERTQRDKDNFSPGQEVFYSCEPGYDLRGAASMRCTPQ
GDWSPAAPTCEVKSCDDFMGQLLNGRVLFPVNLQLGAKVDFVCDEGFQL
KGSSASYCVLAGMESLWNSSVPVCEQIFCPSPPVIPNGRHTGKPLEVFP
FGKAVNYTCDPHPDRGTSFDLIGESTIRCTSDPQGNGVWSSPAPRCGIL
GHCQAPDHFLFAKLKTQTNASDFPIGTSLKYECRPEYYGRPFSITCLDN
LVWSSPKDVCKRKSCKTPPDPVNGMVHVITDIQVGSRINYSCTTGHRLI
GHSSAECILSGNAAHWSTKPPICQRIPCGLPPTIANGDFISTNRENFHY
GSVVTYRCNPGSGGRKVFELVGEPSIYCTSNDDQVGIWSGPAPQCIIPN
KCTPPNVENGILVSDNRSLFSLNEVVEFRCQPGFVMKGPRRVKCQALNK
WEPELPSCSRVCQPPPDVLHAERTQRDKDNFSPGQEVFYSCEPGYDLRG
AASMRCTPQGDWSPAAPTCEVKSCDDFMGQLLNGRVLFPVNLQLGAKVD
FVCDEGFQLKGSSASYCVLAGMESLWNSSVPVCEQIFCPSPPVIPNGRH
TGKPLEVFPFGKAVNYTCDPHPDRGTSFDLIGESTIRCTSDPQGNGVWS
SPAPRCGILGHCQAPDHFLFAKLKTQTNASDFPIGTSLKYECRPEYYGR
PFSITCLDNLVWSSPKDVCKRKSCKTPPDPVNGMVHVITDIQVGSRINY
SCTTGHRLIGHSSAECILSGNTAHWSTKPPICQRIPCGLPPTIANGDFI
STNRENFHYGSVVTYRCNLGSRGRKVFELVGEPSIYCTSNDDQVGIWSG
PAPQCIIPNKCTPPNVENGILVSDNRSLFSLNEVVEFRCQPGFVMKGPR
RVKCQALNKWEPELPSCSRVCQPPPEILHGEHTPSHQDNFSPGQEVFYS
CEPGYDLRGAASLHCTPQGDWSPEAPRCAVKSCDDFLGQLPHGRVLFPL
NLQLGAKVSFVCDEGFRLKGSSVSHCVLVGMRSLWNNSVPVCEHIFCPN
PPAILNGRHTGTPSGDIPYGKEISYTCDPHPDRGMTFNLIGESTIRCTS
DPHGNGVWSSPAPRCELSVRAGHCKTPEQFPFASPTIPINDFEFPVGTS
LNYECRPGYFGKMFSISCLENLVWSSVEDNCRRKSCGPPPEPFNGMVHI
NTDTQFGSTVNYSCNEGFRLIGSPSTTCLVSGNNVTWDKKAPICEIISC
EPPPTISNGDFYSNNRTSFHNGTVVTYQCHTGPDGEQLFELVGERSIYC
TSKDDQVGVWSSPPPRCISTNKCTAPEVENAIRVPGNRSFFSLTEIIRF
RCQPGFVMVGSHTVQCQTNGRWGPKLPHCSRVCQPPPEILHGEHTLSHQ
DNFSPGQEVFYSCEPSYDLRGAASLHCTPQGDWSPEAPRCTVKSCDDFL
GQLPHGRVLLPLNLQLGAKVSFVCDEGFRLKGRSASHCVLAGMKALWNS
SVPVCEQIFCPNPPAILNGRHTGTPFGDIPYGKEISYACDTHPDRGMTF
NLIGESSIRCTSDPQGNGVWSSPAPRCELSVPAACPHPPKIQNGHYIGG
HVSLYLPGMTISYTCDPGYLLVGKGFIFCTDQGIWSQLDHYCKEVNCSF
PLFMNGISKELEMKKVYHYGDYVTLKCEDGYTLEGSPWSQCQADDRWDP
PLAKCTSRAHDALIVGTLSGTIFFILLIIFLSWIILKHRKGNNAHENPK
EVAIHLHSQGGSSVHPRTLQTNEENSRVLP
Full-length human factor H
(SEQ ID NO: 52)
MRLLAKIICLMLWAICVAEDCNELPPRRNTEILTGSWSDQTYPEGTQAI
YKCRPGYRSLGNVIMVCRKGEWVALNPLRKCQKRPCGHPGDTPFGTFTL
TGGNVFEYGVKAVYTCNEGYQLLGEINYRECDTDGWTNDIPICEVVKCL
PVTAPENGKIVSSAMEPDREYHFGQAVRFVCNSGYKIEGDEEMHCSDDG
FWSKEKPKCVEISCKSPDVINGSPISQKIIYKENERFQYKCNMGYEYSE
RGDAVCTESGWRPLPSCEEKSCDNPYIPNGDYSPLRIKHRTGDEITYQC
RNGFYPATRGNTAKCTSTGWIPAPRCTLKPCDYPDIKHGGLYHENMRRP
YFPVAVGKYYSYYCDEHFETPSGSYWDHIHCTQDGWSPAVPCLRKCYFP
YLENGYNQNYGRKFVQGKSIDVACHPGYALPKAQTTVTCMENGWSPTPR
CIRVKTCSKSSIDIENGFISESQYTYALKEKAKYQCKLGYVTADGETSG
SITCGKDGWSAQPTCIKSCDIPVFMNARTKNDFTWFKLNDTLDYECHDG
YESNTGSTTGSIVCGYNGWSDLPICYERECELPKIDVHLVPDRKKDQYK
VGEVLKFSCKPGFfiVGPNSVQCYHFGLSPDLPICKEQVQSCGPPPELL
NGNVKEKTKEEYGHSEVVEYYCNPRFLMKGPNKIQCVDGEWTTLPVCIV
EESTCGDIPELEHGWAQLSSPPYYYGDSVEFNCSESFTMIGHRSITCIH
GVWTQLPQCVAIDKLKKCKSSNLIILEEHLKNKKEFDHNSNIRYRCRGK
EGWIHTVCINGRWDPEVNCSMAQIQLCPPPPQIPNSHNMTTTLNYRDGE
KVSVLCQENYLIQEGEEITCKDGRWQSIPLCVEKIPCSQPPQIEHGTIN
SSRSSQESYAHGTKLSYTCEGGFRISEENETTCYMGKWSSPPQCEGLPC
KSPPEISHGVVAHMSDSYQYGEEVTYKCFEGFGIDGPAIAKCLGEKWSH
PPSCIKTDCLSLPSFENAIPMGEKKDVYKAGEQVTYTCATYYKMDGASN
VTCINSRWTGRPTCRDTSCVNPPTVQNAYIVSRQMSKYPSGERVRYQCR
SPYEMFGDEEVMCLNGNWTEPPQCKDSTGKCGPPPPIDNGDITSFPLSV
YAPASSVEYQCQNLYQLEGNKRITCRNGQWSEPPKCLHPCVISREIMEN
YNIALRWTAKQKLYSRTGESVEFVCKRGYRLSSRSHTLRTTCWDGKLEY
PTCAKR
Full-length mouse factor H
(SEQ ID NO: 53)
MRLSARIIWLILWTVCAAEDCKGPPPRENSEILSGSWSEQLYPEGTQATY
KCRPGYRTLGTIVKVCKNGKWVASNPSRICRKKPCGHPGDTPFGSFRLAV
GSQFEFGAKVVYTCDDGYQLLGEIDYRECGADGWINDIPLCEVVKCLPVT
ELENGRIVSGAAETDQEYYFGQVVRFECNSGFKIEGHKEIHCSENGLWSN
EKPRCVEILCTPPRVENGDGINVKPVYKENERYHYKCKHGYVPKERGDAV
CTGSGWSSQPFCEEKRCSPPYILNGIYTPHRIIHRSDDEIRYECNYGFYP
VTGSTVSKCTPTGWIPVPRCTLKPCEFPQFKYGRLYYEESLRPNFPVSIG
NKYSYKCDNGFSPPSGYSWDYLRCTAQGWEPEVPCVRKCVFHYVENGDSA
YWEKVYVQGQSLKVQCYNGYSLQNGQDTMTCTENGWSPPPKCIRIKTCSA
SDIHIDNGFLSESSSIYALNRETSYRCKQGYVTNTGEISGSITCLQNGWS
PQPSCIKSCDMPVFENSITKNTRTWFKLNDKLDYECLVGFENEYKHTKGS
ITCTYYGWSDTPSCYERECSVPTLDRKLVVSPRKEKYRVGDLLEFSCHSG
HRVGPDSVQCYHFGWSPGFPTCKGQVASCAPPLEILNGEINGAKKVEYSH
GEVVKYDCKPRFLLKGPNKIQCVDGNWTTLPVCIEEERTCGDIPELEHGS
AKCSVPPYHHGDSVEFICEENFfMIGHGSVSCISGKWTQLPKCVATDQLE
KCRVLKSTGIEAIKPKLTEFfHNSTMDYKCRDKQEYERSICINGKWDPEP
NCTSKTSCPPPPQIPNTQVIETTVKYLDGEKLSVLCQDNYLTQDSEEMVC
KDGRWQSLPRCIEKIPCSQPPTIEHGSINLPRSSEERRDSIESSSHEHGT
TFSYVCDDGFRIPEENRITCYMGKWSTPPRCVGLPCGPPPSIPLGTVSLE
LESYQHGEEVTYHCSTGFGIDGPAFIICEGGKWSDPPKCIKTDCDVLPTV
KNAIIRGKSKKSYRTGEQVTFRCQSPYQMNGSDTVTCVNSRWIGQPVCKD
NSCVDPPHVPNATIVTRTKNKYLHGDRVRYECNKPLELFGQVEVMCENGI
WTEKPKCRDSTGKCGPPPPIDNGDITSLSLPVYEPLSSVEYQCQKYYLLK
GKKTITCTNGKWSEPPTCLHACVIPENIMESHNIILKWRHTEKIYSHSGE
DIEFGCKYGYYKARDSPPFRTKCINGTINYPTCV
Signal peptide of the human CD5 protein
(SEQ ID NO: 54)
MPMGSLQPLATLYLLGMLVAS
Signal peptide of the human CR2 protein
(SEQ ID NO: 55)
MGAAGLLGVFLALVAPG
Signal peptide of the human CR2 protein
(SEQ ID NO: 56)
MGAAGLLGVFLALVAPGVLG
B4 scFV nucleic acid sequence
(SEQ ID NO: 57)
GACACGTGATCAGCCGCCACCATGCCCATGGGGTCTCTGCAACCGCTGGC
CACCTTGTACCTGCTGGGGATGCTGGTCGCTTCCGTGCTAGCGCATCATC
ATCATCATCATGTGAAACTGCAGGAGTCAGGGGCTGAGCTTGTGAAGCCT
GGGGCTTCAGTGAAGCTGTCCTGCAAGGCTTCTGGCTACACCTTCACCAG
CTACTGGATGCACTGGGTGAAGCAGAGGCCTGGACGAGGCCTTGAGTGGA
TTGGAAGGATTGGTCCTAATAGTGGTGGTACTAAGTACAATGAGAAGTTC
AAGAGCAAGGCCACACTGACTGTAGACAAACCCTCCAGCACAGCCTACAT
GCAGCTCAGCAGCCTGACATCTGAGGACTCTGCGGTCTATTATTGTGCAA
GAAGAATGGTAAAGGGGTGCTATGGACTACTGGGGCCAAGGGACCACGGT
CACCGTCTCCTCAAAGGGCGAATTCCAGCACACTGGCGGCCGTTACTAGT
GGATCCGAGCTCGGTACCAAGCTTGGCGTCAGGAGGCGGTGGCGGCTCGG
GTGGCGGCGGCTCTTGGATATCTGCAGAATTCGCCCTTGACATTGAGCTC
ACCCAGTCTCCAACCACCATGGCTGCATCTCCCGGGGAGAAGATCACTAT
CACCTGCAGTGCCAGCTCAAGTATAAGTTCCAATTACTTGCATTGGTATC
AGCAGAAGCCAGGATTCTCCCCTAAACTCTTGATTTATAGGACATCCAAT
CTGGCTTCTGGAGTCCCAGCTCGCTTCAGTGGCAGTGGGTCTGGGACCTC
TTACTCTCTCACAATTGGCACCATGGAGGCTGAAGATGTTGCCACTTACT
ACTGCCAGCAGGGTAGTAGTATACCACGTACACGTTCGGAGGGGGCACCA
AGCTGGAAATAATAGACTAGTCGTGCG
C2 scFV nucleic acid sequence
(SEQ ID NO:58)
GACACGAAGCTTGCCGCCACCATGCCCATGGGGTCTCTGCAACCGCTGGC
CACCTTGTACCTGCTGGGGATGCTGGTCGCTTCCGTGCTAGCGCATCATC
ATCATCATCATGTCAAGCTGCAGGAGTCTGGGACTGAACTGGTGAAGCCT
GGGGCTTCAGTGAAGCTGTCCTGCAAGGCTTCTGGCTACACCTTCACCAG
CTACTGGATGCACTGGGTGAAGCAGAGGCCTGGACAAGGCCTTGAGTGGA
TTGGAAATATTAATCCTAGCAATGGTGGTACTAACTACAATGAGAAGTTC
AAGAGCAAGGCCACACTGACTGTAGACAAATCCTCCAGCACAGCCTACAT
GCAGCTCAGCAGCCTGACATCTGAGGACTCTGCGGTCTATTATTGTGCAA
GAAGAGGCATACGGTTACGACACTTTGACTACTGGGGCCAAGGGACCACG
GTCACCGTCTCCTCAAGGGCGAATTCTGCAGATATCCATCACACTGGCGG
CCGCTCGAGCATGCATCTAGAGGGCCCAATTCGCCCTATAGTGAGTCGTA
TATCAGGAGGCGGTGGCGGCTCGGGTGGCGGCGGCTCTTGGATATCTGCA
GAATTCGCCCTTGACATTGTGATGACACAGTCTCCTGCTTCCTTAGCTGT
ATCTCTGGGGCAGAGGGCCACCATCTCATACAGGGCCAGCAAAAGTGTCA
GTACATCTGGCTATAGTTATATGCACTGGAACCAACAGAAACCAGGACAG
CCACCCAGACTCCTCATCTATCTTGTATCCAACCTAGAATCTGGGGTCCC
TGCCAGGTTCAGTGGCAGTGGGTCTGGGACAGACTTCACCCTCAACATCC
ATCCTGTGGAGGAGGAGGATGCTGCAACCTATTACTGTCAGCACATTAGG
GAGCTTACACGTTCGGAGGGGGGACCAAGCTGGAAATAATAGCCCGGGCG
TGCG

1. A method of inhibiting complement-mediated inflammation in a tissue having non-ischemic injury in an individual, comprising administering to the individual an effective amount of a composition comprising a construct, wherein the construct comprises (a) an antibody or a fragment thereof, wherein the antibody or a fragment thereof specifically binds to Annexin IV or a phospholipid; and (b) a complement inhibitor.

2. A method of treating an inflammatory disease in an individual, comprising administering to the individual an effective amount of a composition comprising a construct, wherein the construct comprises (a) an antibody or a fragment thereof, wherein the antibody or a fragment thereof specifically binds to Annexin IV or a phospholipid; and (b) a complement inhibitor.

3-5. (canceled)

6. A method of detecting complement-mediated injury in a tissue of an individual, comprising administering to the individual an effective amount of a composition comprising a construct, wherein the construct comprises (a) an antibody or a fragment thereof, wherein the antibody or a fragment thereof specifically binds to Annexin IV or phospholipid; and (b) a detectable moiety, wherein the presence of the detectable moiety at the tissue is indicative of a complement-mediated tissue injury.

7. (canceled)

8. The method of claim 6, wherein the detectable moiety is selected from the group consisting of radioisotopes, fluorescent dyes, electron-dense reagents, enzymes, biotins, paramagnetic agents, magnetic agents, and nanoparticles.

9. The method of claim 6, wherein the tissue injury results from any of from inflammatory disorders, transplant rejection, pregnancy-related diseases, adverse drug reactions, autoimmune or immune complex disorders.

10. The method of claim 6, wherein the tissue is eye, joint, kidney, brain, heart, spinal cord, or liver.

11. (canceled)

12. The method of claim 2, wherein the disease is an ocular disease, arthritis, or renal injury.

13-26. (canceled)

27. A construct comprising: (a) an antibody or a fragment thereof, wherein the antibody or a fragment thereof specifically binds to Annexin IV or a phospholipid; and (b) a complement modulator or a detectable moiety, wherein the antibody or fragment thereof comprises: (i) a light chain variable domain comprising a sequence of SEQ ID NO:1 or 7, a sequence of SEQ ID NO:2 or 8, or a sequence of SEQ ID NO:3 or 9; and/or (ii) heavy chain variable domain comprising a sequence of SEQ ID NO:4 or 10, a sequence of SEQ ID NO:5 or 11, or a sequence of SEQ ID NO:6 or 12; or
(i) a light chain variable domain comprising a sequence of SEQ ID NO:25 or 31, a sequence of SEQ ID NO:26 or 32, or a sequence of SEQ ID NO:27 or 33; and/or (ii) heavy chain variable domain comprising a sequence of SEQ ID NO:28, a sequence of SEQ ID NO:29, or a sequence of SEQ ID NO:30.

28-32. (canceled)

33. The construct of claim 27, wherein the antibody or fragment is an scFv having the sequence of SEQ ID NO:17 or 18.

34-41. (canceled)

42. The construct of claim 27, wherein the antibody or fragment thereof competitively inhibits the binding of monoclonal antibody C2 to phospholipid.

43. (canceled)

44. (canceled)

45. The construct of claim 27, wherein the phospholipid is phosphatidylethanolamine, cardiolipin, phosphatidylcholine, or.

46-48. (canceled)

49. The construct of claim 27, wherein the complement inhibitor is selected from the group consisting of an anti-C5 antibody, anti-MASP antibody, an Eculizumab, an pexelizumab, an anti-C3b antibody, an anti-C6 antibody, an anti-C7 antibody, an anti-factor B antibody, an anti-factor D antibody, and an anti-properdin antibody, a membrane cofactor protein, a decay accelerating factor, a CD59, a Crry, a CR1, a factor H, a Factor I, a linear peptide, a cyclic peptide, a compstatin, an N-acetylaspartylglutamic acid, or a biologically active fragment of any the preceding.

50. The construct of claim 27, wherein the complement inhibitor is a specific inhibitor of the alternative pathway, or the lectin pathway.

51. (canceled)

52. The construct of claim 27, wherein the construct comprises a detectable moiety.

53. The construct of claim 27, wherein the detectable moiety is selected from the group consisting of radioisotopes, fluorescent dyes, electron-dense reagents, enzymes, biotins, paramagnetic agents, magnetic agents, and nanoparticles.

54. The construct of claim 27, wherein the construct is a fusion protein.

55. (canceled)

56. The construct of claim 27 and a pharmaceutically acceptable excipient.

57-66. (canceled)

67. The method of claim 2, wherein the disease is wet age-related macular degeneration, dry age-related macular degeneration, cytomegalovirus retinitis, macular edema, uveitis (anterior and posterior), glaucoma, open/wide-angle glaucoma, close/narrow-angle glaucoma, retinitis pigmentosa, proliferative vitreoretinopathy, retinal detachment, corneal wound healing, corneal transplants, and ocular melanoma.

68. The method of claim 2, wherein the disease is wet age-related macular degeneration or dry age-related macular degeneration.

69-81. (canceled)

82. The construct of claim 27, wherein the antibody or fragment thereof is a single-chain variable fragment (scFv).

83-88. (canceled)
